SEQUENCE LISTING (357 sequences)

SEQ ID NO: 1 - protein, 1073 aa, Homo sapiens
  CHAIN 1..1073: note = human GUCY2C
MKTLLLDLALWSLLFQPGWLSFSSQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQILMIAVFTLTGAVVLLLLVALLMLRKYRKDYELRQKKWSHIPPENIFPLETNETNHVSLKIDDDKRRDTIQRLRQCKYDKKRVILKDLKHNDGNFTEKQKIELNKLLQIDYYNLTKFYGTVKLDTMIFGVIEYCERGSLREVLNDTISYPDGTFMDWEFKISVLYDIAKGMSYLHSSKTEVHGRLKSTNCVVDSRMVVKITDFGCNSILPPKKDLWTAPEHLRQANISQKGDVYSYGIIAQEIILRKETFYTLSCRDRNEKIFRVENSNGMKPFRPDLFLETAEEKELEVYLLVKNCWEEDPEKRPDFKKIETTLAKIFGLFHDQKNESYMDTLIRRLQLYSRNLEHLVEERTQLYKAERDRADRLNFMLLPRLVVKSLKEKGFVEPELYEEVTIYFSDIVGFTTICKYSTPMEVVDMLNDIYKSFDHIVDHHDVYKVETIGDAYMVASGLPKRNGNRHAIDIAKMALEILSFMGTFELEHLPGLPIWIRIGVHSGPCAAGVVGIKMPRYCLFGDTVNTASRMESTGLPLRIHVSGSTIAILKRTECQFLYEVRGETYLKGRGNETTYWLTGMKDQKFNLPTPPTVENQQRLQAEFSDMIANSLQKRQAAGIRSQKPRRVASYKKGTLEYLQLNTTDKESTYF

SEQ ID NO: 2 - protein, 639 aa, synthetic construct
  CHAIN 1..639: note = hGUCY2C ECD-hFc
SQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 413 aa, synthetic construct
  CHAIN 1..413: note = hGUCY2C ECD-His
SQVSQNCHNGSYEISVLMMGNSAFAEPLKNLEDAVNEGLEIVRGRLQNAGLNVTVNATFMYSDGLIHNSGDCRSSTCEGLDLLRKISNAQRMGCVLIGPSCTYSTFQMYLDTELSYPMISAGSFGLSCDYKETLTRLMSPARKLMYFLVNFWKTNDLPFKTYSWSTSYVYKNGTETEDCFWYLNALEASVSYFSHELGFKVVLRQDKEFQDILMDHNRKSNVIIMCGGPEFLYKLKGDRAVAEDIVIILVDLFNDQYFEDNVTAPDYMKNVLVLTLSPGNSLLNSSFSRNLSPTKRDFALAYLNGILLFGHMLKIFLENGENITTPKFAHAFRNLTFEGYDGPVTLDDWGDVDSTMVLLYTSVDTKKYKVLLTYDTHVNKTYPVDMSPTFTWKNSKLPNDITGRGPQHHHHHH

SEQ ID NO: 4 - protein, 5 aa, synthetic construct
  DOMAIN 1..5: note = M81-HCDR1
DHNMN

SEQ ID NO: 5 - protein, 17 aa, synthetic construct
  DOMAIN 1..17: note = M81-HCDR2
VMNPKFGTINYNQKFKD

SEQ ID NO: 6 - protein, 9 aa, synthetic construct
  DOMAIN 1..9: note = M81-HCDR3
DDYPNYFDY

SEQ ID NO: 7 - protein, 10 aa, synthetic construct
  DOMAIN 1..10: note = M81-LCDR1
SASSSVNYMY

SEQ ID NO: 8 - protein, 7 aa, synthetic construct
  DOMAIN 1..7: note = M81-LCDR2
RTSNLAS

SEQ ID NO: 9 - protein, 9 aa, synthetic construct
  DOMAIN 1..9: note = M81-LCDR3
QQYHDYPPT

SEQ ID NO: 10 - protein, 5 aa, synthetic construct
  DOMAIN 1..5: note = M146-HCDR1
DYNMN

SEQ ID NO: 11 - protein, 17 aa, synthetic construct
  DOMAIN 1..17: note = M146-HCDR2
LINPKYGTTNYNQKFKG

SEQ ID NO: 12 - protein, 9 aa, synthetic construct
  DOMAIN 1..9: note = M146-HCDR3
DDYPAWFAY

SEQ ID NO: 13 - protein, 10 aa, synthetic construct
  DOMAIN 1..10: note = M146-LCDR1
SASSSLNYMY

SEQ ID NO: 14 - protein, 9 aa, synthetic construct
  DOMAIN 1..9: note = M146-LCDR3
QQYHSYPPT

SEQ ID NO: 15 - protein, 118 aa, synthetic construct
  DOMAIN 1..118: note = M81 VH
EFQLQQSGPELVKPGASVKISCKASGYSFTDHNMNWVKQSNGKSLEWIGVMNPKFGTINYNQKFKDKATLTVDQSSSTAYMQLNSLTSEDSAVYYCVRDDYPNYFDYWGQGTTLTVSS

SEQ ID NO: 16 - protein, 106 aa, synthetic construct
  DOMAIN 1..106: note = M81 VL
QIVLTQSPAIMSASPGEKVTISCSASSSVNYMYWYQQKPGSSPKSWIYRTSNLASGVSARFSGSGSGTSYSLTISSMEAEDAATYYCQQYHDYPPTFGGGTKLEIK

SEQ ID NO: 17 - protein, 118 aa, synthetic construct
  DOMAIN 1..118: note = M146 VH
EFQLQQSGPELVKPGASVKISCKASGYSFTDYNMNWVKQSNGKSLEWIGLINPKYGTTNYNQKFKGKATLTVDQSSSTAYMQLNSLTSEDSAVYYCAIDDYPAWFAYWGQGTLVTVSA

SEQ ID NO: 18 - protein, 106 aa, synthetic construct
  DOMAIN 1..106: note = M146 VL
QIVLTQSPAIMSASPGEKVTISCSASSSLNYMYWYQQKPGSSPKSWIYRTSNLASGVPARFSGSGSGTSYSLTISSMEAEDAATYYCQQYHSYPPTFGAGTKLELK

SEQ ID NO: 19 - protein, 5 aa, synthetic construct
  DOMAIN 1..5: note = P3-C7-HCDR1
DHYIH

SEQ ID NO: 20 - protein, 17 aa, synthetic construct
  DOMAIN 1..17: note = P3-C7-HCDR2
VINPYNDGATYNQKFKG

SEQ ID NO: 21 - protein, 11 aa, synthetic construct
  DOMAIN 1..11: note = P3-C7-HCDR3
PPRAYGNYAGY

SEQ ID NO: 22 - protein, 16 aa, synthetic construct
  DOMAIN 1..16: note = P3-C7-LCDR1
RSSQSLVHSNGNTYLH

SEQ ID NO: 23 - protein, 7 aa, synthetic construct
  DOMAIN 1..7: note = P3-C7-LCDR2
KVSNRFS

SEQ ID NO: 24 - protein, 9 aa, synthetic construct
  DOMAIN 1..9: note = P3-C7-LCDR3
SQSTHVPYT

SEQ ID NO: 25 - protein, 120 aa, synthetic construct
  DOMAIN 1..120: note = P3-C7 VH
EVQLQQSGPVLLKPGASVKMSCKASGYTFTDHYIHWVKLSHGKSLEWIGVINPYNDGATYNQKFKGKATLTVDKSSSTAYMELNSLTSEDSAVYYCARPPRAYGNYAGYWGQGTTLTVSS

SEQ ID NO: 26 - protein, 112 aa, synthetic construct
  DOMAIN 1..112: note = P3-C7 VL
DVVMTQTPLSLPVSLGDQASISCRSSQSLVHSNGNTYLHWYLQKPGQSPKLLIYKVSNRFSGVPDRFIGSGSRTDFTLKISRVEAEDLGVYFCSQSTHVPYTFGGGTRLEIK

SEQ ID NO: 27 - protein, 9 aa, synthetic construct
  DOMAIN 1..9: note = h81 H1-HCDR3
DNYPNYFDY

SEQ ID NO: 28 - protein, 7 aa, synthetic construct
  DOMAIN 1..7: note = h81 L4-LCDR2
RTSNDAS

SEQ ID NO: 29 - protein, 7 aa, synthetic construct
  DOMAIN 1..7: note = h81 L5-LCDR2
RTSNLAQ

SEQ ID NO: 30 - protein, 7 aa, synthetic construct
  DOMAIN 1..7: note = h81 L6-LCDR2
RTQNLAS

SEQ ID NO: 31 - protein, 106 aa, synthetic construct
  DOMAIN 1..106: note = h81 L1
EIVLTQSPATLSLSPGERATLSCSASSSVNYMYWYQQKPGQAPKSWIYRTSNLASGVPARFSGSGSGTDYTLTISSLEPEDFAVYYCQQYHDYPPTFGGGTKVEIK

SEQ ID NO: 32 - protein, 106 aa, synthetic construct
  DOMAIN 1..106: note = h81 L2
EIVLTQSPDFQSVTPGEKVTITCSASSSVNYMYWYQQKPDQSPKSWIYRTSNLASGVPSRFSGSGSGTDFTLTISSLEAEDAATYYCQQYHDYPPTFGGGTKVEIK

SEQ ID NO: 33 - protein, 106 aa, synthetic construct
  DOMAIN 1..106: note = h81 L3
DIQLTQSPSFLSASPGDRVTITCSASSSVNYMYWYQQKPGSSPKSWIYRTSNLASGVPSRFSGSGSGTEYTLTISSLQPEDFATYYCQQYHDYPPTFGGGTKVEIK

SEQ ID NO: 34 - protein, 106 aa, synthetic construct
  DOMAIN 1..106: note = h81 L4
DIQLTQSPSFLSASPGDRVTITCSASSSVNYMYWYQQKPGSSPKSWIYRTSNDASGVPSRFSGSGSGTEYTLTISSLQPEDFATYYCQQYHDYPPTFGGGTKVEIK

SEQ ID NO: 35 - protein, 106 aa, synthetic construct
  DOMAIN 1..106: note = h81 L5
DIQLTQSPSFLSASPGDRVTITCSASSSVNYMYWYQQKPGSSPKSWIYRTSNLAQGVPSRFSGSGSGTEYTLTISSLQPEDFATYYCQQYHDYPPTFGGGTKVEIK

SEQ ID NO: 36 - protein, 106 aa, synthetic construct
  DOMAIN 1..106: note = h81 L6
DIQLTQSPSFLSASPGDRVTITCSASSSVNYMYWYQQKPGSSPKSWIYRTQNLASGVPSRFSGSGSGTEYTLTISSLQPEDFATYYCQQYHDYPPTFGGGTKVEIK

SEQ ID NO: 37 - protein, 118 aa, synthetic construct
  DOMAIN 1..118: note = h81 H1
EVQLVQSGAEVKKPGASVKVSCKASGYTFTDHNMNWVKQAPGQRLEWMGVMNPKFGTINYNQKFKDKATITRDTSASTAYMELSSLRSEDTAVYYCARDNYPNYFDYWGQGTTVTVSS

SEQ ID NO: 38 - protein, 118 aa, synthetic construct
  DOMAIN 1..118: note = h81 H2
EVQLVQSGAEVKKPGASVKVSCKASGYTFTDHNMNWVKQAPGQRLEWMGVMNPKFGTINYNQKFKDKATITRDTSASTAYMELSSLRSEDTAVYYCARDDYPNYFDYWGQGTTVTVSS

SEQ ID NO: 39 - protein, 7 aa, synthetic construct
  DOMAIN 1..7: note = h146 L1-LCDR2
RTSQLAS

SEQ ID NO: 40 - protein, 9 aa, synthetic construct
  DOMAIN 1..9: note = h146 H1-HCDR3
DNYPAWFAY

SEQ ID NO: 41 - protein, 106 aa, synthetic construct
  DOMAIN 1..106: note = h146 L1
EIVLTQSPATLSLSPGERATLSCSASSSLNYMYWYQQKPGSSPRPWIYRTSQLASGVPARFSGSGSGTDYTLTISSLEPEDFAVYYCQQYHSYPPTFGGGTKVEIK

SEQ ID NO: 42 - protein, 118 aa, synthetic construct
  DOMAIN 1..118: note = h146 H1
EFQLVQSGAEVKKPGASVKVSCKASGYSFTDYNMNWVRQAPGQRLEWMGLINPKYGTTNYNQKFKGRVTLTVDQSSSTAYMELSSLRSEDTAVYYCARDNYPAWFAYWGQGTTVTVSS

SEQ ID NO: 43 - protein, 16 aa, synthetic construct
  DOMAIN 1..16: note = hP3-C7 L3-LCDR1
RSSQSLVHSQGNTYLH

SEQ ID NO: 44 - protein, 16 aa, synthetic construct
  DOMAIN 1..16: note = hP3-C7 L4-LCDR1
RSSQSLVHSSGNTYLH

SEQ ID NO: 45 - protein, 16 aa, synthetic construct
  DOMAIN 1..16: note = hP3-C7 L5-LCDR1
RSSQSLVHSTGNTYLH

SEQ ID NO: 46 - protein, 16 aa, synthetic construct
  DOMAIN 1..16: note = hP3-C7 L6-LCDR1
RSSQSLVHSNVNTYLH

SEQ ID NO: 47 - protein, 16 aa, synthetic construct
  DOMAIN 1..16: note = hP3-C7 L7/hP3-C7 L8-LCDR1
RSSQSLVHSNQNTYLH

SEQ ID NO: 48 - protein, 112 aa, synthetic construct
  DOMAIN 1..112: note = hP3-C7 L1
DIVMTQTPLSLPVTPGEPASISCRSSQSLVHSNGNTYLHWYLQKPGQSPQLLIYKVSNRFSGVPDRFSGSGSGTDFTLKISRVEAEDVGVYYCSQSTHVPYTFGGGTKVEIK

SEQ ID NO: 49 - protein, 112 aa, synthetic construct
  DOMAIN 1..112: note = hP3-C7 L2
DVVMTQTPLSLPVTPGEPASISCRSSQSLVHSNGNTYLHWYLQKPGQSPQLLIYKVSNRFSGVPDRFSGSGSRTDFTLKISRVEAEDVGVYYCSQSTHVPYTFGGGTKVEIK

SEQ ID NO: 50 - protein, 112 aa, synthetic construct
  DOMAIN 1..112: note = hP3-C7 L3
DVVMTQTPLSLPVTPGEPASISCRSSQSLVHSQGNTYLHWYLQKPGQSPQLLIYKVSNRFSGVPDRFSGSGSRTDFTLKISRVEAEDVGVYYCSQSTHVPYTFGGGTKVEIK

SEQ ID NO: 51 - protein, 112 aa, synthetic construct
  DOMAIN 1..112: note = hP3-C7 L4
DVVMTQTPLSLPVTPGEPASISCRSSQSLVHSSGNTYLHWYLQKPGQSPQLLIYKVSNRFSGVPDRFSGSGSRTDFTLKISRVEAEDVGVYYCSQSTHVPYTFGGGTKVEIK

SEQ ID NO: 52 - protein, 112 aa, synthetic construct
  DOMAIN 1..112: note = hP3-C7 L5
DVVMTQTPLSLPVTPGEPASISCRSSQSLVHSTGNTYLHWYLQKPGQSPQLLIYKVSNRFSGVPDRFSGSGSRTDFTLKISRVEAEDVGVYYCSQSTHVPYTFGGGTKVEIK

SEQ ID NO: 53 - protein, 112 aa, synthetic construct
  DOMAIN 1..112: note = hP3-C7 L5
DVVMTQTPLSLPVTPGEPASISCRSSQSLVHSNVNTYLHWYLQKPGQSPQLLIYKVSNRFSGVPDRFSGSGSRTDFTLKISRVEAEDVGVYYCSQSTHVPYTFGGGTKVEIK

SEQ ID NO: 54 - protein, 112 aa, synthetic construct
  DOMAIN 1..112: note = hP3-C7 L7
DVVMTQTPLSLPVTPGEPASISCRSSQSLVHSNQNTYLHWYLQKPGQSPQLLIYKVSNRFSGVPDRFSGSGSRTDFTLKISRVEAEDVGVYYCSQSTHVPYTFGGGTKVEIK

SEQ ID NO: 55 - protein, 112 aa, synthetic construct
  DOMAIN 1..112: note = hP3-C7 L8
DVVMTQSPSSLSASVGDRVTITCRSSQSLVHSNQNTYLHWYQQKPGKAPKLLIYKVSNRFSGVPSRFSGSGSRTDFTLKISSLQPEDFATYYCSQSTHVPYTFGGGTKVEIK

SEQ ID NO: 56 - protein, 120 aa, synthetic construct
  DOMAIN 1..120: note = hP3-C7 H1
EVQLVQSGAEVKKPGASVKVSCKASGYTFTDHYIHWVRQAPGQGLEWMGVINPYNDGATYNQKFKGRVTMTVDKSTSTVYMELSSLRSEDTAVYYCARPPRAYGNYAGYWGQGTTVTVSS

SEQ ID NO: 57 - protein, 120 aa, synthetic construct
  DOMAIN 1..120: note = hP3-C7 H2
EVQLVQSGAEVKKPGASVKVSCKASGYTFTDHYIHWVRLAPGKGLEWMGVINPYNDGATYNQKFKGRVTLTVDKSTSTVYMELSSLRSEDTAVYYCARPPRAYGNYAGYWGQGTTVTVSS

SEQ ID NO: 58 - protein, 330 aa, synthetic construct [sequence omitted]

SEQ ID NO: 59 - protein, 107 aa, synthetic construct [sequence omitted]

SEQ ID NO: 60 - protein, 448 aa, synthetic construct
  CHAIN 1..448: note = 81 L3H1 heavy chain
EVQLVQSGAEVKKPGASVKVSCKASGYTFTDHNMNWVKQAPGQRLEWMGVMNPKFGTINYNQKFKDKATITRDTSASTAYMELSSLRSEDTAVYYCARDNYPNYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 61 - protein, 213 aa, synthetic construct
  CHAIN 1..213: note = 81 L3H1 light chain
DIQLTQSPSFLSASPGDRVTITCSASSSVNYMYWYQQKPGSSPKSWIYRTSNLASGVPSRFSGSGSGTEYTLTISSLQPEDFATYYCQQYHDYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - protein, 448 aa, synthetic construct
  CHAIN 1..448: note = 146 L1H1 heavy chain
EFQLVQSGAEVKKPGASVKVSCKASGYSFTDYNMNWVRQAPGQRLEWMGLINPKYGTTNYNQKFKGRVTLTVDQSSSTAYMELSSLRSEDTAVYYCARDNYPAWFAYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 213 aa, synthetic construct
  CHAIN 1..213: note = 146 L1H1 light chain
EIVLTQSPATLSLSPGERATLSCSASSSLNYMYWYQQKPGSSPRPWIYRTSQLASGVPARFSGSGSGTDYTLTISSLEPEDFAVYYCQQYHSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 64 - protein, 450 aa, synthetic construct
  CHAIN 1..450: note = P3-C7 L7H1 heavy chain
EVQLVQSGAEVKKPGASVKVSCKASGYTFTDHYIHWVRQAPGQGLEWMGVINPYNDGATYNQKFKGRVTMTVDKSTSTVYMELSSLRSEDTAVYYCARPPRAYGNYAGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 65 - protein, 219 aa, synthetic construct
  CHAIN 1..219: note = P3-C7 L7H1 light chain
DVVMTQTPLSLPVTPGEPASISCRSSQSLVHSNQNTYLHWYLQKPGQSPQLLIYKVSNRFSGVPDRFSGSGSRTDFTLKISRVEAEDVGVYYCSQSTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 66 - protein, 219 aa, synthetic construct
  CHAIN 1..219: note = P3-C7 L8H1 light chain
DVVMTQSPSSLSASVGDRVTITCRSSQSLVHSNQNTYLHWYQQKPGKAPKLLIYKVSNRFSGVPSRFSGSGSRTDFTLKISSLQPEDFATYYCSQSTHVPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 452 aa, synthetic construct
  CHAIN 1..452: note = 1608 heavy chain
EVQLVESGGGLVQPGGSLRLSCAASGFTFSSYWMHWVRQAPGKGLEWIGEIKPSNELTNVHEKFKDRFTISVDKAKNSAYLQMNSLRAEDTAVYYCTRTITTTEGYWFFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 217 aa, synthetic construct
  CHAIN 1..217: note = 1608 light chain
DIQLTQSPSSLSASVGDRVTITCRASESVDYYGSSLLQWYQQKPGKAPKLLIYAASKLASGVPSRFSGSGSGTDFTLTISSLQPEDFATYYCQQTRKAYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 69 - protein, 449 aa, synthetic construct
  CHAIN 1..449: note = 5F9-HC
QVQLQQWGAGLLKPSETLSLTCAVFGGSFSGYYWSWIRQPPGKGLEWIGEINHRGNTNDNPSLKSRVTISVDTSKNQFALKLSSVTAADTAVYYCARERGYTYGNFDHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 214 aa, synthetic construct
  CHAIN 1..214: note = 5F9-LC
EIVMTQSPATLSVSPGERATLSCRASQSVSRNLAWYQQKPGQAPRLLIYGASTRATGIPARFSGSGSGTEFTLTIGSLQSEDFAVYYCQQYKTWPRTFGQGTNVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 71 - protein, 5 aa, synthetic construct
  DOMAIN 1..5: note = S107E-HCDR1
KYAMN

SEQ ID NO: 72 - protein, 19 aa, synthetic construct
  DOMAIN 1..19: note = S107E-HCDR2
RIRSKYNNYATYYADSVKD

SEQ ID NO: 73 - protein, 14 aa, synthetic construct
  DOMAIN 1..14: note = S107E-HCDR3
HGNFGNEYISYWAY

SEQ ID NO: 74 - protein, 14 aa, synthetic construct
  DOMAIN 1..14: note = S107E-LCDR1
GSSTGAVTSGNYPN

SEQ ID NO: 75 - protein, 7 aa, synthetic construct
  DOMAIN 1..7: note = S107E-LCDR2
GTKFLAP

SEQ ID NO: 76 - protein, 9 aa, synthetic construct
  DOMAIN 1..9: note = S107E-LCDR3
VLWYSNRWV

SEQ ID NO: 77 - protein, 125 aa, synthetic construct
  DOMAIN 1..125: note = S107E-VH
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSS

SEQ ID NO: 78 - protein, 109 aa, synthetic construct
  DOMAIN 1..109: note = S107E-VL
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVL

SEQ ID NO: 79 - protein, 221 aa, synthetic construct
  DOMAIN 1..221: note = IgG1Fc(Knob-DI L234A/L235A/G237A/Y349C/T366W)
CPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 80 - protein, 221 aa, synthetic construct
  DOMAIN 1..221: note = IgG1Fc(Hole-DI L234A/L235A/G237A/S354C/T366S/L368A/Y407V)
CPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 81 - protein, 8 aa, synthetic construct
  DOMAIN 1..8: note = linker1
GGGSGGGG

SEQ ID NO: 82 - skipped

SEQ ID NO: 83 - protein, 9 aa, synthetic construct
  DOMAIN 1..9: note = linker3
GGGGSGGGG

SEQ ID NO: 84 - protein, 103 aa, synthetic construct
  DOMAIN 1..103: note = IgG1(CH1)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 85 - protein, 227 aa, synthetic construct
  DOMAIN 1..227: note = IgG1Fc(Knob L234A/L235A/S354C/T366W)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 227 aa, synthetic construct
  DOMAIN 1..227: note = IgG1Fc(Hole L234A/L235A/Y349C/T366S/L368A/Y407V)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 87 - protein, 105 aa, synthetic construct
  DOMAIN 1..105: note = CL(Lambda)
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 88 - protein, 14 aa, synthetic construct
  DOMAIN 1..14: note = N103Q N106Q-HCDR3
HGQFGQSYISYWAY

SEQ ID NO: 89 - protein, 125 aa, synthetic construct
  DOMAIN 1..125: note = N103QN106Q-VH
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGQFGQSYISYWAYWGQGTLVTVSS

SEQ ID NO: 90 - protein, 5 aa, synthetic construct
  DOMAIN 1..5: note = linker4
GGGGS

SEQ ID NO: 91 - protein, 455 aa, synthetic construct
  CHAIN 1..455: note = Format 2-1
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 214 aa, synthetic construct
  CHAIN 1..214: note = Format 2-2
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTEC

SEQ ID NO: 93 - protein, 455 aa, synthetic construct
  CHAIN 1..455: note = Format 3-3
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 94 - protein, 227 aa, synthetic construct
  DOMAIN 1..227: note = IgG1Fc (L234A/L235A)
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 15 aa, synthetic construct
  DOMAIN 1..15: note = linker5
GGGGSGGGGSGGGGS

SEQ ID NO: 96 - skipped

SEQ ID NO: 97 - protein, 10 aa, synthetic construct
  DOMAIN 1..10: note = linker7
GGGGSGGGGS

SEQ ID NO: 98 - protein, 212 aa, synthetic construct
  CHAIN 1..212: note = Format 5-3
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 99 - protein, 467 aa, synthetic construct
  CHAIN 1..467: note = P3-C7 L7H1-1-1
DVVMTQTPLSLPVTPGEPASISCRSSQSLVHSNQNTYLHWYLQKPGQSPQLLIYKVSNRFSGVPDRFSGSGSRTDFTLKISRVEAEDVGVYYCSQSTHVPYTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 100 - protein, 460 aa, synthetic construct
  CHAIN 1..460: note = P3-C7 L7H1-1-2
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGEVQLVQSGAEVKKPGASVKVSCKASGYTFTDHYIHWVRQAPGQGLEWMGVINPYNDGATYNQKFKGRVTMTVDKSTSTVYMELSSLRSEDTAVYYCARPPRAYGNYAGYWGQGTTVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 101 - protein, 467 aa, synthetic construct
  CHAIN 1..467: note = P3-C7 L8H1-1-1
DVVMTQSPSSLSASVGDRVTITCRSSQSLVHSNQNTYLHWYQQKPGKAPKLLIYKVSNRFSGVPSRFSGSGSRTDFTLKISSLQPEDFATYYCSQSTHVPYTFGGGTKVEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 102 - protein, 461 aa, synthetic construct
  CHAIN 1..461: note = M81-1-1
QIVLTQSPAIMSASPGEKVTISCSASSSVNYMYWYQQKPGSSPKSWIYRTSNLASGVSARFSGSGSGTSYSLTISSMEAEDAATYYCQQYHDYPPTFGGGTKLEIKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 103 - protein, 458 aa, synthetic construct
  CHAIN 1..458: note = M81-1-2
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGEFQLQQSGPELVKPGASVKISCKASGYSFTDHNMNWVKQSNGKSLEWIGVMNPKFGTINYNQKFKDKATLTVDQSSSTAYMQLNSLTSEDSAVYYCVRDDYPNYFDYWGQGTTLTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 104 - protein, 461 aa, synthetic construct
  CHAIN 1..461: note = M146-1-1
QIVLTQSPAIMSASPGEKVTISCSASSSLNYMYWYQQKPGSSPKSWIYRTSNLASGVPARFSGSGSGTSYSLTISSMEAEDAATYYCQQYHSYPPTFGAGTKLELKGGGSGGGGEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 105 - protein, 458 aa, synthetic construct
  CHAIN 1..458: note = M146-1-2
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGGSGGGGEFQLQQSGPELVKPGASVKISCKASGYSFTDYNMNWVKQSNGKSLEWIGLINPKYGTTNYNQKFKGKATLTVDQSSSTAYMQLNSLTSEDSAVYYCAIDDYPAWFAYWGQGTLVTVSAGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 106 - protein, 449 aa, synthetic construct
  CHAIN 1..449: note = P3-C7 L7H1-2-3
EVQLVQSGAEVKKPGASVKVSCKASGYTFTDHYIHWVRQAPGQGLEWMGVINPYNDGATYNQKFKGRVTMTVDKSTSTVYMELSSLRSEDTAVYYCARPPRAYGNYAGYWGQGTTVTVSSGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 107 - protein, 215 aa, synthetic construct
  CHAIN 1..215: note = P3-C7 L7H1-2-4
DVVMTQTPLSLPVTPGEPASISCRSSQSLVHSNQNTYLHWYLQKPGQSPQLLIYKVSNRFSGVPDRFSGSGSRTDFTLKISRVEAEDVGVYYCSQSTHVPYTFGGGTKVEIKGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 108 - protein, 447 aa, synthetic construct
  CHAIN 1..447: note = M81-2-3
EFQLQQSGPELVKPGASVKISCKASGYSFTDHNMNWVKQSNGKSLEWIGVMNPKFGTINYNQKFKDKATLTVDQSSSTAYMQLNSLTSEDSAVYYCVRDDYPNYFDYWGQGTTLTVSSGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 109 - protein, 209 aa, synthetic construct
  CHAIN 1..209: note = M81-2-4
QIVLTQSPAIMSASPGEKVTISCSASSSVNYMYWYQQKPGSSPKSWIYRTSNLASGVSARFSGSGSGTSYSLTISSMEAEDAATYYCQQYHDYPPTFGGGTKLEIKGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 110 - protein, 447 aa, synthetic construct
  CHAIN 1..447: note = 81 L1H1-2-3
EVQLVQSGAEVKKPGASVKVSCKASGYTFTDHNMNWVKQAPGQRLEWMGVMNPKFGTINYNQKFKDKATITRDTSASTAYMELSSLRSEDTAVYYCARDNYPNYFDYWGQGTTVTVSSGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 111 - protein, 209 aa, synthetic construct
  CHAIN 1..209: note = 81 L1H1-2-4
EIVLTQSPATLSLSPGERATLSCSASSSVNYMYWYQQKPGQAPKSWIYRTSNLASGVPARFSGSGSGTDYTLTISSLEPEDFAVYYCQQYHDYPPTFGGGTKVEIKGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 112 - protein, 209 aa, synthetic construct
  CHAIN 1..209: note = 81 L2H1-2-4
EIVLTQSPDFQSVTPGEKVTITCSASSSVNYMYWYQQKPDQSPKSWIYRTSNLASGVPSRFSGSGSGTDFTLTISSLEAEDAATYYCQQYHDYPPTFGGGTKVEIKGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 113 - protein, 209 aa, synthetic construct
  CHAIN 1..209: note = 81 L3H1-2-4
DIQLTQSPSFLSASPGDRVTITCSASSSVNYMYWYQQKPGSSPKSWIYRTSNLASGVPSRFSGSGSGTEYTLTISSLQPEDFATYYCQQYHDYPPTFGGGTKVEIKGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 114 - protein, 209 aa, synthetic construct
  CHAIN 1..209: note = 81 L4H1-2-4
DIQLTQSPSFLSASPGDRVTITCSASSSVNYMYWYQQKPGSSPKSWIYRTSNDASGVPSRFSGSGSGTEYTLTISSLQPEDFATYYCQQYHDYPPTFGGGTKVEIKGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 115 - protein, 209 aa, synthetic construct
  CHAIN 1..209: note = 81 L5H1-2-4
DIQLTQSPSFLSASPGDRVTITCSASSSVNYMYWYQQKPGSSPKSWIYRTSNLAQGVPSRFSGSGSGTEYTLTISSLQPEDFATYYCQQYHDYPPTFGGGTKVEIKGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 116 - protein, 209 aa, synthetic construct
  CHAIN 1..209: note = 81 L6H1-2-4
DIQLTQSPSFLSASPGDRVTITCSASSSVNYMYWYQQKPGSSPKSWIYRTQNLASGVPSRFSGSGSGTEYTLTISSLQPEDFATYYCQQYHDYPPTFGGGTKVEIKGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 117 - protein, 447 aa, synthetic construct
  CHAIN 1..447: note = M146-2-3
EFQLQQSGPELVKPGASVKISCKASGYSFTDYNMNWVKQSNGKSLEWIGLINPKYGTTNYNQKFKGKATLTVDQSSSTAYMQLNSLTSEDSAVYYCAIDDYPAWFAYWGQGTLVTVSAGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 118 - protein, 209 aa, synthetic construct
  CHAIN 1..209: note = M146-2-4
QIVLTQSPAIMSASPGEKVTISCSASSSLNYMYWYQQKPGSSPKSWIYRTSNLASGVPARFSGSGSGTSYSLTISSMEAEDAATYYCQQYHSYPPTFGAGTKLELKGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 119 - protein, 447 aa, synthetic construct
  CHAIN 1..447: note = 146 L1H1-2-3
EFQLVQSGAEVKKPGASVKVSCKASGYSFTDYNMNWVRQAPGQRLEWMGLINPKYGTTNYNQKFKGRVTLTVDQSSSTAYMELSSLRSEDTAVYYCARDNYPAWFAYWGQGTTVTVSSGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 120 - protein, 209 aa, synthetic construct
  CHAIN 1..209: note = 146 L1H1-2-4
EIVLTQSPATLSLSPGERATLSCSASSSLNYMYWYQQKPGSSPRPWIYRTSQLASGVPARFSGSGSGTDYTLTISSLEPEDFAVYYCQQYHSYPPTFGGGTKVEIKGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 121 - protein, 449 aa, synthetic construct
  CHAIN 1..449: note = P3-C7 L7H1-3-1
EVQLVQSGAEVKKPGASVKVSCKASGYTFTDHYIHWVRQAPGQGLEWMGVINPYNDGATYNQKFKGRVTMTVDKSTSTVYMELSSLRSEDTAVYYCARPPRAYGNYAGYWGQGTTVTVSSGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 122 - protein, 447 aa, synthetic construct
  DOMAIN 1..447: note = 81 L1H1-3-1
EVQLVQSGAEVKKPGASVKVSCKASGYTFTDHNMNWVKQAPGQRLEWMGVMNPKFGTINYNQKFKDKATITRDTSASTAYMELSSLRSEDTAVYYCARDNYPNYFDYWGQGTTVTVSSGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 123 - protein, 447 aa, synthetic construct
  CHAIN 1..447: note = 146 L1H1-3-1
EFQLVQSGAEVKKPGASVKVSCKASGYSFTDYNMNWVRQAPGQRLEWMGLINPKYGTTNYNQKFKGRVTLTVDQSSSTAYMELSSLRSEDTAVYYCARDNYPAWFAYWGQGTTVTVSSGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 702 aa, synthetic construct
  CHAIN 1..702: note = P3-C7 L7H1-4-1
EVQLVQSGAEVKKPGASVKVSCKASGYTFTDHYIHWVRQAPGQGLEWMGVINPYNDGATYNQKFKGRVTMTVDKSTSTVYMELSSLRSEDTAVYYCARPPRAYGNYAGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGGGGSGGGGSGGGGSQTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 687 aa, synthetic construct
  CHAIN 1..687: note = P3-C7 L7H1-5-1
EVQLVQSGAEVKKPGASVKVSCKASGYTFTDHYIHWVRQAPGQGLEWMGVINPYNDGATYNQKFKGRVTMTVDKSTSTVYMELSSLRSEDTAVYYCARPPRAYGNYAGYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGGGSGGGGSEVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNEYISYWAYWGQGTLVTVSSGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 461 aa, synthetic construct
  CHAIN 1..461: note = GUCY2c-1608 -Knob
DIQLTQSPSSLSASVGDRVTITCRASESVDYYGSSLLQWYQQKPGKAPKLLIYAASKLASGVPSRFSGSGSGTDFTLTISSLQPEDFATYYCQQTRKAYTFGQGTKLEIKGGGSGGGGEVQLVESGGGLVQPGGSLRLSCAASGFTFSDYYMTWVRQAPGKGLEWVAFIRNQARGYTSDHNPSVKGRFTISRDNAKNSLYLQMNSLRAEDTAVYYCARDRPSYYVLDYWGQGTTVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 127 - protein, 465 aa, synthetic construct
  CHAIN 1..465: note = GUCY2c-1608 -Hole
DIQMTQSPSSLSASVGDRVTITCTSSQSLFNVRSQKNYLAWYQQKPGKAPKLLIYWASTRESGVPSRFSGSGSGTDFTLTISSLQPEDFATYYCKQSYDLFTFGGGTKVEIKGGGGSGGGGEVQLVESGGGLVQPGGSLRLSCAASGFTFSSYWMHWVRQAPGKGLEWIGEIKPSNELTNVHEKFKDRFTISVDKAKNSAYLQMNSLRAEDTAVYYCTRTITTTEGYWFFDVWGQGTLVTVSSGCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 128 - protein, 5 aa, synthetic construct
  DOMAIN 1..5: note = GUCY2C-HCDR1
SYWMH

SEQ ID NO: 129 - protein, 17 aa, synthetic construct
  DOMAIN 1..17: note = GUCY2C-HCDR2
EIKPSNELTNVHEKFKD

SEQ ID NO: 130 - protein, 13 aa, synthetic construct
  DOMAIN 1..13: note = GUCY2C-HCDR3
TITTTEGYWFFDV

SEQ ID NO: 131 - protein, 15 aa, synthetic construct
  DOMAIN 1..15: note = GUCY2C-LCDR1
RASESVDYYGSSLLQ

SEQ ID NO: 132 - protein, 7 aa, synthetic construct
  DOMAIN 1..7: note = GUCY2C-LCDR2
AASKLAS

SEQ ID NO: 133 - protein, 8 aa, synthetic construct
  DOMAIN 1..8: note = GUCY2C-LCDR3
QQTRKAYT

SEQ ID NO: 134 - protein, 5 aa, synthetic construct
  DOMAIN 1..5: note = CD3-HCDR1
DYYMT

SEQ ID NO: 135 - protein, 19 aa, synthetic construct
  DOMAIN 1..19: note = CD3-HCDR2
FIRNQARGYTSDHNPSVKG

SEQ ID NO: 136 - protein, 10 aa, synthetic construct
  DOMAIN 1..10: note = CD3-HCDR3
DRPSYYVLDY

SEQ ID NO: 137 - protein, 17 aa, synthetic construct
  DOMAIN 1..17: note = CD3-LCDR1
TSSQSLFNVRSQKNYLA

SEQ ID NO: 138 - protein, 7 aa, synthetic construct
  DOMAIN 1..7: note = CD3-LCDR2
WASTRES

SEQ ID NO: 139 - protein, 8 aa, synthetic construct
  DOMAIN 1..8: note = CD3-LCDR3
KQSYDLFT

SEQ ID NO: 140 - protein, 5 aa, synthetic construct
  DOMAIN 1..5: note = EGFR-HCDR1
TYGMH

SEQ ID NO: 141 - protein, 17 aa, synthetic construct
  DOMAIN 1..17: note = EGFR-HCDR2
VIWDDGSYKYYGDSVKG

SEQ ID NO: 142 - protein, 16 aa, synthetic construct
  DOMAIN 1..16: note = EGFR-HCDR3
DGITMVRGVMKDYFDY

SEQ ID NO: 143 - protein, 11 aa, synthetic construct
  DOMAIN 1..11: note = EGFR-LCDR1
RASQDISSALV

SEQ ID NO: 144 - protein, 7 aa, synthetic construct
  DOMAIN 1..7: note = EGFR-LCDR2
DASSLES

SEQ ID NO: 145 - protein, 9 aa, synthetic construct
  DOMAIN 1..9: note = EGFR-LCDR3
QQFNSYPLT

SEQ ID NO: 146 - protein, 5 aa, synthetic construct
  DOMAIN 1..5: note = CD28-HCDR1
SYWIT

SEQ ID NO: 147 - protein, 17 aa, synthetic construct
  DOMAIN 1..17: note = CD28-HCDR2
DIYPGSGRTNYNEMFKN

SEQ ID NO: 148 - protein, 12 aa, synthetic construct
  DOMAIN 1..12: note = CD28-HCDR3
RHYGTGYESFDV

SEQ ID NO: 149 - protein, 11 aa, synthetic construct
  DOMAIN 1..11: note = CD28-LCDR1
RASQDISNYLN

SEQ ID NO: 150 - protein, 7 aa, synthetic construct
  DOMAIN 1..7: note = CD28-LCDR2
YTSRLHS

SEQ ID NO: 151 - protein, 9 aa, synthetic construct
  DOMAIN 1..9: note = CD28-LCDR3
QQGNTLPWT

SEQ ID NO: 152 - protein, 125 aa, synthetic construct [sequence omitted]

SEQ ID NO: 153 - protein, 107 aa, synthetic construct
  DOMAIN 1..107: note = EGFR-VL
DIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIK

SEQ ID NO: 154 - protein, 121 aa, synthetic construct
  DOMAIN 1..121: note = CD28-VH
EVQLVQSGAEVKKPGASVKVSCKASGYTLTSYWITWVRQAPGQGLEWMGDIYPGSGRTNYNEMFKNRVTMTVDTSTSTAYMELSSLRSEDTAVYYCARRHYGTGYESFDVWGQGTTVTVSS

SEQ ID NO: 155 - protein, 107 aa, synthetic construct
  DOMAIN 1..107: note = CD28-VL
DIQMTQSPSSLSASVGDRVTITCRASQDISNYLNWYQQKPDGTIKLLIYYTSRLHSGVPSRFSGSGSGTDYTFTISSLQPEDIATYYCQQGNTLPWTFGGGTKVEIK

SEQ ID NO: 156 - protein, 451 aa, synthetic construct
  CHAIN 1..451: note = 97H2L3OT-ZAL-1
EVQLVQSGAEVKKPGASVKVSCKASGYTLTSYWITWVRQAPGQGLEWMGDIYPGSGRTNYNEMFKNRVTMTVDTSTSTAYMELSSLRSEDTAVYYCARRHYGTGYESFDVWGQGTTVTVSSGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSIDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 209 aa, synthetic construct
  CHAIN 1..209: note = 97H2L3OT-ZAL-2
DIQMTQSPSSLSASVGDRVTITCRASQDISNYLNWYQQKPDGTIKLLIYYTSRLHSGVPSRFSGSGSGTDYTFTISSLQPEDIATYYCQQGNTLPWTFGGGTKVEIKGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 158 - protein, 455 aa, synthetic construct
  CHAIN 1..455: note = 97H2L3OT-ZAL-3
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 159 - protein, 214 aa, synthetic construct
  CHAIN 1..214: note = 97H2L3OT-ZAL-4
DIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 160 - protein, 5 aa, synthetic construct
  DOMAIN 1..5: note = CD28-HCDR1
SHWIQ

SEQ ID NO: 161 - protein, 17 aa, synthetic construct
  DOMAIN 1..17: note = CD28-HCDR2
EIFPGSGSTHYNEKFKG

SEQ ID NO: 162 - protein, 12 aa, synthetic construct
  DOMAIN 1..12: note = CD28-HCDR3
LHYGTSYDAMDY

SEQ ID NO: 163 - protein, 9 aa, synthetic construct
  DOMAIN 1..9: note = CD28-LCDR3
QQGYTLPRT

SEQ ID NO: 164 - protein, 121 aa, synthetic construct
  DOMAIN 1..121: note = CD28-VH
EVQLVQSGAEVKKPGASVKVSCKAPGYTFTSHWIQWVRQAPGQGLEWMGEIFPGSGSTHYNEKFKGRVTMTVDTSTSTAYMELSSLRSEDTAVYYCARLHYGTSYDAMDYWGQGTTVTVSS

SEQ ID NO: 165 - protein, 107 aa, synthetic construct
  DOMAIN 1..107: note = CD28-VL
DIQMTQSPSSLSASVGDRVTITCRASQDISNYLNWYQQKPGKAVKLLIYYTSRLHSGVPSRFSGSGSGTDFTFTISSLQPEDIATYYCQQGYTLPRTFGGGTKVEIK

SEQ ID NO: 166 - protein, 451 aa, synthetic construct
  CHAIN 1..451: note = 129H4L1OT-ZAL-1
EVQLVQSGAEVKKPGASVKVSCKAPGYTFTSHWIQWVRQAPGQGLEWMGEIFPGSGSTHYNEKFKGRVTMTVDTSTSTAYMELSSLRSEDTAVYYCARLHYGTSYDAMDYWGQGTTVTVSSGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSIDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 167 - protein, 209 aa, synthetic construct
  CHAIN 1..209: note = 129H4L1OT-ZAL-2
DIQMTQSPSSLSASVGDRVTITCRASQDISNYLNWYQQKPGKAVKLLIYYTSRLHSGVPSRFSGSGSGTDFTFTISSLQPEDIATYYCQQGYTLPRTFGGGTKVEIKGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 168 - protein, 5 aa, synthetic construct
  DOMAIN 1..5: note = CD28-HCDR1
GYYMN

SEQ ID NO: 169 - protein, 17 aa, synthetic construct
  DOMAIN 1..17: note = CD28-HCDR2
EINPSTGDTTYNQKFKA

SEQ ID NO: 170 - protein, 10 aa, synthetic construct
  DOMAIN 1..10: note = CD28-HCDR3
PYYDLTPFLY

SEQ ID NO: 171 - protein, 11 aa, synthetic construct
  DOMAIN 1..11: note = CD28-LCDR1
LASQTIGTWLA

SEQ ID NO: 172 - protein, 7 aa, synthetic construct
  DOMAIN 1..7: note = CD28-LCDR2
AATTGAD

SEQ ID NO: 173 - protein, 9 aa, synthetic construct
  DOMAIN 1..9: note = CD28-LCDR3
QQLYSTPYT

SEQ ID NO: 174 - protein, 119 aa, synthetic construct
  DOMAIN 1..119: note = CD28-VH
EVQLVQSGAEVKKPGASVKVSCKASGYSFTGYYMNWVRQAPGQRLEWMGEINPSTGDTTYNQKFKAKVTLTVDKSASTAYMELSSLRSEDTAVYYCASPYYDLTPFLYWGQGTLVTVSS

SEQ ID NO: 175 - protein, 107 aa, synthetic construct
  DOMAIN 1..107: note = CD28-VL
DIQMTQSPSSVSASVGDRVTITCLASQTIGTWLAWYQQKPGKSPKLLIYAATTGADGVPSRFSGSGSGTKFTLTISSLQPEDFATYYCQQLYSTPYTFGGGTKVEIK

SEQ ID NO: 176 - protein, 449 aa, synthetic construct
  CHAIN 1..449: note = 94H6L12-ZALOT-1
EVQLVQSGAEVKKPGASVKVSCKASGYSFTGYYMNWVRQAPGQRLEWMGEINPSTGDTTYNQKFKAKVTLTVDKSASTAYMELSSLRSEDTAVYYCASPYYDLTPFLYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 177 - protein, 214 aa, synthetic construct
  DOMAIN 1..214: note = 94H6L12-ZALOT-2
DIQMTQSPSSVSASVGDRVTITCLASQTIGTWLAWYQQKPGKSPKLLIYAATTGADGVPSRFSGSGSGTKFTLTISSLQPEDFATYYCQQLYSTPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 178 - protein, 454 aa, synthetic construct
  DOMAIN 1..454: note = 94H6L12-ZALOT-3
QVQLVESGGGVVQPGRSLRLSCAASGFTFSTYGMHWVRQAPGKGLEWVAVIWDDGSYKYYGDSVKGRFTISRDNSKNTLYLQMNSLRAEDTAVYYCARDGITMVRGVMKDYFDYWGQGTLVTVSSGGGGSSGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEADKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 179 - protein, 210 aa, synthetic construct
  CHAIN 1..210: note = 94H6L12-ZALOT-4
DIQLTQSPSSLSASVGDRVTITCRASQDISSALVWYQQKPGKAPKLLIYDASSLESGVPSRFSGSESGTDFTLTISSLQPEDFATYYCQQFNSYPLTFGGGTKVEIKGGGGSGIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 180 - protein, 118 aa, synthetic construct
  DOMAIN 1..118: note = VH2-L
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 181 - protein, 108 aa, synthetic construct
  DOMAIN 1..108: note = VH2-L
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 182 - protein, 98 aa, synthetic construct
  DOMAIN 1..98: note = T.0
GIPPKIEALPSDISIDEGKVLTVACAFTGEPTPEVTWSCGGRKIHSQEQGRFHIENTDDLTTLIIMDVQKQDGGLYTLSLGNEFGSDSATVNIHIRSI

SEQ ID NO: 183 - protein, 98 aa, synthetic construct
  DOMAIN 1..98: note = T.1
GIPPKIECLPSDISIDEGKVLTVASAFTGEPTPEVTWSTGGRKIHSQEQGRFHIENTDDLTTLIIMDVQKQDGGLYTLSLGNEFGSDSATVNIHIRSI

SEQ ID NO: 184 - protein, 98 aa, synthetic construct
  DOMAIN 1..98: note = T.2
GIPPKIEALPSDISIDEGKCLTVASAFTGEPTPEVTWSTGGRKIHSQEQGRFHIENTDDLTTLIIMDVQKQDGGLYTLSLGNEFGSDSATVNIHIRSI

SEQ ID NO: 185 - protein, 98 aa, synthetic construct
  DOMAIN 1..98: note = T.3
GIPPKIEALPSDISIDEGKVLTVASCFTGEPTPEVTWSTGGRKIHSQEQGRFHIENTDDLTTLIIMDVQKQDGGLYTLSLGNEFGSDSATVNIHIRSI

SEQ ID NO: 186 - protein, 98 aa, synthetic construct
  DOMAIN 1..98: note = T.4
GIPPKIEALPSDISIDEGKVLCVASAFTGEPTPEVTWSTGGRKIHSQEQGRFHIENTDDLTTLIIMDVQKQDGGLYTLSLGNEFGSDSATVNIHIRSI

SEQ ID NO: 187 - protein, 103 aa, synthetic construct
  DOMAIN 1..103: note = T.5
KAGIRGIPPKIECLPSDISIDEGKVLTVASAFTGEPTPEVTWSTGGRKIHSQEQGRFHIENTDDLTTLIIMDVQKQDGGLYTLSLGNEFGSDSATVNIHIRSI

SEQ ID NO: 188 - protein, 103 aa, synthetic construct
  DOMAIN 1..103: note = T.6
KAGIRGIPPKIEALPSDISIDEGKVLTVACAFTGEPTPEVTWSCGGRKIHSQEQGRFHIENTDDLTTLIIMDVQKQDGGLYTLSLGNEFGSDSATVNIHIRSI

SEQ ID NO: 189 - protein, 98 aa, synthetic construct
  DOMAIN 1..98: note = T.7
GIPPKIECLPSDISIDEGKVLTVASAFTGEPTPEVTWSTGGRKIHSQEQGRFHIENTDDLTTLIISDVQKQDGGLYSLSLGNEFGSDSATVNIHIRSI

SEQ ID NO: 190 - protein, 98 aa, synthetic construct
  DOMAIN 1..98: note = T.8
GIPPKIECLPSDISIDEGKVLTVASAFTGEPTPEVTWSTGGRKIHSQEQGRFHIENTDDLTTLIIKDVQRQDGGLYTLTLRNEFGSDSATVNIHIRSI

SEQ ID NO: 191 - protein, 98 aa, synthetic construct
  DOMAIN 1..98: note = T.9
GIWPKIECLPIDLSIDEGKVLMVASAFTGEPTPEVTWSTGGRKIHSQEQGRFHIENTDDLTTLIIMDVQKQDGGLYTLSLGMEFGSDSATVNIHIRSI

SEQ ID NO: 192 - protein, 98 aa, synthetic construct
  DOMAIN 1..98: note = T.10
GIPPKIECLPIDISIDEGKVLTVASAFTGEPTPEVTWSTGGRKIHSQEQGRFHIENTDDLTTLIIKDVQKQDGGLYTLTLRNEFGSDSATVNIHIRSI

SEQ ID NO: 193 - protein, 98 aa, synthetic construct
  DOMAIN 1..98: note = T.11
GIPPKIECLPSDISIDEGKVLTVASAFTGEPTPEVTWSTSGKKISSEEGGRFHIESTEDLTTLIIMDVQKQDGGVYTLSLGNDLGSDSATVNIHIRSI

SEQ ID NO: 194 - protein, 98 aa, synthetic construct
  DOMAIN 1..98: note = T.12
GIPPKIECLPSDISIDEGKVLTVASAFTGEPTPEVTWSTGGRKIHSEEGGRFHIESTEDLTTLIIMDVQKQDGGVYTLSLGNEFGSDSATVNIHIRSI

SEQ ID NO: 195 - protein, 98 aa, synthetic construct
  DOMAIN 1..98: note = T.13
GIPPKIECLPSDISIDEGKVLTVASAFTGEPTPEVTWSTGGRKIHSQEQGRFHIESTEDLTTLIIMDVQKQDGGVYTLSLGNEFGSDSATVNIHIRSI

SEQ ID NO: 196 - protein, 98 aa, synthetic construct
  DOMAIN 1..98: note = T.14
GIPPKIECLPSDISIDEGKVLTVASAFTGEPTPEVTWSTGGRKIHSQEQGRFHIESTEDLTTLIISDVQKQDGGLYSLSLGNEFGSDSATVNIHIRSI

SEQ ID NO: 197 - protein, 103 aa, synthetic construct
  DOMAIN 1..103: note = T.15
KAGIRGIPPKIECLPSDISIDEGKVLTVASAFTGEPTPEVTWSTGGRKIHSQEQGRFHIESTEDLTTLIISDVQKQDGGLYSLSLGNEFGSDSATVNIHIRSI

SEQ ID NO: 198 - protein, 98 aa, synthetic construct
  DOMAIN 1..98: note = T.16
GIPPKIECLPIDISIDEGKVLTVASAFTGEPTPEVTWSTGGRKIHSQEQGRFHIENTDDSTTLTIKDVQKQDGGLYTLTLRNEFGSDSATVNIHIRSI

SEQ ID NO: 199 - protein, 98 aa, synthetic construct
  DOMAIN 1..98: note = T.17
GIPPKIECLPIDISIDEGKCLTVASAFTGEPTPEVTWSTGGRKIHSQEQGRFHIENTDDSTTLTIKDVQKQDGGLYTLTLRNEFGSDSATVNIHIRSI

SEQ ID NO: 200 - protein, 98 aa, synthetic construct
  DOMAIN 1..98: note = T.18
GIPPKIECLPIDISIDEGKVLTVASCFTGEPTPEVTWSTGGRKIHSQEQGRFHIENTDDSTTLTIKDVQKQDGGLYTLTLRNEFGSDSATVNIHIRSI

SEQ ID NO: 201 - protein, 97 aa, synthetic construct
  DOMAIN 1..97: note = O.0
SGAPRFLTRPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 202 - protein, 98 aa, synthetic construct
  DOMAIN 1..98: note = OL.0
QGSPPCFLRFPRPVRVVSGAEAELKCVVLGEPPPVVVWEKGGQQLAASERLSFPADGAEHGLLLTAALPTDAGVYVCRARNAAGEAYAAAAVTVLEPP

SEQ ID NO: 203 - protein, 97 aa, synthetic construct
  DOMAIN 1..97: note = O.1
SGAPRFLTRPKAFVVSVGKDATLSCQIVGNPTPQVSWEKDQQPVAAGARFRLAQDGDLYRLTILDLALGDSGQYVCRARNAIGEAFACVGLQVDAEA

SEQ ID NO: 204 - protein, 97 aa, synthetic construct
  DOMAIN 1..97: note = O.2
SGCPRFLTRPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 205 - protein, 97 aa, synthetic construct
  DOMAIN 1..97: note = O.3
SGAPRFLTCPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 206 - protein, 97 aa, synthetic construct
  DOMAIN 1..97: note = O.4
SGAPRFLTRPKAFVVSVGKDATLSSQIVGNPTPQVSWEKDQQPVAAGARFRLAQDGDLYRLTILDLALGDSGQYVSRARNAIGEAFACVGLQVDAEA

SEQ ID NO: 207 - protein, 97 aa, synthetic construct
  DOMAIN 1..97: note = O.5
SGCPRFLTRPKAFVVSVGKDATLSSQIVGNPTPQVSWEKDQQPVAAGARFRLAQDGDLYRLTILDLALGDSGQYVSRARNAIGEAFAAVGLQVDAEA

SEQ ID NO: 208 - protein, 97 aa, synthetic construct
  DOMAIN 1..97: note = O.6
SGAPRFLTCPKAFVVSVGKDATLSSQIVGNPTPQVSWEKDQQPVAAGARFRLAQDGDLYRLTILDLALGDSGQYVSRARNAIGEAFAAVGLQVDAEA

SEQ ID NO: 209 - protein, 97 aa, synthetic construct
  DOMAIN 1..97: note = O.7
SGAPRFKTRPKAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 210 - protein, 97 aa, synthetic construct
  DOMAIN 1..97: note = O.8
SGAPRFKTRPKAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 211 - protein, 97 aa, synthetic construct
  DOMAIN 1..97: note = O.9
SGAPRFLTRPLAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 212 - protein, 97 aa, synthetic construct
  DOMAIN 1..97: note = O.10
SGAPRFLTRPLAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 213 - protein, 102 aa, synthetic construct
  DOMAIN 1..102: note = O.11
DQPQFSGAPRFLTRPKAFVVSVGKDATLSSQIVGNPTPQVSWEKDQQPVAAGARFRLAQDGDLYRLTILDLALGDSGQYVSRARNAIGEAFACVGLQVDAEA

SEQ ID NO: 214 - protein, 102 aa, synthetic construct
  DOMAIN 1..102: note = O.12
DQPQFSGAPRFKTRPKAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 215 - protein, 102 aa, synthetic construct
  DOMAIN 1..102: note = O.13
DQPQFSGAPRFKTRPKAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 216 - protein, 102 aa, synthetic construct
  DOMAIN 1..102: note = O.14
DQPQFSGAPRFRTRPKAFVVSVGKDATLSSQIVGNPTPQVSWEKDQQPVAAGARFRLAQDGDLYRLKILDLALGDSGQYVSRARNAIGEAFACVGLQVDAEA

SEQ ID NO: 217 - protein, 102 aa, synthetic construct
  DOMAIN 1..102: note = O.15
DQPQFSGAPRFRTRPKAFVVSVGKDATLSSQIVGNPTPQVSWEKDQQPVAAGARFRLAQDGDLYRLHILDLALGDSGQYVSRARNAIGEAFACVGLQVDAEA

SEQ ID NO: 218 - protein, 102 aa, synthetic construct
  DOMAIN 1..102: note = O.16
DQPQFSGAPRFLTRPKAFVVSVGKDATLSCQIVGNPTPQVSWEKDQQPVAAGARFRLAQDGDLYRLTILDLALGDSGQYVCRARNAIGEAFAAVGLQVDAEA

SEQ ID NO: 219 - protein, 98 aa, synthetic construct
  DOMAIN 1..98: note = OL.1
QGSPPEFLRFPRPVRVVSGAEAELKCVVLGEPPPVVVWEKGGQQLAASERLSFPADGAEHGLLLTAALPTDAGCYVCRARNAAGEAYAAAAVTVLEPP

SEQ ID NO: 220 - protein, 98 aa, synthetic construct
  DOMAIN 1..98: note = OL.2
QGSPPEFLRFPRPVRVVSGAEAELKCVVLGEPPPVVVWEKGGQQLAASERLSFPADGAEHGLLLTAALPTDAGVYVCRARNAACEAYAAAAVTVLEPP

SEQ ID NO: 221 - protein, 98 aa, synthetic construct
  DOMAIN 1..98: note = OL.3
QGSPPEFLRFPRPVRVVSGAEAELKCVVLGEPPPVVVWEKGGQQLAASERLSFPADGAEHGLLLTAALPTDAGVYVCRARNAAGECYAAAAVTVLEPP

SEQ ID NO: 222 - protein, 98 aa, synthetic construct
  DOMAIN 1..98: note = OL.4
QGSPPEFLRFPRPVRVVSGAEAELKSVVLGEPPPVVVWEKGGQQLAASERLSFPADGAEHGLLLTAALPTDAGCYVSRARNAAGEAYAAAAVTVLEPP

SEQ ID NO: 223 - protein, 98 aa, synthetic construct
  DOMAIN 1..98: note = OL.5
QGSPPEFLRFPRPVRVVSGAEAELKSVVLGEPPPVVVWEKGGQQLAASERLSFPADGAEHGLLLTAALPTDAGVYVSRARNAACEAYAAAAVTVLEPP

SEQ ID NO: 224 - protein, 98 aa, synthetic construct
  DOMAIN 1..98: note = OL.6
QGSPPEFLRFPRPVRVVSGAEAELKSVVLGEPPPVVVWEKGGQQLAASERLSFPADGAEHGLLLTAALPTDAGVYVSRARNAAGECYAAAAVTVLEPP

SEQ ID NO: 225 - protein, 97 aa, synthetic construct
  DOMAIN 1..97: note = O.17
SGAPRFLTRPKSYTVSVGKDASLSSQIVGNPTPQVSWEKDQQPVAAGARFRLAQDGDLYRLKILDLALGDSGQYVSRARNAIGEAFACVGLQVDAEA

SEQ ID NO: 226 - protein, 97 aa, synthetic construct
  DOMAIN 1..97: note = O.18
SEAPRFLTRPLAFVVSEGKDATLSSQIVGDPPPEVTWEKDQQPITAGARFRLAQDGDVYRLEILDLQLSDSGQYVSRARNAIGEAFACVGLQVDAEG

SEQ ID NO: 227 - protein, 97 aa, synthetic construct
  DOMAIN 1..97: note = O.19
SGAPRFLTRPLAFVVSVGKLAMLSSQIVGNPTPQVSWEKDQQPVAAGARFRLLQDGDLYRLKILDLALGDSGQYVSRARNAIGEAFACVGLQVDAEA

SEQ ID NO: 228 - protein, 97 aa, synthetic construct
  DOMAIN 1..97: note = O.20
SGAPRFLTRPLAFVVSVGKDATLSSQIVGNPTPQVSWEKDKQPVTAGARFRLAQDGDLYRLKILDLQLSDSGQYVSRARNAIGEAFACLGLQVDAEA

SEQ ID NO: 229 - protein, 97 aa, synthetic construct
  DOMAIN 1..97: note = O.21
SGAPRFLTRPLAFVVSVGKDATLSSQIVGNPTPQVSWEKDQLPVTAGARFRLAQDGDLYRLKILDLTLSDSGQYVSRARNAIGEAFACVGLEVGAEA

SEQ ID NO: 230 - protein, 97 aa, synthetic construct
  DOMAIN 1..97: note = O.22
SGAPRFLTRPLSYVVSVGKDASLSSQIVGNPTPQVSWEKDQLPVTAGARFRLAQDGDLYRLKILDLQLSDSGQYVSRARNAIGEAFACVGLEVGAEA

SEQ ID NO: 231 - protein, 102 aa, synthetic construct
  DOMAIN 1..102: note = O.23
DQPQFSGAPRFLTRPLSYVVSVGKDASLSSQIVGNPTPQVSWEKDQLPVTAGARFRLAQDGDLYRLKILDLQLSDSGQYVSRARNAIGEAFACVGLEVGAEA

SEQ ID NO: 232 - protein, 97 aa, synthetic construct
  DOMAIN 1..97: note = O.24
SGAPRFLTRPKAFVVSVGKDATLSSQIVGNPFPQVSWEKDKQPVTAGVRFRLAQDGDLYRLKILDLQLSDSGQYVSRARNAIGEAFACLGLQVDAEA

SEQ ID NO: 233 - protein, 97 aa, synthetic construct
  DOMAIN 1..97: note = O.25
SGAPRFLTRPKASVVSVGKDATLSSQIVGNPFPQVSWEKDKQPVTAGVRFRLAQDGDLYRLKILDLQLSDSGQYVSRARNAHGEAFACLGLQVDAEA

SEQ ID NO: 234 - protein, 97 aa, synthetic construct
  DOMAIN 1..97: note = O.26
SGCPRFLTRPKASVVSVGKDATLSSQIVGNPFPQVSWEKDKQPVTAGVRFRLAQDGDLYRLKILDLQLSDSGQYVSRARNAHGEAFACLGLQVDAEA

SEQ ID NO: 235 - protein, 97 aa, synthetic construct
  DOMAIN 1..97: note = O.27
SGAPRFLTCPKASVVSVGKDATLSSQIVGNPFPQVSWEKDKQPVTAGVRFRLAQDGDLYRLKILDLQLSDSGQYVSRARNAHGEAFACLGLQVDAEA

SEQ ID NO: 236 - protein, 97 aa, synthetic construct
  DOMAIN 1..97: note = O.28
SGAPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 237 - protein, 97 aa, synthetic construct
  DOMAIN 1..97: note = O.29
SGCPRFLTRPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 238 - protein, 97 aa, synthetic construct
  DOMAIN 1..97: note = O.30
SGAPRFLTCPKASVVSVGKDATLSCQIVGNPFPQVSWEKDKQPVTAGVRFRLAQDGDLYRLKILDLQLSDSGQYVCRARNAHGEAFACLGLQVDAEA

SEQ ID NO: 239 - protein, 97 aa, synthetic construct
  DOMAIN 1..97: note = O.31
SGAPRFLTRPKASVVSVGKDATLSSQIVGNPFPQVSWEKDKQPVTAGVRFRLAQDGDLYRLKILDCQLSDSGQYVSRARNAHGEAFACLGLQCDAEA

SEQ ID NO: 240 - protein, 97 aa, synthetic construct
  DOMAIN 1..97: note = O.32
SGCPRFLTRPKASVVSVGKDATLSSQIVGNPFPQVSWEKDKQPVTAGVRFRLAQDGDLYRLKILDCQLSDSGQYVSRARNAHGEAFACLGLQCDAEA

SEQ ID NO: 241 - protein, 97 aa, synthetic construct
  DOMAIN 1..97: note = O.33
SGAPRFLTCPKASVVSVGKDATLSSQIVGNPFPQVSWEKDKQPVTAGVRFRLAQDGDLYRLKILDCQLSDSGQYVSRARNAHGEAFACLGLQCDAEA

SEQ ID NO: 242 - protein, 455 aa, synthetic construct
  CHAIN 1..455: note = Format 2-1 (N103QN106Q)
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGQFGQSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 243 - protein, 5 aa, synthetic construct
  DOMAIN 1..5: note = linker
ASTKG

SEQ ID NO: 244 - protein, 5 aa, synthetic construct
RTVAS

SEQ ID NO: 245 - skipped

SEQ ID NO: 246 - protein, 4 aa, synthetic construct
  DOMAIN 1..4: note = linker
GGGS

SEQ ID NO: 247 - protein, 4 aa, synthetic construct
  DOMAIN 1..4: note = linker
GGGG

SEQ ID NO: 248 - protein, 227 aa, synthetic construct
  DOMAIN 1..227: note = IgG1Fc-Knob L234A/L235A/S354C/T366W
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 249 - protein, 227 aa, synthetic construct
  DOMAIN 1..227: note = IgG1Fc-Hole L234A/L235A/Y349C/T366S/L368A/Y407V
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 250 - protein, 5 aa, synthetic construct
  DOMAIN 1..5: note = M42-HCDR1
SYAVH

SEQ ID NO: 251 - protein, 16 aa, synthetic construct
  DOMAIN 1..16: note = M42-HCDR2
VIWTGGNTRSNAAFMS

SEQ ID NO: 252 - protein, 10 aa, synthetic construct
  DOMAIN 1..10: note = M42-HCDR3
NSLLDYAMDY

SEQ ID NO: 253 - protein, 12 aa, synthetic construct
  DOMAIN 1..12: note = M42-LCDR1
RASSFVGSGYLH

SEQ ID NO: 254 - protein, 7 aa, synthetic construct
  DOMAIN 1..7: note = M42-LCDR2
STSKLAS

SEQ ID NO: 255 - protein, 9 aa, synthetic construct
  DOMAIN 1..9: note = M42-LCDR3
QQSSGYPLT

SEQ ID NO: 256 - protein, 5 aa, synthetic construct
  DOMAIN 1..5: note = M61-HCDR1
DYYMS

SEQ ID NO: 257 - protein, 17 aa, synthetic construct
  DOMAIN 1..17: note = M61-HCDR2
DINPNNGGTSDNQKFKG

SEQ ID NO: 258 - protein, 11 aa, synthetic construct
  DOMAIN 1..11: note = M61-HCDR3
GRGMVARYFDI

SEQ ID NO: 259 - protein, 12 aa, synthetic construct
  DOMAIN 1..12: note = M61-LCDR1
SASSSISSNFLH

SEQ ID NO: 260 - protein, 9 aa, synthetic construct
  DOMAIN 1..9: note = M61-LCDR3
QQGSGLPLT

SEQ ID NO: 261 - protein, 5 aa, synthetic construct
  DOMAIN 1..5: note = M64-HCDR1
DYGMH

SEQ ID NO: 262 - protein, 17 aa, synthetic construct
  DOMAIN 1..17: note = M64-HCDR2
YISSGSSTIYYADTVKG

SEQ ID NO: 263 - protein, 14 aa, synthetic construct
  DOMAIN 1..14: note = M64-HCDR3
TRVYFYGSYWYFDV

SEQ ID NO: 264 - protein, 10 aa, synthetic construct
  DOMAIN 1..10: note = M64-LCDR1
SASSSVSYMY

SEQ ID NO: 265 - protein, 7 aa, synthetic construct
  DOMAIN 1..7: note = M64-LCDR2
DTSKLAS

SEQ ID NO: 266 - protein, 10 aa, synthetic construct
  DOMAIN 1..10: note = M64-LCDR3
QQWSSDPPYT

SEQ ID NO: 267 - protein, 5 aa, synthetic construct
  DOMAIN 1..5: note = M93-HCDR1
RYWMH

SEQ ID NO: 268 - protein, 17 aa, synthetic construct
  DOMAIN 1..17: note = M93-HCDR2
MIDPSDSYTKYNQKFMG

SEQ ID NO: 269 - protein, 11 aa, synthetic construct
  DOMAIN 1..11: note = M93-HCDR3
SIKVAGWYFDV

SEQ ID NO: 270 - protein, 11 aa, synthetic construct
  DOMAIN 1..11: note = M93-LCDR1
KASQDVSSAVA

SEQ ID NO: 271 - protein, 7 aa, synthetic construct
  DOMAIN 1..7: note = M93-LCDR2
SASYRST

SEQ ID NO: 272 - protein, 9 aa, synthetic construct
  DOMAIN 1..9: note = M93-LCDR3
QQHFSTTLT

SEQ ID NO: 273 - protein, 118 aa, synthetic construct
  DOMAIN 1..118: note = M42VH
QIQLKQSGPGLVQPSQSLSITCTVSGFSLTSYAVHWVRQSPGKGLEWLGVIWTGGNTRSNAAFMSRLTITKDNSKSQVFFKMNSLQADDTAIYFCAKNSLLDYAMDYWGQGTSVTVSS

SEQ ID NO: 274 - protein, 108 aa, synthetic construct
  DOMAIN 1..108: note = M42VL
ENVLTQSPVIMSASPGEKVTMTCRASSFVGSGYLHWYQQKSGASPKLWIYSTSKLASGVPARFSGSGSGTSFFLTISRVEIEDAAAYYCQQSSGYPLTFGGGSKLEIK

SEQ ID NO: 275 - protein, 120 aa, synthetic construct
  DOMAIN 1..120: note = M61VH
EVQLQQSGPELVRPGASVKISCTASGYTITDYYMSWVKQSHGKSPEWIGDINPNNGGTSDNQKFKGKATLTVDKSSSTAYMEYRSLTSEDSAVYYCARGRGMVARYFDIWGTGTTVTVSS

SEQ ID NO: 276 - protein, 108 aa, synthetic construct
  DOMAIN 1..108: note = M61VL
EILLTQSPTTMAASPGEKITITCSASSSISSNFLHWYQEKPGFSPKLLIYRTSNLASGVPARFSGSGSGTSYSLTIGSMEAEDVATYYCQQGSGLPLTFGAGTKLELK

SEQ ID NO: 277 - protein, 123 aa, synthetic construct
  DOMAIN 1..123: note = M64VH
EVQLVESGGGLVKPGGSLKLSCAASGFTFSDYGMHWVRQAPEKGLEWVAYISSGSSTIYYADTVKGRFTISRDNAKNTLFLQMTSLRSEDTAMYYCARTRVYFYGSYWYFDVWGTGTTVTVSS

SEQ ID NO: 278 - protein, 107 aa, synthetic construct
  DOMAIN 1..107: note = M64VL
QIVLTQSPAIMSASPGEKVTMTCSASSSVSYMYWYQQKSGTSPKRWIYDTSKLASGVPARFSGSGSGTSYSLTISSLEAEDAATYYCQQWSSDPPYTFGGGTKLEIK

SEQ ID NO: 279 - protein, 120 aa, synthetic construct
  DOMAIN 1..120: note = M93VH
QVQLQQPGAELVRPGTSVKLSCKASGYTFTRYWMHGVKRRPGQGLEWIGMIDPSDSYTKYNQKFMGKAILTVDTSSSTAYMQFSSLTSEDSAVYYCARSIKVAGWYFDVWGTGTTVTVSS

SEQ ID NO: 280 - protein, 107 aa, synthetic construct
  DOMAIN 1..107: note = M93VL
DIVMTQSHKFMSTSVGDRVSITCKASQDVSSAVAWYQQKPGQSPKLLIYSASYRSTGVPDRFTGSGSGTDFTFTISSVQAEDLAIYYCQQHFSTTLTFGAGTKLELK

SEQ ID NO: 281 - protein, 5 aa, synthetic construct
  DOMAIN 1..5: note = P1-D2-HCDR1
DHNMN

SEQ ID NO: 282 - protein, 17 aa, synthetic construct
  DOMAIN 1..17: note = P1-D2-HCDR2
VINPKYGTISNNQKFKG

SEQ ID NO: 283 - protein, 9 aa, synthetic construct
  DOMAIN 1..9: note = P1-D2-HCDR3
DDYPNYFDY

SEQ ID NO: 284 - protein, 10 aa, synthetic construct
  DOMAIN 1..10: note = P1-D2-LCDR1
SANSSVNYIY

SEQ ID NO: 285 - protein, 9 aa, synthetic construct
  DOMAIN 1..9: note = P1-D2-LCDR3
QQYHSFPPT

SEQ ID NO: 286 - protein, 5 aa, synthetic construct
  DOMAIN 1..5: note = P1-D3-HCDR1
TYGMS

SEQ ID NO: 287 - protein, 17 aa, synthetic construct
  DOMAIN 1..17: note = P1-D3-HCDR2
WINTYSGVPTYADDFKG

SEQ ID NO: 288 - protein, 13 aa, synthetic construct
  DOMAIN 1..13: note = P1-D3-HCDR3
RGYSNLYYYALDY

SEQ ID NO: 289 - protein, 10 aa, synthetic construct
  DOMAIN 1..10: note = P1-D3-LCDR1
SASSSVNYMH

SEQ ID NO: 290 - protein, 9 aa, synthetic construct
  DOMAIN 1..9: note = P1-D3-LCDR3
QQWSSNPST

SEQ ID NO: 291 - protein, 5 aa, synthetic construct
  DOMAIN 1..5: note = P1-C6-HCDR1
SYGIT

SEQ ID NO: 292 - protein, 17 aa, synthetic construct
  DOMAIN 1..17: note = P1-C6-HCDR2
EIYPRSGNTYYNEKFKG

SEQ ID NO: 293 - protein, 13 aa, synthetic construct
  DOMAIN 1..13: note = P1-C6-HCDR3
AITTVVGDWYFDV

SEQ ID NO: 294 - protein, 15 aa, synthetic construct
  DOMAIN 1..15: note = P1-C6-LCDR1
RASESVSLIGTNLIH

SEQ ID NO: 295 - protein, 7 aa, synthetic construct
  DOMAIN 1..7: note = P1-C6-LCDR2
HASNLEA

SEQ ID NO: 296 - protein, 8 aa, synthetic construct
  DOMAIN 1..8: note = P1-C6-LCDR3
LQSRKIRT

SEQ ID NO: 297 - protein, 17 aa, synthetic construct
  DOMAIN 1..17: note = P1-D9-HCDR2
HISSGSSTIYYADTVKG

SEQ ID NO: 298 - protein, 12 aa, synthetic construct
  DOMAIN 1..12: note = P1-D9-HCDR3
SRNYYGSRIFDY

SEQ ID NO: 299 - protein, 7 aa, synthetic construct
  DOMAIN 1..7: note = P1-D9-LCDR2
LTSNLAS

SEQ ID NO: 300 - protein, 9 aa, synthetic construct
  DOMAIN 1..9: note = P1-D9-LCDR3
QQWSSNPPT

SEQ ID NO: 301 - protein, 17 aa, synthetic construct
  DOMAIN 1..17: note = P1-F10-HCDR2
HISSGSSTIYYADTVKG

SEQ ID NO: 302 - protein, 12 aa, synthetic construct
  DOMAIN 1..12: note = P1-F10-HCDR3
SRNYYGSRVFDY

SEQ ID NO: 303 - protein, 10 aa, synthetic construct
  DOMAIN 1..10: note = P1-F10-LCDR1
SASSSVNYMY

SEQ ID NO: 304 - protein, 9 aa, synthetic construct
  DOMAIN 1..9: note = P1-F10-LCDR3
QQWNSNPPT

SEQ ID NO: 305 - protein, 5 aa, synthetic construct
  DOMAIN 1..5: note = P2-C8-HCDR1
DYYMH

SEQ ID NO: 306 - protein, 17 aa, synthetic construct
  DOMAIN 1..17: note = P2-C8-HCDR2
LINPYNGVTSYNQKFKG

SEQ ID NO: 307 - protein, 17 aa, synthetic construct
  DOMAIN 1..17: note = P2-B9-HCDR2
MIHPKSDSVNNNEKFKN

SEQ ID NO: 308 - protein, 10 aa, synthetic construct
  DOMAIN 1..10: note = P2-B9-HCDR3
GGDSGYSMDY

SEQ ID NO: 309 - protein, 16 aa, synthetic construct
  DOMAIN 1..16: note = P2-B9-LCDR1
KASKKVTIFGSISALH

SEQ ID NO: 310 - protein, 7 aa, synthetic construct
  DOMAIN 1..7: note = P2-B9-LCDR2
NGAKLES

SEQ ID NO: 311 - protein, 9 aa, synthetic construct
  DOMAIN 1..9: note = P2-B9-LCDR3
LQNKEVPYT

SEQ ID NO: 312 - protein, 5 aa, synthetic construct
  DOMAIN 1..5: note = P3-D2-HCDR1
DYFMH

SEQ ID NO: 313 - protein, 17 aa, synthetic construct
  DOMAIN 1..17: note = P3-D2-HCDR2
LINPYSGGTTYNQKFKG

SEQ ID NO: 314 - protein, 11 aa, synthetic construct
  DOMAIN 1..11: note = P3-D2-HCDR3
PPRAYGDYAGY

SEQ ID NO: 315 - protein, 9 aa, synthetic construct
  DOMAIN 1..9: note = P3-D2-LCDR3
SQSTHVPLT

SEQ ID NO: 316 - protein, 13 aa, synthetic construct
  DOMAIN 1..13: note = P3-B4-HCDR3
RAGSSLYYYAMDY

SEQ ID NO: 317 - protein, 10 aa, synthetic construct
  DOMAIN 1..10: note = P3-B4-LCDR1
SASSSVSYML

SEQ ID NO: 318 - protein, 5 aa, synthetic construct
  DOMAIN 1..5: note = P3-E4-HCDR1
NYWMH

SEQ ID NO: 319 - protein, 17 aa, synthetic construct
  DOMAIN 1..17: note = P3-E4-HCDR2
EIDPSDGYTDYNQKFKG

SEQ ID NO: 320 - protein, 13 aa, synthetic construct
  DNA_BIND 1..13: note = P3-E4-HCDR3
SLRGPITFGPFPY

SEQ ID NO: 321 - protein, 15 aa, synthetic construct
  DOMAIN 1..15: note = P3-E4-LCDR1
RASESVSIHGTHLMH

SEQ ID NO: 322 - protein, 7 aa, synthetic construct
  DOMAIN 1..7: note = P3-E4-LCDR2
AASNLES

SEQ ID NO: 323 - protein, 9 aa, synthetic construct
  DOMAIN 1..9: note = P3-E4-LCDR3
QHSIEDPRT

SEQ ID NO: 324 - protein, 5 aa, synthetic construct
  DOMAIN 1..5: note = P3-F6-HCDR1
DYYMN

SEQ ID NO: 325 - protein, 17 aa, synthetic construct
  DOMAIN 1..17: note = P3-F6-HCDR2
DINPNNGGTSYNQKFKG

SEQ ID NO: 326 - protein, 10 aa, synthetic construct
  DOMAIN 1..10: note = P3-F6-HCDR3
KTWLPYASVY

SEQ ID NO: 327 - protein, 10 aa, synthetic construct
  DOMAIN 1..10: note = P3-F6-LCDR3
SQSTHVPPYT

SEQ ID NO: 328 - protein, 17 aa, synthetic construct
  DOMAIN 1..17: note = P3-D9-HCDR2
HISSGSSTIYYGDTVKG

SEQ ID NO: 329 - protein, 7 aa, synthetic construct
  DOMAIN 1..7: note = P3-D9-LCDR2
LTSSLAS

SEQ ID NO: 330 - protein, 118 aa, synthetic construct
  DOMAIN 1..118: note = P1-D2 VH
EFQLQQSGPELVKPGASVKISCKASGYSFTDHNMNWVKQSNGKSLEWIGVINPKYGTISNNQKFKGKATLTVDQSSSTAYMQLNSLTAEDSAVYYCARDDYPNYFDYWGQGTTLTVSS

SEQ ID NO: 331 - protein, 106 aa, synthetic construct
  DOMAIN 1..106: note = P1-D2 VL
QIVLTQSPAIMSASPGEKVTISCSANSSVNYIYWYQQKPGSSPKPWIYRTSNLASGVPARFSGSGSGTSYSLTISSMEAEDAATYYCQQYHSFPPTFGGGTKLEIK

SEQ ID NO: 332 - protein, 122 aa, synthetic construct
  DOMAIN 1..122: note = P1-D3 VH
QIQLIQSGPELKKPGETVKISCKASGYTFTTYGMSWVKQAPGKGLKWMGWINTYSGVPTYADDFKGRFAFSLETSANTAYLQINNLKNEDTATYFCARRGYSNLYYYALDYWGQGASVTVSS

SEQ ID NO: 333 - protein, 106 aa, synthetic construct
  DOMAIN 1..106: note = P1-D3 VL
QIVLTQSPAIMSASPGEKVTMTCSASSSVNYMHWYQQKSGTSPKRWIYDTSKLASGVPTRFSGSGSGTSYSLTISSMEAEDAATYFCQQWSSNPSTFGGGTKLEIK

SEQ ID NO: 334 - protein, 122 aa, synthetic construct
  DOMAIN 1..122: note = P1-C6 VH
QVQLQQSGAELARPGASVKLSCKASGYTFTSYGITWVKRRTGQGLGWIGEIYPRSGNTYYNEKFKGKATLTADKSSSTAYMELRSLTSEDSAVYFCARAITTVVGDWYFDVWGTGTTVTVSS

SEQ ID NO: 335 - protein, 110 aa, synthetic construct
  DOMAIN 1..110: note = P1-C6 VL
DIVLTQSPASLAMSLGKRATISCRASESVSLIGTNLIHWYQQKPGQPPKLLIHHASNLEAGVPARFSGSGSRTDFTLTIDPVEEDDVAIYYCLQSRKIRTFGGGTKLEIK

SEQ ID NO: 336 - protein, 121 aa, synthetic construct
  DOMAIN 1..121: note = P1-D9 VH
EVQLVESGGGLVKPGGSLKLSCAASGFSFSDYGMHWVRQAPEKGLEWIAHISSGSSTIYYADTVKGRFTISRDNAKNTLFLQMTSLRSEDTAMYYCARSRNYYGSRIFDYWGQGTTLTVSS

SEQ ID NO: 337 - protein, 106 aa, synthetic construct
  DOMAIN 1..106: note = P1-D9 VL
QIVLTQSPARMSASPGEKVTMTCSASSSVSYMYWYQQKPRSSPKPWIYLTSNLASGVPARFSGSGSGTSYSLTISSMEAEDAATYYCQQWSSNPPTFGSGTKLEIK

SEQ ID NO: 338 - protein, 121 aa, synthetic construct
  DOMAIN 1..121: note = P1-F10 VH
EVQLVESGGGLVKPGGSLKLSCAASGFAFSDYGMHWVRQAPEKGLEWVAHISSGSSTIYYADTVKGRFTISRDNVKNTLFLQMTSLRSEDTAMYYCARSRNYYGSRVFDYWGQGTPLTVSS

SEQ ID NO: 339 - protein, 106 aa, synthetic construct
  DOMAIN 1..106: note = P1-F10 VL
QIVLTQSPALMSVSPGEKVTMTCSASSSVNYMYWYQQKPRSSPKPWIYLTSNLASGVPARFSGSGSGTSYSLTISSMEAEDAATYYCQQWNSNPPTFGSGTKLEIK

SEQ ID NO: 340 - protein, 120 aa, synthetic construct
  DOMAIN 1..120: note = P2-C8 VH
EVQLQQSGPVLVKPGASVKMSCKVSGYTFTDYYMHWVKQSHGKSLEWIGLINPYNGVTSYNQKFKGKATLTVDKSSSTAYMELNSLTSEDSAVYYCARPPRAYGNYAGYWGQGTTLTVSS

SEQ ID NO: 341 - protein, 112 aa, synthetic construct
  DOMAIN 1..112: note = P2-C8 VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPYTFGGGTKLEIK

SEQ ID NO: 342 - protein, 119 aa, synthetic construct
  DOMAIN 1..119: note = P2-B9 VH
QVQLQQPGAELVKPGASVKLSCKASGYTFSSYWMHWVKQRPGQGLEWIGMIHPKSDSVNNNEKFKNKATLTVDKSSSTVYLQLSSLTSEDSAVYSCARGGDSGYSMDYWGQGTSVTVSS

SEQ ID NO: 343 - protein, 119 aa, synthetic construct
  DOMAIN 1..119: note = P2-B9 VL
DIVLTQSPASLAVSLGQKATISCKASKKVTIFGSISALHWYQQKPGQPPKLIYNGAKLESGVSARFSDSGSQNRSPFGNQLSFTLTIDPVEADDAATYYCLQNKEVPYTFGGGTKLEIK

SEQ ID NO: 344 - protein, 120 aa, synthetic construct
  DOMAIN 1..120: note = P3-D2 VH
EVQLQQSGPVLVKPGASVKMSCKASGYTFTDYFMHWVKQSHGKSLEWIGLINPYSGGTTYNQKFKGKATLTVDKSSSTAYMELNSLTSEDSAVYYCARPPRAYGDYAGYWGQGTTLTVSS

SEQ ID NO: 345 - protein, 112 aa, synthetic construct
  DOMAIN 1..112: note = P3-D2 VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPLTFGTGTKLEIK

SEQ ID NO: 346 - protein, 122 aa, synthetic construct
  DOMAIN 1..122: note = P3-B4 VH
QIQLVQSGPELKKPGEIVKISCKASGYTFTTYGMSWVKQAPGKGLKWMGWINTYSGVPTYADDFKGRFAFSLETSASTAYLQINNLKNEDTATYFCARRAGSSLYYYAMDYWGQGTSVTVSS

SEQ ID NO: 347 - protein, 106 aa, synthetic construct
  DOMAIN 1..106: note = P3-B4 VL
QIVLTQSPAIMSASPGEKVTMTCSASSSVSYMLWYQQKSGTSPKRWIYDTSKLASGVPARFSGSGSGTSYSLTISSMEAEDAATYYCQQWSSNPPTFGGGTKLEIK

SEQ ID NO: 348 - protein, 122 aa, synthetic construct
  DOMAIN 1..122: note = P3-E4 VH
QVQLQQPGAELVMPGAAVKLSCKASGYTFINYWMHWLKQRPGQGLEWIGEIDPSDGYTDYNQKFKGKATLTVDKSSSTAYMQLSSLTSEDSAVYYCAISLRGPITFGPFPYWGQGTLVTVSS

SEQ ID NO: 349 - protein, 111 aa, synthetic construct
  DOMAIN 1..111: note = P3-E4 VL
DIVLTQSPASLAVSLGQRATISCRASESVSIHGTHLMHWYQQKPGQPPKLLIYAASNLESGVPARFSGSGSETDFTLNIHPVAEEDAATYFCQHSIEDPRTFGGGTKLEIK

SEQ ID NO: 350 - protein, 119 aa, synthetic construct
  DOMAIN 1..119: note = P3-F6 VH
EVQLQQSGPELVKPGASVKISCKASGYTFTDYYMNWVKQSHGKSLEWIGDINPNNGGTSYNQKFKGKATLTVDKSSSTAYMELRSLTSEDSAVYYCARKTWLPYASVYWGQGTSVTVSS

SEQ ID NO: 351 - protein, 113 aa, synthetic construct
  DOMAIN 1..113: note = P3-F6 VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPPYTFGGGTKLEIK

SEQ ID NO: 352 - protein, 121 aa, synthetic construct
  DOMAIN 1..121: note = P3-D9 VH
EVQLVESGGGLVKPGGSLKLSCAASGFAFSDYGMHWVRQAPEKGLEWVAHISSGSSTIYYGDTVKGRFTISRDNAKNTLFLQMTSLRSEDTAMYYCTRSRNYYGSRVFDYWGQGTTLTVSS

SEQ ID NO: 353 - protein, 106 aa, synthetic construct
  DOMAIN 1..106: note = P3-D9 VL
QIVLTQSPALMSVSPGEKVTMTCSASSSVSYMYWYQQKPRSSPKPWIYLTSSLASGVPARFSGSGSGTSYSLTINNVEAEDAATYYCQQWSSNPPTFGSGTKVEIK

SEQ ID NO: 354 - protein, 448 aa, synthetic construct
  CHAIN 1..448: note = M81CHI-heavy chain
EFQLQQSGPELVKPGASVKISCKASGYSFTDHNMNWVKQSNGKSLEWIGVMNPKFGTINYNQKFKDKATLTVDQSSSTAYMQLNSLTSEDSAVYYCVRDDYPNYFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 355 - protein, 213 aa, synthetic construct
  DOMAIN 1..213: note = M81CHI-light chain
QIVLTQSPAIMSASPGEKVTISCSASSSVNYMYWYQQKPGSSPKSWIYRTSNLASGVSARFSGSGSGTSYSLTISSMEAEDAATYYCQQYHDYPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 356 - protein, 450 aa, synthetic construct
  CHAIN 1..450: note = P3-C7 CHI-heavy chain
EVQLQQSGPVLLKPGASVKMSCKASGYTFTDHYIHWVKLSHGKSLEWIGVINPYNDGATYNQKFKGKATLTVDKSSSTAYMELNSLTSEDSAVYYCARPPRAYGNYAGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 357 - protein, 219 aa, synthetic construct
  DOMAIN 1..219: note = P3-C7 CHI-light chain
DVVMTQTPLSLPVSLGDQASISCRSSQSLVHSNGNTYLHWYLQKPGQSPKLLIYKVSNRFSGVPDRFIGSGSRTDFTLKISRVEAEDLGVYFCSQSTHVPYTFGGGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0028421 A1
(43) Pub. Date: Jan. 29, 2026

(54) ANTIGEN-BINDING MOLECULE SPECIFICALLY BINDING TO GUCY2C AND CD3 AND PHARMACEUTICAL USE THEREOF

(71) Applicants: JIANGSU HENGRUI PHARMACEUTICALS CO., LTD., Lianyungang (CN); SHANGHAI HENGRUI PHARMACEUTICAL CO., LTD., Shanghai (CN)

(72) Inventors: Xin YE, Shanghai (CN); Yuxiao CHEN, Shanghai (CN); Abula AMINA, Shanghai (CN); Hua YING, Shanghai (CN); Weikang TAO, Shanghai (CN)

(21) Appl. No.: 18/996,788

(22) PCT Filed: Aug. 4, 2023
(86) PCT No.: PCT/CN2023/111149

§ 371 Date: Jan. 17, 2025

(30) Foreign Application Priority Data
Aug. 5, 2022 (CN) 202210937340.1
May 16, 2023 (CN) 202310549681.6

(51) Int. Cl. C07K 16/40; A61K 39/00; A61P 35/00; C07K 16/28

(52) U.S. Cl. CPC C07K 16/40; A61P 35/00; C07K 16/2809; C12Y 406/01002; A61K 2039/505; C07K 2317/24; C07K 2317/31; C07K 2317/92

(57) ABSTRACT

Provided are an antigen-binding molecule specifically binding to GUCY2C and CD3 and pharmaceutical use thereof. The antigen-binding molecule can be used for treating tumor.

### TECHNICAL FIELD

The present disclosure relates to the field of biotechnology, and more specifically, the present disclosure relates to an antigen-binding molecule that specifically binds to GUCY2C and CD3 and medical use thereof.

### BACKGROUND

The statements herein merely provide background information related to the present disclosure and may not necessarily constitute the prior art.

GUCY2C (Guanylyl Cyclase C) belongs to the family of transmembrane guanylate cyclases. It consists of an extracellular domain, a single transmembrane domain, and an intracellular catalytic domain and is anchored to the cell membrane as a homodimer or trimer. Its main function is to catalyze GTP to form cGMP so as to maintain normal physiological functions such as intestinal homeostasis (Michaela Kuhn, Physiological Reviews: (2016), 96(2):751-804). In normal tissues, GUCY2C is expressed only in intestinal epithelial cells, with no or very low expression in other tissues. Meanwhile, GUCY2C is expressed in gastrointestinal malignancies, including in no less than 90% of colorectal cancer cases at various stages and no less than 50% of gastric or gastroesophageal junction cancer cases (Ruth Birbe M D., Human Pathology, (2005), 36(2):170-179; Hadi Danaee, PLoS One, (2017), 12(12): e0189953). In addition, because the expression polarity at the cellular level and the enrichment degree of blood vessels in normal intestinal tissues are significantly different from those in intestinal cancer, the probability of GUCY2C in the intestinal cancers contacting the blood vessels is higher than that in the normal tissues (Divya Mathur, Clinical Cancer Research, (2020), 26(9): 2188-2202). Therefore, GUCY2C is a specific and highly potential target for gastrointestinal cancer, especially intestinal cancer.

The bispecific antibody targeting CD3 and TAA is a novel immunotherapy, and it can bind to T cells and tumor cells simultaneously and simulate the interaction between MHC and TCR, leading to the formation of a lytic synapse in T cells, which then release perform and granzymes to specifically kill tumor cells. Activated T cells can release cytokines, initiate other immune cells, and expand immune responses against tumors, ultimately leading to T cell proliferation and a cascade for killing tumor cells.

### SUMMARY

The present disclosure provides an antigen-binding molecule that specifically binds to GUCY2C and CD3 and an antibody that specifically binds to GUCY2C.

In one aspect, the present disclosure provides an antigen-binding molecule that specifically binds to GUCY2C and CD3. The antigen-binding molecule comprises at least one antigen-binding moiety that specifically binds to GUCY2C and at least one antigen-binding moiety that specifically binds to CD3, wherein the antigen-binding moiety that specifically binds to GUCY2C comprises a heavy chain variable region GUCY2C-VH and a light chain variable region GUCY2C-VL, and the antigen-binding moiety that specifically binds to CD3 comprises a heavy chain variable region CD3-VH and a light chain variable region CD3-VL.

In some embodiments, provided is the antigen-binding molecule according to the above, wherein:
(i) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 25, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 54, 26, 50, 51, 52, or 53, respectively; or
(ii) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 37 or 15, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 16, 34, 35, or 36, respectively; or
(iii) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 42 or 17, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 41 or 18, respectively; or
iv-1) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 273, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 274, respectively; or
iv-2) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 275, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 276, respectively; or
iv-3) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 277, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 278, respectively; or
iv-4) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 279, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 280, respectively; or
iv-5) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 330, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 331, respectively; or
iv-6) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 332, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 333, respectively; or
iv-7) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 334, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 335, respectively; or
iv-8) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 336, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 337, respectively; or
iv-9) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 338, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 339, respectively; or
iv-10) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 340, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 341, respectively; or
iv-11) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 342, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 343, respectively; or
iv-12) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 344, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 345, respectively; or
iv-13) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 346, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 347, respectively; or
iv-14) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 348, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 349, respectively; or
iv-15) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 350, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 351, respectively; or
iv-16) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 352, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 353, respectively.

In some embodiments, provided is the antigen-binding molecule according to the above, wherein:
(i) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 25, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 54, respectively, or
(ii) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 37, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 16, respectively, or
(iii) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 42, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 41, respectively.

In some embodiments, the GUCY2C-HCDR1, the GUCY2C-HCDR2, the GUCY2C-HCDR3, the GUCY2C-LCDR1, the GUCY2C-LCDR2, and the GUCY2C-LCDR3 are defined according to the Kabat, IMGT, Chothia, AbM, or Contact numbering scheme.

In some embodiments, provided is the antigen-binding molecule according to any one of the above, wherein:
i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 47, 22, 43, 44, 45, or 46, a GUCY2C-LCDR2 comprising the amino acid sequence of SEQ ID NO: 23, and a GUCY2C-LCDR3 comprising the amino acid sequence of SEQ ID NO: 24; or
ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27 or 6; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, 28, 29, or 30, and a GUCY2C-LCDR3 comprising the amino acid sequence of SEQ ID NO: 9; or
iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40 or 12; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39 or 8, and a GUCY2C-LCDR3 comprising the amino acid sequence of SEQ ID NO: 14; or
iv-1) the GUCY2C-VH comprises: a GUCY2C-HCDR1 comprising the amino acid sequence of SEQ ID NO: 250, a GUCY2C-HCDR2 comprising the amino acid sequence of SEQ ID NO: 251, and a GUCY2C-HCDR3 comprising the amino acid sequence of SEQ ID NO: 252; and the GUCY2C-VL comprises: a GUCY2C-LCDR1 comprising the amino acid sequence of SEQ ID NO: 253, a GUCY2C-LCDR2 comprising the amino acid sequence of SEQ ID NO: 254, and a GUCY2C-LCDR3 comprising the amino acid sequence of SEQ ID NO: 255; or
iv-2) the GUCY2C-VH comprises: a GUCY2C-HCDR1 comprising the amino acid sequence of SEQ ID NO: 256, a GUCY2C-HCDR2 comprising the amino acid sequence of SEQ ID NO: 257, and a GUCY2C-HCDR3 comprising the amino acid sequence of SEQ ID NO: 258; and the GUCY2C-VL comprises: a GUCY2C-LCDR1 comprising the amino acid sequence of SEQ ID NO: 259, a GUCY2C-LCDR2 comprising the amino acid sequence of SEQ ID NO: 8, and a GUCY2C-LCDR3 comprising the amino acid sequence of SEQ ID NO: 260; or
iv-3) the GUCY2C-VH comprises: a GUCY2C-HCDR1 comprising the amino acid sequence of SEQ ID NO: 261, a GUCY2C-HCDR2 comprising the amino acid sequence of SEQ ID NO: 262, and a GUCY2C-HCDR3 comprising the amino acid sequence of SEQ ID NO: 263; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 265, and a GUCY2C-LCDR3 comprising the amino acid sequence of SEQ ID NO: 266; or
iv-4) the GUCY2C-VH comprises: a GUCY2C-HCDR1 comprising the amino acid sequence of SEQ ID NO: 267, a GUCY2C-HCDR2 comprising the amino acid sequence of SEQ ID NO: 268, and a GUCY2C-HCDR3 comprising the amino acid sequence of SEQ ID NO: 269; and the GUCY2C-VL comprises: a GUCY2C-LCDR1 comprising the amino acid sequence of SEQ ID NO: 270, a GUCY2C-LCDR2 comprising the amino acid sequence of SEQ ID NO: 271, and a GUCY2C-LCDR3 comprising the amino acid sequence of SEQ ID NO: 272; or
iv-5) the GUCY2C-VH comprises: a GUCY2C-HCDR1 comprising the amino acid sequence of SEQ ID NO: 281, a GUCY2C-HCDR2 comprising the amino acid sequence of SEQ ID NO: 282, and a GUCY2C-HCDR3 comprising the amino acid sequence of SEQ ID NO: 283; and the GUCY2C-VL comprises: a GUCY2C-LCDR1 comprising the amino acid sequence of SEQ ID NO: 284, a GUCY2C-LCDR2 comprising the amino acid sequence of SEQ ID NO: 8, and a GUCY2C-LCDR3 comprising the amino acid sequence of SEQ ID NO: 285; or
iv-6) the GUCY2C-VH comprises: a GUCY2C-HCDR1 comprising the amino acid sequence of SEQ ID NO: 286, a GUCY2C-HCDR2 comprising the amino acid sequence of SEQ ID NO: 287, and a GUCY2C-HCDR3 comprising the amino acid sequence of SEQ ID NO: 288; and the GUCY2C-VL comprises: a GUCY2C-LCDR1 comprising the amino acid sequence of SEQ ID NO: 289, a GUCY2C-LCDR2 comprising the amino acid sequence of SEQ ID NO: 265, and a GUCY2C-LCDR3 comprising the amino acid sequence of SEQ ID NO: 290; or
iv-7) the GUCY2C-VH comprises: a GUCY2C-HCDR1 comprising the amino acid sequence of SEQ ID NO: 291, a GUCY2C-HCDR2 comprising the amino acid sequence of SEQ ID NO: 292, and a GUCY2C-HCDR3 comprising the amino acid sequence of SEQ ID NO: 293; and the GUCY2C-VL comprises: a GUCY2C-LCDR1 comprising the amino acid sequence of SEQ ID NO: 294, a GUCY2C-LCDR2 comprising the amino acid sequence of SEQ ID NO: 295, and a GUCY2C-LCDR3 comprising the amino acid sequence of SEQ ID NO: 296; or
iv-8) the GUCY2C-VH comprises: a GUCY2C-HCDR1 comprising the amino acid sequence of SEQ ID NO: 261, a GUCY2C-HCDR2 comprising the amino acid sequence of SEQ ID NO: 297, and a GUCY2C-HCDR3 comprising the amino acid sequence of SEQ ID NO: 298; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 299, and a GUCY2C-LCDR3 comprising the amino acid sequence of SEQ ID NO: 300; or
iv-9) the GUCY2C-VH comprises: a GUCY2C-HCDR1 comprising the amino acid sequence of SEQ ID NO: 261, a GUCY2C-HCDR2 comprising the amino acid sequence of SEQ ID NO: 301, and a GUCY2C-HCDR3 comprising the amino acid sequence of SEQ ID NO: 302; and the GUCY2C-VL comprises: a GUCY2C-LCDR1 comprising the amino acid sequence of SEQ ID NO: 303, a GUCY2C-LCDR2 comprising the amino acid sequence of SEQ ID NO: 299, and a GUCY2C-LCDR3 comprising the amino acid sequence of SEQ ID NO: 304; or
iv-10) the GUCY2C-VH comprises: a GUCY2C-HCDR1 comprising the amino acid sequence of SEQ ID NO: 305, a GUCY2C-HCDR2 comprising the amino acid sequence of SEQ ID NO: 306, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 24; or
iv-11) the GUCY2C-VH comprises: a GUCY2C-HCDR1 comprising the amino acid sequence of SEQ ID NO: 128, a GUCY2C-HCDR2 comprising the amino acid sequence of SEQ ID NO: 307, and a GUCY2C-HCDR3 comprising the amino acid sequence of SEQ ID NO: 308; and the GUCY2C-VL comprises: a GUCY2C-LCDR1 comprising the amino acid sequence of SEQ ID NO: 309, a GUCY2C-LCDR2 comprising the amino acid sequence of SEQ ID NO: 310, and a GUCY2C-LCDR3 comprising the amino acid sequence of SEQ ID NO: 311; or
iv-12) the GUCY2C-VH comprises: a GUCY2C-HCDR1 comprising the amino acid sequence of SEQ ID NO: 312, a GUCY2C-HCDR2 comprising the amino acid sequence of SEQ ID NO: 313, and a GUCY2C-HCDR3 comprising the amino acid sequence of SEQ ID NO: 314; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 315; or
iv-13) the GUCY2C-VH comprises: a GUCY2C-HCDR1 comprising the amino acid sequence of SEQ ID NO: 286, a GUCY2C-HCDR2 comprising the amino acid sequence of SEQ ID NO: 287, and a GUCY2C-HCDR3 comprising the amino acid sequence of SEQ ID NO: 316; and the GUCY2C-VL comprises: a GUCY2C-LCDR1 comprising the amino acid sequence of SEQ ID NO: 317, a GUCY2C-LCDR2 comprising the amino acid sequence of SEQ ID NO: 265, and a GUCY2C-LCDR3 comprising the amino acid sequence of SEQ ID NO: 300; or
iv-14) the GUCY2C-VH comprises: a GUCY2C-HCDR1 comprising the amino acid sequence of SEQ ID NO: 318, a GUCY2C-HCDR2 comprising the amino acid sequence of SEQ ID NO: 319, and a GUCY2C-HCDR3 comprising the amino acid sequence of SEQ ID NO: 320; and the GUCY2C-VL comprises: a GUCY2C-LCDR1 comprising the amino acid sequence of SEQ ID NO: 321, a GUCY2C-LCDR2 comprising the amino acid sequence of SEQ ID NO: 322, and a GUCY2C-LCDR3 comprising the amino acid sequence of SEQ ID NO: 323; or
iv-15) the GUCY2C-VH comprises: a GUCY2C-HCDR1 comprising the amino acid sequence of SEQ ID NO: 324, a GUCY2C-HCDR2 comprising the amino acid sequence of SEQ ID NO: 325, and a GUCY2C-HCDR3 comprising the amino acid sequence of SEQ ID NO: 326; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 327; or
iv-16) the GUCY2C-VH comprises: a GUCY2C-HCDR1 comprising the amino acid sequence of SEQ ID NO: 261, a GUCY2C-HCDR2 comprising the amino acid sequence of SEQ ID NO: 328, and a GUCY2C-HCDR3 comprising the amino acid sequence of SEQ ID NO: 302; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 329, and a GUCY2C-LCDR3 comprising the amino acid sequence of SEQ ID NO: 300.

In some embodiments, provided is the antigen-binding molecule according to any one of the above, wherein:
i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 47, a GUCY2C-LCDR2 comprising the amino acid sequence of SEQ ID NO: 23, and a GUCY2C-LCDR3 comprising the amino acid sequence of SEQ ID NO: 24; or
ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, and a GUCY2C-LCDR3 comprising the amino acid sequence of SEQ ID NO: 9; or
iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39, and a GUCY2C-LCDR3 comprising the amino acid sequence of SEQ ID NO: 14.

In some embodiments, provided is the antigen-binding molecule according to any one of the above, wherein:
i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 24; or
ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 6; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, and a GUCY2C-LCDR3 comprising the amino acid sequence of SEQ ID NO: 9; or
iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 12; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 8, and a GUCY2C-LCDR3 comprising the amino acid sequence of SEQ ID NO: 14.

In some embodiments, provided is the antigen-binding molecule according to any one of the above, wherein:
i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 43, a GUCY2C-LCDR2 comprising the amino acid sequence of SEQ ID NO: 23, and a GUCY2C-LCDR3 comprising the amino acid sequence of SEQ ID NO: 24; or
the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 44, a GUCY2C-LCDR2 comprising the amino acid sequence of SEQ ID NO: 23, and a GUCY2C-LCDR3 comprising the amino acid sequence of SEQ ID NO: 24; or
the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 45, a GUCY2C-LCDR2 comprising the amino acid sequence of SEQ ID NO: 23, and a GUCY2C-LCDR3 comprising the amino acid sequence of SEQ ID NO: 24; or
ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 6; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 28, and a GUCY2C-LCDR3 comprising the amino acid sequence of SEQ ID NO: 9; or
the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 6; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 30, and a GUCY2C-LCDR3 comprising the amino acid sequence of SEQ ID NO: 9.

In some embodiments, provided is the antigen-binding molecule according to any one of the above, wherein the GUCY2C-HCDR1, the GUCY2C-HCDR2, the GUCY2C-HCDR3, the GUCY2C-LCDR1, the GUCY2C-LCDR2, and the GUCY2C-LCDR3 are defined according to the Kabat numbering scheme.

In some embodiments, provided is the antigen-binding molecule according to any one of the above, wherein the GUCY2C-VH and the GUCY2C-VL are humanized and each comprise the FR region of a human antibody.

In some embodiments, provided is the antigen-binding molecule according to any one of the above, wherein:
(i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21, and the FR region of the GUCY2C-VH comprises one or more amino acid substitutions selected from the group consisting of 1E, 39L, 43K, 69L, 71V, and 73K; and the GUCY2C-VL comprises: a GUCY2C-LCDR1 comprising the amino acid sequence of SEQ ID NO: 47, 22, 43, 44, 45, or 46, a GUCY2C-LCDR2 comprising the amino acid sequence of SEQ ID NO: 23, and a GUCY2C-LCDR3 comprising the amino acid sequence of SEQ ID NO: 24, and the FR region of the GUCY2C-VL comprises one or more amino acid substitutions selected from the group consisting of 2V, 3V, 68R, and 74K; or
(ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27 or 6, and the FR region of the GUCY2C-VH comprises one or more amino acid substitutions selected from the group consisting of 1E, 38K, 66K, and 67A; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, 28, 29, or 30, and a GUCY2C-LCDR3 comprising the amino acid sequence of SEQ ID NO: 9, and the FR region of the GUCY2C-VL comprises one or more amino acid substitutions selected from the group consisting of 15P, 16G, 42S, 43S, 45K, 46S, 47W, 49Y, 58V, 71Y, and 76S; or
(iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40 or 12, and the FR region of the GUCY2C-VH comprises one or more amino acid substitutions selected from the group consisting of 1E, 2F, 28S, 69L, 71V, 73Q, and 75S; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39 or 8, and a GUCY2C-LCDR3 comprising the amino acid sequence of SEQ ID NO: 14, and the FR region of the GUCY2C-VL comprises one or more amino acid substitutions selected from the group consisting of 42S, 43S, 46P, 47W, 58V, and 71Y.

In some embodiments, provided is the antigen-binding molecule according to any one of the above, wherein:
(i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21, and the FR region of the GUCY2C-VH comprises one or more amino acid substitutions selected from the group consisting of 1E, 39L, 43K, 69L, 71V, and 73K; and the GUCY2C-VL comprises: a GUCY2C-LCDR1 comprising the amino acid sequence of SEQ ID NO: 47, a GUCY2C-LCDR2 comprising the amino acid sequence of SEQ ID NO: 23, and a GUCY2C-LCDR3 comprising the amino acid sequence of SEQ ID NO: 24, and the FR region of the GUCY2C-VL comprises one or more amino acid substitutions selected from the group consisting of 2V, 3V, 68R, and 74K; or
(ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27, and the FR region of the GUCY2C-VH comprises one or more amino acid substitutions selected from the group consisting of 1E, 38K, 66K, and 67A; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, and a GUCY2C-LCDR3 comprising the amino acid sequence of SEQ ID NO: 9, and the FR region of the GUCY2C-VL comprises one or more amino acid substitutions selected from the group consisting of 15P, 16G, 42S, 43S, 45K, 46S, 47W, 49Y, 58V, 71Y, and 76S; or
(iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40, and the FR region of the GUCY2C-VH comprises one or more amino acid substitutions selected from the group consisting of 1E, 2F, 28S, 69L, 71V, 73Q, and 75S; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39, and a GUCY2C-LCDR3 comprising the amino acid sequence of SEQ ID NO: 14, and the FR region of the GUCY2C-VL comprises one or more amino acid substitutions selected from the group consisting of 42S, 43S, 46P, 47W, 58V, and 71Y.

In some embodiments, provided is the antigen-binding molecule according to any one of the above, wherein:
the CD3-VH comprises: a CD3-HCDR1 comprising the amino acid sequence of SEQ ID NO: 71, a CD3-HCDR2 comprising the amino acid sequence of SEQ ID NO: 72, and a CD3-HCDR3 comprising the amino acid sequence of SEQ ID NO: 73 or 88; and the CD3-VL comprises: a CD3-LCDR1 comprising the amino acid sequence of SEQ ID NO: 74, a CD3-LCDR2 comprising the amino acid sequence of SEQ ID NO: 75, and a CD3-LCDR3 comprising the amino acid sequence of SEQ ID NO: 76.

In some embodiments, provided is the antigen-binding molecule according to any one of the above, wherein:
the CD3-VH comprises: a CD3-HCDR1 comprising the amino acid sequence of SEQ ID NO: 71, a CD3-HCDR2 comprising the amino acid sequence of SEQ ID NO: 72, and a CD3-HCDR3 comprising the amino acid sequence of SEQ ID NO: 73; and the CD3-VL comprises: a CD3-LCDR1 comprising the amino acid sequence of SEQ ID NO: 74, a CD3-LCDR2 comprising the amino acid sequence of SEQ ID NO: 75, and a CD3-LCDR3 comprising the amino acid sequence of SEQ ID NO: 76.

In some embodiments, provided is the antigen-binding molecule according to any one of the above, wherein:
the CD3-VH comprises: a CD3-HCDR1 comprising the amino acid sequence of SEQ ID NO: 71, a CD3-HCDR2 comprising the amino acid sequence of SEQ ID NO: 72, and a CD3-HCDR3 comprising the amino acid sequence of SEQ ID NO: 88; and the CD3-VL comprises: a CD3-LCDR1 comprising the amino acid sequence of SEQ ID NO: 74, a CD3-LCDR2 comprising the amino acid sequence of SEQ ID NO: 75, and a CD3-LCDR3 comprising the amino acid sequence of SEQ ID NO: 76.

In some embodiments, provided is the antigen-binding molecule according to any one of the above, wherein:
i) the GUCY2C-VH comprises the amino acid sequence of SEQ ID NO: 56, 25, or 57, and the GUCY2C-VL comprises the amino acid sequence of SEQ ID NO: 54, 55, 26, 48, 49, 50, 51, 52, or 53; or
ii) the GUCY2C-VH comprises the amino acid sequence of SEQ ID NO: 37, 15, or 38, and the GUCY2C-VL comprises the amino acid sequence of SEQ ID NO: 33, 16, 31, 32, 34, 35, or 36; or
iii) the GUCY2C-VH comprises the amino acid sequence of SEQ ID NO: 42 or 17, and the GUCY2C-VL comprises the amino acid sequence of SEQ ID NO: 41 or 18; or
iv-1) the GUCY2C-VH comprises the amino acid sequence of SEQ ID NO: 273, and the GUCY2C-VL comprises the amino acid sequence of SEQ ID NO: 274; or
iv-2) the GUCY2C-VH comprises the amino acid sequence of SEQ ID NO: 275, and the GUCY2C-VL comprises the amino acid sequence of SEQ ID NO: 276; or
iv-3) the GUCY2C-VH comprises the amino acid sequence of SEQ ID NO: 277, and the GUCY2C-VL comprises the amino acid sequence of SEQ ID NO: 278; or
iv-4) the GUCY2C-VH comprises the amino acid sequence of SEQ ID NO: 279, and the GUCY2C-VL comprises the amino acid sequence of SEQ ID NO: 280; or
iv-5) the GUCY2C-VH comprises the amino acid sequence of SEQ ID NO: 330, and the GUCY2C-VL comprises the amino acid sequence of SEQ ID NO: 331; or
iv-6) the GUCY2C-VH comprises the amino acid sequence of SEQ ID NO: 332, and the GUCY2C-VL comprises the amino acid sequence of SEQ ID NO: 333; or
iv-7) the GUCY2C-VH comprises the amino acid sequence of SEQ ID NO: 334, and the GUCY2C-VL comprises the amino acid sequence of SEQ ID NO: 335; or
iv-8) the GUCY2C-VH comprises the amino acid sequence of SEQ ID NO: 336, and the GUCY2C-VL comprises the amino acid sequence of SEQ ID NO: 337; or
iv-9) the GUCY2C-VH comprises the amino acid sequence of SEQ ID NO: 338, and the GUCY2C-VL comprises the amino acid sequence of SEQ ID NO: 339; or
iv-10) the GUCY2C-VH comprises the amino acid sequence of SEQ ID NO: 340, and the GUCY2C-VL comprises the amino acid sequence of SEQ ID NO: 341; or
iv-11) the GUCY2C-VH comprises the amino acid sequence of SEQ ID NO: 342, and the GUCY2C-VL comprises the amino acid sequence of SEQ ID NO: 343; or
iv-12) the GUCY2C-VH comprises the amino acid sequence of SEQ ID NO: 344, and the GUCY2C-VL comprises the amino acid sequence of SEQ ID NO: 345; or
iv-13) the GUCY2C-VH comprises the amino acid sequence of SEQ ID NO: 346, and the GUCY2C-VL comprises the amino acid sequence of SEQ ID NO: 347; or
iv-14) the GUCY2C-VH comprises the amino acid sequence of SEQ ID NO: 348, and the GUCY2C-VL comprises the amino acid sequence of SEQ ID NO: 349; or
iv-15) the GUCY2C-VH comprises the amino acid sequence of SEQ ID NO: 350, and the GUCY2C-VL comprises the amino acid sequence of SEQ ID NO: 351; or
iv-16) the GUCY2C-VH comprises the amino acid sequence of SEQ ID NO: 352, and the GUCY2C-VL comprises the amino acid sequence of SEQ ID NO: 353.

In some embodiments, provided is the antigen-binding molecule according to any one of the above, wherein:
i) the GUCY2C-VH comprises the amino acid sequence of SEQ ID NO: 56, and the GUCY2C-VL comprises the amino acid sequence of SEQ ID NO: 54, 55, 48, 49, 50, 51, or 52, or
the GUCY2C-VH comprises the amino acid sequence of SEQ ID NO: 25, and the GUCY2C-VL comprises the amino acid sequence of SEQ ID NO: 26, or
ii) the GUCY2C-VH comprises the amino acid sequence of SEQ ID NO: 37, and the GUCY2C-VL comprises the amino acid sequence of SEQ ID NO: 33, or
the GUCY2C-VH comprises the amino acid sequence of SEQ ID NO: 38, and the GUCY2C-VL comprises the amino acid sequence of SEQ ID NO: 33, 32, 34, or 36, or
the GUCY2C-VH comprises the amino acid sequence of SEQ ID NO: 15, and the GUCY2C-VL comprises the amino acid sequence of SEQ ID NO: 16, or
iii) the GUCY2C-VH comprises the amino acid sequence of SEQ ID NO: 42, and the GUCY2C-VL comprises the amino acid sequence of SEQ ID NO: 41, or
the GUCY2C-VH comprises the amino acid sequence of SEQ ID NO: 17, and the GUCY2C-VL comprises the amino acid sequence of SEQ ID NO: 18.

In some embodiments, provided is the antigen-binding molecule according to any one of the above, wherein:
i) the GUCY2C-VH comprises the amino acid sequence of SEQ ID NO: 56, and the GUCY2C-VL comprises the amino acid sequence of SEQ ID NO: 54, or
the GUCY2C-VH comprises the amino acid sequence of SEQ ID NO: 56, and the GUCY2C-VL comprises the amino acid sequence of SEQ ID NO: 55; or
ii) the GUCY2C-VH comprises the amino acid sequence of SEQ ID NO: 37, and the GUCY2C-VL comprises the amino acid sequence of SEQ ID NO: 33; or
iii) the GUCY2C-VH comprises the amino acid sequence of SEQ ID NO: 42, and the GUCY2C-VL comprises the amino acid sequence of SEQ ID NO: 41.

In some embodiments, provided is the antigen-binding molecule according to any one of the above, wherein the amino acid sequence of the GUCY2C-VH is set forth in SEQ ID NO: 56, and the amino acid sequence of the GUCY2C-VL is set forth in SEQ ID NO: 54.

In some embodiments, provided is the antigen-binding molecule according to any one of the above, wherein the amino acid sequence of the GUCY2C-VH is set forth in SEQ ID NO: 56, and the amino acid sequence of the GUCY2C-VL is set forth in SEQ ID NO: 55.

In some embodiments, provided is the antigen-binding molecule according to any one of the above, wherein the amino acid sequence of the GUCY2C-VH is set forth in SEQ ID NO: 37, and the amino acid sequence of the GUCY2C-VL is set forth in SEQ ID NO: 33.

In some embodiments, provided is the antigen-binding molecule according to any one of the above, wherein the amino acid sequence of the GUCY2C-VH is set forth in SEQ ID NO: 42, and the amino acid sequence of the GUCY2C-VL is set forth in SEQ ID NO: 41.

In some embodiments, provided is the antigen-binding molecule according to any one of the above, wherein:
the CD3-VH comprises the amino acid sequence of SEQ ID NO: 77 or 89, and the CD3-VL comprises the amino acid sequence of SEQ ID NO: 78.

In some embodiments, provided is the antigen-binding molecule according to any one of the above, wherein:
the amino acid sequence of the CD3-VH is set forth in SEQ ID NO: 77, and the amino acid sequence of the CD3-VL is set forth in SEQ ID NO: 78.

In some embodiments, provided is the antigen-binding molecule according to any one of the above, wherein:
the amino acid sequence of the CD3-VH is set forth in SEQ ID NO: 89, and the amino acid sequence of the CD3-VL is set forth in SEQ ID NO: 78.

In some embodiments, provided is the antigen-binding molecule according to any one of the above, wherein the antigen-binding moiety that specifically binds to GUCY2C or the antigen-binding moiety that specifically binds to CD3 comprises a Titin chain and an Obscurin chain capable of forming a dimer.

In some embodiments, provided is the antigen-binding molecule according to any one of the above, wherein the antigen-binding moiety that specifically binds to GUCY2C comprises a Titin chain and an Obscurin chain capable of forming a dimer.

In some embodiments, provided is the antigen-binding molecule according to any one of the above, wherein the antigen-binding moiety that specifically binds to CD3 comprises a Titin chain and an Obscurin chain capable of forming a dimer.

In some embodiments, provided is the antigen-binding molecule according to any one of the above, wherein the Titin chain comprises an amino acid sequence selected from the group consisting of SEQ ID NO: 182 to SEQ ID NO: 200, and the Obscurin chain comprises an amino acid sequence selected from the group consisting of SEQ ID NO: 201 to SEQ ID NO: 241.

In some embodiments, provided is the antigen-binding molecule according to any one of the above, wherein the Titin chain comprises the amino acid sequence of SEQ ID NO: 198.

In some embodiments, provided is the antigen-binding molecule according to any one of the above, wherein the Obscurin chain comprises the amino acid sequence of SEQ ID NO: 236.

In some embodiments, provided is the antigen-binding molecule according to any one of the above, wherein the antigen-binding molecule comprises an Fc region.

In some embodiments, provided is the antigen-binding molecule according to any one of the above, wherein the antigen-binding molecule comprises an IgG Fc region.

In some embodiments, provided is the antigen-binding molecule according to any one of the above, wherein the antigen-binding molecule comprises an IgG1 Fc region.

In some embodiments, provided is the antigen-binding molecule according to any one of the above, wherein the antigen-binding molecule comprises an Fc region, and the Fc region comprises one or more amino acid substitutions capable of reducing the binding of the Fc region to an Fcγ receptor.

In some embodiments, provided is the antigen-binding molecule according to any one of the above, wherein the antigen-binding molecule comprises an Fc region; the Fc region is a human IgG1 Fc region, and the amino acids at positions 234 and 235 are A, as numbered according to the EU index.

In some embodiments, provided is the antigen-binding molecule according to any one of the above, wherein the antigen-binding molecule comprises an Fc region; the Fc region comprises a first subunit Fc1 and a second subunit Fc2 capable of associating with each other, and the Fc1 and the Fc2 each independently have one or more amino acid substitutions that reduce homodimerization of the Fc region.

In some embodiments, provided is the antigen-binding molecule according to any one of the above, wherein the antigen-binding molecule comprises an Fc region; the Fc region comprises a first subunit Fc1 and a second subunit Fc2 capable of associating with each other, the Fc1 has a knob structure according to the knob-into-hole technique, and the Fc2 has a hole structure according to the knob-into-hole technique.

In some embodiments, provided is the antigen-binding molecule according to any one of the above, wherein the antigen-binding molecule comprises an Fc region; the Fc region comprises a first subunit Fc1 and a second subunit Fc2 capable of associating with each other, the Fc1 has a knob structure according to the knob-into-hole technique, and the Fc2 has a hole structure according to the knob-into-hole technique; for the Fc1, the amino acid at position 366 is W, and for the Fc2, the amino acid at position 366 is S, the amino acid at position 368 is A, and the amino acid at position 407 is V, as numbered according to the EU index.

In some embodiments, provided is the antigen-binding molecule according to any one of the above, wherein the antigen-binding molecule comprises an Fc region; the Fc region comprises a first subunit Fc1 and a second subunit Fc2 capable of associating with each other, the Fc1 comprises the amino acid sequence of SEQ ID NO: 79 or 85, and the Fc2 comprises the amino acid sequence of SEQ ID NO: 80 or 86.

In some embodiments, provided is the antigen-binding molecule according to any one of the above, wherein the antigen-binding molecule comprises an Fc region; the Fc region comprises a first subunit Fc1 and a second subunit Fc2 capable of associating with each other, the Fc1 has a knob structure according to the knob-into-hole technique, and the Fc2 has a hole structure according to the knob-into-hole technique; for the Fc1, the amino acid at position 354 is C and the amino acid at position 366 is W, and for the Fc2, the amino acid at position 349 is C, the amino acid at position 366 is S, the amino acid at position 368 is A, and the amino acid at position 407 is V, as numbered according to the EU index.

In some embodiments, provided is the antigen-binding molecule according to any one of the above, wherein the antigen-binding molecule comprises an Fc region; the Fc region comprises a first subunit Fc1 and a second subunit Fc2 capable of associating with each other, the Fc1 comprises the amino acid sequence of SEQ ID NO: 85, and the Fc2 comprises the amino acid sequence of SEQ ID NO: 86.

In some embodiments, provided is the antigen-binding molecule according to any one of the above, wherein the antigen-binding molecule comprises an Fc region; the Fc region comprises a first subunit Fc1 and a second subunit Fc2 capable of associating with each other, the Fc1 has a knob structure according to the knob-into-hole technique, and the Fc2 has a hole structure according to the knob-into-hole technique; for the Fc1, the amino acid at position 237 is A, the amino acid at position 349 is C, and the amino acid at position 366 is W, and for the Fc2, the amino acid at position 237 is A, the amino acid at position 354 is C, the amino acid at position 366 is S, the amino acid at position 368 is A, and the amino acid at position 407 is V, as numbered according to the EU index.

In some embodiments, provided is the antigen-binding molecule according to any one of the above, wherein the antigen-binding molecule comprises an Fc region; the Fc region comprises a first subunit Fc1 and a second subunit Fc2 capable of associating with each other, the Fc1 comprises the amino acid sequence of SEQ ID NO: 79, and the Fc2 comprises the amino acid sequence of SEQ ID NO: 80.

In some embodiments, provided is the antigen-binding molecule according to any one of the above, wherein the antigen-binding molecule comprises one antigen-binding moiety that specifically binds to GUCY2C and one antigen-binding moiety that specifically binds to CD3.

In some embodiments, provided is the antigen-binding molecule according to any one of the above, wherein the antigen-binding molecule comprises one antigen-binding moiety that specifically binds to GUCY2C and one antigen-binding moiety that specifically binds to CD3, and the antigen-binding moieties that specifically bind to GUCY2C and CD3 are both scFvs.

In some embodiments, provided is the antigen-binding molecule according to any one of the above, which comprises one first chain having a structure represented by formula (a) and one second chain having a structure represented by formula (b):

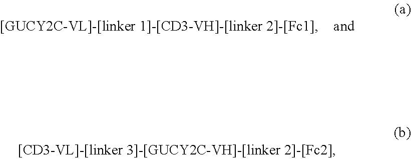

wherein the linker 1, the linker 2, and the linker 3 are identical or different peptide linkers; or the linker 1, the linker 2, and the linker 3 are absent;
the structures represented by formulas (a) and (b) are arranged from N-terminus to C-terminus.

In some embodiments, provided is the antigen-binding molecule according to any one of the above, wherein:
i) the GUCY2C-VH comprises the amino acid sequence of SEQ ID NO: 56, and the GUCY2C-VL comprises the amino acid sequence of SEQ ID NO: 54; and the CD3-VH comprises the amino acid sequence of SEQ ID NO: 77, and the CD3-VL comprises the amino acid sequence of SEQ ID NO: 78; or
the GUCY2C-VH comprises the amino acid sequence of SEQ ID NO: 56, and the GUCY2C-VL comprises the amino acid sequence of SEQ ID NO: 55; and the CD3-VH comprises the amino acid sequence of SEQ ID NO: 77, and the CD3-VL comprises the amino acid sequence of SEQ ID NO: 78; or
ii) the GUCY2C-VH comprises the amino acid sequence of SEQ ID NO: 15, and the GUCY2C-VL comprises the amino acid sequence of SEQ ID NO: 16; and the CD3-VH comprises the amino acid sequence of SEQ ID NO: 77, and the CD3-VL comprises the amino acid sequence of SEQ ID NO: 78; or
iii) the GUCY2C-VH comprises the amino acid sequence of SEQ ID NO: 17, and the GUCY2C-VL comprises the amino acid sequence of SEQ ID NO: 18; and the CD3-VH comprises the amino acid sequence of SEQ ID NO: 77, and the CD3-VL comprises the amino acid sequence of SEQ ID NO: 78.

In some embodiments, provided is the antigen-binding molecule according to any one of the above, wherein:
the amino acid sequence of the GUCY2C-VH is set forth in SEQ ID NO: 56, and the amino acid sequence of the GUCY2C-VL is set forth in SEQ ID NO: 54; and the amino acid sequence of the CD3-VH is set forth in SEQ ID NO: 77, and the amino acid sequence of the CD3-VL is set forth in SEQ ID NO: 78.

In some embodiments, provided is the antigen-binding molecule according to any one of the above, wherein:
the amino acid sequence of the GUCY2C-VH is set forth in SEQ ID NO: 56, and the amino acid sequence of the GUCY2C-VL is set forth in SEQ ID NO: 55; and the amino acid sequence of the CD3-VH is set forth in SEQ ID NO: 77, and the amino acid sequence of the CD3-VL is set forth in SEQ ID NO: 78.

In some embodiments, provided is the antigen-binding molecule according to any one of the above, wherein:
the amino acid sequence of the GUCY2C-VH is set forth in SEQ ID NO: 15, and the amino acid sequence of the GUCY2C-VL is set forth in SEQ ID NO: 16; and the amino acid sequence of the CD3-VH is set forth in SEQ ID NO: 77, and the amino acid sequence of the CD3-VL is set forth in SEQ ID NO: 78.

In some embodiments, provided is the antigen-binding molecule according to any one of the above, wherein the linker 1 and the linker 2 are both peptide linkers known in the art, so long as the antigen-binding molecule is capable of exhibiting the desired antigen binding activity. For example, the peptide linkers may be flexible peptides having 1-50 or 3-20 amino acid residues. In some embodiments, the peptide linkers each independently have the structure of L₁-(GGGGS)n-L₂, wherein L₁ is a bond, A, G (SEQ ID NO: 82), GS, GGG (SEQ ID NO: 96), GGS (SEQ ID NO: 245), GGGS (SEQ ID NO: 246), or GGGG (SEQ ID NO: 247), n is 0, 1, 2, 3, 4, 5, 6, 7, 8, 9, or 10, L₂ is a bond, G (SEQ ID NO: 82), GG, GGG (SEQ ID NO: 96), or GGGG (SEQ ID NO: 247), and the peptide linkers are not bonds. In some embodiments, the amino acid sequence of the linker 1 is set forth in SEQ ID NO: 81, the amino acid sequence of the linker 2 is set forth in SEQ ID NO: 82, and the amino acid sequence of the linker 3 is set forth in SEQ ID NO: 83.

In some embodiments, provided is the antigen-binding molecule according to any one of the above, wherein the amino acid sequence of the Fc1 is set forth in SEQ ID NO: 79, and the amino acid sequence of the Fc2 is set forth in SEQ ID NO: 80.

In some embodiments, provided is the antigen-binding molecule according to any one of the above, wherein the antigen-binding molecule comprises:
i) one first chain comprising the amino acid sequence of SEQ ID NO: 99 and one second chain comprising the amino acid sequence of SEQ ID NO: 100; or
one first chain comprising the amino acid sequence of SEQ ID NO: 101 and one second chain comprising the amino acid sequence of SEQ ID NO: 100; or
ii) one first chain comprising the amino acid sequence of SEQ ID NO: 102 and one second chain comprising the amino acid sequence of SEQ ID NO: 103; or
iii) one first chain comprising the amino acid sequence of SEQ ID NO: 104 and one second chain comprising the amino acid sequence of SEQ ID NO: 105.

In some embodiments, provided is the antigen-binding molecule according to any one of the above, wherein the antigen-binding molecule comprises: one first chain having the amino acid sequence set forth in SEQ ID NO: 99 and one second chain having the amino acid sequence set forth in SEQ ID NO: 100.

In some embodiments, provided is the antigen-binding molecule according to any one of the above, wherein the antigen-binding molecule comprises: one first chain having the amino acid sequence set forth in SEQ ID NO: 101 and one second chain having the amino acid sequence set forth in SEQ ID NO: 100.

In some embodiments, provided is the antigen-binding molecule according to any one of the above, wherein the antigen-binding molecule comprises: one first chain having the amino acid sequence set forth in SEQ ID NO: 102 and one second chain having the amino acid sequence set forth in SEQ ID NO: 103.

In some embodiments, provided is the antigen-binding molecule according to any one of the above, wherein the antigen-binding molecule comprises one antigen-binding moiety that specifically binds to CD3 and one antigen-binding moiety that specifically binds to GUCY2C; the antigen-binding moiety that specifically binds to CD3 is a Fab, and the antigen-binding moiety that specifically binds to GUCY2C is a replaced Fab comprising a Titin chain and an Obscurin chain capable of forming a dimer.

In some embodiments, provided is the antigen-binding molecule according to any one of the above, wherein the antigen-binding molecule comprises one first chain having a structure represented by formula (c), one second chain having a structure represented by formula (d), one third chain having a structure represented by formula (e), and one fourth chain having a structure represented by formula (f):

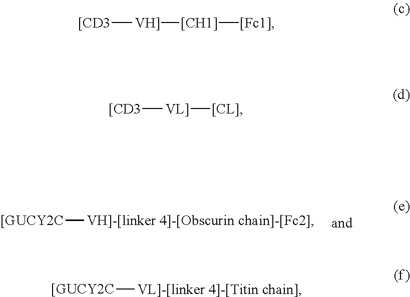

wherein the linker 4 is a peptide linker; or the linker 4 is absent;
the structures represented by formulas (c), (d), (e), and (f) are arranged from the N-terminus to the C-terminus.

In some embodiments, provided is the antigen-binding molecule according to any one of the above, wherein:
i) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 33; or
the CD3-VH comprises the amino acid sequence of SEQ ID NO: 89, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 33; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 15, and the GUCY2C-VL comprises the amino acid sequence of SEQ ID NO: 16; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 31; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 32; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 34; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 35; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 36; or
ii) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 54; or
iii) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 42, and the GUCY2C-VL comprises the amino acid sequence of SEQ ID NO: 41; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 17, and the GUCY2C-VL comprises the amino acid sequence of SEQ ID NO: 18.

In some embodiments, provided is the antigen-binding molecule according to any one of the above, wherein:
the amino acid sequence of the CD3-VH is set forth in SEQ ID NO: 77, and the amino acid sequence of the CD3-VL is set forth in SEQ ID NO: 78; and the amino acid sequence of the GUCY2C-VH is set forth in SEQ ID NO: 37, and the amino acid sequence of the GUCY2C-VL is set forth in SEQ ID NO: 33.

In some embodiments, provided is the antigen-binding molecule according to any one of the above, wherein:
the amino acid sequence of the CD3-VH is set forth in SEQ ID NO: 89, and the amino acid sequence of the CD3-VL is set forth in SEQ ID NO: 78; and the amino acid sequence of the GUCY2C-VH is set forth in SEQ ID NO: 37, and the amino acid sequence of the GUCY2C-VL is set forth in SEQ ID NO: 33.

In some embodiments, provided is the antigen-binding molecule according to any one of the above, wherein:
the amino acid sequence of the CD3-VH is set forth in SEQ ID NO: 77, and the amino acid sequence of the CD3-VL is set forth in SEQ ID NO: 78; and the amino acid sequence of the GUCY2C-VH is set forth in SEQ ID NO: 42, and the amino acid sequence of the GUCY2C-VL is set forth in SEQ ID NO: 41.

In some embodiments, provided is the antigen-binding molecule according to any one of the above, wherein the Titin chain and the Obscurin chain are any Titin chain and Obscurin chain in Table 3-1 and Table 3-2 of the present disclosure that can form a dimer. In some embodiments, provided is the antigen-binding molecule according to any one of the above, wherein the Titin chain comprises the amino acid sequence of SEQ ID NO: 198. In some embodiments, provided is the antigen-binding molecule according to any one of the above, wherein the Obscurin chain comprises the amino acid sequence of SEQ ID NO: 236.

In some embodiments, provided is the antigen-binding molecule according to any one of the above, wherein the linkers 4 are all peptide linkers known in the art, so long as the antigen-binding molecule is capable of exhibiting the desired antigen binding activity. For example, the peptide linkers may be flexible peptides comprising 1-50 or 3-20 amino acid residues. In some embodiments, the peptide linker is 3-15 amino acid residues in length. In some embodiments, the peptide linkers each independently have a structure of (GGGGS)n, wherein n is 1, 2, or 3. In some embodiments, the sequence of the linker 4 is GGGGS (SEQ ID NO: 90).

In some embodiments, provided is the antigen-binding molecule according to any one of the above, wherein the CH1 is the CH1 sequence of an IgG. In some embodiments, the CH1 is the CH1 of an IgG1. In some embodiments, the CH1 comprises the amino acid sequence of SEQ ID NO: 84.

In some embodiments, provided is the antigen-binding molecule according to any one of the above, wherein the CL is the light chain constant region of an antibody. In some embodiments, provided is the antigen-binding molecule according to any one of the above, wherein the CL is a kappa or lamada light chain constant region. In some embodiments, the CL comprises the amino acid sequence of SEQ ID NO: 87.

In some embodiments, provided is the antigen-binding molecule according to any one of the above, wherein the amino acid sequence of the Fc1 is set forth in SEQ ID NO: 85, and the amino acid sequence of the Fc2 is set forth in SEQ ID NO: 86.

In some embodiments, provided is the antigen-binding molecule according to any one of the above, wherein the antigen-binding molecule comprises:
i) one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 113; or
one first chain comprising the amino acid sequence of SEQ ID NO: 242, one second chain comprising the amino acid sequence of SEQ ID NO: 92, one third chain comprising the amino acid sequence of SEQ ID NO: 110, and one fourth chain comprising the amino acid sequence of SEQ ID NO: 113; or
one first chain comprising the amino acid sequence of SEQ ID NO: 91, one second chain comprising the amino acid sequence of SEQ ID NO: 92, one third chain comprising the amino acid sequence of SEQ ID NO: 108, and one fourth chain comprising the amino acid sequence of SEQ ID NO: 109; or
one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 111; or
one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 112; or
one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 114; or
one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 115; or
one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 116; or
ii) one first chain comprising the amino acid sequence of SEQ ID NO: 91, one second chain comprising the amino acid sequence of SEQ ID NO: 92, one third chain comprising the amino acid sequence of SEQ ID NO: 106, and one fourth chain comprising the amino acid sequence of SEQ ID NO: 107; or
iii) one first chain comprising the amino acid sequence of SEQ ID NO: 91, one second chain comprising the amino acid sequence of SEQ ID NO: 92, one third chain comprising the amino acid sequence of SEQ ID NO: 119, and one fourth chain comprising the amino acid sequence of SEQ ID NO: 120; or
one first chain comprising the amino acid sequence of SEQ ID NO: 91, one second chain comprising the amino acid sequence of SEQ ID NO: 92, one third chain comprising the amino acid sequence of SEQ ID NO: 117, and one fourth chain comprising the amino acid sequence of SEQ ID NO: 118.

In some embodiments, provided is the antigen-binding molecule according to any one of the above, wherein the antigen-binding molecule comprises: one first chain having the amino acid sequence set forth in SEQ ID NO: 91, one second chain having the amino acid sequence set forth in SEQ ID NO: 92, one third chain having the amino acid sequence set forth in SEQ ID NO: 110, and one fourth chain having the amino acid sequence set forth in SEQ ID NO: 113.

In some embodiments, provided is the antigen-binding molecule according to any one of the above, wherein the antigen-binding molecule comprises: one first chain having the amino acid sequence set forth in SEQ ID NO: 242, one second chain having the amino acid sequence set forth in SEQ ID NO: 92, one third chain having the amino acid sequence set forth in SEQ ID NO: 110, and one fourth chain having the amino acid sequence set forth in SEQ ID NO: 113.

In some embodiments, provided is the antigen-binding molecule according to any one of the above, wherein the antigen-binding molecule comprises: one first chain having the amino acid sequence set forth in SEQ ID NO: 91, one second chain having the amino acid sequence set forth in SEQ ID NO: 92, one third chain having the amino acid sequence set forth in SEQ ID NO: 119, and one fourth chain having the amino acid sequence set forth in SEQ ID NO: 120.

In some embodiments, provided is the antigen-binding molecule according to any one of the above, wherein the antigen-binding molecule comprises one antigen-binding moiety that specifically binds to GUCY2C and one antigen-binding moiety that specifically binds to CD3; the antigen-binding moiety that specifically binds to GUCY2C is a replaced Fab comprising a Titin chain and an Obscurin chain capable of forming a dimer, and the antigen-binding moiety that specifically binds to CD3 is a Fab.

In some embodiments, provided is the antigen-binding molecule according to any one of the above, wherein the antigen-binding molecule comprises one first chain having a structure represented by formula (g), one second chain having a structure represented by formula (f), one third chain having a structure represented by formula (h), and one fourth chain having a structure represented by formula (d):

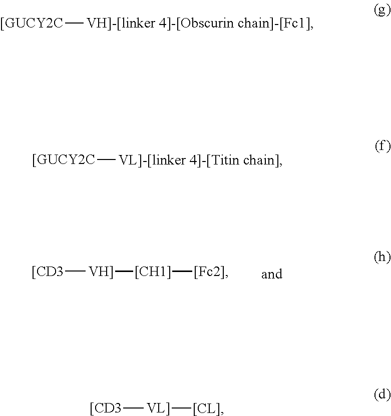

wherein the linker 4 is a peptide linker; or the linker 4 is absent;
the structures represented by formulas (g), (f), (h), and (d) are arranged from N-terminus to C-terminus.

In some embodiments, provided is the antigen-binding molecule according to any one of the above, wherein:
i) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 54; or
ii) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 31; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 32; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 33; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 34; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 35; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 36; or
iii) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 42, and the GUCY2C-VL comprises the amino acid sequence of SEQ ID NO: 41.

In some embodiments, provided is the antigen-binding molecule according to any one of the above, wherein:
the amino acid sequence of the CD3-VH is set forth in SEQ ID NO: 77, and the amino acid sequence of the CD3-VL is set forth in SEQ ID NO: 78; and the amino acid sequence of the GUCY2C-VH is set forth in SEQ ID NO: 56, and the amino acid sequence of the GUCY2C-VL is set forth in SEQ ID NO: 54.

In some embodiments, provided is the antigen-binding molecule according to any one of the above, wherein the Titin chain and the Obscurin chain are any Titin chain and Obscurin chain in Table 3-1 and Table 3-2 of the present disclosure that can form a dimer. In some embodiments, provided is the antigen-binding molecule according to any one of the above, wherein the Titin chain comprises the amino acid sequence of SEQ ID NO: 198. In some embodiments, provided is the antigen-binding molecule according to any one of the above, wherein the Obscurin chain comprises the amino acid sequence of SEQ ID NO: 236.

In some embodiments, provided is the antigen-binding molecule according to any one of the above, wherein the linkers 4 are all peptide linkers known in the art, so long as the antigen-binding molecule is capable of exhibiting the desired antigen binding activity. For example, the peptide linkers may be flexible peptides comprising 1-50 or 3-20 amino acid residues. In some embodiments, the peptide linker is 3-15 amino acid residues in length. In some embodiments, the peptide linkers each independently have a structure of (GGGGS)n, wherein n is 1, 2, or 3. In some embodiments, the sequence of the linker 4 is GGGGS (SEQ ID NO: 90).

In some embodiments, provided is the antigen-binding molecule according to any one of the above, wherein the CH1 is the CH1 sequence of an IgG. In some embodiments, the CH1 is the CH1 of an IgG1. In some embodiments, the CH1 comprises the amino acid sequence of SEQ ID NO: 84.

In some embodiments, provided is the antigen-binding molecule according to any one of the above, wherein the CL is the light chain constant region of an antibody. In some embodiments, provided is the antigen-binding molecule according to any one of the above, wherein the CL is a kappa or lamada light chain constant region. In some embodiments, the CL comprises the amino acid sequence of SEQ ID NO: 87.

In some embodiments, provided is the antigen-binding molecule according to any one of the above, wherein the amino acid sequence of the Fc1 is set forth in SEQ ID NO: 85, and the amino acid sequence of the Fc2 is set forth in SEQ ID NO: 86.

In some embodiments, provided is the antigen-binding molecule according to any one of the above, wherein the antigen-binding molecule comprises:
i) one first chain comprising the amino acid sequence of SEQ ID NO: 121, one second chain comprising the amino acid sequence of SEQ ID NO: 107, one third chain comprising the amino acid sequence of SEQ ID NO: 93, and one fourth chain comprising the amino acid sequence of SEQ ID NO: 92; or
ii) one first chain comprising the amino acid sequence of SEQ ID NO: 122, one second chain comprising the amino acid sequence of SEQ ID NO: 111, one third chain comprising the amino acid sequence of SEQ ID NO: 93, and one fourth chain comprising the amino acid sequence of SEQ ID NO: 92; or
one first chain comprising the amino acid sequence of SEQ ID NO: 122, one second chain comprising the amino acid sequence of SEQ ID NO: 112, one third chain comprising the amino acid sequence of SEQ ID NO: 93, and one fourth chain comprising the amino acid sequence of SEQ ID NO: 92; or
one first chain comprising the amino acid sequence of SEQ ID NO: 122, one second chain comprising the amino acid sequence of SEQ ID NO: 113, one third chain comprising the amino acid sequence of SEQ ID NO: 93, and one fourth chain comprising the amino acid sequence of SEQ ID NO: 92; or
one first chain comprising the amino acid sequence of SEQ ID NO: 122, one second chain comprising the amino acid sequence of SEQ ID NO: 114, one third chain comprising the amino acid sequence of SEQ ID NO: 93, and one fourth chain comprising the amino acid sequence of SEQ ID NO: 92; or
one first chain comprising the amino acid sequence of SEQ ID NO: 122, one second chain comprising the amino acid sequence of SEQ ID NO: 115, one third chain comprising the amino acid sequence of SEQ ID NO: 93, and one fourth chain comprising the amino acid sequence of SEQ ID NO: 92; or
one first chain comprising the amino acid sequence of SEQ ID NO: 122, one second chain comprising the amino acid sequence of SEQ ID NO: 116, one third chain comprising the amino acid sequence of SEQ ID NO: 93, and one fourth chain comprising the amino acid sequence of SEQ ID NO: 92; or
iii) one first chain comprising the amino acid sequence of SEQ ID NO: 123, one second chain comprising the amino acid sequence of SEQ ID NO: 120, one third chain comprising the amino acid sequence of SEQ ID NO: 93, and one fourth chain comprising the amino acid sequence of SEQ ID NO: 92.

In some embodiments, provided is the antigen-binding molecule according to any one of the above, wherein the antigen-binding molecule comprises: one first chain having the amino acid sequence set forth in SEQ ID NO: 121, one second chain having the amino acid sequence set forth in SEQ ID NO: 107, one third chain having the amino acid sequence set forth in SEQ ID NO: 93, and one fourth chain having the amino acid sequence set forth in SEQ ID NO: 92.

In some embodiments, provided is the antigen-binding molecule according to any one of the above, wherein the antigen-binding molecule comprises two antigen-binding moieties that specifically bind to GUCY2C and two antigen-binding moieties that specifically bind to CD3, and the antigen-binding moieties that specifically bind to GUCY2C and CD3 are all scFvs.

In some embodiments, provided is the antigen-binding molecule according to any one of the above, wherein the antigen-binding molecule comprises two first chains having a structure represented by formula (i) and two second chains having a structure represented by formula (j):

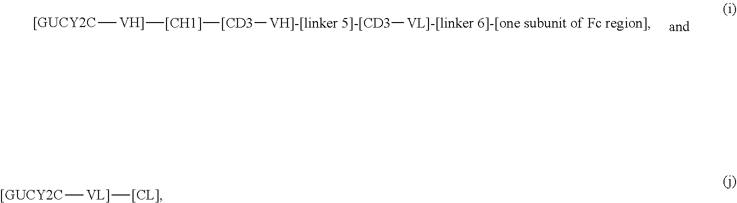

wherein the linker 5 and the linker 6 are peptide linkers; or the linker 5 and the linker 6 are absent;
the structures represented by formulas (i) and (j) are arranged from N-terminus to C-terminus.

In some embodiments, provided is the antigen-binding molecule according to any one of the above, wherein:
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 54; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 55.

In some embodiments, provided is the antigen-binding molecule according to any one of the above, wherein:
the amino acid sequence of the CD3-VH is set forth in SEQ ID NO: 77, and the amino acid sequence of the CD3-VL is set forth in SEQ ID NO: 78; and the amino acid sequence of the GUCY2C-VH is set forth in SEQ ID NO: 56, and the amino acid sequence of the GUCY2C-VL is set forth in SEQ ID NO: 54.

In some embodiments, provided is the antigen-binding molecule according to any one of the above, wherein the linker 5 and the linker 6 are both peptide linkers known in the art, so long as the antigen-binding molecule is capable of exhibiting the desired antigen binding activity. For example, the peptide linkers may be flexible peptides comprising 1-50 or 3-20 amino acid residues. In some embodiments, the peptide linkers each independently have the structure of L₁-(GGGGS)n-L₂, wherein L₁ is a bond, A, G, GS, GGG (SEQ ID NO: 96), GGS (SEQ ID NO: 245), GGGS (SEQ ID NO: 246), or GGGG (SEQ ID NO: 247), n is 0, 1, 2, 3, 4, 5, 6, 7, 8, 9, or 10, L₂ is a bond, G, GG, GGG (SEQ ID NO: 96), or GGGG (SEQ ID NO: 247), and the peptide linkers are not bonds. In some embodiments, the peptide linker is 3-15 amino acid residues in length. In some embodiments, the peptide linkers each independently have a structure of (GGGGS)n, wherein n is 1, 2, or 3. In some embodiments, the amino acid sequence of the linker 5 is set forth in SEQ ID NO: 95, and the amino acid sequence of the linker 6 is set forth in SEQ ID NO: 96.

In some embodiments, provided is the antigen-binding molecule according to any one of the above, wherein the CH1 is the CH1 sequence of an IgG. In some embodiments, the CH1 is the CH1 of an IgG1. In some embodiments, the CH1 comprises the amino acid sequence of SEQ ID NO: 84.

In some embodiments, provided is the antigen-binding molecule according to any one of the above, wherein the CL is the light chain constant region of an antibody. In some embodiments, provided is the antigen-binding molecule according to any one of the above, wherein the CL is a kappa or lamada light chain constant region. In some embodiments, the CL comprises the amino acid sequence of SEQ ID NO: 59.

In some embodiments, provided is the antigen-binding molecule according to any one of the above, wherein the amino acid sequence of one subunit of the Fc region is set forth in SEQ ID NO: 94.

In some embodiments, provided is the antigen-binding molecule according to any one of the above, wherein the antigen-binding molecule comprises: two first chains comprising the amino acid sequence of SEQ ID NO: 124 and two second chains comprising the amino acid sequence of SEQ ID NO: 65; or two first chains comprising the amino acid sequence of SEQ ID NO: 124 and two second chains comprising the amino acid sequence of SEQ ID NO: 66.

In some embodiments, provided is the antigen-binding molecule according to any one of the above, wherein the antigen-binding molecule comprises: two first chains having the amino acid sequence set forth in SEQ ID NO: 124 and two second chains having the amino acid sequence set forth in SEQ ID NO: 65.

In some embodiments, provided is the antigen-binding molecule according to any one of the above, wherein the antigen-binding molecule comprises two antigen-binding moieties that specifically bind to GUCY2C and two antigen-binding moieties that specifically bind to CD3; the antigen-binding moieties that specifically bind to GUCY2C are Fabs, and the antigen-binding moieties that specifically bind to CD3 are replaced Fabs comprising a Titin chain and an Obscurin chain capable of forming a dimer.

In some embodiments, provided is the antigen-binding molecule according to any one of the above, wherein the antigen-binding molecule comprises two first chains having a structure represented by formula (k), two second chains having a structure represented by formula (j), and two third chains having a structure represented by formula (l):

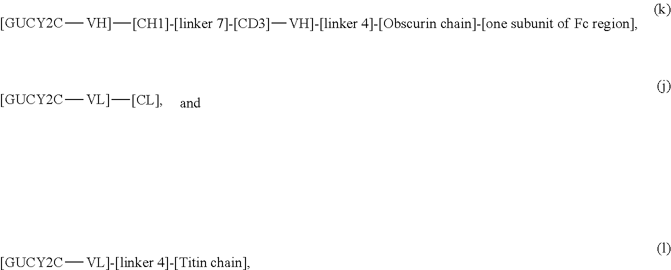

wherein the linker 4 and the linker 7 are peptide linkers; or the linker 4 and the linker 7 are absent;
the structures represented by formulas (k), (j), and (l) are arranged from N-terminus to C-terminus.

In some embodiments, provided is the antigen-binding molecule according to any one of the above, wherein:
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 54; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 55.

In some embodiments, provided is the antigen-binding molecule according to any one of the above, wherein:
the amino acid sequence of the CD3-VH is set forth in SEQ ID NO: 77, and the amino acid sequence of the CD3-VL is set forth in SEQ ID NO: 78; and the amino acid sequence of the GUCY2C-VH is set forth in SEQ ID NO: 56, and the amino acid sequence of the GUCY2C-VL is set forth in SEQ ID NO: 54.

In some embodiments, provided is the antigen-binding molecule according to any one of the above, wherein the linker 4 and the linker 7 are both peptide linkers known in the art, so long as the antigen-binding molecule is capable of exhibiting the desired antigen binding activity. In some embodiments, the peptide linker is 3-15 amino acid residues in length. In some embodiments, the peptide linkers each independently have a structure of (GGGGS)n, wherein n is 1, 2, or 3. In some embodiments, the amino acid sequence of the linker 4 is set forth in SEQ ID NO: 90, and the amino acid sequence of the linker 7 is set forth in SEQ ID NO: 97.

In some embodiments, provided is the antigen-binding molecule according to any one of the above, wherein the Titin chain and the Obscurin chain are any Titin chain and Obscurin chain in Table 3-1 and Table 3-2 of the present disclosure that can form a dimer. In some embodiments, provided is the antigen-binding molecule according to any one of the above, wherein the Titin chain comprises the amino acid sequence of SEQ ID NO: 198. In some embodiments, provided is the antigen-binding molecule according to any one of the above, wherein the Obscurin chain comprises the amino acid sequence of SEQ ID NO: 236.

In some embodiments, provided is the antigen-binding molecule according to any one of the above, wherein the CH1 is the CH1 sequence of an IgG. In some embodiments, the CH1 is the CH1 of an IgG1. In some embodiments, the CH1 comprises the amino acid sequence of SEQ ID NO: 84.

In some embodiments, provided is the antigen-binding molecule according to any one of the above, wherein the CL is the light chain constant region of an antibody. In some embodiments, provided is the antigen-binding molecule according to any one of the above, wherein the CL is a kappa or lamada light chain constant region. In some embodiments, the CL comprises the amino acid sequence of SEQ ID NO: 59.

In some embodiments, the amino acid sequence of one subunit of the Fc region is set forth in SEQ ID NO: 86.

In some embodiments, provided is the antigen-binding molecule according to any one of the above, wherein the antigen-binding molecule comprises:
two first chains comprising the amino acid sequence of SEQ ID NO: 125, two second chains comprising the amino acid sequence of SEQ ID NO: 65, and two third chains comprising the amino acid sequence of SEQ ID NO: 98; or
two first chains comprising the amino acid sequence of SEQ ID NO: 125, two second chains comprising the amino acid sequence of SEQ ID NO: 66, and two third chains comprising the amino acid sequence of SEQ ID NO: 98.

In some embodiments, provided is the antigen-binding molecule according to any one of the above, wherein the antigen-binding molecule comprises: two first chains having the amino acid sequence set forth in SEQ ID NO: 125, two second chains having the amino acid sequence set forth in SEQ ID NO: 65, and two third chains having the amino acid sequence set forth in SEQ ID NO: 98.

In another aspect, the present disclosure also provides an anti-GUCY2C antibody capable of specifically binding to GUCY2C. The antibody comprises a heavy chain variable region GUCY2C-VH and a light chain variable region GUCY2C-VL, wherein:
(i) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 25, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 54, 26, 50, 51, 52, or 53, respectively; or
(ii) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 37 or 15, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 16, 34, 35, or 36, respectively; or
(iii) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 42 or 17, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 41 or 18, respectively; or
iv-1) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 273, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 274, respectively; or
iv-2) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 275, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 276, respectively; or
iv-3) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 277, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 278, respectively; or
iv-4) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 279, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 280, respectively; or
iv-5) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 330, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 331, respectively; or
iv-6) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 332, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 333, respectively; or
iv-7) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 334, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 335, respectively; or
iv-8) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 336, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 337, respectively; or
iv-9) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 338, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 339, respectively; or
iv-10) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 340, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 341, respectively; or
iv-11) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 342, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 343, respectively; or
iv-12) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 344, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 345, respectively; or
iv-13) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 346, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 347, respectively; or
iv-14) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 348, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 349, respectively; or
iv-15) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 350, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 351, respectively; or
iv-16) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 352, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 353, respectively.

In some embodiments, provided is the anti-GUCY2C antibody according to the above, wherein:
(i) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 25, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 54, respectively, or
(ii) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 37, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 16, respectively, or
(iii) GUCY2C-HCDR1, GUCY2C-HCDR2, and GUCY2C-HCDR3 in the GUCY2C-VH comprise the amino acid sequences of GUCY2C-HCDR1, GUCY2C-HCDR2, and GUCY2C-HCDR3 in SEQ ID NO: 42, respectively, and GUCY2C-LCDR1, GUCY2C-LCDR2, and GUCY2C-LCDR3 in the GUCY2C-VL comprise the amino acid sequences of GUCY2C-LCDR1, GUCY2C-LCDR2, and GUCY2C-LCDR3 in SEQ ID NO: 41, respectively.

In some embodiments, the GUCY2C-HCDR1, the GUCY2C-HCDR2, the GUCY2C-HCDR3, the GUCY2C-LCDR1, the GUCY2C-LCDR2, and the GUCY2C-LCDR3 are defined according to the Kabat, IMGT, Chothia, AbM, or Contact numbering scheme. In some embodiments, provided is the anti-GUCY2C antibody according to any one of the above, wherein:
i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 47, 22, 43, 44, 45, or 46, a GUCY2C-LCDR2 comprising the amino acid sequence of SEQ ID NO: 23, and a GUCY2C-LCDR3 comprising the amino acid sequence of SEQ ID NO: 24; or
ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27 or 6; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, 28, 29, or 30, and a GUCY2C-LCDR3 comprising the amino acid sequence of SEQ ID NO: 9; or
iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40 or 12; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39 or 8, and a GUCY2C-LCDR3 comprising the amino acid sequence of SEQ ID NO: 14; or
iv-1) the GUCY2C-VH comprises: a GUCY2C-HCDR1 comprising the amino acid sequence of SEQ ID NO: 250, a GUCY2C-HCDR2 comprising the amino acid sequence of SEQ ID NO: 251, and a GUCY2C-HCDR3 comprising the amino acid sequence of SEQ ID NO: 252; and the GUCY2C-VL comprises: a GUCY2C-LCDR1 comprising the amino acid sequence of SEQ ID NO: 253, a GUCY2C-LCDR2 comprising the amino acid sequence of SEQ ID NO: 254, and a GUCY2C-LCDR3 comprising the amino acid sequence of SEQ ID NO: 255; or
iv-2) the GUCY2C-VH comprises: a GUCY2C-HCDR1 comprising the amino acid sequence of SEQ ID NO: 256, a GUCY2C-HCDR2 comprising the amino acid sequence of SEQ ID NO: 257, and a GUCY2C-HCDR3 comprising the amino acid sequence of SEQ ID NO: 258; and the GUCY2C-VL comprises: a GUCY2C-LCDR1 comprising the amino acid sequence of SEQ ID NO: 259, a GUCY2C-LCDR2 comprising the amino acid sequence of SEQ ID NO: 8, and a GUCY2C-LCDR3 comprising the amino acid sequence of SEQ ID NO: 260; or
iv-3) the GUCY2C-VH comprises: a GUCY2C-HCDR1 comprising the amino acid sequence of SEQ ID NO: 261, a GUCY2C-HCDR2 comprising the amino acid sequence of SEQ ID NO: 262, and a GUCY2C-HCDR3 comprising the amino acid sequence of SEQ ID NO: 263; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 265, and a GUCY2C-LCDR3 comprising the amino acid sequence of SEQ ID NO: 266; or
iv-4) the GUCY2C-VH comprises: a GUCY2C-HCDR1 comprising the amino acid sequence of SEQ ID NO: 267, a GUCY2C-HCDR2 comprising the amino acid sequence of SEQ ID NO: 268, and a GUCY2C-HCDR3 comprising the amino acid sequence of SEQ ID NO: 269; and the GUCY2C-VL comprises: a GUCY2C-LCDR1 comprising the amino acid sequence of SEQ ID NO: 270, a GUCY2C-LCDR2 comprising the amino acid sequence of SEQ ID NO: 271, and a GUCY2C-LCDR3 comprising the amino acid sequence of SEQ ID NO: 272; or
iv-5) the GUCY2C-VH comprises: a GUCY2C-HCDR1 comprising the amino acid sequence of SEQ ID NO: 281, a GUCY2C-HCDR2 comprising the amino acid sequence of SEQ ID NO: 282, and a GUCY2C-HCDR3 comprising the amino acid sequence of SEQ ID NO: 283; and the GUCY2C-VL comprises: a GUCY2C-LCDR1 comprising the amino acid sequence of SEQ ID NO: 284, a GUCY2C-LCDR2 comprising the amino acid sequence of SEQ ID NO: 8, and a GUCY2C-LCDR3 comprising the amino acid sequence of SEQ ID NO: 285; or
iv-6) the GUCY2C-VH comprises: a GUCY2C-HCDR1 comprising the amino acid sequence of SEQ ID NO: 286, a GUCY2C-HCDR2 comprising the amino acid sequence of SEQ ID NO: 287, and a GUCY2C-HCDR3 comprising the amino acid sequence of SEQ ID NO: 288; and the GUCY2C-VL comprises: a GUCY2C-LCDR1 comprising the amino acid sequence of SEQ ID NO: 289, a GUCY2C-LCDR2 comprising the amino acid sequence of SEQ ID NO: 265, and a GUCY2C-LCDR3 comprising the amino acid sequence of SEQ ID NO: 290; or
iv-7) the GUCY2C-VH comprises: a GUCY2C-HCDR1 comprising the amino acid sequence of SEQ ID NO: 291, a GUCY2C-HCDR2 comprising the amino acid sequence of SEQ ID NO: 292, and a GUCY2C-HCDR3 comprising the amino acid sequence of SEQ ID NO: 293; and the GUCY2C-VL comprises: a GUCY2C-LCDR1 comprising the amino acid sequence of SEQ ID NO: 294, a GUCY2C-LCDR2 comprising the amino acid sequence of SEQ ID NO: 295, and a GUCY2C-LCDR3 comprising the amino acid sequence of SEQ ID NO: 296; or
iv-8) the GUCY2C-VH comprises: a GUCY2C-HCDR1 comprising the amino acid sequence of SEQ ID NO: 261, a GUCY2C-HCDR2 comprising the amino acid sequence of SEQ ID NO: 297, and a GUCY2C-HCDR3 comprising the amino acid sequence of SEQ ID NO: 298; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 299, and a GUCY2C-LCDR3 comprising the amino acid sequence of SEQ ID NO: 300; or
iv-9) the GUCY2C-VH comprises: a GUCY2C-HCDR1 comprising the amino acid sequence of SEQ ID NO: 261, a GUCY2C-HCDR2 comprising the amino acid sequence of SEQ ID NO: 301, and a GUCY2C-HCDR3 comprising the amino acid sequence of SEQ ID NO: 302; and the GUCY2C-VL comprises: a GUCY2C-LCDR1 comprising the amino acid sequence of SEQ ID NO: 303, a GUCY2C-LCDR2 comprising the amino acid sequence of SEQ ID NO: 299, and a GUCY2C-LCDR3 comprising the amino acid sequence of SEQ ID NO: 304; or
iv-10) the GUCY2C-VH comprises: a GUCY2C-HCDR1 comprising the amino acid sequence of SEQ ID NO: 305, a GUCY2C-HCDR2 comprising the amino acid sequence of SEQ ID NO: 306, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 24; or
iv-11) the GUCY2C-VH comprises: a GUCY2C-HCDR1 comprising the amino acid sequence of SEQ ID NO: 128, a GUCY2C-HCDR2 comprising the amino acid sequence of SEQ ID NO: 307, and a GUCY2C-HCDR3 comprising the amino acid sequence of SEQ ID NO: 308; and the GUCY2C-VL comprises: a GUCY2C-LCDR1 comprising the amino acid sequence of SEQ ID NO: 309, a GUCY2C-LCDR2 comprising the amino acid sequence of SEQ ID NO: 310, and a GUCY2C-LCDR3 comprising the amino acid sequence of SEQ ID NO: 311; or
iv-12) the GUCY2C-VH comprises: a GUCY2C-HCDR1 comprising the amino acid sequence of SEQ ID NO: 312, a GUCY2C-HCDR2 comprising the amino acid sequence of SEQ ID NO: 313, and a GUCY2C-HCDR3 comprising the amino acid sequence of SEQ ID NO: 314; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 315; or
iv-13) the GUCY2C-VH comprises: a GUCY2C-HCDR1 comprising the amino acid sequence of SEQ ID NO: 286, a GUCY2C-HCDR2 comprising the amino acid sequence of SEQ ID NO: 287, and a GUCY2C-HCDR3 comprising the amino acid sequence of SEQ ID NO: 316; and the GUCY2C-VL comprises: a GUCY2C-LCDR1 comprising the amino acid sequence of SEQ ID NO: 317, a GUCY2C-LCDR2 comprising the amino acid sequence of SEQ ID NO: 265, and a GUCY2C-LCDR3 comprising the amino acid sequence of SEQ ID NO: 300; or
iv-14) the GUCY2C-VH comprises: a GUCY2C-HCDR1 comprising the amino acid sequence of SEQ ID NO: 318, a GUCY2C-HCDR2 comprising the amino acid sequence of SEQ ID NO: 319, and a GUCY2C-HCDR3 comprising the amino acid sequence of SEQ ID NO: 320; and the GUCY2C-VL comprises: a GUCY2C-LCDR1 comprising the amino acid sequence of SEQ ID NO: 321, a GUCY2C-LCDR2 comprising the amino acid sequence of SEQ ID NO: 322, and a GUCY2C-LCDR3 comprising the amino acid sequence of SEQ ID NO: 323; or
iv-15) the GUCY2C-VH comprises: a GUCY2C-HCDR1 comprising the amino acid sequence of SEQ ID NO: 324, a GUCY2C-HCDR2 comprising the amino acid sequence of SEQ ID NO: 325, and a GUCY2C-HCDR3 comprising the amino acid sequence of SEQ ID NO: 326; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 327; or
iv-16) the GUCY2C-VH comprises: a GUCY2C-HCDR1 comprising the amino acid sequence of SEQ ID NO: 261, a GUCY2C-HCDR2 comprising the amino acid sequence of SEQ ID NO: 328, and a GUCY2C-HCDR3 comprising the amino acid sequence of SEQ ID NO: 302; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 329, and a GUCY2C-LCDR3 comprising the amino acid sequence of SEQ ID NO: 300.

In some embodiments, provided is the anti-GUCY2C antibody according to any one of the above, wherein:
i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 47, a GUCY2C-LCDR2 comprising the amino acid sequence of SEQ ID NO: 23, and a GUCY2C-LCDR3 comprising the amino acid sequence of SEQ ID NO: 24; or
ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, and a GUCY2C-LCDR3 comprising the amino acid sequence of SEQ ID NO: 9; or
iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39, and a GUCY2C-LCDR3 comprising the amino acid sequence of SEQ ID NO: 14.

In some embodiments, provided is the anti-GUCY2C antibody according to any one of the above, wherein:
i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 24; or
ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 6; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, and a GUCY2C-LCDR3 comprising the amino acid sequence of SEQ ID NO: 9; or
iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 12; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 8, and a GUCY2C-LCDR3 comprising the amino acid sequence of SEQ ID NO: 14.

In some embodiments, provided is the anti-GUCY2C antibody according to any one of the above, wherein:
i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 43, a GUCY2C-LCDR2 comprising the amino acid sequence of SEQ ID NO: 23, and a GUCY2C-LCDR3 comprising the amino acid sequence of SEQ ID NO: 24; or
the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 44, a GUCY2C-LCDR2 comprising the amino acid sequence of SEQ ID NO: 23, and a GUCY2C-LCDR3 comprising the amino acid sequence of SEQ ID NO: 24; or
the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 45, a GUCY2C-LCDR2 comprising the amino acid sequence of SEQ ID NO: 23, and a GUCY2C-LCDR3 comprising the amino acid sequence of SEQ ID NO: 24; or
ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 6; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 28, and a GUCY2C-LCDR3 comprising the amino acid sequence of SEQ ID NO: 9; or
the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 6; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 30, and a GUCY2C-LCDR3 comprising the amino acid sequence of SEQ ID NO: 9.

In some embodiments, provided is the anti-GUCY2C antibody according to any one of the above, wherein the GUCY2C-HCDR1, the GUCY2C-HCDR2, the GUCY2C-HCDR3, the GUCY2C-LCDR1, the GUCY2C-LCDR2, and the GUCY2C-LCDR3 are defined according to the Kabat numbering scheme.

In some embodiments, provided is the anti-GUCY2C antibody according to any one of the above, wherein the anti-GUCY2C antibody is a murine antibody, a chimeric antibody, or a humanized antibody, preferably a humanized antibody.

In some embodiments, provided is the anti-GUCY2C antibody according to any one of the above, wherein the anti-GUCY2C antibody comprises a heavy chain constant region and a light chain constant region; preferably, the heavy chain constant region is a human IgG1 constant region, and the light chain constant region is selected from the group consisting of constant regions of human antibody K and k chains; more preferably, the heavy chain constant region comprises the amino acid sequence of SEQ ID NO: 58, and the light chain constant region comprises the amino acid sequence of SEQ ID NO: 59.

In some embodiments, provided is the anti-GUCY2C antibody according to any one of the above, wherein the GUCY2C-VH and the GUCY2C-VL are humanized and each comprise the FR region of a human antibody.

In some embodiments, provided is the anti-GUCY2C antibody according to any one of the above, wherein:
(i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21, and the FR region of the GUCY2C-VH comprises one or more amino acid substitutions selected from the group consisting of 1E, 39L, 43K, 69L, 71V, and 73K; and the GUCY2C-VL comprises: a GUCY2C-LCDR1 comprising the amino acid sequence of SEQ ID NO: 47, 22, 43, 44, 45, or 46, a GUCY2C-LCDR2 comprising the amino acid sequence of SEQ ID NO: 23, and a GUCY2C-LCDR3 comprising the amino acid sequence of SEQ ID NO: 24, and the FR region of the GUCY2C-VL comprises one or more amino acid substitutions selected from the group consisting of 2V, 3V, 68R, and 74K; or
(ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27 or 6, and the FR region of the GUCY2C-VH comprises one or more amino acid substitutions selected from the group consisting of 1E, 38K, 66K, and 67A; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, 28, 29, or 30, and a GUCY2C-LCDR3 comprising the amino acid sequence of SEQ ID NO: 9, and the FR region of the GUCY2C-VL comprises one or more amino acid substitutions selected from the group consisting of 15P, 16G, 42S, 43S, 45K, 46S, 47W, 49Y, 58V, 71Y, and 76S; or
(iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40 or 12, and the FR region of the GUCY2C-VH comprises one or more amino acid substitutions selected from the group consisting of 1E, 2F, 28S, 69L, 71V, 73Q, and 75S; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39 or 8, and a GUCY2C-LCDR3 comprising the amino acid sequence of SEQ ID NO: 14, and the FR region of the GUCY2C-VL comprises one or more amino acid substitutions selected from the group consisting of 42S, 43S, 46P, 47W, 58V, and 71Y.

In some embodiments, provided is the anti-GUCY2C antibody according to any one of the above, wherein:
(i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21, and the FR region of the GUCY2C-VH comprises one or more amino acid substitutions selected from the group consisting of 1E, 39L, 43K, 69L, 71V, and 73K; and the GUCY2C-VL comprises: a GUCY2C-LCDR1 comprising the amino acid sequence of SEQ ID NO: 47, a GUCY2C-LCDR2 comprising the amino acid sequence of SEQ ID NO: 23, and a GUCY2C-LCDR3 comprising the amino acid sequence of SEQ ID NO: 24, and the FR region of the GUCY2C-VL comprises one or more amino acid substitutions selected from the group consisting of 2V, 3V, 68R, and 74K; or
(ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27, and the FR region of the GUCY2C-VH comprises one or more amino acid substitutions selected from the group consisting of 1E, 38K, 66K, and 67A; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, and a GUCY2C-LCDR3 comprising the amino acid sequence of SEQ ID NO: 9, and the FR region of the GUCY2C-VL comprises one or more amino acid substitutions selected from the group consisting of 15P, 16G, 42S, 43S, 45K, 46S, 47W, 49Y, 58V, 71Y, and 76S; or
(iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40, and the FR region of the GUCY2C-VH comprises one or more amino acid substitutions selected from the group consisting of 1E, 2F, 28S, 69L, 71V, 73Q, and 75S; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39, and a GUCY2C-LCDR3 comprising the amino acid sequence of SEQ ID NO: 14, and the FR region of the GUCY2C-VL comprises one or more amino acid substitutions selected from the group consisting of 42S, 43S, 46P, 47W, 58V, and 71Y.

In some embodiments, provided is the anti-GUCY2C antibody according to any one of the above, wherein:
i) the GUCY2C-VH comprises the amino acid sequence of SEQ ID NO: 56, 25, or 57, and the GUCY2C-VL comprises the amino acid sequence of SEQ ID NO: 54, 55, 26, 48, 49, 50, 51, 52, or 53; or
ii) the GUCY2C-VH comprises the amino acid sequence of SEQ ID NO: 37, 15, or 38, and the GUCY2C-VL comprises the amino acid sequence of SEQ ID NO: 33, 16, 31, 32, 34, 35, or 36; or
iii) the GUCY2C-VH comprises the amino acid sequence of SEQ ID NO: 42 or 17, and the GUCY2C-VL comprises the amino acid sequence of SEQ ID NO: 41 or 18; or
iv-1) the GUCY2C-VH comprises the amino acid sequence of SEQ ID NO: 273, and the GUCY2C-VL comprises the amino acid sequence of SEQ ID NO: 274; or
iv-2) the GUCY2C-VH comprises the amino acid sequence of SEQ ID NO: 275, and the GUCY2C-VL comprises the amino acid sequence of SEQ ID NO: 276; or
iv-3) the GUCY2C-VH comprises the amino acid sequence of SEQ ID NO: 277, and the GUCY2C-VL comprises the amino acid sequence of SEQ ID NO: 278; or
iv-4) the GUCY2C-VH comprises the amino acid sequence of SEQ ID NO: 279, and the GUCY2C-VL comprises the amino acid sequence of SEQ ID NO: 280; or
iv-5) the GUCY2C-VH comprises the amino acid sequence of SEQ ID NO: 330, and the GUCY2C-VL comprises the amino acid sequence of SEQ ID NO: 331; or
iv-6) the GUCY2C-VH comprises the amino acid sequence of SEQ ID NO: 332, and the GUCY2C-VL comprises the amino acid sequence of SEQ ID NO: 333; or
iv-7) the GUCY2C-VH comprises the amino acid sequence of SEQ ID NO: 334, and the GUCY2C-VL comprises the amino acid sequence of SEQ ID NO: 335; or
iv-8) the GUCY2C-VH comprises the amino acid sequence of SEQ ID NO: 336, and the GUCY2C-VL comprises the amino acid sequence of SEQ ID NO: 337; or
iv-9) the GUCY2C-VH comprises the amino acid sequence of SEQ ID NO: 338, and the GUCY2C-VL comprises the amino acid sequence of SEQ ID NO: 339; or
iv-10) the GUCY2C-VH comprises the amino acid sequence of SEQ ID NO: 340, and the GUCY2C-VL comprises the amino acid sequence of SEQ ID NO: 341; or
iv-11) the GUCY2C-VH comprises the amino acid sequence of SEQ ID NO: 342, and the GUCY2C-VL comprises the amino acid sequence of SEQ ID NO: 343; or
iv-12) the GUCY2C-VH comprises the amino acid sequence of SEQ ID NO: 344, and the GUCY2C-VL comprises the amino acid sequence of SEQ ID NO: 345; or
iv-13) the GUCY2C-VH comprises the amino acid sequence of SEQ ID NO: 346, and the GUCY2C-VL comprises the amino acid sequence of SEQ ID NO: 347; or
iv-14) the GUCY2C-VH comprises the amino acid sequence of SEQ ID NO: 348, and the GUCY2C-VL comprises the amino acid sequence of SEQ ID NO: 349; or
iv-15) the GUCY2C-VH comprises the amino acid sequence of SEQ ID NO: 350, and the GUCY2C-VL comprises the amino acid sequence of SEQ ID NO: 351; or
iv-16) the GUCY2C-VH comprises the amino acid sequence of SEQ ID NO: 352, and the GUCY2C-VL comprises the amino acid sequence of SEQ ID NO: 353.

In some embodiments, provided is the antigen-binding molecule according to any one of the above, wherein:
i) the GUCY2C-VH comprises the amino acid sequence of SEQ ID NO: 56, and the GUCY2C-VL comprises the amino acid sequence of SEQ ID NO: 54, 55, 48, 49, 50, 51, or 52, or
the GUCY2C-VH comprises the amino acid sequence of SEQ ID NO: 25, and the GUCY2C-VL comprises the amino acid sequence of SEQ ID NO: 26, or
ii) the GUCY2C-VH comprises the amino acid sequence of SEQ ID NO: 37, and the GUCY2C-VL comprises the amino acid sequence of SEQ ID NO: 33, or
the GUCY2C-VH comprises the amino acid sequence of SEQ ID NO: 38, and the GUCY2C-VL comprises the amino acid sequence of SEQ ID NO: 33, 32, 34, or 36, or
the GUCY2C-VH comprises the amino acid sequence of SEQ ID NO: 15, and the GUCY2C-VL comprises the amino acid sequence of SEQ ID NO: 16, or
iii) the GUCY2C-VH comprises the amino acid sequence of SEQ ID NO: 42, and the GUCY2C-VL comprises the amino acid sequence of SEQ ID NO: 41, or
the GUCY2C-VH comprises the amino acid sequence of SEQ ID NO: 17, and the GUCY2C-VL comprises the amino acid sequence of SEQ ID NO: 18.

In some embodiments, provided is the antigen-binding molecule according to any one of the above, wherein:
i) the GUCY2C-VH comprises the amino acid sequence of SEQ ID NO: 56, and the GUCY2C-VL comprises the amino acid sequence of SEQ ID NO: 54, or
the GUCY2C-VH comprises the amino acid sequence of SEQ ID NO: 56, and the GUCY2C-VL comprises the amino acid sequence of SEQ ID NO: 55; or
ii) the GUCY2C-VH comprises the amino acid sequence of SEQ ID NO: 37, and the GUCY2C-VL comprises the amino acid sequence of SEQ ID NO: 33; or
iii) the GUCY2C-VH comprises the amino acid sequence of SEQ ID NO: 42, and the GUCY2C-VL comprises the amino acid sequence of SEQ ID NO: 41.

In some embodiments, provided is the antigen-binding molecule according to any one of the above, wherein the amino acid sequence of the GUCY2C-VH is set forth in SEQ ID NO: 56, and the amino acid sequence of the GUCY2C-VL is set forth in SEQ ID NO: 54.

In some embodiments, provided is the antigen-binding molecule according to any one of the above, wherein the amino acid sequence of the GUCY2C-VH is set forth in SEQ ID NO: 56, and the amino acid sequence of the GUCY2C-VL is set forth in SEQ ID NO: 55.

In some embodiments, provided is the antigen-binding molecule according to any one of the above, wherein the amino acid sequence of the GUCY2C-VH is set forth in SEQ ID NO: 37, and the amino acid sequence of the GUCY2C-VL is set forth in SEQ ID NO: 33.

In some embodiments, provided is the antigen-binding molecule according to any one of the above, wherein the amino acid sequence of the GUCY2C-VH is set forth in SEQ ID NO: 42, and the amino acid sequence of the GUCY2C-VL is set forth in SEQ ID NO: 41.

In some embodiments, provided is the anti-GUCY2C antibody according to any one of the above, wherein the anti-GUCY2C antibody comprises a heavy chain and a light chain, wherein:
the heavy chain comprises an amino acid sequence having at least 85% sequence identity to SEQ ID NO: 64, and the light chain comprises an amino acid sequence having at least 85% sequence identity to SEQ ID NO: 65 or 66; or
the heavy chain comprises an amino acid sequence having at least 85% sequence identity to SEQ ID NO: 60, and the light chain comprises an amino acid sequence having at least 85% sequence identity to SEQ ID NO: 61; or
the heavy chain comprises an amino acid sequence having at least 85% sequence identity to SEQ ID NO: 62, and the light chain comprises an amino acid sequence having at least 85% sequence identity to SEQ ID NO: 63.

In some embodiments, provided is the anti-GUCY2C antibody according to any one of the above, wherein the anti-GUCY2C antibody comprises a heavy chain and a light chain, wherein:
the heavy chain comprises the amino acid sequence of SEQ ID NO: 64, and the light chain comprises the amino acid sequence of SEQ ID NO: 65 or 66; or
the heavy chain comprises the amino acid sequence of SEQ ID NO: 60, and the light chain comprises the amino acid sequence of SEQ ID NO: 61; or
the heavy chain comprises the amino acid sequence of SEQ ID NO: 62, and the light chain comprises the amino acid sequence of SEQ ID NO: 63.

In some embodiments, provided is the anti-GUCY2C antibody according to any one of the above, wherein the anti-GUCY2C antibody comprises a heavy chain and a light chain; the amino acid sequence of the heavy chain is set forth in SEQ ID NO: 64, and the amino acid sequence of the light chain is set forth in SEQ ID NO: 65.

In some embodiments, provided is the anti-GUCY2C antibody according to any one of the above, wherein the anti-GUCY2C antibody comprises a heavy chain and a light chain; the amino acid sequence of the heavy chain is set forth in SEQ ID NO: 64, and the amino acid sequence of the light chain is set forth in SEQ ID NO: 66.

In some embodiments, provided is the anti-GUCY2C antibody according to any one of the above, wherein the anti-GUCY2C antibody comprises a heavy chain and a light chain; the amino acid sequence of the heavy chain is set forth in SEQ ID NO: 60, and the amino acid sequence of the light chain is set forth in SEQ ID NO: 61.

In some embodiments, provided is the anti-GUCY2C antibody according to any one of the above, wherein the anti-GUCY2C antibody comprises a heavy chain and a light chain; the amino acid sequence of the heavy chain is set forth in SEQ ID NO: 62, and the amino acid sequence of the light chain is set forth in SEQ ID NO: 63.

In some embodiments, provided is the anti-GUCY2C antibody according to any one of the above, wherein the anti-GUCY2C antibody is an antibody fragment, and the antibody fragment is Fab, Fab′, F(ab′)2, Fd, Fv, scFv, dsFv, or dAb.

In some embodiments, provided is the anti-GUCY2C antibody according to any one of the above, wherein the anti-GUCY2C antibody is a bispecific antibody.

In some embodiments, provided is the anti-GUCY2C antibody according to any one of the above, wherein the anti-GUCY2C antibody is a bispecific antibody that specifically binds to GUCY2C and CD3.

In another aspect, the present disclosure provides a pharmaceutical composition comprising: a therapeutically effective amount of the antigen-binding molecule according to any one of the above or the anti-GUCY2C antibody according to any one of the above, and one or more pharmaceutically acceptable carriers, diluents, buffers, or excipients.

In another aspect, the present disclosure also provides an isolated nucleic acid encoding the antigen-binding molecule according to any one of the above or the anti-GUCY2C antibody according to any one of the above.

In another aspect, the present disclosure also provides a vector comprising the isolated nucleic acid according to the above.

In another aspect, the present disclosure also provides a host cell comprising the isolated nucleic acid according to the above.

In another aspect, the present disclosure also provides a method for treating a disease, wherein the method comprises administering to a subject a therapeutically effective amount of the antigen-binding molecule according to any one of the above or the anti-GUCY2C antibody according to any one of the above or the composition according to any one of the above.

In another aspect, the present disclosure also provides use of the antigen-binding molecule according to any one of the above or the antibody according to any one of the above or the composition thereof in the preparation of a medicament for treating a disease.

In another aspect, the present disclosure also provides the antigen-binding molecule according to any one of the above or the anti-GUCY2C antibody according to any one of the above or the composition according to any one of the above for use as a medicament. In some embodiments, the medicament is used for treating a disease.

In some embodiments, the disease according to any one of the above is a proliferative disease or a tumor. In some embodiments, the disease according to any one of the above is a tumor, which is gastrointestinal cancer (gastric cancer (gastric adenocarcinoma) and intestinal cancer (small intestine cancer and colorectal cancer (colon cancer and rectal cancer))), esophageal cancer (i.e., esophagus cancer), anal cancer, liver cancer (hepatocellular carcinoma), gallbladder cancer, bile duct cancer, pancreatic cancer, prostate cancer, renal cancer (renal cell carcinoma), urothelial cancer, breast cancer, bladder cancer, thymus cancer, melanoma, glioma (glioblastoma), sarcoma (osteosarcoma), ovarian cancer, thyroid cancer, cervical squamous cell cancer, endometrial cancer, lung cancer (non-small cell lung cancer (lung squamous cell cancer and lung adenocarcinoma) and small cell lung cancer), skin cancer, head and neck cancer (head and neck squamous cell cancer), brain cancer, glioblastoma multiforme, gastroesophageal cancer (gastroesophageal adenocarcinoma), adenocarcinomas metastatic to the liver, multiple myeloma, lymphoma (Hodgkin's lymphoma, non-Hodgkin's lymphoma, or B-cell lymphoma), or leukemia (such as acute myeloid leukemia (AML), acute lymphoblastic leukemia (ALL), and chronic lymphocytic leukemia (CLL)).

In some embodiments, the cells of the tumor according to the above or vascular endothelial cells close to the tumor express GUCY2C.

In some embodiments, the tumor according to the above is a solid tumor.

In some embodiments, the tumor according to the above is a solid tumor, which is gastrointestinal cancer (gastric cancer (gastric adenocarcinoma) and intestinal cancer (small intestine cancer and colorectal cancer (colon cancer and rectal cancer))), esophageal cancer (i.e., esophagus cancer), anal cancer, liver cancer (hepatocellular carcinoma), gallbladder cancer, bile duct cancer, pancreatic cancer, prostate cancer, renal cancer (renal cell carcinoma), urothelial cancer, breast cancer, bladder cancer, thymus cancer, melanoma, glioma (glioblastoma), sarcoma (osteosarcoma), ovarian cancer, thyroid cancer, cervical squamous cell cancer, adenocarcinoma of endometrium, lung cancer (non-small cell lung cancer (lung squamous cell cancer and lung adenocarcinoma), and small cell lung cancer), skin cancer, head and neck cancer (head and neck squamous cell cancer), brain cancer, glioblastoma multiforme, gastroesophageal cancer (gastroesophageal adenocarcinoma), or adenocarcinomas metastatic to the liver.

In some embodiments, the tumor according to the above is gastrointestinal cancer; preferably gastric cancer or intestinal cancer, and more preferably gastric adenocarcinoma, small intestine cancer, or colorectal cancer.

In some embodiments, the tumor according to the above is a non-solid tumor.

In some embodiments, the tumor according to the above is a non-solid tumor, which is multiple myeloma, lymphoma (Hodgkin's lymphoma, non-Hodgkin's lymphoma, or B-cell lymphoma), or leukemia (acute myeloid leukemia (AML), acute lymphoblastic leukemia (ALL), and chronic lymphocytic leukemia (CLL)).

In some embodiments, the aforementioned disease is a disease associated with GUCY2C; in some embodiments, the aforementioned disease is a disease expressing GUCY2C.

In some embodiments, provided is the method for treating a disease according to the above, which further comprises using a second therapeutic agent. In some embodiments, the second therapeutic agent includes an anti-tumor agent, radiation therapy, an antibody-drug conjugate, a bispecific antibody, a bispecific antibody conjugated to an anti-tumor agent, an immune checkpoint inhibitor, or a combination thereof.

In some embodiments, provided is the method for treating a disease according to the above, wherein the second therapeutic agent is administered simultaneously, sequentially, or separately with the antigen-binding molecule according to any one of the embodiments in the present disclosure.

In some embodiments, provided is the method for treating a disease according to the above, wherein the second therapeutic agent is a bispecific antibody that specifically binds to CD28 and EGFR.

In some embodiments, provided is the method for treating a disease according to the above, wherein the second therapeutic agent is a bispecific antibody that specifically binds to CD28 and EGFR, which comprises at least one antigen-binding moiety that specifically binds to CD28 and at least one antigen-binding moiety that specifically binds to EGFR.

In some embodiments, provided is the method for treating a disease according to the above, wherein the second therapeutic agent is a bispecific antibody that specifically binds to CD28 and EGFR, which comprises at least one antigen-binding moiety that specifically binds to CD28 and at least one antigen-binding moiety that specifically binds to EGFR; the antigen-binding moiety that specifically binds to CD28 comprises a heavy chain variable region CD28-VH and a light chain variable region CD28-VL, and the antigen-binding moiety that specifically binds to EGFR comprises a heavy chain variable region EGFR-VH and a light chain variable region EGFR-VL.

In some embodiments, provided is the method for treating a disease according to the above, wherein:
i) the CD28-VH comprises: a CD28-HCDR1 comprising the amino acid sequence of SEQ ID NO: 146, a CD28-HCDR2 comprising the amino acid sequence of SEQ ID NO: 147, and a CD28-HCDR3 comprising the amino acid sequence of SEQ ID NO: 148; and the CD28-VL comprises: a CD28-LCDR1 comprising the amino acid sequence of SEQ ID NO: 149, a CD28-LCDR2 comprising the amino acid sequence of SEQ ID NO: 150, and a CD28-LCDR3 comprising the amino acid sequence of SEQ ID NO: 151; or
ii) the CD28-VH comprises: a CD28-HCDR1 comprising the amino acid sequence of SEQ ID NO: 160, a CD28-HCDR2 comprising the amino acid sequence of SEQ ID NO: 161, and a CD28-HCDR3 comprising the amino acid sequence of SEQ ID NO: 162; and the CD28-VL comprises: a CD28-LCDR1 comprising the amino acid sequence of SEQ ID NO: 149, a CD28-LCDR2 comprising the amino acid sequence of SEQ ID NO: 150, and a CD28-LCDR3 comprising the amino acid sequence of SEQ ID NO: 163; or
iii) the CD28-VH comprises: a CD28-HCDR1 comprising the amino acid sequence of SEQ ID NO: 168, a CD28-HCDR2 comprising the amino acid sequence of SEQ ID NO: 169, and a CD28-HCDR3 comprising the amino acid sequence of SEQ ID NO: 170; and the CD28-VL comprises: a CD28-LCDR1 comprising the amino acid sequence of SEQ ID NO: 171, a CD28-LCDR2 comprising the amino acid sequence of SEQ ID NO: 172, and a CD28-LCDR3 comprising the amino acid sequence of SEQ ID NO: 173.

In some embodiments, provided is the method for treating a disease according to the above, wherein:
i) the CD28-VH comprises the amino acid sequence of SEQ ID NO: 154, and the CD28-VL comprises the amino acid sequence of SEQ ID NO: 155; or
ii) the CD28-VH comprises the amino acid sequence of SEQ ID NO: 164, and the CD28-VL comprises the amino acid sequence of SEQ ID NO: 165; or
iii) the CD28-VH comprises the amino acid sequence of SEQ ID NO: 174, and the CD28-VL comprises the amino acid sequence of SEQ ID NO: 175.

In some embodiments, provided is the method for treating a disease according to the above, wherein the antigen-binding moiety that specifically binds to EGFR comprises a heavy chain variable region EGFR-VH and a light chain variable region EGFR-VL, wherein:
the EGFR-VH comprises: an EGFR-HCDR1 comprising the amino acid sequence of SEQ ID NO: 140, an EGFR-HCDR2 comprising the amino acid sequence of SEQ ID NO: 141, and an EGFR-HCDR3 comprising the amino acid sequence of SEQ ID NO: 142; and the EGFR-VL comprises: an EGFR-LCDR1 comprising the amino acid sequence of SEQ ID NO: 143, an EGFR-LCDR2 comprising the amino acid sequence of SEQ ID NO: 144, and an EGFR-LCDR3 comprising the amino acid sequence of SEQ ID NO: 145.

In some embodiments, provided is the method for treating a disease according to the above, wherein the EGFR-VH comprises the amino acid sequence of SEQ ID NO: 152, and the EGFR-VL comprises the amino acid sequence of SEQ ID NO: 153.

In some embodiments, provided is the method for treating a disease according to the above, wherein the bispecific antibody that specifically binds to CD28 and EGFR comprises an Fc region. In some embodiments, the Fc region is an IgG Fc region. In some embodiments, the Fc region is an IgG1 Fc region. In some embodiments, the Fc region comprises one or more amino acid substitutions capable of reducing the binding of the Fc region to an Fcγ receptor. In some embodiments, the Fc region is a human IgG1 Fc region, and the amino acids at positions 234 and 235 are A, as numbered according to the EU index.

In some embodiments, provided is the method for treating a disease according to the above, wherein the bispecific antibody that specifically binds to CD28 and EGFR comprises an Fc region comprising a first subunit Fc1 and a second subunit Fc2 capable of associating with each other, and the Fc1 and the Fc2 each independently have one or more amino acid substitutions that reduce homodimerization of the Fc region. In some embodiments, the Fc1 has a knob structure according to the knob-into-hole technique, and the Fc2 has a hole structure according to the knob-into-hole technique. In some embodiments, for the Fc1, the amino acid at position 366 is W, and for the Fc2, the amino acid at position 366 is S, the amino acid at position 368 is A, and the amino acid at position 407 is V, as numbered according to the EU index. In some embodiments, for the Fc1, the amino acid at position 354 is C and the amino acid at position 366 is W, and for the Fc2, the amino acid at position 349 is C, the amino acid at position 366 is S, the amino acid at position 368 is A, and the amino acid at position 407 is V, as numbered according to the EU index. In some embodiments, the Fc1 comprises the amino acid sequence of SEQ ID NO: 248, and the Fc2 comprises the amino acid sequence of SEQ ID NO: 249.

In some embodiments, provided is the antigen-binding molecule according to any one of the above, wherein the Titin chain and the Obscurin chain are any Titin chain and Obscurin chain in Table 3-1 and Table 3-2 of the present disclosure that can form a dimer. In some embodiments, provided is the antigen-binding molecule according to any one of the above, wherein the Titin chain comprises the amino acid sequence of SEQ ID NO: 198. In some embodiments, provided is the antigen-binding molecule according to any one of the above, wherein the Obscurin chain comprises the amino acid sequence of SEQ ID NO: 236.

In some embodiments, provided is the antigen-binding molecule according to any one of the above, wherein the linkers 8 are all peptide linkers known in the art, so long as the antigen-binding molecule is capable of exhibiting the desired antigen binding activity. For example, the peptide linkers may be flexible peptides comprising 1-50 or 3-20 amino acid residues. In some embodiments, the peptide linker is 3-15 amino acid residues in length. In some embodiments, the peptide linkers each independently have a structure of (GGGGS)n, wherein n is 1, 2, or 3. In some embodiments, the sequence of the linker 8 is GGGGS (SEQ ID NO: 90).

In some embodiments, provided is the antigen-binding molecule according to any one of the above, wherein the CH1 is the CH1 sequence of an IgG. In some embodiments, the CH1 is the CH1 of an IgG1. In some embodiments, the CH1 comprises the amino acid sequence of SEQ ID NO: 84.

In some embodiments, provided is the antigen-binding molecule according to any one of the above, wherein the CL is the light chain constant region of an antibody. In some embodiments, provided is the antigen-binding molecule according to any one of the above, wherein the CL is a kappa or lamada light chain constant region. In some embodiments, the CL comprises the amino acid sequence of SEQ ID NO: 59.

In some embodiments, provided is the method for treating a disease according to the above, wherein the bispecific antibody that specifically binds to CD28 and EGFR comprises one antigen-binding moiety that specifically binds to CD28 and one antigen-binding moiety that specifically binds to EGFR, the antigen-binding moiety that specifically binds to CD28 is a replaced Fab comprising a Titin chain and an Obscurin chain capable of forming a dimer, and the antigen-binding moiety that specifically binds to EGFR is a Fab.

In some embodiments, provided is the method for treating a disease according to the above, wherein
the bispecific antibody that specifically binds to CD28 and EGFR comprises one first chain having a structure represented by formula (m), a second chain having a structure represented by formula (n), a third chain having a structure represented by formula (o), and a fourth chain having a structure represented by formula (p):

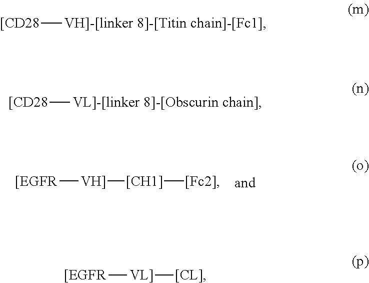

wherein the linker 8 is a peptide linker; or the linker 8 is absent;
the structures represented by formulas (m), (n), (o), and (p) are arranged from N-terminus to C-terminus.

In some embodiments, provided is the method for treating a disease according to the above, wherein the bispecific antibody that specifically binds to CD28 and EGFR comprises: one first chain comprising the amino acid sequence of SEQ ID NO: 156, one second chain comprising the amino acid sequence of SEQ ID NO: 157, one third chain comprising the amino acid sequence of SEQ ID NO: 158, and one fourth chain comprising the amino acid sequence of SEQ ID NO: 159; or
the bispecific antibody that specifically binds to CD28 and EGFR comprises: one first chain comprising the amino acid sequence of SEQ ID NO: 166, one second chain comprising the amino acid sequence of SEQ ID NO: 167, one third chain comprising the amino acid sequence of SEQ ID NO: 158, and one fourth chain comprising the amino acid sequence of SEQ ID NO: 159.

In some embodiments, provided is the method for treating a disease according to the above, wherein the bispecific antibody that specifically binds to CD28 and EGFR comprises: one first chain having the amino acid sequence set forth in SEQ ID NO: 156, one second chain having the amino acid sequence set forth in SEQ ID NO: 157, one third chain having the amino acid sequence set forth in SEQ ID NO: 158, and one fourth chain having the amino acid sequence set forth in SEQ ID NO: 159.

In some embodiments, provided is the method for treating a disease according to the above, wherein the bispecific antibody that specifically binds to CD28 and EGFR comprises: one first chain having the amino acid sequence set forth in SEQ ID NO: 166, one second chain having the amino acid sequence set forth in SEQ ID NO: 167, one third chain having the amino acid sequence set forth in SEQ ID NO: 158, and one fourth chain having the amino acid sequence set forth in SEQ ID NO: 159.

In some embodiments, provided is the method for treating a disease according to the above, wherein the bispecific antibody that specifically binds to CD28 and EGFR comprises one antigen-binding moiety that specifically binds to CD28 and one antigen-binding moiety that specifically binds to EGFR, the antigen-binding moiety that specifically binds to CD28 is a Fab, and the antigen-binding moiety that specifically binds to EGFR is a replaced Fab comprising a Titin chain and an Obscurin chain capable of forming a dimer.

In some embodiments, provided is the method for treating a disease according to the above, wherein the bispecific antibody that specifically binds to CD28 and EGFR comprises one first chain having a structure represented by formula (q), a second chain having a structure represented by formula (r), a third chain having a structure represented by formula (s), and a fourth chain having a structure represented by formula (t):

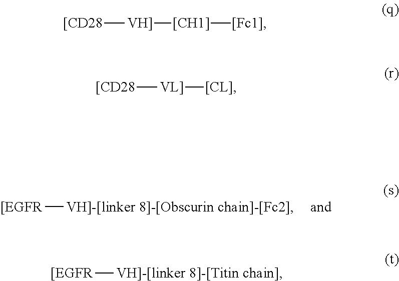

wherein the linker 8 is a peptide linker; or the linker 8 is absent;
the structures represented by formulas (q), (r), (s), and (t) are arranged from N-terminus to C-terminus.

In some embodiments, provided is the method for treating a disease according to the above, wherein the bispecific antibody that specifically binds to CD28 and EGFR comprises: one first chain comprising the amino acid sequence of SEQ ID NO: 176, one second chain comprising the amino acid sequence of SEQ ID NO: 177, one third chain comprising the amino acid sequence of SEQ ID NO: 178, and one fourth chain comprising the amino acid sequence of SEQ ID NO: 179.

In some embodiments, provided is the method for treating a disease according to the above, wherein the bispecific antibody that specifically binds to CD28 and EGFR comprises: one first chain having the amino acid sequence set forth in SEQ ID NO: 176, one second chain having the amino acid sequence set forth in SEQ ID NO: 177, one third chain having the amino acid sequence set forth in SEQ ID NO: 178, and one fourth chain having the amino acid sequence set forth in SEQ ID NO: 179.

The antigen-binding molecule provided by the present disclosure has the following characteristics: good therapeutic activity, safety, pharmacokinetic properties, and druggability (e.g., stability).

### BRIEF DESCRIPTION OF THE DRAWINGS

FIG. 1A: a structural schematic diagram of Format 1 of GUCY2C-CD3 bispecific antibody; FIG. 1B: a structural schematic diagram of Format 2 of GUCY2C-CD3 bispecific antibody, wherein Ob represents Obscurin; FIG. 1C: a structural schematic diagram of Format 3 of GUCY2C-CD3 bispecific antibody, wherein Ob represents Obscurin; FIG. 1D: a structural schematic diagram of Format 4 of GUCY2C-CD3 bispecific antibody; FIG. 1E: a structural schematic diagram of Format 5 of GUCY2C-CD3 bispecific antibody, wherein Ob represents Obscurin.

FIGS. 2A-2C: the ability of GUCY2C monoclonal antibodies to bind to HCT116/hGUCY2C cells; FIG. 2D: the ability of GUCY2C monoclonal antibodies to bind to HT-55 cells; FIG. 2E: the ability of GUCY2C monoclonal antibodies to bind to T84 cells.

FIG. 3A: the ability of GUCY2C-CD3 bispecific antibodies to bind to HCT116/hGUCY2C cells; FIG. 3B: the ability of GUCY2C-CD3 bispecific antibodies to bind to HT55 cells; FIG. 3C: the ability of GUCY2C-CD3 bispecific antibodies to bind to Jurkat cells.

FIG. 4A: the ability of antibodies to activate Jurkat/NFAT cells tested using HCT116/hGUCY2C as target cells; FIG. 4B: the ability of antibodies to activate Jurkat/NFAT cells tested using LS1034 as target cells.

FIG. 5A: the results for IFN-γ release under the combined action of the GUCY2C-CD3 bispecific antibody and LS1034 cells; FIGS. 5B and 5C: the results for IFN-γ release under the combined action of the GUCY2C-CD3 bispecific antibody and LS174T cells.

FIG. 6A: the results for IL-6 release under the combined action of the GUCY2C-CD3 bispecific antibody and LS1034 cells; FIGS. 6B and 6C: the results for IL-6 release under the combined action of the GUCY2C-CD3 bispecific antibody and LS174T cells.

### DETAILED DESCRIPTION

### Terminology

The terminology used herein is for the purpose of describing embodiments only and is not intended to be limiting. Unless defined otherwise, all technical and scientific terms used herein have the same meaning as commonly understood by one of ordinary skill in the art to which the present disclosure pertains.

Unless the context clearly requires otherwise, throughout the description and the claims, the words “comprise”, “have”, “include”, and the like are to be understood in an inclusive sense as opposed to an exclusive or exhaustive sense; that is to say, in the sense of “including, but not limited to”. Unless otherwise stated, “comprise” includes “consist of”. For example, for a GUCY2C-HCDR1 comprising the amino acid sequence of SEQ ID NO: 1, it specifically encompasses a GUCY2C-HCDR1 whose amino acid sequence is set forth in SEQ ID NO: 1.

The three-letter and single-letter codes used in the present disclosure for amino acids are as described in J Biol. Chem, 243, p 3558 (1968).

The term “and/or”, e.g., “X and/or Y”, should be understood to mean “X and Y” or “X or Y” and should be used to provide explicit support for both meanings or either meaning.

The term “CD28” (cluster of differentiation 28, Tp44) refers to any CD28 protein from any vertebrate source, including mammals such as primates (e.g., humans), non-human primates (e.g., cynomolgus monkeys), and rodents (e.g., mice and rats). CD28 is expressed on T cells and provides costimulatory signals required for T cell activation and survival. In addition to the T cell receptor (TCR), stimulation of T cells by CD28 may also provide an effective signal for the production of various interleukins. CD28 is a receptor of the CD80 (B7.1) and CD86 (B7.2) proteins and is the only B7 receptor constitutively expressed on naive T cells. The amino acid sequence of human CD28 is shown in UniProt (www.uniprot.org) under the accession number P10747.

The term “amino acid” refers to naturally occurring and synthetic amino acids, as well as amino acid analogs and amino acid mimics that function in a manner similar to the naturally occurring amino acids. Naturally occurring amino acids are those encoded by genetic codes and those amino acids later modified, e.g., hydroxyproline, γ-carboxyglutamic acid, and O-phosphoserine. Amino acid analogs refer to compounds that have a substantially identical chemical structure (i.e., an a carbon that binds to hydrogen, carboxyl, amino, and an R group) to naturally occurring amino acids, e.g., homoserine, norleucine, methionine sulfoxide, and methioninemethyl sulfonium. Such analogs have a modified R group (e.g., norleucine) or a modified peptide skeleton, but retain a substantially identical chemical structure to naturally occurring amino acids. Amino acid mimics refer to chemical compounds that have a different structure from amino acids, but function in a manner similar to naturally occurring amino acids.

The term “amino acid mutation” includes amino acid substitutions, deletions, insertions, and modifications. Any combination of substitutions, deletions, insertions, and modifications can be made to obtain a final construct, as long as the final construct possesses the desired properties, such as reduced binding to the Fc receptor. Amino acid sequence deletions and insertions include deletions and insertions at the amino terminus and/or the carboxyl terminus of a polypeptide chain. Specific amino acid mutations may be amino acid substitutions. In one embodiment, the amino acid mutation is a non-conservative amino acid substitution, i.e., replacement of one amino acid with another amino acid having different structural and/or chemical properties. Amino acid substitutions include replacement with non-naturally occurring amino acids or with derivatives of the 20 native amino acids (e.g., 4-hydroxyproline, 3-methylhistidine, ornithine, homoserine, and 5-hydroxylysine). Amino acid mutations can be generated using genetic or chemical methods well known in the art. Genetic methods may include site-directed mutagenesis, PCR, gene synthesis, and the like. It is contemplated that methods for altering amino acid side chain groups other than genetic engineering, such as chemical modification, may also be used. Various names may be used herein to indicate the same amino acid mutation. Herein, the expression of “position+amino acid residue” may be used to denote an amino acid residue at a specific position. For example, 366W indicates that the amino acid residue at position 366 is W. T366W indicates that the amino acid residue at position 366 is mutated from the original T to W.

The term “antigen-binding molecule” is used in the broadest sense and encompasses a variety of molecules that specifically bind to an antigen, including but not limited to antibodies, other polypeptides having antigen binding activity, and antibody fusion proteins in which the two are fused, as long as they exhibit desired antigen binding activity. The antigen-binding molecule herein comprises a variable region (VH) and a variable region (VL) which together comprise an antigen-binding domain. Illustratively, the antigen-binding molecules herein are bispecific antigen-binding molecules (e.g., bispecific antibodies).

The term “antibody” is used in the broadest sense and encompasses a variety of antibody structures, including but not limited to monoclonal antibodies, polyclonal antibodies; monospecific antibodies, multispecific antibodies (e.g., bispecific antibodies), and full-length antibodies, and antibody fragments (or antigen-binding fragments, or antigen-binding moieties), as long as they exhibit the desired antigen binding activity. For example, a native IgG antibody is a heterotetrameric glycoprotein of about 150,000 Daltons composed of two identical light chains and two identical heavy chains linked by a disulfide bond. From the N-terminus to the C-terminus, each heavy chain has one variable region (VH), also known as variable heavy domain or heavy chain variable region, followed by three constant domains (CH1, CH2, and CH3). Similarly, from the N-terminus to the C-terminus, each light chain has one variable region (VL), also known as variable light domain or light chain variable domain, followed by one constant light domain (light chain constant region, CL).

The term “bispecific antibody” refers to an antibody (including an antibody or an antigen-binding fragment thereof, such as a single-chain antibody) capable of specifically binding to two different antigens or at least two different epitopes of the same antigen. Bispecific antibodies of various structures have been disclosed in the prior art; the bispecific antibodies can be classified into IgG-like bispecific antibodies and antibody-fragment-type bispecific antibodies according to the integrity of IgG molecules; the bispecific antibodies can be classified into bivalent, trivalent, tetravalent or higher-valent bispecific antibodies according to the number of the antigen-binding regions; the bispecific antibodies can be classified into symmetric bispecific antibodies and asymmetric bispecific antibodies according to the presence of symmetry in their structures. Among them, the bispecific antibodies based on antibody fragments, such as Fab fragments lacking Fc fragments, formed by binding 2 or more Fab fragments in one molecule, have low immunogenicity, small molecular weight and high tumor tissue permeability, and typical antibody structures of this type are, e.g., F(ab)₂, scFv-Fab, and (scFv)₂-Fab; IgG-like bispecific antibodies (e.g., having Fc fragments) have relatively large molecular weight, and the Fc fragments facilitate the purification of the antibodies and increase their solubility and stability, and the Fc fragments may further bind to the receptor FcRn and increase the serum half-life of the antibodies, and typical structural models of bispecific antibodies are, e.g., KiH, CrossMAb, Triomab quadroma, FcΔAdp, ART-Ig, BiMAb, Biclonics, BEAT, DuoBody, Azymetric, XmAb, 2:1 TCBs, 1Fab-IgG TDB, FynomAb, two-in-one/DAF, scFv-Fab-IgG, DART-Fc, LP-DART, CODV-Fab-TL, HLE-BiTE, F(ab)2-CrossMAb, IgG-(scFv)2, Bs4Ab, DVD-Ig, Tetravalent-DART-Fe, (scFv)4-Fc, CODV-Ig, mAb2, and F(ab)4-CrossMAb (see Aran F. Labrijn et al., Nature Reviews Drug Discovery, volume 18, pages 585-608 (2019); Chen S1 et al., J Immunol Res., Feb. 11, 2019; 2019:4516041).

The term “variable region” or “variable domain” refers to a domain in an antigen-binding molecule that binds to an antigen. Herein, in the antigen-binding moiety that specifically binds to GUCY2C a heavy chain variable region is denoted by GUCY2C-VH, and a light chain variable region is denoted by GUCY2C-VL; in the antigen-binding moiety that specifically binds to CD3, a heavy chain variable region is denoted by CD3-VH, and a light chain variable region is denoted by CD3-VL. VH and VL each comprise four conserved framework regions (FRs) and three complementarity determining regions (CDRs). The term “complementarity determining region” or “CDR” refers to a region in the variable domain that primarily contributes to antigen binding; “framework” or “FR” refers to variable domain residues other than CDR residues. A VH comprises 3 CDRs: HCDR1, HCDR2, and HCDR3; a VL comprises 3 CDRs: LCDR1, LCDR2, and LCDR3. Herein, the 3 CDRs in GUCY2C-VH are denoted by GUCY2C-HCDR1, GUCY2C-HCDR2, and GUCY2C-HCDR3, respectively; the 3 CDRs in GUCY2C-VL are denoted by GUCY2C-LCDR1, GUCY2C-LCDR2, and GUCY2C-LCDR3, respectively; the 3 CDRs in CD3-VH are denoted by CD3-HCDR1, CD3-HCDR2, and CD3-HCDR3, respectively; the 3 CDRs in CD3-VL are denoted by CD3-LCDR1, CD3-LCDR2, and CD3-LCDR3, respectively. Each VH and VL is, when viewed from N-terminus to C-terminus: FR1, CDR1, FR2, CDR2, FR3, CDR3, FR4. A single VH or VL may be sufficient to provide antigen binding specificity.

The amino acid sequence boundaries of the CDRs can be determined by a variety of well-known schemes, for example, the “Kabat” numbering scheme (see Kabat et al. (1991), Sequences of Proteins of Immunological Interest, 5th ed., Public Health Service, National Institutes of Health, Bethesda, MD), the “Chothia” numbering scheme, the “ABM” numbering scheme, the “Contact” numbering scheme (see Martin, A C R. Protein Sequence and Structure Analysis of Antibody Variable Domains[J]. 2001), and the ImMunoGenTics (IMGT) numbering scheme (Lefranc, M. P. et al., Dev. Comp. Immunol., 27, 55-77 (2003); Front Immunol., Oct. 16, 2018; 9: 2278), and the like. The corresponding relationships between the various numbering schemes are well known to those skilled in the art. The numbering schemes of the present disclosure are shown in Table 1 below.

TABLE 1
Relationships between CDR numbering schemes

| CDR | IMGT | Kabat | AbM | Chothia | Contact |
|---|---|---|---|---|---|
| HCDR1 | 27-38 | 31-35 | 26-35 | 26-32 | 30-35 |
| HCDR2 | 56-65 | 50-65 | 50-58 | 52-56 | 47-58 |
| HCDR3 | 105-117 | 95-102 | 95-102 | 95-102 | 93-101 |
| LCDR1 | 27-38 | 24-34 | 24-34 | 24-34 | 30-36 |
| LCDR2 | 56-65 | 50-56 | 50-56 | 50-56 | 46-55 |
| LCDR3 | 105-117 | 89-97 | 89-97 | 89-97 | 89-96 |

Unless otherwise stated, the “Kabat” numbering scheme is applied to the sequences of the variable regions and CDRs in examples of the present disclosure.

The term “antibody fragment” refers to a molecule different from an intact antibody, which comprises a moiety of an intact antibody that retains the antigen binding ability of the intact antibody. Examples of antibody fragments include, but are not limited to, Fv, Fab, Fab′, Fab′-SH, F(ab′)2, single-domain antibodies, single-chain Fab (scFab), diabodies, linear antibodies, single-chain antibody molecules (e.g., scFv), and multispecific antibodies (e.g., bispecific antibodies) formed from antibody fragments.

The term “Fc region” or “fragment crystallizable region” is used to define the C-terminal region of the heavy chain of an antibody, including native Fc regions and engineered Fc regions. In some embodiments, the Fc region comprises two subunits, which may be identical or different. In some embodiments, the Fc region of the human IgG heavy chain is defined as extending from the amino acid residue at position Cys226 or from Pro230 to its carboxyl terminus. Suitable native sequence Fc regions for the antibodies described herein include human IgG1, IgG2 (IgG2A, IgG2B), IgG3, and IgG4. Unless otherwise stated, the numbering scheme for the Fc region is the EU index.

The term “Titin chain” refers to a peptide fragment of 78-118 amino acids in length comprising a Titin Ig-like 152 domain in a Titin protein or a functional variant thereof, wherein the Titin chain is capable of binding to an Obscurin Ig-like 1 domain or an Obscurin-like Ig-like 1 domain to form a dimerized complex. The term “Obscurin chain” refers to a peptide fragment of 87-117 amino acids in length comprising an Obscurin Ig-like 1 domain in an Obscurin protein or a functional variant thereof, or a peptide fragment of 78-118 amino acids in length comprising an Obscurin-like Ig-like 1 domain in an Obscurin-like 1 protein or a functional variant thereof, wherein the Obscurin chain is capable of binding to a Titin Ig-like 152 domain to form a dimerized complex. The Titin chain and the Obscurin chain of the present disclosure can be used to replace the CH1 and CL in a Fab to form a replaced Fab (Fab-S), and the replacement does not affect the binding of the antigen-binding molecule to an antigen.

The term “chimeric” antibody refers to an antibody in which a portion of the heavy and/or light chain is derived from a particular source or species and the remainder of the heavy and/or light chain is derived from a different source or species.

The term “humanized” antibody refers to an antibody that retains the reactivity of a non-human antibody while having low immunogenicity in humans. For example, this can be achieved by retaining the non-human CDRs and replacing the remainder of the antibody with its human counterparts (i.e., the constant regions and the framework region portion of the variable regions).

The term “affinity” refers to the overall strength of the non-covalent interaction between a single binding site of a molecule (e.g., an antibody) and its binding ligand (e.g., an antigen). Unless otherwise indicated, as used herein, “binding affinity” refers to an internal binding affinity that reflects a 1:1 interaction between members of a binding pair (e.g., an antibody and an antigen). The affinity of a molecule X for its ligand Y can be generally expressed by the equilibrium dissociation constant (KD). Affinity can be determined by conventional methods known in the art, including those described herein. The term “kassoc” or “ka” refers to the association rate of a particular antibody-antigen interaction, while the term “kdis” or “kd” as used herein refers to the dissociation rate of a particular antibody-antigen interaction. As used herein, the term “KD” refers to the equilibrium dissociation constant, which is the ratio of kd to ka (i.e., kd/ka) and expressed as molar concentration (M). The KD value of an antibody can be measured using methods known in the art, such as surface plasmon resonance, ELISA, or solution equilibrium titration (SET).

The term “monoclonal antibody” refers to a population of substantially homogeneous antibodies, that is, the amino acid sequences of the antibody molecules comprised in the population are identical, except for a small number of natural mutations that may exist. In contrast, polyclonal antibody formulations generally comprise a plurality of different antibodies having different amino acid sequences in their variable domains, which are generally specific for different epitopes. “Monoclonal” refers to the characteristics of an antibody obtained from a population of substantially homogeneous antibodies and should not be construed as requiring the production of the antibody by any particular method. In some embodiments, the antibody provided by the present disclosure is a monoclonal antibody.

The term “antigen” refers to a molecule or a portion of a molecule that is capable of being bound by a selective binding agent of an antigen-binding protein (e.g., an antibody). An antigen may have one or more epitopes capable of interacting with different antigen-binding proteins (e.g., antibodies).

The term “epitope” refers to an area or region on an antigen that is capable of specifically binding to an antibody or an antigen-binding fragment thereof. Epitopes can be formed by contiguous amino acids (linear epitopes) or comprise non-contiguous amino acids (conformational epitopes), e.g., non-contiguous amino acids that are in spatial proximity to each other due to folding of an antigen (i.e., by tertiary folding of an antigen of a protein nature). The difference between the conformational epitope and the linear epitope is that in the presence of denaturing solvents, the binding of the antibody to the conformational epitope is lost. An epitope comprises at least 3, at least 4, at least 5, at least 6, at least 7, or 8-10 amino acids in a unique spatial conformation. Screening for antibodies that bind to particular epitopes (i.e., those that bind to identical epitopes) can be performed using routine methods in the art, such as but not limited to, alanine scanning, peptide blotting (see Meth. Mol. Biol. 248 (2004) 443-463), peptide cleavage analysis, epitope excision, epitope extraction, chemical modification of the antigen (see Prot. Sci. 9 (2000) 487-496), and cross-blocking (see “Antibodies”, Harlow and Lane (Cold Spring Harbor Press, Cold Spring Harb., NY)).

The term “capable of specifically binding”, “specifically binding”, or “binding” means that an antibody is capable of binding to a certain antigen or epitope with higher affinity than to other antigens or epitopes. Generally, an antibody binds to an antigen or epitope with an equilibrium dissociation constant (KD) of about 1×10⁻⁷ M or less (e.g., about 1×10⁻⁸ M or less). In some embodiments, the KD for the binding of an antibody to an antigen is 10% or less (e.g., 1%) of the KD for the binding of the antibody to a non-specific antigen (e.g., BSA or casein). KD may be determined using known methods, for example, by a FACS or surface plasmon resonance assay. However, an antibody that specifically binds to an antigen or an epitope within the antigen may have cross-reactivity to other related antigens, e.g., to corresponding antigens from other species (homologous), such as humans or monkeys, e.g., Macacafascicularis (cynomolgus, cyno), Pan troglodytes (chimpanzee, chimp), or Callithrix jacchus (commonmarmoset, marmoset).

The term “not bind” means that the antibody is not capable of binding to a certain antigen or an epitope within the antigen in the manner of the specific binding described above, for example, when the antibody binds to the antigen or the epitope within the antigen with an equilibrium dissociation constant (KD) of about 1×10⁻⁶ M or greater.

The term “antigen-binding moiety” refers to a polypeptide molecule specifically binding to a target antigen. A specific antigen-binding moiety includes an antigen-binding domain of an antibody, e.g., comprises a heavy chain variable region and a light chain variable region. The term “antigen-binding moiety that specifically binds to GUCY2C” refers to a moiety that is capable of binding to GUCY2C with sufficient affinity, such that a molecule containing the moiety can be used as a diagnostic agent and/or therapeutic agent targeting GUCY2C. For example, the antigen-binding moiety that specifically binds to GUCY2C has the following equilibrium dissociation constant (KD): <about 10 nM, as measured by a surface plasmon resonance assay. Antigen-binding moieties include antibody fragments as defined herein, e.g., a Fab, a replaced Fab, or an scFv.

The term “linker” refers to a linker unit that links two polypeptide fragments. Herein, the linkers present in the same structural formula may be identical or different. The linker may be a peptide linker comprising one or more amino acids, typically about 1-30, 2-24, or 3-15 amino acids. The linkers used herein may be identical or different. When “-” appears in a structural formula, it means that the units on both sides are directly linked by a covalent bond.

“Tm” is the temperature of dissolution and denaturation (endogenous fluorescence). When the protein is denatured (by heating or by a denaturant), the tertiary structure is opened and the aromatic amino acid microenvironment changes, resulting in a change in the emission fluorescence spectrum. In the present disclosure, Tm1 refers to the temperature at which the fluorescence value changes to half of the maximum value.

“Tonset” is the onset temperature of denaturation. It refers to the temperature at which the protein begins to denature, i.e., the temperature at which the fluorescence value begins to change.

“Tagg” is the onset temperature of aggregation. The temperature at which the sample begins to aggregate is monitored by detecting aggregates at two wavelengths of 266 nm and 473 nm by static light scattering. Tagg 266 refers to the onset temperature of aggregation monitored at 266 nm.

The term “nucleic acid” is used interchangeably herein with the term “polynucleotide” and refers to deoxyribonucleotide or ribonucleotide and a polymer thereof in either single-stranded or double-stranded form. The term encompasses nucleic acids comprising known nucleotide analogs or modified backbone residues or linkages, which are synthetic, naturally occurring, and non-naturally occurring, have similar binding properties to the reference nucleic acid, and are metabolized in a manner similar to the reference nucleotide. Examples of such analogs include, but are not limited to, phosphorothioate, phosphoramidate, methylphosphonate, chiral-methylphosphonate, 2-O-methyl ribonucleotide, and peptide-nucleic acid (PNA).

“Isolated nucleic acid” refers to a nucleic acid molecule that has been separated from components of its natural environment. The isolated nucleic acid includes a nucleic acid molecule comprised in a cell that generally comprises the nucleic acid molecule, but the nucleic acid molecule is present extrachromosomally or at a chromosomal position different from its natural chromosomal position. An isolated nucleic acid encoding the antigen-binding molecule refers to one or more nucleic acid molecules encoding antibody heavy and light chains (or fragments thereof), including such nucleic acid molecule(s) in a single vector or separate vectors and such nucleic acid molecule(s) present at one or more locations in a host cell. Unless otherwise stated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions may be obtained by generating sequences in which the third position of one or more selected (or all) codons is substituted with degenerate bases and/or deoxyinosine residues.

The terms “polypeptide” and “protein” are used interchangeably herein and refer to a polymer of amino acid residues. The terms apply to amino acid polymers in which one or more amino acid residues are corresponding artificial chemical mimics of naturally occurring amino acids, as well as to naturally occurring amino acid polymers and non-naturally occurring amino acid polymers. Unless otherwise stated, a particular polypeptide sequence also implicitly encompasses conservatively modified variants thereof.

The term “sequence identity” refers to the degree (percentage) to which the amino acids/nucleic acids of two sequences are identical at equivalent positions when the two sequences are optimally aligned. In the alignment process, gaps may be allowed to be introduced, if necessary, to achieve the maximum percent sequence identity, but any conservative substitution is not considered a part that constitutes sequence identity. To determine percent sequence identity, alignments can be accomplished by techniques known to those skilled in the art, for example, using publicly available computer software, such as BLAST, BLAST-2, ALIGN, ALIGN-2, or Megalign (DNASTAR) software. Those skilled in the art can determine parameters suitable for measuring alignment, including any algorithms required to achieve maximum alignment of the full length of the aligned sequences.

The term “fusion” or “linkage” means that components (e.g., antigen-binding moieties and Fc domains) are covalently linked directly or via a linker.

The term “vector” means a polynucleotide molecule capable of transporting another polynucleotide linked thereto. One type of vector is a “plasmid”, which refers to a circular double-stranded DNA loop into which additional DNA segments can be ligated. Another type of vector is a viral vector, such as an adeno-associated viral vector (AAV or AAV2), wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. The term “expression vector” or “expression construct” refers to a vector that is applied to transform a host cell and comprises a nucleic acid sequence that directs and/or controls (along with the host cell) the expression of one or more heterologous coding regions operably linked thereto. Expression constructs may include, but are not limited to, sequences that affect or control transcription and translation and affect RNA splicing of a coding region operably linked thereto in the presence of an intron.

The terms “host cell”, “host cell line”, and “host cell culture” are used interchangeably and refer to cells into which exogenous nucleic acids have been introduced, including progenies of such cells. Host cells include “transformants” and “transformed cells”, which include primary transformed cells and progeny derived therefrom, regardless of the number of passages. Progeny may not be completely identical to parent cells in terms of nucleic acid content and may contain mutations. As used herein, the term includes mutant progenies that have the same function or biological activity as the cells screened or selected from the primary transformed cells. Host cells include prokaryotic and eukaryotic host cells, wherein eukaryotic host cells include, but are not limited to, mammalian cells, insect cell lines, plant cells, and fungal cells. Mammalian host cells include human, mouse, rat, canine, monkey, porcine, goat, bovine, equine, and hamster cells, including but not limited to, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, and HEK-293 cells. Fungal cells include yeast and filamentous fungal cells, including, for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia, Saccharomyces cerevisiae, Saccharomyces, Hansenula polymorpha, Kluyveromyces, Kluyveromyces lactis, Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Physcomitrella patens, and Neurospora crassa. Pichia, any Saccharomyces, Hansenula polymorpha, any Kluyveromyces, Candida albicans, any Aspergillus, Trichoderma reesei, Chrysosporium lucknowense, any Fusarium, Yarrowia lipolytica, and Neurospora crassa. The host cell of the present patent does not include objects not authorized by the Patent Laws.

As used in the present application, the expressions “cell”, “cell line”, and “cell culture” are used interchangeably, and all such designations include their progenies. Thus, the words “transformant” and “transformed cell” include the primary subject cell and cultures derived therefrom, regardless of the passage number. It is also understood that not all progeny have precisely identical DNA contents due to deliberate or inadvertent mutations. Variant progeny that have the identical function or biological activity as the original transformed cell from which they were selected are included.

“Optional” or “optionally” means that the event or circumstance subsequently described may, but does not necessarily, occur, and this description includes instances where the event or circumstance occurs or does not occur.

The term “pharmaceutical composition” refers to a mixture comprising one or more of the antigen-binding molecules or antibodies described herein and other chemical components; the other components are, for example, physiological/pharmaceutically acceptable carriers and excipients.

The term “pharmaceutically acceptable carrier” refers to an ingredient in a pharmaceutical formulation that is different from the active ingredient and is not toxic to the subject. Pharmaceutically acceptable carriers include, but are not limited to, buffers, excipients, stabilizers, or preservatives.

The term “subject” or “individual” includes humans and non-human animals. Non-human animals include all vertebrates (e.g., mammals and non-mammals) such as non-human primates (e.g., cynomolgus monkeys), sheep, dogs, cows, chickens, amphibians, and reptiles. Unless clearly indicated, the terms “patient” and “subject” are used interchangeably herein. As used herein, the term “cynomolgus monkey (cyno)” or “cynomolgus” refers to Macaca fascicularis. In certain embodiments, the individual or subject is a human.

“Administrating” or “giving”, when applied to animals, humans, experimental subjects, cells, tissues, organs, or biological fluids, refers to contact of an exogenous drug, a therapeutic agent, a diagnostic agent, or a composition with the animals, humans, subjects, cells, tissues, organs, or biological fluids.

The term “sample” refers to a collection of similar fluids, cells or tissues isolated from a subject, as well as fluids, cells or tissues present within a subject. Exemplary samples are biological fluids (such as blood; serum; serosal fluids; plasma; lymph; urine; saliva; cystic fluids; tears; excretions; sputum; mucosal secretions of secretory tissue and organs; vaginal secretions; ascites; fluids in the pleura, pericardium, peritoneum, abdominal cavity, and other body cavities; fluids collected from bronchial lavage; synovial fluids; liquid solutions in contact with a subject or biological source, e.g., cell and organ culture media (including cell or organ conditioned culture media); lavage fluids; and the like), tissue biopsy samples, fine needle punctures, surgically excised tissues, organ cultures, or cell cultures.

“Treatment” or “treat” (and grammatical variations thereof) refers to clinical intervention intended to be applied to the treated individual, which may be performed either for prophylactic purposes or during the course of clinical pathology. Desirable effects of the treatment include, but are not limited to, preventing the occurrence or recurrence of a disease, alleviating symptoms, alleviating/reducing any direct or indirect pathological consequences of the disease, preventing metastasis, decreasing the rate of disease progression, ameliorating or alleviating the disease state, and regressing or improving prognosis. In some embodiments, the molecule of the present disclosure is used to delay the development of or slow the progression of disease.

The terms “recurrence”, “relapse”, or “relapsed” refers to the return of a cancer or disease after clinical assessment of the disappearance of disease. A diagnosis of distant metastasis or local recurrence can be considered a relapse.

The term “refractory” or “resistant” refers to a cancer or disease that has not responded to treatment.

“Effective amount” is generally an amount sufficient to reduce the severity and/or frequency of symptoms, eliminate symptoms and/or underlying causes, prevent the appearance of symptoms and/or their underlying causes, and/or ameliorate or alleviate damage (e.g., lung disease) caused by or associated with a disease state. In some examples, the effective amount is a therapeutically effective amount or a prophylactically effective amount. “Therapeutically effective amount” is an amount sufficient to treat a disease state or symptom, particularly a state or symptom associated with the disease state, or to otherwise prevent, hinder, delay, or reverse the progression of the disease state or any other undesirable symptoms associated with the disease in any way. “Prophylactically effective amount” is an amount that, when administered to a subject, will have a predetermined prophylactic effect, e.g., preventing or delaying the onset (or recurrence) of the disease state, or reducing the likelihood of the onset (or recurrence) of the disease state or associated symptoms. A complete therapeutic or prophylactic effect does not necessarily occur after administration of one dose and may occur after administration of a series of doses. Thus, a therapeutically or prophylactically effective amount may be administered in one or more doses. “Therapeutically effective amount” and “prophylactically effective amount” may vary depending on a variety of factors such as the disease state, age, sex, and weight of the individual, and the ability of a therapeutic agent or combination of therapeutic agents to elicit a desired response in the individual. Exemplary indicators of an effective therapeutic agent or combination of therapeutic agents include, for example, improved health of a patient.

The term “immune checkpoint” refers to the group of molecules on the cell surface of CD4 T cells and CD8 T cells. These molecules can effectively act as “brakes” that down-regulate or inhibit anti-tumor immune responses. Immune checkpoint molecules include, but are not limited to, programmed death receptor 1 (PD-1), cytotoxic T lymphocyte antigen 4 (CTLA-4), B7H1, B7H4, OX-40, CD137, CD40, and LAG-3, which directly inhibit immune cells.

Immune checkpoint inhibitors for use in the present disclosure are substances that inhibit the function of immune checkpoint molecules. The immune checkpoint inhibitor is not particularly limited as long as the inhibitor is a substance that can inhibit the function (signal) of the immune checkpoint molecule. Inhibitors that may be used as immune checkpoint inhibitors in the methods of the present disclosure include, but are not limited to, inhibitors of PD-1, PD-L2, CTLA-4, TIM-3, LAG-3, VISTA, BTLA, TIGIT, LAIR1, CD160, 2B4, CEACAM (e.g., CEACAM-1, CEACAM-3, and/or CEACAM-5), and/or TGFRβ. Inhibition of the inhibitory molecules can be performed by inhibition at the DNA, RNA or protein level. In embodiments, an inhibitory nucleic acid (e.g., dsRNA, siRNA, or shRNA) can be used to inhibit the expression of an inhibitory molecule. In other embodiments, the inhibitor of an inhibitory signal is a polypeptide that binds to an inhibitory molecule, e.g., a soluble ligand or an antibody. Examples of immune checkpoint inhibitors used in the present disclosure may include, but are not particularly limited to, anti-PD-1 antibodies, anti-PD-L1 antibodies, and anti-CTLA-4 antibodies, and anti-PD-1 antibodies and anti-PD-L1 antibodies may be preferred.

### Antigen-Binding Molecules of the Present Disclosure

The present disclosure provides an antigen-binding molecule having a number of advantageous properties, such as good in vitro killing activity, therapeutic activity, safety, pharmacokinetic properties, and druggability (e.g., yield, purity, and stability).

### Exemplary Antigen-Binding Molecules

The antigen-binding molecule of the present disclosure includes antigen-binding molecules (e.g., bispecific antibodies) that specifically bind to GUCY2C and CD3 and anti-GUCY2C antibodies. Particularly, the antigen-binding molecule of the present disclosure has any one of the following properties:
a. High affinity for GUCY2C. In some embodiments, the GUCY2C monoclonal antibody binds to human GUCY2C with a KD value of less than 100 nM (e.g., less than 80 nM, less than 50 nM, less than 10 nM, less than 8 nM, less than 1 nM, or less than 0.5 nM) as measured by Biacore.
b. Good specific binding to GUCY2C on the surface of cells. In some embodiments, the binding activity of the GUCY2C monoclonal antibody is better than 1608. In some embodiments, the GUCY2C-CD3 bispecific antibody of the present disclosure has good specific binding activity for cells expressing different levels of GUCY2C, and the binding activity is better than that of 1608-BsAb; the GUCY2C-CD3 bispecific antibody of the present disclosure has good specific binding activity for cells expressing Jurkat, and the binding activity is better than that of 1608-BsAb.
c. No non-specific binding to non-target cells (e.g., T cells). In some embodiments, the GUCY2C monoclonal antibody does not non-specifically bind to Jurkat cells that do not express GUCY2C; 1608 significantly binds to Jurkat cells. In some embodiments, the GUCY2C monoclonal antibody of the present disclosure does not non-specifically bind to PBMCs and T cells that do not express GUCY2C, and the control antibody 1608 does significantly non-specifically bind to PBMCs and T cells. The target specificity of the GUCY2C monoclonal antibody is better than that of 1608.
d. The antigen epitope bound by the GUCY2C monoclonal antibody is significantly different from that bound by 1608, and thus the GUCY2C monoclonal antibody can not compete with 1608 for binding to the GUCY2C antigen.
e. Good binding effect with human GUCY2C protein. In some embodiments, the GUCY2C-CD3 bispecific antibody binds to human GUCY2C with a KD value of less than 1 nM (e.g., less than 0.5 nM, less than 0.2 nM, or less than 0.1 nM), and the EC₅₀ value measured by ELISA.
f. The GUCY2C-CD3 bispecific antibody exhibits good cytotoxic activity for target cells with different expression levels.
g. The GUCY2C-CD3 bispecific antibody can effectively induce the release of IFN-γ related to cytotoxic activity, and the release level of cytokine IL-6 related to inflammation is relatively low.
h. Stronger in vivo therapeutic activity. In some embodiments, the GUCY2C-CD3 bispecific antibody, or the GUCY2C-CD3 bispecific antibody in combination with an EGFR-CD28 bispecific antibody, has good in vivo therapeutic activity. When the GUCY2C-CD3 bispecific antibody is used in combination with the EGFR-CD28 bispecific antibody, the respective drug effect of the two can be effectively improved.

The present disclosure provides an antigen-binding molecule comprising at least one antigen-binding moiety that specifically binds to GUCY2C and at least one antigen-binding moiety that specifically binds to CD3, wherein the antigen-binding moiety that specifically binds to GUCY2C comprises a heavy chain variable region GUCY2C-VH and a light chain variable region GUCY2C-VL, and the antigen-binding moiety that specifically binds to CD3 comprises a heavy chain variable region CD3-VH and a light chain variable region CD3-VL.

Specifically, the examples herein disclose the following antibody series: P3-C7, 81, and 146. Antibody P3-C7 is described below as an example of the antibodies herein.

Provided is an antigen-binding molecule that specifically binds to GUCY2C and CD3 or an anti-GUCY2C antibody, wherein:
the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 47, 22, 43, 44, 45, or 46, a GUCY2C-LCDR2 comprising the amino acid sequence of SEQ ID NO: 23, and a GUCY2C-LCDR3 comprising the amino acid sequence of SEQ ID NO: 24.

Provided is an antigen-binding molecule that specifically binds to GUCY2C and CD3 or an anti-GUCY2C antibody, wherein:
the GUCY2C-VH comprises: a GUCY2C-HCDR1 having the amino acid sequence set forth in SEQ ID NO: 19, a GUCY2C-HCDR2 having the amino acid sequence set forth in SEQ ID NO: 20, and a GUCY2C-HCDR3 having the amino acid sequence set forth in SEQ ID NO: 21; and the GUCY2C-VL comprises: a GUCY2C-LCDR1 having the amino acid sequence set forth in SEQ ID NO: 47, a GUCY2C-LCDR2 having the amino acid sequence set forth in SEQ ID NO: 23, and a GUCY2C-LCDR3 having the amino acid sequence set forth in SEQ ID NO: 24.

In some embodiments, provided is the antigen-binding molecule or the anti-GUCY2C antibody according to the above, wherein the GUCY2C-VH and/or the GUCY2C-VL are murine or humanized. In some embodiments, the GUCY2C-VH and/or the GUCY2C-VL are humanized.

In some embodiments, the humanized GUCY2C-VH comprises an FR1, an FR2, and an FR3 derived from humanized IGHV1-46*01 and an FR4 derived from IGHJ6*01, and it is unsubstituted or has one or more amino acid substitutions selected from the group consisting of 1E, 39L, 43K, 69L, 71V, and 73K; and/or the humanized GUCY2C-VL comprises an FR1, an FR2, and an FR3 derived from IGKV2-40*01 or IGKV1-39*01 and an FR4 derived from IGKJ4*01, and it is unsubstituted or has one or more amino acid substitutions selected from the group consisting of 2V, 3V, 68R, and 74K. In some embodiments, the amino acid positions in the above variable regions are defined according to the Kabat numbering scheme.

In some embodiments, provided is the antigen-binding molecule or the anti-GUCY2C antibody according to any one of the above, wherein the GUCY2C-VH comprises the amino acid sequence of SEQ ID NO: 56, 25, or 57, and the GUCY2C-VL comprises the amino acid sequence of SEQ ID NO: 54, 55, 26, 48, 49, 50, 51, 52, or 53.

In some embodiments, provided is the antigen-binding molecule or the anti-GUCY2C antibody according to any one of the above, wherein:
the GUCY2C-VH comprises the amino acid sequence of SEQ ID NO: 56, and the GUCY2C-VL comprises the amino acid sequence of SEQ ID NO: 54, 55, 48, 49, 50, 51, or 52, or
the GUCY2C-VH comprises the amino acid sequence of SEQ ID NO: 25, and the GUCY2C-VL comprises the amino acid sequence of SEQ ID NO: 26.

In some embodiments, provided is the antigen-binding molecule or the anti-GUCY2C antibody according to any one of the above, wherein:
the amino acid sequence of the GUCY2C-VH is set forth in SEQ ID NO: 56, and the amino acid sequence of the GUCY2C-VL is set forth in SEQ ID NO: 54, or
the amino acid sequence of the GUCY2C-VH is set forth in SEQ ID NO: 56, and the amino acid sequence of the GUCY2C-VL is set forth in SEQ ID NO: 55.

In some embodiments, provided is the antigen-binding molecule according to the above, wherein the CD3-VH comprises: a CD3-HCDR1 comprising the amino acid sequence of SEQ ID NO: 71, a CD3-HCDR2 comprising the amino acid sequence of SEQ ID NO: 72, and a CD3-HCDR3 comprising the amino acid sequence of SEQ ID NO: 73 or 88; and the CD3-VL comprises: a CD3-LCDR1 comprising the amino acid sequence of SEQ ID NO: 74, a CD3-LCDR2 comprising the amino acid sequence of SEQ ID NO: 75, and a CD3-LCDR3 comprising the amino acid sequence of SEQ ID NO: 76.

In some embodiments, provided is the antigen-binding molecule according to any one of the above, wherein the CD3-VH and/or the CD3-VL are murine or humanized.

In some embodiments, the CD3-VH and/or the CD3-VL are humanized.

In some embodiments, the CD3-VH comprises the amino acid sequence of SEQ ID NO: 77 or 89, and the CD3-VL comprises the amino acid sequence of SEQ ID NO: 78.

In some embodiments, the amino acid sequence of the CD3-VH is set forth in SEQ ID NO: 77, and the amino acid sequence of the CD3-VL is set forth in SEQ ID NO: 78.

In some embodiments, the above variable regions and CDRs are defined according to the Kabat numbering scheme.

The antibody series 81 and 146 disclosed according to the examples herein have a technical solution range similar to that of the antibody series P3-C7 described above.

### Structure of Antigen-Binding Molecule

The bispecific antigen-binding molecule of the present disclosure is not limited to a particular molecular structure, as long as it has the desired antigen binding function. For example, the bispecific antigen-binding molecule herein may be bivalent (1+1), trivalent (2+1), or tetravalent (2+2). The antigen-binding moieties in the antigen-binding molecule may be any antibody fragments having antigen binding activity, and they are fused via a peptide linker. The peptide linkers of the present disclosure (e.g., linkers 1 through 11) can be any suitable peptide chains, as long as the antigen-binding molecules are capable of exhibiting the desired antigen binding activity. For example, the peptide linkers may be flexible peptides comprising 1-50 or 3-20 amino acid residues. In some embodiments, the peptide linkers each independently have the structure of L₁-(GGGGS)n-L₂, wherein L₁ is a bond, A, G (SEQ ID NO: 82), GS, GGG (SEQ ID NO: 96), GGS (SEQ ID NO: 245), GGGS (SEQ ID NO: 246), or GGGG (SEQ ID NO: 247), n is 0, 1, 2, 3, 4, 5, 6, 7, 8, 9, or 10, L₂ is a bond, G (SEQ ID NO: 82), GG, GGG (SEQ ID NO: 96), or GGGG (SEQ ID NO: 247), and the peptide linkers are not bonds. In some embodiments, the peptide linker is 3-15 amino acid residues in length. In some embodiments, the peptide linkers each independently have a structure of (GGGGS)n, wherein n is 1, 2, or 3. In some embodiments, the amino acid sequence of the linker 1 is set forth in SEQ ID NO: 81, the amino acid sequence of the linker 2 is set forth in SEQ ID NO: 82, the amino acid sequence of the linker 3 is set forth in SEQ ID NO: 83, the amino acid sequence of the linker 4 is set forth in SEQ ID NO: 90, the amino acid sequence of the linker 5 is set forth in SEQ ID NO: 95, the amino acid sequence of the linker 6 is set forth in SEQ ID NO: 96, and the amino acid sequence of the linker 7 is set forth in SEQ ID NO: 97.

Illustratively, the antigen-binding molecule of the present disclosure comprises one first chain having a structure represented by formula (a) and one second chain having a structure represented by formula (b):

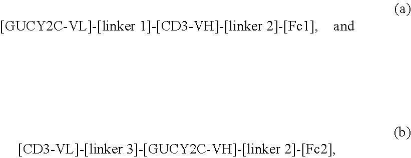

wherein the linker 1, the linker 2, and the linker 3 are identical or different peptide linkers; or the linker 1, the linker 2, and the linker 3 are absent;
the structures represented by formulas (a) and (b) are arranged from N-terminus to C-terminus.

Illustratively, the antigen-binding molecule of the present disclosure comprises one first chain having a structure represented by formula (a) and one second chain having a structure represented by formula (b):

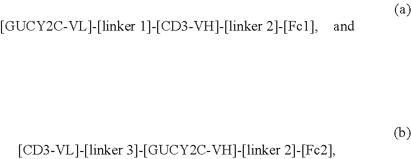

wherein:
the GUCY2C-VH comprises the amino acid sequence of SEQ ID NO: 56, and the GUCY2C-VL comprises the amino acid sequence of SEQ ID NO: 54; and the CD3-VH comprises the amino acid sequence of SEQ ID NO: 77, and the CD3-VL comprises the amino acid sequence of SEQ ID NO: 78; or
the GUCY2C-VH comprises the amino acid sequence of SEQ ID NO: 56, and the GUCY2C-VL comprises the amino acid sequence of SEQ ID NO: 55; and the CD3-VH comprises the amino acid sequence of SEQ ID NO: 77, and the CD3-VL comprises the amino acid sequence of SEQ ID NO: 78;
the linker 1, the linker 2, and the linker 3 are identical or different peptide linkers; or
the linker 1, the linker 2, and the linker 3 are absent;
the amino acid sequence of the Fc1 is set forth in SEQ ID NO: 79, and the amino acid sequence of the Fc2 is set forth in SEQ ID NO: 80;
the structures represented by formulas (a) and (b) are arranged from N-terminus to C-terminus.

Illustratively, the antigen-binding molecule of the present disclosure comprises one first chain having a structure represented by formula (a) and one second chain having a structure represented by formula (b):

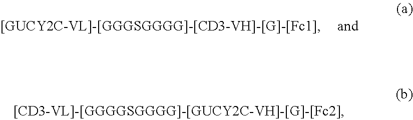

wherein: the structures represented by formulas (a) and (b) are arranged from N-terminus to C-terminus.

Illustratively, the antigen-binding molecule of the present disclosure comprises one first chain having a structure represented by formula (a) and one second chain having a structure represented by formula (b):

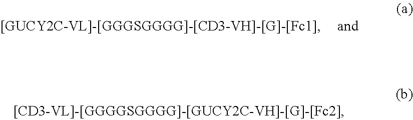

wherein:
the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 47, a GUCY2C-LCDR2 comprising the amino acid sequence of SEQ ID NO: 23, and a GUCY2C-LCDR3 comprising the amino acid sequence of SEQ ID NO: 24;

The CD3-VH comprises: a CD3-HCDR1 set forth in SEQ ID NO: 71, a CD3-HCDR2 set forth in SEQ ID NO: 72, and a CD3-HCDR3 set forth in SEQ ID NO: 73; and the CD3-VL comprises: a CD3-LCDR1 set forth in SEQ ID NO: 74, a CD3-LCDR2 set forth in SEQ ID NO: 75, and a CD3-LCDR3 set forth in SEQ ID NO: 76;
the amino acid sequence of the Fc1 is set forth in SEQ ID NO: 79, and the amino acid sequence of the Fc2 is set forth in SEQ ID NO: 80;
the structures represented by formulas (a) and (b) are arranged from N-terminus to C-terminus.

An exemplary antigen-binding molecule comprises:
one first chain comprising the amino acid sequence of SEQ ID NO: 99 and one second chain comprising the amino acid sequence of SEQ ID NO: 100; or
one first chain comprising the amino acid sequence of SEQ ID NO: 101 and one second chain comprising the amino acid sequence of SEQ ID NO: 100.

Illustratively, the antigen-binding molecule comprises one first chain having a structure represented by formula (c), one second chain having a structure represented by formula (d), one third chain having a structure represented by formula (e), and one fourth chain having a structure represented by formula (f):

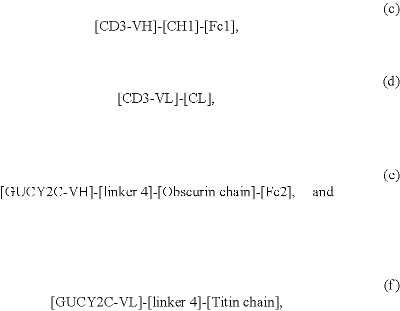

wherein the linker 4 is a peptide linker; or the linker 4 is absent;
the structures represented by formulas (c), (d), (e), and (f) are arranged from the N-terminus to the C-terminus.

Illustratively, the antigen-binding molecule comprises one first chain having a structure represented by formula (c), one second chain having a structure represented by formula (d), one third chain having a structure represented by formula (e), and one fourth chain having a structure represented by formula (f):

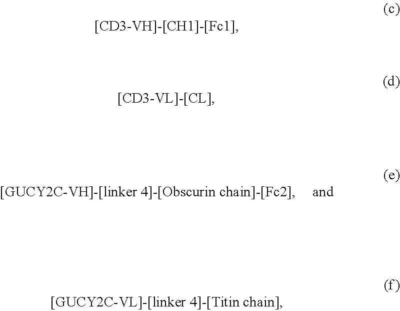

wherein:
the GUCY2C-VH comprises the amino acid sequence of SEQ ID NO: 15, and the GUCY2C-VL comprises the amino acid sequence of SEQ ID NO: 16; and the CD3-VH comprises the amino acid sequence of SEQ ID NO: 77, and the CD3-VL comprises the amino acid sequence of SEQ ID NO: 78;
the linker 4 is a peptide linker; or linker 4 is absent;
the amino acid sequence of the Obscurin chain is set forth in SEQ ID NO: 236, and the amino acid sequence of the Titin chain is set forth in SEQ ID NO: 198;
the amino acid sequence of the Fc1 is set forth in SEQ ID NO: 85, and the amino acid sequence of the Fc2 is set forth in SEQ ID NO: 86;
the amino acid sequence of the CH1 is set forth in SEQ ID NO: 84, and the amino acid sequence of the CL is set forth in SEQ ID NO: 87;
the structures represented by formulas (c), (d), (e), and (f) are arranged from the N-terminus to the C-terminus.

Illustratively, the antigen-binding molecule comprises one first chain having a structure represented by formula (c), one second chain having a structure represented by formula (d), one third chain having a structure represented by formula (e), and one fourth chain having a structure represented by formula (f):

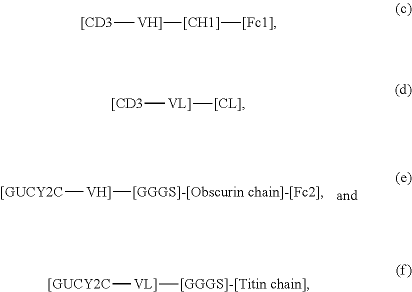

wherein: the structures represented by formulas (c), (d), (e), and (f) are arranged from the N-terminus to the C-terminus.

Illustratively, the antigen-binding molecule comprises one first chain having a structure represented by formula (c), one second chain having a structure represented by formula (d), one third chain having a structure represented by formula (e), and one fourth chain having a structure represented by formula (f):

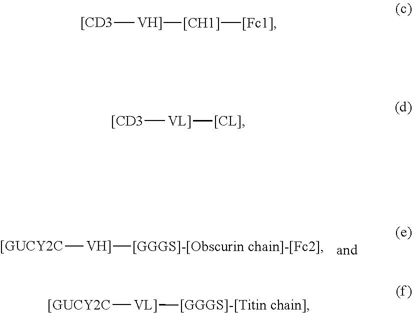

wherein:
The GUCY2C-VH comprises: a GUCY2C-HCDR1 set forth in SEQ ID NO 4, a GUCY2C-HCDR2 set forth in SEQ ID NO 5, and a GUCY2C-HCDR3 set forth in SEQ ID NO: 27; and the GUCY2C-VL comprises: a GUCY2C-LCDR1 set forth in SEQ ID NO 7, a GUCY2C-LCDR2 set forth in SEQ ID NO 8, and a GUCY2C-LCDR3 set forth in SEQ ID NO: 9;
The CD3-VH comprises: a CD3-HCDR1 set forth in SEQ ID NO: 71, a CD3-HCDR2 set forth in SEQ ID NO: 72, and a CD3-HCDR3 set forth in SEQ ID NO: 73; and the CD3-VL comprises: a CD3-LCDR1 set forth in SEQ ID NO: 74, a CD3-LCDR2 set forth in SEQ ID NO: 75, and a CD3-LCDR3 set forth in SEQ ID NO: 76;
the amino acid sequence of the Obscurin chain is set forth in SEQ ID NO: 236, and the amino acid sequence of the Titin chain is set forth in SEQ ID NO: 198;
the amino acid sequence of the Fc1 is set forth in SEQ ID NO: 85, and the amino acid sequence of the Fc2 is set forth in SEQ ID NO: 86;
the amino acid sequence of the CH1 is set forth in SEQ ID NO: 84, and the amino acid sequence of the CL is set forth in SEQ ID NO: 87;
the structures represented by formulas (c), (d), (e), and (f) are arranged from the N-terminus to the C-terminus.

An exemplary antigen-binding molecule comprises: one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 113.

### Variants of Antigen-Binding Molecules

In certain embodiments, amino acid sequence variants of the antigen-binding molecules provided herein are encompassed. For example, it may be desirable to improve the binding affinity and/or other biological properties of the antibody. Amino acid sequence variants of an antibody can be prepared by introducing appropriate modifications into the nucleotide sequence encoding the antibody, or by peptide synthesis. Such modifications include, for example, deletions from, and/or insertions into and/or substitutions of residues within the amino acid sequence of the antigen-binding molecule. Any combination of deletion, insertion, and substitution can be made to obtain the final construct, provided that the final construct possesses the desired characteristics, e.g., antigen binding.

Variants with Substitutions, Insertions, and Deletions

In certain embodiments, antigen-binding molecule variants having one or more amino acid substitutions are provided. Substitutions may be made in the CDRs and FRs. Conservative substitutions are shown in Table 2 under the heading of “Preferred substitution”. More substantial changes are provided in Table 2 under the heading of “exemplary substitution”, and as further described below with reference to amino acid side chain classes. Amino acid substitutions can be introduced into an antibody of interest and the products are screened for a desired activity, e.g., retained/improved antigen binding, reduced immunogenicity, or improved ADCC or CDC.

TABLE 2
Amino acid substitutions

| Original | | Preferred |
|---|---|---|
| residue | Exemplary substitution | substitution |
| Ala(A) | Val; Leu; Ile | Val |
| Arg(R) | Lys; Gln; Asn | Lys |
| Asn(N) | Gln; His; Asp, Lys; Arg | Gln |
| Asp(D) | Glu; Asn | Glu |
| Cys(C) | Ser; Ala | Ser |
| Gln(Q) | Asn; Glu | Asn |
| Glu(E) | Asp; Gln | Asp |
| Gly(G) | Ala | Ala |
| His(H) | Asn; Gln; Lys; Arg | Arg |
| Ile(I) | Leu; Val; Met; Ala; Phe; norleucine | Leu |
| Leu(L) | Norleucine; Ile; Val; Met; Ala; Phe | Ile |
| Lys(K) | Arg; Gln; Asn | Arg |
| Met(M) | Leu; Phe; Ile | Leu |
| Phe(F) | Trp; Leu; Val; Ile; Ala; Tyr | Tyr |
| Pro(P) | Ala | Ala |
| Ser(S) | Thr | Thr |
| Thr(T) | Ser | Ser |
| Trp(W) | Tyr; Phe | Tyr |
| Tyr(Y) | Trp; Phe; Thr; Ser | Phe |
| Val(V) | Ile; Leu; Met; Phe; Ala; norleucine | Leu |

According to common side-chain properties, amino acids can be grouped as follows:
(1) hydrophobic: norleucine, Met, Ala, Val, Leu, Ile;
(2) neutral, hydrophilic: Cys, Ser, Thr, Asn, Gln;
(3) acidic: Asp, Glu;
(4) basic: His, Lys, Arg;
(5) residues affecting chain orientation: Gly, Pro;
(6) aromatic: Trp, Tyr, Phe.

A non-conservative substitution refers to using a member of one class to replace a member of another class.

One type of substitutional variant involves substituting one or more CDR residues of a parent antibody (e.g., a humanized or human antibody). Generally, the resulting variant selected for further study will have changes (e.g., improvements) in certain biological properties (e.g., increased affinity or reduced immunogenicity) relative to the parent antibody, and/or will have substantially retained certain biological properties of the parent antibody. An exemplary substitutional variant is an affinity-matured antibody, which can be conveniently produced, for example, using phage display-based affinity maturation techniques such as those described herein. Briefly, one or more CDR residues are mutated and the variant antibodies are displayed on phages and screened for a particular biological activity (e.g. binding affinity). Changes (e.g., substitutions) can be made in CDRs, for example, to improve antibody affinity. Such changes can be made in CDR “hotspots”, i.e., residues encoded by codons that undergo mutation at high frequency during the somatic maturation process, and/or antigen-contacting residues, and meanwhile, the resulting variant VH or VL is tested for binding affinity. In some embodiments of affinity maturation, diversity is introduced into the variable genes selected for maturation by any one of a variety of methods (e.g., error-prone PCR, chain shuffling, or oligonucleotide-directed mutagenesis). A secondary library is then created. The library is then screened to identify any antibody variants with the desired affinity. Another method for introducing diversity involves CDR-directed methods in which several CDR residues (e.g., 4-6 residues at a time) are randomized. CDR residues involved in antigen binding can be specifically identified, for example, using alanine scanning mutagenesis or modeling.

In certain embodiments, substitutions, insertions or deletions may occur within one or more CDRs, as long as such changes do not substantially reduce the ability of the antibody to bind to the antigen. For example, conservative changes (e.g., conservative substitutions as provided herein) that do not substantially reduce binding affinity may be made in CDRs. In certain embodiments of the variant VH and VL sequences provided above, each CDR is unaltered or contains no more than 1, 2, or 3 amino acid substitutions.

A useful method for identifying residues or regions of an antibody that can be used as mutagenesis targets is called “alanine scanning mutagenesis”. In this method, a residue or group of residues (e.g., charged residues such as Arg, Asp, His, Lys, and Glu) are identified and replaced with a neutral or negatively charged amino acid (e.g., Ala or polyalanine) to determine whether the interaction of the antibody with the antigen is affected. Further substitutions may be introduced at amino acid positions that show functional sensitivity to the initial substitutions. In addition, a crystal structure of an antigen-antibody complex may be studied to identify contact points between the antibody and antigen. These contact residues and neighboring residues may be targeted or eliminated as substitution candidates. Variants may be screened to determine whether they contain the desired properties.

Amino acid sequence insertions include: the fusion of 1 residue or polypeptides of 100 or more residues in length to the amino- and/or carboxy-terminus, and intrasequence insertions of single or multiple amino acid residues. Examples of terminal insertions include an antibody with an N-terminal methionyl residue. Other insertional variants of the antibody molecule include the fusion where an enzyme (or a polypeptide that increases the serum half-life of an antibody) is fused to the N- or C-terminus of the antibody.

### Engineering of Fab

In one aspect, in the antigen-binding molecule of the present disclosure, one of the antigen-binding moiety that specifically binds to GUCY2C and the antigen-binding moiety that specifically binds to CD3 is a replaced Fab, and the replaced Fab comprises a heavy chain variable region, a light chain variable region, a Titin chain, and an Obscurin chain. In the replaced Fab, the original CH1 and CL of the Fab are substituted with the Titin chain and the Obscurin chain. Illustratively, the sequences of the Titin chain and the Obscurin chain are shown in Tables 3-1 and 3-2.

TABLE 3-1
Amino acid sequences of Titin chains

| No. | SEQ ID NO | Amino acid sequence |
|---|---|---|
| T.0 | 182 | GIPPKIEALPSDISIDEGKVLTVACAFTGEPTPEVTWSCGGRKIHSQEQ |
| | | GRFHIENTDDLTTLIIMDVQKQDGGLYTLSLGNEFGSDSATVNIHIRSI |
| T.1 | 183 | GIPPKIECLPSDISIDEGKVLTVASAFTGEPTPEVTWSTGGRKIHSQEQG |
| | | RFHIENTDDLTTLIIMDVQKQDGGLYTLSLGNEFGSDSATVNIHIRSI |
| T.2 | 184 | GIPPKIEALPSDISIDEGKCLTVASAFTGEPTPEVTWSTGGRKIHSQEQG |
| | | RFHIENTDDLTTLIIMDVQKQDGGLYTLSLGNEFGSDSATVNIHIRSI |
| T.3 | 185 | GIPPKIEALPSDISIDEGKVLTVASCFTGEPTPEVTWSTGGRKIHSQEQG |
| | | RFHIENTDDLTTLIIMDVQKQDGGLYTLSLGNEFGSDSATVNIHIRSI |
| T.4 | 186 | GIPPKIEALPSDISIDEGKVLCVASAFTGEPTPEVTWSTGGRKIHSQEQ |
| | | GRFHIENTDDLTTLIIMDVQKQDGGLYTLSLGNEFGSDSATVNIHIRSI |
| T.5 | 187 | KAGIRGIPPKIECLPSDISIDEGKVLTVASAFTGEPTPEVTWSTGGRKIH |
| | | SQEQGRFHIENTDDLTTLIIMDVQKQDGGLYTLSLGNEFGSDSATVNI |
| | | HIRSI |
| T.6 | 188 | KAGIRGIPPKIEALPSDISIDEGKVLTVACAFTGEPTPEVTWSCGGRKIH |
| | | SQEQGRFHIENTDDLTTLIIMDVQKQDGGLYTLSLGNEFGSDSATVNI |
| | | HIRSI |
| T.7 | 189 | GIPPKIECLPSDISIDEGKVLTVASAFTGEPTPEVTWSTGGRKIHSQEQG |
| | | RFHIENTDDLTTLIISDVQKQDGGLYSLSLGNEFGSDSATVNIHIRSI |
| T.8 | 190 | GIPPKIECLPSDISIDEGKVLTVASAFTGEPTPEVTWSTGGRKIHSQEQG |
| | | RFHIENTDDLTTLIIKDVQRQDGGLYTLTLRNEFGSDSATVNIHIRSI |
| T.9 | 191 | GIWPKIECLPIDLSIDEGKVLMVASAFTGEPTPEVTWSTGGRKIHSQEQ |
| | | GRFHIENTDDLTTLIIMDVQKQDGGLYTLSLGMEFGSDSATVNIHIRSI |
| T.10 | 192 | GIPPKIECLPIDISIDEGKVLTVASAFTGEPTPEVTWSTGGRKIHSQEQG |
| | | RFHIENTDDLTTLIIKDVQKQDGGLYTLTLRNEFGSDSATVNIHIRSI |
| T.11 | 193 | GIPPKIECLPSDISIDEGKVLTVASAFTGEPTPEVTWSTSGKKISSEEGG |
| | | RFHIESTEDLTTLIIMDVQKQDGGVYTLSLGNDLGSDSATVNIHIRSI |
| T.12 | 194 | GIPPKIECLPSDISIDEGKVLTVASAFTGEPTPEVTWSTGGRKIHSEEGG |
| | | RFHIESTEDLTTLIIMDVQKQDGGVYTLSLGNEFGSDSATVNIHIRSI |
| T.13 | 195 | GIPPKIECLPSDISIDEGKVLTVASAFTGEPTPEVTWSTGGRKIHSQEQG |
| | | RFHIESTEDLTTLIIMDVQKQDGGVYTLSLGNEFGSDSATVNIHIRSI |
| T.14 | 196 | GIPPKIECLPSDISIDEGKVLTVASAFTGEPTPEVTWSTGGRKIHSQEQG |
| | | RFHIESTEDLTTLIISDVQKQDGGLYSLSLGNEFGSDSATVNIHIRSI |
| T.15 | 197 | KAGIRGIPPKIECLPSDISIDEGKVLTVASAFTGEPTPEVTWSTGGRKIH |
| | | SQEQGRFHIESTEDLTTLIISDVQKQDGGLYSLSLGNEFGSDSATVNIHI |
| | | RSI |
| T.16 | 198 | GIPPKIECLPIDISIDEGKVLTVASAFTGEPTPEVTWSTGGRKIHSQEQG |
| | | RFHIENTDDSTTLTIKDVQKQDGGLYTLTLRNEFGSDSATVNIHIRSI |
| T.17 | 199 | GIPPKIECLPIDISIDEGKCLTVASAFTGEPTPEVTWSTGGRKIHSQEQG |
| | | RFHIENTDDSTTLTIKDVQKQDGGLYTLTLRNEFGSDSATVNIHIRSI |
| T.18 | 200 | GIPPKIECLPIDISIDEGKVLTVASCFTGEPTPEVTWSTGGRKIHSQEQG |
| | | RFHIENTDDSTTLTIKDVQKQDGGLYTLTLRNEFGSDSATVNIHIRSI |

TABLE 3-2
Amino acid sequences of Obscurin chains

| No. | SEQ ID NO | Amino acid sequence |
|---|---|---|
| O.0 | 201 | SGAPRFLTRPKAFVVSVGKDATLSCQIVGNPTPQVSWEKDQQPVAAG |
| | | ARFRLAQDGDLYRLTILDLALGDSGQYVCRARNAIGEAFAAVGLQVD |
| | | AEA |
| OL.0 | 202 | QGSPPCFLRFPRPVRVVSGAEAELKCVVLGEPPPVVVWEKGGQQLA |
| | | ASERLSFPADGAEHGLLLTAALPTDAGVYVCRARNAAGEAYAAAAV |
| | | TVLEPP |
| O.1 | 203 | SGAPRFLTRPKAFVVSVGKDATLSCQIVGNPTPQVSWEKDQQPVAAG |
| | | ARFRLAQDGDLYRLTILDLALGDSGQYVCRARNAIGEAFACVGLQVD |
| | | AEA |
| O.2 | 204 | SGCPRFLTRPKAFVVSVGKDATLSCQIVGNPTPQVSWEKDQQPVAAG |
| | | ARFRLAQDGDLYRLTILDLALGDSGQYVCRARNAIGEAFAAVGLQVD |
| | | AEA |
| O.3 | 205 | SGAPRFLTCPKAFVVSVGKDATLSCQIVGNPTPQVSWEKDQQPVAAG |
| | | ARFRLAQDGDLYRLTILDLALGDSGQYVCRARNAIGEAFAAVGLQVD |
| | | AEA |
| O.4 | 206 | SGAPRFLTRPKAFVVSVGKDATLSSQIVGNPTPQVSWEKDQQPVAAG |
| | | ARFRLAQDGDLYRLTILDLALGDSGQYVSRARNAIGEAFACVGLQVD |
| | | AEA |
| O.5 | 207 | SGCPRFLTRPKAFVVSVGKDATLSSQIVGNPTPQVSWEKDQQPVAAG |
| | | ARFRLAQDGDLYRLTILDLALGDSGQYVSRARNAIGEAFAAVGLQVD |
| | | AEA |
| O.6 | 208 | SGAPRFLTCPKAFVVSVGKDATLSSQIVGNPTPQVSWEKDQQPVAAG |
| | | ARFRLAQDGDLYRLTILDLALGDSGQYVSRARNAIGEAFAAVGLQVD |
| | | AEA |
| O.7 | 209 | SGAPRFKTRPKAFVVSVGKDATLSSQIVGNPTPQVSWEKDQQPVAAG |
| | | ARFRLAQDGDLYRLKILDLALGDSGQYVSRARNAIGEAFACVGLQV |
| | | DAEA |
| O.8 | 210 | SGAPRFKTRPKAFVVSVGKDATLSSQIVGNPTPQVSWEKDQQPVAAG |
| | | ARFRLAQDGDLYRLHILDLALGDSGQYVSRARNAIGEAFACVGLQV |
| | | DAEA |
| O.9 | 211 | SGAPRFLTRPLAFVVSVGKDATLSSQIVGNPTPQVSWEKDQQPVAAG |
| | | ARFRLAQDGDLYRLKILDLALGDSGQYVSRARNAIGEAFACVGLQV |
| | | DAEA |
| O.10 | 212 | SGAPRFLTRPLAFVVSVGKDATLSSQIVGNPTPQVSWEKDQQPVAAG |
| | | ARFRLAQDGDLYRLHILDLALGDSGQYVSRARNAIGEAFACVGLQV |
| | | DAEA |
| O.11 | 213 | DQPQFSGAPRFLTRPKAFVVSVGKDATLSSQIVGNPTPQVSWEKDQQ |
| | | PVAAGARFRLAQDGDLYRLTILDLALGDSGQYVSRARNAIGEAFACV |
| | | GLQVDAEA |
| O.12 | 214 | DQPQFSGAPRFKTRPKAFVVSVGKDATLSSQIVGNPTPQVSWEKDQQ |
| | | PVAAGARFRLAQDGDLYRLKILDLALGDSGQYVSRARNAIGEAFACV |
| | | GLQVDAEA |
| O.13 | 215 | DQPQFSGAPRFKTRPKAFVVSVGKDATLSSQIVGNPTPQVSWEKDQQ |
| | | PVAAGARFRLAQDGDLYRLHILDLALGDSGQYVSRARNAIGEAFACV |
| | | GLQVDAEA |
| O.14 | 216 | DQPQFSGAPRFRTRPKAFVVSVGKDATLSSQIVGNPTPQVSWEKDQQ |
| | | PVAAGARFRLAQDGDLYRLKILDLALGDSGQYVSRARNAIGEAFACV |
| | | GLQVDAEA |
| O.15 | 217 | DQPQFSGAPRFRTRPKAFVVSVGKDATLSSQIVGNPTPQVSWEKDQQ |
| | | PVAAGARFRLAQDGDLYRLHILDLALGDSGQYVSRARNAIGEAFACV |
| | | GLQVDAEA |
| O.16 | 218 | DQPQFSGAPRFLTRPKAFVVSVGKDATLSCQIVGNPTPQVSWEKDQQ |
| | | PVAAGARFRLAQDGDLYRLTILDLALGDSGQYVCRARNAIGEAFAAV |
| | | GLQVDAEA |
| OL.1 | 219 | QGSPPEFLRFPRPVRVVSGAEAELKCVVLGEPPPVVVWEKGGQQLAA |
| | | SERLSFPADGAEHGLLLTAALPTDAGCYVCRARNAAGEAYAAAAVTV |
| | | LEPP |
| OL.2 | 220 | QGSPPEFLRFPRPVRVVSGAEAELKCVVLGEPPPVVVWEKGGQQLAA |
| | | SERLSFPADGAEHGLLLTAALPTDAGVYVCRARNAACEAYAAAAVTV |
| | | LEPP |
| OL.3 | 221 | QGSPPEFLRFPRPVRVVSGAEAELKCVVLGEPPPVVVWEKGGQQLAA |
| | | SERLSFPADGAEHGLLLTAALPTDAGVYVCRARNAAGECYAAAAVT |
| | | VLEPP |
| OL.4 | 222 | QGSPPEFLRFPRPVRVVSGAEAELKSVVLGEPPPVVVWEKGGQQLAA |
| | | SERLSFPADGAEHGLLLTAALPTDAGCYVSRARNAAGEAYAAAAVTV |
| | | LEPP |
| OL.5 | 223 | QGSPPEFLRFPRPVRVVSGAEAELKSVVLGEPPPVVVWEKGGQQLAA |
| | | SERLSFPADGAEHGLLLTAALPTDAGVYVSRARNAACEAYAAAAVTV |
| | | LEPP |
| OL.6 | 224 | QGSPPEFLRFPRPVRVVSGAEAELKSVVLGEPPPVVVWEKGGQQLAA |
| | | SERLSFPADGAEHGLLLTAALPTDAGVYVSRARNAAGECYAAAAVTV |
| | | LEPP |
| O.17 | 225 | SGAPRFLTRPKSYTVSVGKDASLSSQIVGNPTPQVSWEKDQQPVAAG |
| | | ARFRLAQDGDLYRLKILDLALGDSGQYVSRARNAIGEAFACVGLQV |
| | | DAEA |
| O.18 | 226 | SEAPRFLTRPLAFVVSEGKDATLSSQIVGDPPPEVTWEKDQQPITAGA |
| | | RFRLAQDGDVYRLEILDLQLSDSGQYVSRARNAIGEAFACVGLQVDA |
| | | EG |
| O.19 | 227 | SGAPRFLTRPLAFVVSVGKLAMLSSQIVGNPTPQVSWEKDQQPVAAG |
| | | ARFRLLQDGDLYRLKILDLALGDSGQYVSRARNAIGEAFACVGLQVD |
| | | AEA |
| O.20 | 228 | SGAPRFLTRPLAFVVSVGKDATLSSQIVGNPTPQVSWEKDKQPVTAG |
| | | ARFRLAQDGDLYRLKILDLQLSDSGQYVSRARNAIGEAFACLGLQVD |
| | | AEA |
| O.21 | 229 | SGAPRFLTRPLAFVVSVGKDATLSSQIVGNPTPQVSWEKDQLPVTAG |
| | | ARFRLAQDGDLYRLKILDLTLSDSGQYVSRARNAIGEAFACVGLEVG |
| | | AEA |
| O.22 | 230 | SGAPRFLTRPLSYVVSVGKDASLSSQIVGNPTPQVSWEKDQLPVTAG |
| | | ARFRLAQDGDLYRLKILDLQLSDSGQYVSRARNAIGEAFACVGLEVG |
| | | AEA |
| O.23 | 231 | DQPQFSGAPRFLTRPLSYVVSVGKDASLSSQIVGNPTPQVSWEKDQLP |
| | | VTAGARFRLAQDGDLYRLKILDLQLSDSGQYVSRARNAIGEAFACVG |
| | | LEVGAEA |
| O.24 | 232 | SGAPRFLTRPKAFVVSVGKDATLSSQIVGNPFPQVSWEKDKQPVTAG |
| | | VRFRLAQDGDLYRLKILDLQLSDSGQYVSRARNAIGEAFACLGLQVD |
| | | AEA |
| O.25 | 233 | SGAPRFLTRPKASVVSVGKDATLSSQIVGNPFPQVSWEKDKQPVTAG |
| | | VRFRLAQDGDLYRLKILDLQLSDSGQYVSRARNAHGEAFACLGLQV |
| | | DAEA |
| O.26 | 234 | SGCPRFLTRPKASVVSVGKDATLSSQIVGNPFPQVSWEKDKQPVTAG |
| | | VRFRLAQDGDLYRLKILDLQLSDSGQYVSRARNAHGEAFACLGLQV |
| | | DAEA |
| O.27 | 235 | SGAPRFLTCPKASVVSVGKDATLSSQIVGNPFPQVSWEKDKQPVTAG |
| | | VRFRLAQDGDLYRLKILDLQLSDSGQYVSRARNAHGEAFACLGLQV |
| | | DAEA |
| O.28 | 236 | SGAPRFLTRPKASVVSVGKDATLSCQIVGNPFPQVSWEKDKQPVTAG |
| | | VRFRLAQDGDLYRLKILDLQLSDSGQYVCRARNAHGEAFACLGLQV |
| | | DAEA |
| O.29 | 237 | SGCPRFLTRPKASVVSVGKDATLSCQIVGNPFPQVSWEKDKQPVTAG |
| | | VRFRLAQDGDLYRLKILDLQLSDSGQYVCRARNAHGEAFACLGLQV |
| | | DAEA |
| O.30 | 238 | SGAPRFLTCPKASVVSVGKDATLSCQIVGNPFPQVSWEKDKQPVTAG |
| | | VRFRLAQDGDLYRLKILDLQLSDSGQYVCRARNAHGEAFACLGLQV |
| | | DAEA |
| O.31 | 239 | SGAPRFLTRPKASVVSVGKDATLSSQIVGNPFPQVSWEKDKQPVTAG |
| | | VRFRLAQDGDLYRLKILDCQLSDSGQYVSRARNAHGEAFACLGLQC |
| | | DAEA |
| O.32 | 240 | SGCPRFLTRPKASVVSVGKDATLSSQIVGNPFPQVSWEKDKQPVTAG |
| | | VRFRLAQDGDLYRLKILDCQLSDSGQYVSRARNAHGEAFACLGLQC |
| | | DAEA |
| O.33 | 241 | SGAPRFLTCPKASVVSVGKDATLSSQIVGNPFPQVSWEKDKQPVTAG |
| | | VRFRLAQDGDLYRLKILDCQLSDSGQYVSRARNAHGEAFACLGLQC |
| | | DAEA |

### Engineering of Fc Region

In one aspect, the Fc region of the antigen-binding molecule of the present disclosure comprises one or more amino acid substitutions that reduce its binding to an Fc receptor, e.g., its binding to the Fcγ receptor, and reduce or eliminate effector functions. A natural IgG Fc region, specifically an IgG₁ Fc region or an IgG4 Fc region, may cause the antigen-binding molecule of the present disclosure to target cells expressing an Fc receptor, rather than cells expressing an antigen. The engineered Fc region of the present disclosure exhibits reduced binding affinity for an Fc receptor and/or reduced effector functions. In some embodiments, the engineered Fc region shows no less than 50%, 80%, 90%, or 95% reduction in binding affinity for an Fc receptor as compared to the native Fc region. In some embodiments, the Fc receptor is an Fcγ receptor. In some embodiments, the Fc receptor is a human Fcγ receptor, e.g., FcγRI, FcγRIIa, FcγRIIB, or FcγRIIIa. In some embodiments, the engineered Fc region also has reduced binding affinity for a complement (e.g., C1q) as compared to the native Fc region. In some embodiments, the engineered Fc region has no reduced binding affinity for a neonatal Fc receptor (FcRn) as compared to the native Fc region. In some examples, the engineered Fc region has reduced effector functions, which may include, but are not limited to, one or more of the following: reduced complement-dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen presenting cells, reduced binding to NK cells, reduced binding to macrophages, reduced binding to monocytes, reduced binding to polymorphonuclear cells, reduced direct signaling-induced apoptosis, reduced dendritic cell maturation, and reduced T cell priming. For the IgG₁ Fc region, amino acid residue substitutions at positions such as positions 238, 265, 269, 270, 297, 327, and 329 can reduce effector functions. In some embodiments, the Fc region is a human IgG₁ Fc region, and the amino acid residues at positions 234 and 235 are A, as numbered according to the EU index. For the IgG4 Fc region, amino acid residue substitutions at positions such as position 228 can reduce effector functions.

The antigen-binding molecule may also comprise disulfide bond engineering, such as 354C in the first subunit and 349C in the second subunit. To increase the serum half-life of the antigen-binding molecule, 252Y, 254T, and 256E mutations may be introduced.

When the antigen-binding molecule comprises different binding moieties fused to the two subunits of the Fc region, it may lead to undesired homodimerization. To improve yield and purity, it is thus advantageous to introduce modifications that promote heterodimerization in the Fc region of the antigen-binding molecule of the present disclosure. In some embodiments, the Fc region of the present disclosure comprises modifications according to the knob-into-hole (KIH) technique, which involves introducing a knob structure at the interface of the first subunit and a hole structure at the interface of the second subunit. As such, the knob structure can be positioned in the hole structure, thereby promoting the formation of heterodimers and inhibiting the production of homodimers. The knob structure is constructed by substituting a small amino acid side chain at the interface of the first subunit with a larger side chain (e.g., tyrosine or tryptophan). The hole structure is created at the interface of the second subunit by substituting a large amino acid side chain with a smaller amino acid side chain (e.g., alanine or threonine). The knob structure and the hole structure are prepared by altering the nucleic acid encoding the polypeptide, with optional amino acid substitutions shown in Table 4:

TABLE 4
Combinations of KIH mutations

| | | | | | | | | |
|---|---|---|---|---|---|---|---|---|
| First | T366Y | T366W | T394W | F405W | T366W | T366Y | T366W | F405W |
| subunit | | | | | | F405A | F405W | Y407A |
| Second | Y407T | Y407A | F405A | T394S | T366S | T394W | T394W | T366W |
| subunit | | | | | L368A | Y407T | Y407A | T394S |
| | | | | | Y407V | | | |

In addition to the knob-into-hole technique, other techniques for modifying the CH3 domain of the heavy chain to achieve heterodimerization are also known in the art, e.g., WO1996027011A1, WO1998050431A2, EP1870459A1, WO2007110205A2, WO2009089004A1, WO2010129304A2, WO2011143545A1, WO2012058768A1, WO2013157954A1, and WO2013096291A2.

The C-terminus of the Fc region may be an intact C-terminus ending with amino acid residues PGK or a truncated C-terminus, e.g., a truncated C-terminus in which one or two C-terminal amino acid residues are removed. In a preferred aspect, the C-terminus of the heavy chain is a shortened C-terminus ending with PG. Thus, in some embodiments, a composition of intact antibodies may comprise antibody populations with all K447 residues and/or G446+K447 residues removed. In some embodiments, a composition of intact antibodies may comprise antibody populations without a K447 residue and/or G446+K447 residues removed. In some embodiments, a composition of intact antibodies comprises antibody populations having a mixture of antibodies with and without a K447 residue and/or G446+K447 residues.

### Recombination Method

The antigen-binding molecule may be produced using recombination methods. For these methods, one or more isolated nucleic acids encoding the antigen-binding molecule are provided.

In the case of a native antibody, a native antibody fragment or a bispecific antibody with homodimeric heavy chains, two nucleic acids are required, one for the light chain or a fragment thereof and the other for the heavy chain or a fragment thereof. Such nucleic acids encode an amino acid sequence comprising the VL of the antibody and/or an amino acid sequence comprising the VH of the antibody (e.g., the light and/or heavy chain of the antibody). These nucleic acids can be on the same expression vector or different expression vectors.

In the case of a bispecific antibody with heterodimeric heavy chains, four nucleic acids, for example, are required, one for a first light chain, one for a first heavy chain comprising a first heteromonomeric Fc-region polypeptide, one for a second light chain, and one for a second heavy chain comprising a second heteromonomeric Fc-region polypeptide. The four nucleic acids can be contained in one or more nucleic acid molecules or expression vectors. Generally, these nucleic acids are located on two or three expression vectors; that is, one vector can comprise more than one of these nucleic acids.

In one embodiment, the present disclosure provides an isolated nucleic acid encoding the aforementioned antibody. Such nucleic acids may independently encode any one of the polypeptide chains described above. In another aspect, the present disclosure provides one or more vectors (e.g., expression vectors) comprising such nucleic acids. In another aspect, the present disclosure provides a host cell comprising such nucleic acids. In one embodiment, provided is a method for preparing an antigen-binding molecule, wherein the method comprises culturing a host cell comprising a nucleic acid encoding the antibody, as provided above, under conditions suitable for antibody expression, and optionally collecting the antibody from the host cell (or host cell culture medium).

For recombinant production of the antigen-binding molecule, the nucleic acid encoding the protein is isolated and inserted into one or more vectors for further cloning and/or expression in a host cell. Such nucleic acids can be readily isolated and sequenced using conventional procedures (e.g., by using oligonucleotide probes that are capable of binding specifically to genes encoding the heavy and light chains of an antibody), or produced by recombinant methods or obtained by chemical synthesis.

Suitable host cells for cloning or expressing a vector encoding the antibody include prokaryotic or eukaryotic cells described herein. For example, the antibody can be produced in bacteria, particularly when the antibody does not require glycosylation and Fc effector functions. After expression, the antibody can be isolated from the bacterial cell paste in a soluble fraction and can be further purified.

In addition to prokaryotes, eukaryotic microorganisms such as filamentous fungi or yeast are suitable cloning or expression hosts for antibody-encoding vectors, including fungal and yeast strains, whose glycosylation pathways have been “humanized”, such that an antibody with a partially or fully human glycosylation pattern is produced. Suitable host cells for expression of (glycosylated) antibodies may also be derived from multicellular organisms (invertebrates and vertebrates); examples of invertebrate cells include plant and insect cells. A number of baculovirus strains have been identified, which can be used in combination with insect cells, particularly for transfection of Spodoptera frugiperda cells; plant cell cultures may also be used as hosts, see, e.g., U.S. Pat. Nos. 5,959,177, 6,040,498, 6,420,548, 7,125,978 and 6,417,429; vertebrate cells can also be used as hosts, e.g., mammalian cell lines adapted for growth in a suspension. Other examples of suitable mammalian host cell lines include an SV40-transformed monkey kidney CV1 line (COS-7); a human embryonic kidney line (293 or 293T cells); baby hamster kidney cells (BHK); mouse sertoli cells (TM4 cells); monkey kidney cells (CV1); African green monkey kidney cells (VERO-76); human cervical cancer cells (HELA); canine kidney cells (MDCK); buffalo rat liver cells (BRL3A); human lung cells (W138); human liver cells (Hep G2); mouse mammary tumor cells (MMT 060562); TRI cells; MRC 5 cells; and FS4 cells. Other suitable mammalian host cell lines include Chinese hamster ovary (CHO) cells, including DHFR-CHO cells; and myeloma cell lines such as Y0, NSO, and Sp2/0. For reviews of certain mammalian host cell lines suitable for the production of the antibody, see, e.g., Yazaki, P. and Wu, A. M., Methods in Molecular Biology, Vol. 248, Lo, B. K. C. (eds.), Humana Press, Totowa, NJ (2004), pp. 255-268.

### Immunoconjugate

The present disclosure also provides an immunoconjugate comprising an antigen-binding molecule conjugated to one or more cytotoxic agents, such as a chemotherapeutic agent or drug, a growth inhibitor, a toxin (e.g., a protein toxin, an enzymatically active toxin, or a fragment thereof, of bacterial, fungal, plant or animal origin), or a radioisotope.

### Diagnostic and Therapeutic Compositions

In certain embodiments, the antigen-binding molecule provided in the present disclosure can be used to detect the presence of GUCY2C and/or CD3 in a biological sample. As used herein, the term “detection” encompasses quantitative or qualitative detection. In certain embodiments, the biological sample includes a cell or tissue, such as tumor tissue.

In one embodiment, provided is an antigen-binding molecule for use in a diagnostic or detection method. In yet another aspect, provided is a method for detecting the presence of GUCY2C and/or CD3 in a biological sample. In certain embodiments, the method comprises contacting the biological sample with an antigen-binding molecule under suitable conditions and detecting whether a complex is formed between the detection agent and the antigen. Such methods may be in vitro or in vivo methods. In one embodiment, an antigen-binding molecule is used to select a subject suitable for treatment; for example, GUCY2C and/or CD3 are biomarkers for selecting a patient.

Exemplary disorders that can be diagnosed using the antigen-binding molecule of the present disclosure are for example, proliferative diseases or tumors (e.g., cancer).

In certain embodiments, provided is a labeled antigen-binding molecule. Labels include, but are not limited to, labels or moieties that are detected directly (such as fluorescent, chromophoric, electron-dense, chemiluminescent, and radioactive labels), and moieties that are detected indirectly (e.g., moieties that are detected indirectly through an enzymatic reaction or molecular interaction, such as enzymes or ligands).

In an additional aspect, provided is a pharmaceutical composition comprising the antigen-binding molecule, for example, for use in any of the following treatment methods. In one aspect, the pharmaceutical composition comprises any of the antigen-binding molecules provided herein and a pharmaceutically acceptable carrier. In another aspect, the pharmaceutical composition comprises any of the antigen-binding molecules provided herein and at least one additional therapeutic agent.

The pharmaceutical composition of the antigen-binding molecule described in the present disclosure is prepared by: mixing such an antigen-binding molecule having desired purity with one or more optional pharmaceutically acceptable carriers. The pharmaceutical composition is in the form of a lyophilized composition or an aqueous solution. Formulations for in vivo administration are generally sterile. Sterility can be readily achieved, for example, by filtration through a sterile filter membrane.

### Treatment Method and Route of Administration

Any of the antigen-binding molecules provided herein can be used in the treatment method.

In yet another aspect, the present disclosure provides use of the antigen-binding molecule in the manufacture or preparation of a medicament. In one embodiment, the medicament is used for treating a proliferative disease or tumor. In addition, the medicament is present in an amount effective for the diseases described above. In some embodiments, the effective amount is a unit daily dose or a unit weekly dose. In one such embodiment, the use further comprises administering to a subject an effective amount of at least one additional therapeutic agent (e.g., one, two, three, four, five, or six additional therapeutic agents). The “subject” according to any of the above embodiments may be a human.

In yet another aspect, provided is a pharmaceutical composition comprising the antigen-binding molecule, e.g., for any of the above pharmaceutical uses or treatment methods. In another embodiment, the pharmaceutical composition also comprises at least one additional therapeutic agent.

The antigen-binding molecule of the present disclosure may be used alone or in combination with other agents for the treatment. For example, the antigen-binding molecule of the present disclosure may be co-administered with at least one additional therapeutic agent. In some embodiments, the additional therapeutic agent is a bispecific antibody that specifically binds to EGFR and CD28.

The antigen-binding molecule of the present disclosure (and any additional therapeutic agents) may be administered by any suitable means, including parenteral, intrapulmonary, and intranasal administration, and if required for local treatment, intralesional administration. Parenteral infusion includes intramuscular, intravenous, intra-arterial, intraperitoneal, or subcutaneous administration. Administration may be performed by any suitable route, e.g., by injection, such as intravenous or subcutaneous injection, depending in part on whether the administration is short-term or long-term. Various administration schedules are contemplated herein, including but not limited to, single administration or multiple administrations at multiple time points, bolus administration, and pulse infusion.

The antigen-binding molecule of the present disclosure will be formulated, administered, and applied in a manner consistent with Good Medical Practice. Factors considered in this context include the specific disorder being treated, the specific mammal being treated, the clinical state of the individual patient, the cause of the disorder, the site of delivery of the agent, the method of administration, the timing of administration, and other factors known to medical practitioners. The antigen-binding molecule needs not be, but is optionally, formulated along with one or more agents currently used for preventing or treating the disorder. The effective amount of such additional agents depends on the amount of the antigen-binding molecule present in the pharmaceutical composition, the type of the disorder or treatment, and other factors discussed above. These are generally used in the same dose and with routes of administration as described herein, or in about 1 to 99% of the dose described herein, or in any dose and by any route that is empirically/clinically determined to be appropriate.

For the prevention or treatment of a disease, the appropriate dose of the antigen-binding molecule of the present disclosure (when used alone or in combination with one or more additional therapeutic agents) will depend on the type of disease to be treated, the type of therapeutic molecule, the severity and course of the disease, the purpose of the administration (prophylactic or therapeutic), previous treatment, clinical history and response to the therapeutic molecule of the patient, and the discretion of the attending physician. The therapeutic molecule is suitably administered to a patient in one or a series of treatments. Depending on the type and severity of the disease, about 1 μg/kg to 15 mg/kg of the antigen-binding molecule can be an initial candidate dose for administration to the patient, whether, for example, by one or more separate administrations, or by continuous infusion. One typical daily dose may range from about 1 μg/kg to 100 mg/kg or more, depending on the factors mentioned above. Accordingly, taking 50 kg body weight as an example, an exemplary unit daily dose is 50 μg-5 g.

### Article of Manufacture

In another aspect of the present disclosure, provided is an article of manufacture, which comprises materials useful for the treatment, prevention, and/or diagnosis of the disorders described above. The article of manufacture comprises a container and a label or package insert on or associated with the container. Suitable containers include, for example, bottles, vials, syringes, IV solution bags, and the like. The containers may be formed from a variety of materials such as glass or plastic. The container contains a composition effective in the treatment, prevention, and/or diagnosis of a disease, either alone or in combination with another composition, and may have a sterile access port (e.g., the container may be an intravenous solution bag or vial with a stopper pierceable by a hypodermic injection needle). At least one active agent in the composition is the antigen-binding molecule of the present disclosure. The label or package insert indicates that the composition is used to treat the selected condition. Further, the article of manufacture may comprise: (a) a first container containing a composition that comprises the antigen-binding molecule of the present disclosure; and (b) a second container containing a composition that comprises other cytotoxic agents or additional therapeutic agents. The article of manufacture in this embodiment of the present disclosure may further comprise a package insert indicating that the composition may be used to treat a particular condition. Alternatively or additionally, the article of manufacture may further comprise a second (or third) container containing a pharmaceutically acceptable buffer. From a commercial and user standpoint, it may further contain other materials as required, including other buffers, diluents, filters, needles, and syringes.

### EXAMPLES AND TEST EXAMPLES

The present disclosure is further described below with reference to the following examples and test examples, which, however, do not limit the present disclosure. The experimental methods in the examples and test examples of the present disclosure in which specific conditions are not specified are generally performed under conventional conditions such as Antibodies: A Laboratory Manual and Molecular Cloning: A Laboratory Manual by Cold Spring Harbor Laboratory, or under conditions recommended by the manufacturers of the starting materials or commercial products. Reagents without specific origins indicated are commercially available conventional reagents.

### Example 1. Antigen-Binding Molecules Comprising Titin Chain/Obscurin Chain

The Titin chain/Obscurin chain of the present disclosure may be derived from any suitable polypeptide, including polypeptides derived from WO2021139758A1 (incorporated herein by reference in its entirety) and CN202110527339.7 and the patents which refer to it as a priority document (incorporated herein by reference in their entireties). Bispecific antibodies were constructed, where the CL was the kappa light chain constant region in WO2021139758A1, the amino acid sequences of the Titin and Obscurin chains are shown in Tables 3-1 and 3-2, and the linker sequences include GGGGS (SEQ ID NO: 90), ASTKG (SEQ ID NO: 243), or RTVAS (SEQ ID NO: 244). The amino acid sequences of the Fc1, Fc2, and CH1 in this example are shown below.

>Example 1-Fc1
(knob, SEQ ID NO: 85)
DKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIE
KTISKAKGQPREPQVYTLPPCREEMTKNQVSLWCLVKGFYPSDIAVEWESNGQPENN
YKTTPPVLDSDGSFFLYSKLTVDKSRWQQGNVFSCSVMHEALHNHYTQKSLSLSPGK
>Example 1-Fc2
(hole, SEQ ID NO: 86)
DKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIE
KTISKAKGQPREPQVCTLPPSREEMTKNQVSLSCAVKGFYPSDIAVEWESNGQPENN
YKTTPPVLDSDGSFFLVSKLTVDKSRWQQGNVFSCSVMHEALHNHYTQKSLSLSPGK
>Example 1-CH1
(SEQ ID NO: 84)
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSC

1.1. DI bispecific antibodies

DI bispecific antibodies against hNGF and hRANKL, DI-2 to DI-20, which comprise a first heavy chain, a second heavy chain, a first light chain, and a second light chain as described below, were constructed with reference to Example 5 in WO2021139758A1:
the first heavy chain: when viewed from N-terminus to C-terminus, [VH1-I]-[linker 1]-[Obscurin chain]-[Fc2];
the first light chain: when viewed from N-terminus to C-terminus, [VL1-I]-[linker 2]-[Titin chain];
the second heavy chain: when viewed from N-terminus to C-terminus, [VH2-D]-[CH1]-[Fc1]; and
the second light chain: when viewed from N-terminus to C-terminus, [VL2-D]-[CL];
wherein the VH1-I and VL1-I were the heavy chain variable region and the light chain variable region of I0 in WO2021139758A1, respectively, and the VH2-D and VL2-D were the heavy chain variable region and the light chain variable region of DO in WO2021139758A1, respectively. The structures of the Obscurin chain, the Titin chain, the linker 1, and the linker 2 in the DI bispecific antibodies of this example are shown in the following table.

TABLE 5
Obscurin chain/Titin chain and linkers in DI bispecific antibodies

| | First heavy chain | | First light chain | |
|---|---|---|---|---|
| No. | Obscurin chain | Linker 1 | Titin chain | Linker 2 |
| DI-2 | 0.20 | GGGGS | T.10 | GGGGS |
| DI-3 | 0.25 | GGGGS | T.16 | GGGGS |
| DI-4 | 0.26 | GGGGS | T.17 | GGGGS |
| DI-5 | 0.27 | GGGGS | T.18 | GGGGS |
| DI-6 | 0.28 | GGGGS | T.16 | GGGGS |
| DI-7 | 0.29 | GGGGS | T.17 | GGGGS |
| DI-8 | 0.30 | GGGGS | T.18 | GGGGS |
| DI-9 | 0.31 | GGGGS | T.16 | GGGGS |
| DI-10 | 0.32 | GGGGS | T.17 | GGGGS |
| DI-11 | 0.33 | GGGGS | T.18 | GGGGS |
| DI-12 | 0.25 | ASTKG | T.16 | RTVAS |
| DI-13 | 0.26 | ASTKG | T.17 | RTVAS |
| DI-14 | 0.27 | ASTKG | T.18 | RTVAS |
| DI-15 | 0.28 | ASTKG | T.16 | RTVAS |
| DI-16 | 0.29 | ASTKG | T.17 | RTVAS |
| DI-17 | 0.30 | ASTKG | T.18 | RTVAS |
| DI-18 | 0.31 | ASTKG | T.16 | RTVAS |
| DI-19 | 0.32 | ASTKG | T.17 | RTVAS |
| DI-20 | 0.33 | ASTKG | T.18 | RTVAS |

Note: The numbering of the Titin and Obscurin chains in the table is shown in Tables 3-1 and 3-2.

The binding activity of the bispecific antibodies DI-2 to DI-20 for their antigens was assayed by the method of Test Example 4 of WO2021139758A1. The thermal stability of the antibodies was studied. Study method: The antibodies were diluted with PBS to a concentration of 5 mg/mL, and their thermal stability was measured using a high throughput differential scanning fluorimeter (UNCHAINED; model: Unit). The experimental results show that there was no significant change in the antigen binding activity of the engineered bispecific antibodies; additionally, there were significant increases in the Tm1 (° C.) and Tonset (° C.) of DI-4 to DI-8, DI-10 to DI-16, and DI-20 as compared to DI-2, suggesting that the thermal stability of the bispecific antibodies is better.

TABLE 6
Binding activity of DI bispecific antibodies

| | |
|---|---|
| RANKL | NGF |

| | |
|---|---|
| No. | EC₅₀ (nM) |

| | | |
|---|---|---|
| DI-2 | 0.3832 | 6.633 |
| DI-3 | 0.3613 | 5.7730 |
| DI-4 | 0.3959 | 6.2930 |
| DI-5 | 0.3290 | 6.1890 |
| DI-6 | 0.2509 | 5.6720 |
| DI-7 | 0.2557 | 6.6430 |
| DI-8 | 0.3643 | 7.6250 |
| DI-9 | 0.2944 | 8.4950 |
| DI-10 | 0.3460 | 7.1660 |
| DI-11 | 0.3721 | 10.9600 |
| DI-12 | 0.4125 | 6.5156 |
| DI-13 | 0.4440 | 5.5420 |
| DI-14 | 0.4182 | 3.2610 |
| DI-15 | 0.2206 | 5.2800 |
| DI-16 | 0.1474 | 5.5140 |
| DI-17 | 0.2329 | 6.7270 |
| DI-18 | 0.2662 | 5.9080 |
| DI-19 | 0.1843 | 5.9280 |
| DI-20 | 0.3184 | 6.4250 |

TABLE 7
Thermal stability of DI bispecific antibodies

| No. | Tm1 (° C.) | Tonset (° C.) |
|---|---|---|
| DI-2 | 55.6 | 48.3 |
| DI-4 | 60.1 | 52.493 |
| DI-5 | 61 | 51.967 |
| DI-6 | 60.8 | 53.012 |
| DI-7 | 60.34 | 52.003 |
| DI-8 | 60.61 | 50.425 |
| DI-10 | 60.2 | 52.766 |
| DI-11 | 57.35 | — |
| DI-12 | 59.9 | 51.726 |
| DI-13 | 61 | 50.988 |
| DI-14 | 61.2 | 52.191 |
| DI-15 | 60.41 | 50.558 |
| DI-16 | 61.5 | 50.691 |
| DI-20 | 60.7 | 51.859 |

Solutions of the DI bispecific antibodies were prepared using a buffer containing 10 mM acetic acid at pH 5.5 and 9% sucrose, and the solutions were incubated in a 40 CC incubator for four weeks. Following the incubation, the antibody solutions were concentrated to the concentration at the start of the incubation, and the solutions were observed for precipitate formation. The experimental results show that there was a precipitate formed in the solution of the DI-2 bispecific antibody group. DI-3 to DI-7 are more stable than DI-2.

TABLE 8
Precipitate formation in the solutions
of the DI bispecific antibodies

| | | Concentration | |
|---|---|---|---|
| | | after solution | |
| | Initial | concentration | Precipitate formation |
| No. | concentration | at week 4 | in solution |
| DI-2 | 20 mg/mL | 20 mg/mL | Precipitation occurred |
| DI-3 | 20 mg/mL | 20 mg/mL | No precipitation |
| DI-4 | 60 mg/mL | 60 mg/mL | No precipitation |
| DI-5 | 25 mg/mL | 25 mg/mL | No precipitation |
| DI-6 | 60 mg/mL | 60 mg/mL | No precipitation |
| DI-7 | 16 mg/mL | 16 mg/mL | No precipitation |

### 1.2. PL Bispecific Antibodies

PL bispecific antibodies against hPDL1 and hCTLA4, PL-1 to PL-19, which comprise a first heavy chain, a second heavy chain, a first light chain, and a second light chain as described below, were constructed:
the first heavy chain: when viewed from N-terminus to C-terminus, [VH1-P]-[linker 1]-[Obscurin chain]-[Fc1];
the first light chain: when viewed from N-terminus to C-terminus, [VL1-P]-[linker 2]-[Titin chain];
the second heavy chain: when viewed from N-terminus to C-terminus, [VH2-L]-[CH1]-[Fc2]; and
the second light chain: when viewed from N-terminus to C-terminus, [VL2-L]-[CL];
wherein the VH1-P and VL1-P were the heavy chain variable region and the light chain variable region of the h1831K antibody in WO2020177733A1, respectively. The amino acid sequences of the VH2-L and VL2-L are shown below.

>VH2-L
(SEQ ID NO: 180)
QVQLVESGGGVVQPGRSLRLSCAASGFTFSSYTMHWVRQAPGKGLEWVTFISYDGN
NKYYADSVKGRFTISRDNSKNTLYLQMNSLRAEDTAIYYCARTGWLGPFDYWGQGT
LVTVSS
>VL2-L
(SEQ ID NO: 181)
EIVLTQSPGTLSLSPGERATLSCRASQSVGSSYLAWYQQKPGQAPRLLIYGAFSRATGI
PDRFSGSGSGTDFTLTISRLEPEDFAVYYCQQYGSSPWTFGQGTKVEIK

The structures of the Obscurin chain, the Titin chain, the linker 1, and the linker 2 in the PL bispecific antibodies of this example are shown in the following table.

TABLE 9
Obscurin chain/Titin chain and linkers in PL bispecific antibodies

| | First heavy chain | | First light chain | |
|---|---|---|---|---|
| No. | Obscurin chain | Linker 1 | Titin chain | Linker 2 |
| PL-1 | O.20 | GGGGS | T.10 | GGGGS |
| PL-2 | O.25 | GGGGS | T.16 | GGGGS |
| PL-3 | O.26 | GGGGS | T.17 | GGGGS |
| PL-4 | O.27 | GGGGS | T.18 | GGGGS |
| PL-5 | O.28 | GGGGS | T.16 | GGGGS |
| PL-6 | O.29 | GGGGS | T.17 | GGGGS |
| PL-7 | O.30 | GGGGS | T.18 | GGGGS |
| PL-8 | O.31 | GGGGS | T.16 | GGGGS |
| PL-9 | O.32 | GGGGS | T.17 | GGGGS |
| PL-10 | O.33 | GGGGS | T.18 | GGGGS |
| PL-11 | O.25 | ASTKG | T.16 | RTVAS |
| PL-12 | O.26 | ASTKG | T.17 | RTVAS |
| PL-13 | O.27 | ASTKG | T.18 | RTVAS |
| PL-14 | O.28 | ASTKG | T.16 | RTVAS |
| PL-15 | O.29 | ASTKG | T.17 | RTVAS |
| PL-16 | O.30 | ASTKG | T.18 | RTVAS |
| PL-17 | O.31 | ASTKG | T.16 | RTVAS |
| PL-18 | O.32 | ASTKG | T.17 | RTVAS |
| PL-19 | O.33 | ASTKG | T.18 | RTVAS |

Note: The numbering of the Titin and Obscurin chains in the table is shown in Tables 3-1 and 3-2.

The binding activity of PL bispecific antibodies was tested by the ELISA assay with reference to Test Example 4 of WO2021139758A1, wherein hPDL1 and hCTLA4 antigens were purchased from Sino biology. The thermal stability of the antibodies was studied. Method: The antibodies were diluted with PBS to a concentration of 1.4-3 mg/mL, and their thermal stability was measured using a high throughput differential scanning fluorimeter (UNCHAINED; model: Unit). The experimental results show that the PL bispecific antibodies retained good antigen binding activity; additionally, there were significant increases in the Tm1 (° C.), Tagg 266 (° C.), and Tonset (° C.) of PL-2 to PL-19 as compared to PL-1, suggesting that the thermal stability of the bispecific antibodies is belier.

TABLE 10
Binding activity of PL bispecific antibodies

| | |
|---|---|
| hPDL1 | hCTLA4 |

| | |
|---|---|
| No. | EC₅₀ (nM) |

| | | |
|---|---|---|
| PL-1 | 1.6 | 16.5 |
| PL-2 | 0.5 | 8.0 |
| PL-3 | 0.6 | 7.9 |
| PL-4 | 0.7 | 6.5 |
| PL-5 | 0.7 | 6.7 |
| PL-6 | 0.8 | 5.3 |
| PL-7 | 0.5 | 7.3 |
| PL-8 | 1.4 | 14.5 |
| PL-9 | 0.6 | 5.5 |
| PL-10 | 0.6 | 6.9 |
| PL-11 | 0.6 | 5.6 |
| PL-12 | 0.5 | 8.0 |
| PL-13 | 0.5 | 4.4 |
| PL-14 | 1.3 | 6.0 |
| PL-15 | 1.4 | 3.2 |
| PL-16 | 1.7 | 34.9 |
| PL-17 | 1.4 | 6.2 |
| PL-18 | 1.6 | 6.7 |
| PL-19 | 1.6 | 6.5 |

TABLE 11
Thermal stability of PL bispecific antibodies

| No. | Tm1 (° C.) | Tagg 266 (° C.) | Tonset (° C.) |
|---|---|---|---|
| PL-1 | 61.64 | 64.82 | 52.566 |
| PL-2 | 66.20 | 66.55 | 58.317 |
| PL-3 | 64.07 | 68.28 | 57.661 |
| PL-4 | 70.71 | 67.29 | 56.246 |
| PL-5 | 74.56 | 68.11 | 58.407 |
| PL-6 | 70.43 | 70.12 | 61.069 |
| PL-7 | 68.46 | 67.41 | 63.031 |
| PL-8 | 65.00 | — | — |
| PL-9 | 69.24 | 67.83 | 58.597 |
| PL-10 | 69.63 | 68.12 | 56.788 |
| PL-11 | 65.88 | — | 57.976 |
| PL-12 | 65.54 | 67.94 | — |
| PL-13 | 71.85 | 68.17 | 58.581 |
| PL-14 | 74.18 | 69.42 | 58.589 |
| PL-15 | 70.96 | 69.91 | 58.622 |
| PL-16 | 63.48 | 68.98 | 58.702 |
| PL-17 | 70.15 | 69.86 | 56.193 |
| PL-18 | — | 69.43 | — |
| PL-19 | — | 69.52 | 57.766 |

1.3. HJ bispecific antibodies

HJ bispecific antibodies against hIL5 and hTSLP, HJ-3 to Hill, which comprise a first heavy chain, a second heavy chain, a first light chain, and a second light chain as described below, were constructed:
the first heavy chain: when viewed from N-terminus to C-terminus, [VH1-H]-[linker 1]-[Titin chain]-[Fc1];
the first light chain: when viewed from N-terminus to C-terminus, [VL1-H]-[linker 2]-[Obscurin chain];
the second heavy chain: when viewed from N-terminus to C-terminus, [VH2-J]-[CH1]-[Fc2]; and
the second light chain: when viewed from N-terminus to C-terminus, [VL2-J]-[CL];
wherein the VH1-H and VL1-H were the heavy chain variable region and the light chain variable region of H0 in WO2021139758A1, respectively, and the VH2-J and VL2-J were the heavy chain variable region and the light chain variable region of J1 in WO2021139758A1, respectively. The structures of the Obscurin chain, the Titin chain, the linker 1, and the linker 2 in the HJ bispecific antibodies of this example are shown in the following table.

TABLE 12
Obscurin chain/Titin chain and linkers in HJ bispecific antibodies

| | First heavy chain | | First light chain | |
|---|---|---|---|---|
| No. | Titin chain | Linker 1 | Obscurin chain | Linker 2 |
| HJ-3 | T.10 | GGGGS | O.20 | GGGGS |
| HJ-5 | T.16 | GGGGS | O.25 | GGGGS |
| HJ-6 | T.16 | GGGGS | O.27 | GGGGS |
| HJ-7 | T.16 | GGGGS | O.28 | GGGGS |
| HJ-8 | T.16 | ASTKG | O.25 | RTVAS |
| HJ-9 | T.16 | ASTKG | O.27 | RTVAS |
| HJ-10 | T.16 | ASTKG | O.28 | RTVAS |
| HJ-11 | T.16 | ASTKG | O.29 | RTVAS |

The antigen binding activity of HJ bispecific antibodies was assayed with reference to the method in Test Example 4 of WO2021139758A1. The thermal stability of the antibodies was studied. Method: Diluted solutions of the HJ bispecific antibodies were prepared with a buffer containing 10 mM acetic acid at pH 5.5 and 9% sucrose and then concentrated by ultrafiltration to obtain HJ bispecific antibody solutions with different concentrations (the concentrations of the HJ bispecific antibodies are shown in Table 13-2). Subsequently, the concentrated solutions were incubated in a 40° C. incubator. At day 0 (i.e., before the 40° C. incubation, DO), day 7 (the 7th day of the 40° C. incubation, D7), day 14 (the 14th day of the 40° C. incubation, D14), day 21 (the 21st day of the 40° C. incubation, D21), and day 28 (the 28th day of the 40° C. incubation, D28), samples were taken and their SEC purity was tested. Immediately after 28 days of incubation at 40° C., samples were taken and their CE-SDS purity was tested. The experimental results show that there was no significant change in the antigen binding activity of the HJ bispecific antibodies constructed in the present disclosure; additionally, the thermal stability of the HJ-5 to HJ-11 bispecific antibodies is better than that of HJ-3.

TABLE 13-1
Binding activity of HJ bispecific antibodies

| | |
|---|---|
| hIL5 | hTSLP |

| | |
|---|---|
| No. | EC₅₀ (nM) |

| | | |
|---|---|---|
| HJ-3 | 17.38 | 3.234 |
| HJ-5 | 15.96 | 2.639 |
| HJ-6 | 37.05 | 2.884 |
| HJ-7 | 17.69 | 2.182 |
| HJ-8 | 18.74 | 3.332 |
| HJ-9 | 10.86 | 3.184 |
| HJ-10 | 20.81 | 3.173 |
| HJ-11 | 9.464 | 3.005 |

TABLE 13-2
Accelerated stability test results for HJ bispecific antibodies

| | | D0 | D28 | D28Δ | D28 |
|---|---|---|---|---|---|
| | D0 | SEC | SEC | SEC | non-reduced |
| | concentration | purity | purity | purity | CE-SDS |
| No. | (mg/mL) | (%) | (%) | (%) | purity (%) |
| HJ-3 | 50 | 98 | 84 | 14 | 67 |
| HJ-5 | 60.8 | 98.09 | 93.86 | 4.23 | 82.93 |
| HJ-6 | 54 | 98.14 | 89.68 | 8.46 | 80.76 |
| HJ-7 | 56.8 | 97.7 | 92.72 | 4.98 | 85.47 |
| HJ-8 | 62.6 | 93.39 | 84.28 | 9.11 | 73.03 |
| HJ-9 | 53.5 | 90.01 | 85.93 | 4.08 | 80.32 |
| HJ-10 | 69.8 | 90.9 | 88.31 | 2.59 | 80.4 |
| HJ-11 | 50.4 | 92.55 | 90.89 | 1.66 | 82.96 |

### Example 2. Screening and Identification of Anti-Human GUCY2C Murine Antibodies

2.1 Construction of Cell strains

Cell strains CHO-K1/hGUCY2C and HCT116/hGUCY2C were constructed, and the sequences of related proteins are as follows:

Human GUCY2C protein (UniProtKB: P25092):
SEQ ID NO: 1
MKTLLLDLALWSLLFQPGWLSFSSQVSQNCHNGSYEISVLMMGNSAFAEPLKNLED
AVNEGLEIVRGRLQNAGLNVTVNATFMYSDGLIHNSGDCRSSTCEGLDLLRKISNAQ
RMGCVLIGPSCTYSTFQMYLDTELSYPMISAGSFGLSCDYKETLTRLMSPARKLMYFL
VNFWKTNDLPFKTYSWSTSYVYKNGTETEDCFWYLNALEASVSYFSHELGFKVVLR
QDKEFQDILMDHNRKSNVIIMCGGPEFLYKLKGDRAVAEDIVIILVDLFNDQYFEDNV
TAPDYMKNVLVLTLSPGNSLLNSSFSRNLSPTKRDFALAYLNGILLFGHMLKIFLENGE
NITTPKFAHAFRNLTFEGYDGPVTLDDWGDVDSTMVLLYTSVDTKKYKVLLTYDTH
VNKTYPVDMSPTFTWKNSKLPNDITGRGPQILMIAVFTLTGAVVLLLLVALLMLRKYR
KDYELRQKKWSHIPPENIFPLETNETNHVSLKIDDDKRRDTIQRLRQCKYDKKRVILK
DLKHNDGNFTEKQKIELNKLLQIDYYNLTKFYGTVKLDTMIFGVIEYCERGSLREVL
NDTISYPDGTFMDWEFKISVLYDIAKGMSYLHSSKTEVHGRLKSTNCVVDSRMVVKI
TDFGCNSILPPKKDLWTAPEHLRQANISQKGDVYSYGIIAQEIILRKETFYTLSCRDRN
EKIFRVENSNGMKPFRPDLFLETAEEKELEVYLLVKNCWEEDPEKRPDFKKIETTLAK
IFGLFHDQKNESYMDTLIRRLQLYSRNLEHLVEERTQLYKAERDRADRLNFMLLPRLV
VKSLKEKGFVEPELYEEVTIYFSDIVGFTTICKYSTPMEVVDMLNDIYKSFDHIVDHH
DVYKVETIGDAYMVASGLPKRNGNRHAIDIAKMALEILSFMGTFELEHLPGLPIWIRI
GVHSGPCAAGVVGIKMPRYCLFGDTVNTASRMESTGLPLRIHVSGSTIAILKRTECQF
LYEVRGETYLKGRGNETTYWLTGMKDQKFNLPTPPTVENQQRLQAEFSDMIANSLQ
KRQAAGIRSQKPRRVASYKKGTLEYLQLNTTDKESTYF

The full-length gene encoding human GUCY2C was cloned into the mammalian cell expression vector pCDH, and HEK293T cells (ATCC, CRL-11268) were co-transfected with three plasmids, pVSV-G, pCMV-dR8.91 and pCDH-human GUCY2C, to package viruses. After 48 h of transfection, viruses were collected to infect CHO-K1 (ATCC, CCL-61) or HCT116 cells (ATCC, CCL-247). After two weeks of pressure screening, cell subcloning was performed, and cell strains CHO-K1/hGUCY2C and HCT116/hGUCY2C highly expressing GUCY2C were obtained by FACS.

### 2.2 Construction of Fusion Proteins for Immunization or Screening

The human GUCY2C extracellular region was selected, and a His or Fc tag was linked at the C terminus to construct a fusion protein for immunization and screening. The sequences of related proteins are as follows:

hGUCY2C ECD-hFc:
SEQ ID NO: 2
SQVSQNCHNGSYEISVLMMGNSAFAEPLKNLEDAVNEGLEIVRGRLQNAGLNVTVN
ATFMYSDGLIHNSGDCRSSTCEGLDLLRKISNAQRMGCVLIGPSCTYSTFQMYLDTEL
SYPMISAGSFGLSCDYKETLTRLMSPARKLMYFLVNFWKTNDLPFKTYSWSTSYVYK
NGTETEDCFWYLNALEASVSYFSHELGFKVVLRQDKEFQDILMDHNRKSNVIIMCG
GPEFLYKLKGDRAVAEDIVIILVDLFNDQYFEDNVTAPDYMKNVLVLTLSPGNSLLNS
SFSRNLSPTKRDFALAYLNGILLFGHMLKIFLENGENITTPKFAHAFRNLTFEGYDGPV
TLDDWGDVDSTMVLLYTSVDTKKYKVLLTYDTHVNKTYPVDMSPTFTWKNSKLPN
DITGRGPQEPKSSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKC
KVSNKALPAPIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHEALHNHY
TQKSLSLSPGK
hGUCY2C ECD-His:
SEQ ID NO: 3
SQVSQNCHNGSYEISVLMMGNSAFAEPLKNLEDAVNEGLEIVRGRLQNAGLNVTVN
ATFMYSDGLIHNSGDCRSSTCEGLDLLRKISNAQRMGCVLIGPSCTYSTFQMYLDTEL
SYPMISAGSFGLSCDYKETLTRLMSPARKLMYFLVNFWKTNDLPFKTYSWSTSYVYK
NGTETEDCFWYLNALEASVSYFSHELGFKVVLRQDKEFQDILMDHNRKSNVIIMCG
GPEFLYKLKGDRAVAEDIVIILVDLFNDQYFEDNVTAPDYMKNVLVLTLSPGNSLLNS
SFSRNLSPTKRDFALAYLNGILLFGHMLKIFLENGENITTPKFAHAFRNLTFEGYDGPV
TLDDWGDVDSTMVLLYTSVDTKKYKVLLTYDTHVNKTYPVDMSPTFTWKNSKLPN
DITGRGPQHHHHHH

2.3 Screening and identification of anti-GUCY2C murine antibodies

In the present disclosure, monoclonal antibodies against human GUCY2C were prepared by hybridoma fusion or Single B technology, and the obtained antibodies specifically bind to human GUCY2C with high affinity and can have cross binding with cynomolgus monkey GUCY2C and have good binding activity for human GUCY2C on the cell surface.

hGUCY2C ECD-hFc, hGUCY2C ECD-His, the CHO-K1/hGUCY2C cell, or the HCT116/hGUCY2C cell was used as the immunogen. For immunization with protein, 50 μg of protein was used in the primary immunization and 25 μg of protein was used in each boost immunization; cross-immunizations were performed using the adjuvants TiterMax® Gold Adjuvant (Sigma Cat No. T2684) and Thermo Imject® Alum (Thermo Cat No. 77161). For immunization with cells, the immunization was performed according to 1×10⁷ cells/mouse/immunization. After primary immunization and 4-7 times of boost immunization, mice with high antibody titer in serum were selected for spleen cell fusion or single B cell screening.

After fusion, the hybridoma culture supernatant was assayed based on the growth density of hybridoma cells. The hybridomas with good binding activity for human GUCY2C and antigens on the cell surface were selected by screening. Hybridoma cells were harvested, and the RNA was extracted using NucleoZol (MN) (following the procedures in the kit instructions) and reverse-transcribed (PrimeScript™ Reverse Transcriptase, Takara, cat #2680A). The cDNA obtained by the reverse transcription was amplified by PCR using mouse Ig-Primer Set (Novagen, TB326 Rev.B 0503) and then sent for sequencing, and the amino acid sequences of the CDRs and variable regions of the antibodies were obtained.

TABLE 14
CDRs of anti-GUCY2C antibodies resulting from hybridoma fusion screening

| Antibody | Heavy chain CDR | | Light chain CDR | |
|---|---|---|---|---|
| M81 | HCDR1 | DHNMN | LCDR1 | SASSSVNYMY |
| | | (SEQ ID NO: 4) | | (SEQ ID NO: 7) |
| | HCDR2 | VMNPKFGTINYNQKFKD | LCDR2 | RTSNLAS |
| | | (SEQ ID NO: 5) | | (SEQ ID NO: 8) |
| | HCDR3 | DDYPNYFDY | LCDR3 | QQYHDYPPT |
| | | (SEQ ID NO: 6) | | (SEQ ID NO: 9) |
| M146 | HCDR1 | DYNMN | LCDR1 | SASSSLNYMY |
| | | (SEQ ID NO: 10) | | (SEQ ID NO: 13) |
| | HCDR2 | LINPKYGTTNYNQKFKG | LCDR2 | RTSNLAS |
| | | (SEQ ID NO: 11) | | (SEQ ID NO: 8) |
| | HCDR3 | DDYPAWFAY | LCDR3 | QQYHSYPPT |
| | | (SEQ ID NO: 12) | | (SEQ ID NO: 14) |
| M42 | HCDR1 | SYAVH | LCDR1 | RASSFVGSGYLH |
| | | (SEQ ID NO: 250) | | (SEQ ID NO: 253) |
| | HCDR2 | VIWTGGNTRSNAAFMS | LCDR2 | STSKLAS |
| | | (SEQ ID NO: 251) | | (SEQ ID NO: 254) |
| | HCDR3 | NSLLDYAMDY | LCDR3 | QQSSGYPLT |
| | | (SEQ ID NO: 252) | | (SEQ ID NO: 255) |
| M61 | HCDR1 | DYYMS | LCDR1 | SASSSISSNFLH |
| | | (SEQ ID NO: 256) | | (SEQ ID NO: 259) |
| | HCDR2 | DINPNNGGTSDNQKFKG | LCDR2 | RTSNLAS |
| | | (SEQ ID NO: 257) | | (SEQ ID NO: 8) |
| | HCDR3 | GRGMVARYFDI | LCDR3 | QQGSGLPLT |
| | | (SEQ ID NO: 258) | | (SEQ ID NO: 260) |
| M64 | HCDR1 | DYGMH | LCDR1 | SASSSVSYMY |
| | | (SEQ ID NO: 261) | | (SEQ ID NO: 264) |
| | HCDR2 | YISSGSSTIYYADTVKG | LCDR2 | DTSKLAS |
| | | (SEQ ID NO: 262) | | (SEQ ID NO: 265) |
| | HCDR3 | TRVYFYGSYWYFDV | LCDR3 | QQWSSDPPYT |
| | | (SEQ ID NO: 263) | | (SEQ ID NO: 266) |
| M93 | HCDR1 | RYWMH | LCDR1 | KASQDVSSAVA |
| | | (SEQ ID NO: 267) | | (SEQ ID NO: 270) |
| | HCDR2 | MIDPSDSYTK YNQKFMG | LCDR2 | SASYRST |
| | | (SEQ ID NO: 268) | | (SEQ ID NO: 271) |
| | HCDR3 | SIKVAGWYFDV | LCDR3 | QQHFSTTLT |
| | | (SEQ ID NO: 269) | | (SEQ ID NO: 272) |

>M81 VH:
SEQ ID NO: 15
EFQLQQSGPELVKPGASVKISCKASGYSFTDHNMNWVKQSNGKSLEWIGVMNPKFG
TINYNQKFKDKATLTVDQSSSTAYMQLNSLTSEDSAVYYCVRDDYPNYFDYWGQGT
TLTVSS
>M81 VL:
SEQ ID NO: 16
QIVLTQSPAIMSASPGEKVTISCSASSSVNYMYWYQQKPGSSPKSWIYRTSNLASGVS
ARFSGSGSGTSYSLTISSMEAEDAATYYCQQYHDYPPTFGGGTKLEIK
>M146 VH:
SEQ ID NO: 17
EFQLQQSGPELVKPGASVKISCKASGYSFTDYNMNWVKQSNGKSLEWIGLINPKYGT
TNYNQKFKGKATLTVDQSSSTAYMQLNSLTSEDSAVYYCAIDDYPAWFAYWGQGTLV
TVSA
>M146 VL:
SEQ ID NO: 18
QIVLTQSPAIMSASPGEKVTISCSASSSLNYMYWYQQKPGSSPKSWIYRTSNLASGVP
ARFSGSGSGTSYSLTISSMEAEDAATYYCQQYHSYPPTFGAGTKLELK
>M42VH:
SEQ ID NO: 273
QIQLKQSGPGLVQPSQSLSITCTVSGFSLTSYAVHWVRQSPGKGLEWLGVIWTGGNTR
SNAAFMSRLTITKDNSKSQVFFKMNSLQADDTAIYFCAKNSLLDYAMDYWGQGTSV
TVSS
>M42VL:
SEQ ID NO: 274
ENVLTQSPVIMSASPGEKVTMTCRASSFVGSGYLHWYQQKSGASPKLWIYSTSKLAS
GVPARFSGSGSGTSFFLTISRVEIEDAAAYYCQQSSGYPLTFGGGSKLEIK
>M61VH:
SEQ ID NO: 275
EVQLQQSGPELVRPGASVKISCTASGYTITDYYMSWVKQSHGKSPEWIGDINPNNGG
TSDNQKFKGKATLTVDKSSSTAYMEYRSLTSEDSAVYYCARGRGMVARYFDIWGTGT
TVTVSS
>M61VL:
SEQ ID NO: 276
EILLTQSPTTMAASPGEKITITCSASSSISSNFLHWYQEKPGFSPKLLIYRTSNLASGVPA
RFSGSGSGTSYSLTIGSMEAEDVATYYCQQGSGLPLTFGAGTKLELK
>M64VH:
SEQ ID NO: 277
EVQLVESGGGLVKPGGSLKLSCAASGFTFSDYGMHWVRQAPEKGLEWVAYISSGSST
IYYADTVKGRFTISRDNAKNTLFLQMTSLRSEDTAMYYCARTRVYFYGSYWYFDVW
GTGTTVTVSS
>M64VL:
SEQ ID NO: 278
QIVLTQSPAIMSASPGEKVTMTCSASSSVSYMYWYQQKSGTSPKRWIYDTSKLASGV
PARFSGSGSGTSYSLTISSLEAEDAATYYCQQWSSDPPYTFGGGTKLEIK
>M93VH:
SEQ ID NO: 279
QVQLQQPGAELVRPGTSVKLSCKASGYTFTRYWMHGVKRRPGQGLEWIGMIDPSDS
YTKYNQKFMGKAILTVDTSSSTAYMQFSSLTSEDSAVYYCARSIKVAGWYFDVWGT
GTTVTVSS
>M93VL:
SEQ ID NO: 280
DIVMTQSHKFMSTSVGDRVSITCKASQDVSSAVAWYQQKPGQSPKLLIYSASYRSTG
VPDRFTGSGSGTDFTFTISSVQAEDLAIYYCQQHFSTTLTFGAGTKLELK

Note: The single underlined parts are CDRs according to the Kabat scheme, and the rest are FRs.

Screening of Single B cells was mainly performed by using forward and reverse screening, 10*genomic, and bioinformatics techniques to obtain a series of antibody variable region sequences. The CDR classification was performed on the obtained sequences, and murine variable region sequences were selected to be connected to the human antibody constant region sequences to express chimeric antibodies, which were allowed to bind to hGUCY2C ECD-His and cynoGUCY2C ECD-His (ACRO #GUC-C52H6); such methods as surface plasmon resonance and cell binding were used for screening to select antibody sequences specifically binding to hGUCY2C ECD and cynoGUCY2C ECD. The sequences of the heavy chain variable regions and light chain variable regions of the antibodies resulting from screening were as follows:

TABLE 15
CDRs of anti-GUCY2C antibodies resulting from Single B screening

| Antibody | Heavy chain CDR | | Light chain CDR | |
|---|---|---|---|---|
| P3-C7 | HCDR1 | DHYIH | LCDR1 | RSSQSLVHSNGNTYLH |
| | | (SEQ ID NO: 19) | | (SEQ ID NO: 22) |
| | HCDR2 | VINPYNDGATYNQKFKG | LCDR2 | KVSNRFS |
| | | (SEQ ID NO: 20) | | (SEQ ID NO: 23) |
| | HCDR3 | PPRAYGNYAGY | LCDR3 | SQSTHVPYT |
| | | (SEQ ID NO: 21) | | (SEQ ID NO: 24) |
| P1-D2 | HCDR1 | DHNMN | LCDR1 | SANSSVNYIY |
| | | (SEQ ID NO: 281) | | (SEQ ID NO: 284) |
| | HCDR2 | VINPK YGTISNNQKFKG | LCDR2 | RTSNLAS |
| | | (SEQ ID NO: 282) | | (SEQ ID NO: 8) |
| | HCDR3 | DDYPNYFDY | LCDR3 | QQYHSFPPT |
| | | (SEQ ID NO: 283) | | (SEQ ID NO: 285) |
| P1-D3 | HCDR1 | TYGMS | LCDR1 | SASSSVNYMH |
| | | (SEQ ID NO: 286) | | (SEQ ID NO: 289) |
| | HCDR2 | WINTYSGVPTYADDFKG | LCDR2 | DTSKLAS |
| | | (SEQ ID NO: 287) | | (SEQ ID NO: 265) |
| | HCDR3 | RGYSNLYYYALDY | LCDR3 | QQWSSNPST |
| | | (SEQ ID NO: 288) | | (SEQ ID NO: 290) |
| P1-C6 | HCDR1 | SYGIT | LCDR1 | RASESVSLIGTNLIH |
| | | (SEQ ID NO: 291) | | (SEQ ID NO: 294) |
| | HCDR2 | EIYPRSGNTYYNEKFKG | LCDR2 | HASNLEA |
| | | (SEQ ID NO: 292) | | (SEQ ID NO: 295) |
| | HCDR3 | AITTVVGDWYFDV | LCDR3 | LQSRKIRT |
| | | (SEQ ID NO: 293) | | (SEQ ID NO: 296) |
| P1-D9 | HCDR1 | DYGMH | LCDR1 | SASSSVSYMY |
| | | (SEQ ID NO: 261) | | (SEQ ID NO: 264) |
| | HCDR2 | HISSGSSTIYYADTVKG | LCDR2 | LTSNLAS |
| | | (SEQ ID NO: 297) | | (SEQ ID NO: 299) |
| | HCDR3 | SRNYYGSRIFDY | LCDR3 | QQWSSNPPT |
| | | (SEQ ID NO: 298) | | (SEQ ID NO: 300) |
| P1-F10 | HCDR1 | DYGMH | LCDR1 | SASSSVNYMY |
| | | (SEQ ID NO: 261) | | (SEQ ID NO: 303) |
| | HCDR2 | HISSGSSTIYYADTVKG | LCDR2 | LTSNLAS |
| | | (SEQ ID NO: 301) | | (SEQ ID NO: 299) |
| | HCDR3 | SRNYYGSRVFDY | LCDR3 | QQWNSNPPT |
| | | (SEQ ID NO: 302) | | (SEQ ID NO: 304) |
| P2-C8 | HCDR1 | DYYMH | LCDR1 | RSSQSLVHSNGNTYLH |
| | | (SEQ ID NO: 305) | | (SEQ ID NO: 22) |
| | HCDR2 | LINPYNGVTSYNQKFKG | LCDR2 | KVSNRFS |
| | | (SEQ ID NO: 306) | | (SEQ ID NO: 23) |
| | HCDR3 | PPRAYGNYAGY | LCDR3 | SQSTHVPYT |
| | | (SEQ ID NO: 21) | | (SEQ ID NO: 24) |
| P2-B9 | HCDR1 | SYWMH | LCDR1 | KASKK VTIFGSISALH |
| | | (SEQ ID NO: 128) | | (SEQ ID NO: 309) |
| | HCDR2 | MIHPKSDSVNNNEKFKN | LCDR2 | NGAKLES |
| | | (SEQ ID NO: 307) | | (SEQ ID NO: 310) |
| | HCDR3 | GGDSGYSMDY | LCDR3 | LQNKEVPYT |
| | | (SEQ ID NO: 308) | | (SEQ ID NO: 311) |
| P3-D2 | HCDR1 | DYFMH | LCDR1 | RSSQSLVHSNGNTYLH |
| | | (SEQ ID NO: 312) | | (SEQ ID NO: 22) |
| | HCDR2 | LINPYSGGTTYNQKFKG | LCDR2 | KVSNRFS |
| | | (SEQ ID NO: 313) | | (SEQ ID NO: 23) |
| | HCDR3 | PPRAYGDYAGY | LCDR3 | SQSTHVPLT |
| | | (SEQ ID NO: 314) | | (SEQ ID NO: 315) |
| P3-B4 | HCDR1 | TYGMS | LCDR1 | SASSSVSYML |
| | | (SEQ ID NO: 286) | | (SEQ ID NO: 317) |
| | HCDR2 | WINTYSGVPTYADDFKG | LCDR2 | DTSKLAS |
| | | (SEQ ID NO: 287) | | (SEQ ID NO: 265) |
| | HCDR3 | RAGSSLYYYAMDY | LCDR3 | QQWSSNPPT |
| | | (SEQ ID NO: 316) | | (SEQ ID NO: 300) |
| P3-E4 | HCDR1 | NYWMH | LCDR1 | RASESVSIHGTHLMH |
| | | (SEQ ID NO: 318) | | (SEQ ID NO: 321) |
| | HCDR2 | EIDPSDGYTDYNQKFKG | LCDR2 | AASNLES |
| | | (SEQ ID NO: 319) | | (SEQ ID NO: 322) |
| | HCDR3 | SLRGPITFGPFPY | LCDR3 | QHSIEDPRT |
| | | (SEQ ID NO: 320) | | (SEQ ID NO: 323) |
| P3-F6 | HCDR1 | DYYMN | LCDR1 | RSSQSLVHSNGNTYLH |
| | | (SEQ ID NO: 324) | | (SEQ ID NO: 22) |
| | HCDR2 | DINPNNGGTSYNQKFKG | LCDR2 | KVSNRFS |
| | | (SEQ ID NO: 325) | | (SEQ ID NO: 23) |
| | HCDR3 | KTWLPYASVY | LCDR3 | SQSTHVPPYT |
| | | (SEQ ID NO: 326) | | (SEQ ID NO: 327) |
| P3-D9 | HCDR1 | DYGMH | LCDR1 | SASSSVSYMY |
| | | (SEQ ID NO: 261) | | (SEQ ID NO: 264) |
| | HCDR2 | HISSGSSTIYYGDTVKG | LCDR2 | LTSSLAS |
| | | (SEQ ID NO: 328) | | (SEQ ID NO: 329) |
| | HCDR3 | SRNYYGSRVFDY | LCDR3 | QQWSSNPPT |
| | | (SEQ ID NO: 302) | | (SEQ ID NO: 300) |

>P3-C7 VH:
SEQ ID NO: 25
EVQLQQSGPVLLKPGASVKMSCKASGYTFTDHYIHWVKLSHGKSLEWIGVINPYND
GATYNQKFKGKATLTVDKSSSTAYMELNSLTSEDSAVYYCARPPRAYGNYAGYWGQ
GTTLTVSS
>P3-C7 VL:
SEQ ID NO: 26
DVVMTQTPLSLPVSLGDQASISCRSSQSLVHSNGNTYLHWYLQKPGQSPKLLIYKVS
NRFSGVPDRFIGSGSRTDFTLKISRVEAEDLGVYFCSQSTHVPYTFGGGTRLEIK
>P1-D2 VH
SEQ ID NO: 330
EFQLQQSGPELVKPGASVKISCKASGYSFTDHNMNWVKQSNGKSLEWIGVINPKYGT
ISNNQKFKGKATLTVDQSSSTAYMQLNSLTAEDSAVYYCARDDYPNYFDYWGQGTT
LTVSS
>P1-D2 VL
SEQ ID NO: 331
QIVLTQSPAIMSASPGEKVTISCSANSSVNYIYWYQQKPGSSPKPWIYRTSNLASGVPA
RFSGSGSGTSYSLTISSMEAEDAATYYCQQYHSFPPTFGGGTKLEIK
>P1-D3 VH
SEQ ID NO: 332
QIQLIQSGPELKKPGETVKISCKASGYTFTTYGMSWVKQAPGKGLKWMGWINTYSG
VPTYADDFKGRFAFSLETSANTAYLQINNLKNEDTATYFCARRGYSNLYYYALDYWG
QGASVTVSS
>P1-D3 VL
SEQ ID NO: 333
QIVLTQSPAIMSASPGEKVTMTCSASSSVNYMHWYQQKSGTSPKRWIYDTSKLASGV
PTRFSGSGSGTSYSLTISSMEAEDAATYFCQQWSSNPSTFGGGTKLEIK
>P1-C6 VH
SEQ ID NO: 334
QVQLQQSGAELARPGASVKLSCKASGYTFTSYGITWVKRRTGQGLGWIGEIYPRSGN
TYYNEKFKGKATLTADKSSSTAYMELRSLTSEDSAVYFCARAITTVVGDWYFDVWGT
GTTVTVSS
>P1-C6 VL
SEQ ID NO: 335
DIVLTQSPASLAMSLGKRATISCRASESVSLIGTNLIHWYQQKPGQPPKLLIHHASNLE
AGVPARFSGSGSRTDFTLTIDPVEEDDVAIYYCLQSRKIRTFGGGTKLEIK
>P1-D9 VH
SEQ ID NO: 336
EVQLVESGGGLVKPGGSLKLSCAASGFSFSDYGMHWVRQAPEKGLEWIAHISSGSSTI
YYADTVKGRFTISRDNAKNTLFLQMTSLRSEDTAMYYCARSRNYYGSRIFDYWGQG
TTLTVSS
>P1-D9 VL
SEQ ID NO: 337
QIVLTQSPARMSASPGEKVTMTCSASSSVSYMYWYQQKPRSSPKPWIYLTSNLASGV
PARFSGSGSGTSYSLTISSMEAEDAATYYCQQWSSNPPTFGSGTKLEIK
>P1-F10 VH
SEQ ID NO: 338
EVQLVESGGGLVKPGGSLKLSCAASGFAFSDYGMHWVRQAPEKGLEWVAHISSGSS
TIYYADTVKGRFTISRDNVKNTLFLQMTSLRSEDTAMYYCARSRNYYGSRVFDYWG
QGTPLTVSS
>P1-F10 VL
SEQ ID NO: 339
QIVLTQSPALMSVSPGEKVTMTCSASSSVNYMYWYQQKPRSSPKPWIYLTSNLASGV
PARFSGSGSGTSYSLTISSMEAEDAATYYCQQWNSNPPTFGSGTKLEIK
>P2-C8 VH
SEQ ID NO: 340
EVQLQQSGPVLVKPGASVKMSCKVSGYTFTDYYMHWVKQSHGKSLEWIGLINPYN
GVTSYNQKFKGKATLTVDKSSSTAYMELNSLTSEDSAVYYCARPPRAYGNYAGYWG
QGTTLTVSS
>P2-C8 VL
SEQ ID NO: 341
DVVMTQTPLSLPVSLGDQASISCRSSQSLVHSNGNTYLHWYLQKPGQSPKLLIYKVS
NRFSGVPDRFSGSGSGTDFTLKISRVEAEDLGVYFCSQSTHVPYTFGGGTKLEIK
>P2-B9 VH
SEQ ID NO: 342
QVQLQQPGAELVKPGASVKLSCKASGYTFSSYWMHWVKQRPGQGLEWIGMIHPKS
DSVNNNEKFKNKATLTVDKSSSTVYLQLSSLTSEDSAVYSCARGGDSGYSMDYWGQ
GTSVTVSS
>P2-B9 VL
SEQ ID NO: 343
DIVLTQSPASLAVSLGQKATISCKASKKVTIFGSISALHWYQQKPGQPPKLIYNGAKLE
SGVSARFSDSGSQNRSPFGNQLSFTLTIDPVEADDAATYYCLQNKEVPYTFGGGTKLE
IK
>P3-D2 VH
SEQ ID NO: 344
EVQLQQSGPVLVKPGASVKMSCKASGYTFTDYFMHWVKQSHGKSLEWIGLINPYSG
GTTYNQKFKGKATLTVDKSSSTAYMELNSLTSEDSAVYYCARPPRAYGDYAGYWGQ
GTTLTVSS
>P3-D2 VL
SEQ ID NO: 345
DVVMTQTPLSLPVSLGDQASISCRSSQSLVHSNGNTYLHWYLQKPGQSPKLLIYKVS
NRFSGVPDRFSGSGSGTDFTLKISRVEAEDLGVYFCSQSTHVPLTFGTGTKLEIK
>P3-B4 VH
SEQ ID NO: 346
QIQLVQSGPELKKPGEIVKISCKASGYTFTTYGMSWVKQAPGKGLKWMGWINTYSG
VPTYADDFKGRFAFSLETSASTAYLQINNLKNEDTATYFCARRAGSSLYYYAMDYWG
QGTSVTVSS
>P3-B4 VL
SEQ ID NO: 347
QIVLTQSPAIMSASPGEKVTMTCSASSSVSYMLWYQQKSGTSPKRWIYDTSKLASGVP
ARFSGSGSGTSYSLTISSMEAEDAATYYCQQWSSNPPTFGGGTKLEIK
>P3-E4 VH
SEQ ID NO: 348
QVQLQQPGAELVMPGAAVKLSCKASGYTFINYWMHWLKQRPGQGLEWIGEIDPSD
GYTDYNQKFKGKATLTVDKSSSTAYMQLSSLTSEDSAVYYCAISLRGPITFGPFPYWG
QGTLVTVSS
>P3-E4 VL
SEQ ID NO: 349
DIVLTQSPASLAVSLGQRATISCRASESVSIHGTHLMHWYQQKPGQPPKLLIYAASNLE
SGVPARFSGSGSETDFTLNIHPVAEEDAATYFCQHSIEDPRTFGGGTKLEIK
>P3-F6 VH
SEQ ID NO: 350
EVQLQQSGPELVKPGASVKISCKASGYTFTDYYMNWVKQSHGKSLEWIGDINPNNG
GTSYNQKFKGKATLTVDKSSSTAYMELRSLTSEDSAVYYCARKTWLPYASVYWGQG
TSVTVSS
>P3-F6 VL
SEQ ID NO: 351
DVVMTQTPLSLPVSLGDQASISCRSSQSLVHSNGNTYLHWYLQKPGQSPKLLIYKVS
NRFSGVPDRFSGSGSGTDFTLKISRVEAEDLGVYFCSQSTHVPPYTFGGGTKLEIK
>P3-D9 VH
SEQ ID NO: 352
EVQLVESGGGLVKPGGSLKLSCAASGFAFSDYGMHWVRQAPEKGLEWVAHISSGSS
TIYYGDTVKGRFTISRDNAKNTLFLQMTSLRSEDTAMYYCTRSRNYYGSRVFDYWG
QGTTLTVSS
>P3-D9 VL
SEQ ID NO: 353
QIVLTQSPALMSVSPGEKVTMTCSASSSVSYMYWYQQKPRSSPKPWIYLTSSLASGVP
ARFSGSGSGTSYSLTINNVEAEDAATYYCQQWSSNPPTFGSGTKVEIK

Note:
The single underlined parts are CDRs according to the Kabat scheme, and the rest are FRs.

The variable region sequences of the murine anti-GUCY2C antibodies were combined with the constant regions set forth in SEQ ID NO: 58 and SEQ ID NO: 59 to obtain chimeric antibodies. Illustratively, M81CHI or P3-C7 CHI represents a chimeric antibody comprising M81 or P3-C7 murine heavy chain variable region and light chain variable region and the constant regions, and the specific sequences are shown below. This applies similarly to the remaining chimeric antibodies M146 CHI, M42 CHI, M61 CHI, M64 CHI, M93 CHI, P1-D2 CHI, P1-D3 CHI, P1-C6 CHI, P1-D9 CHI, P1-F10 CHI, P2-C8 CHI, P2-B9 CHI, P3-D2 CHI, P3-B4 CHI, P3-E4 CHI, P3-F6 CHI, and P3-D9 CHI; their full-length sequences may be unambiguously determined and therefore are not all listed for the sake of brevity.

Illustratively, the full-length sequences of the anti-GUCY2C chimeric antibodies are as follows:

M81CH1 heavy chain:
SEQ ID NO: 354
EFQLQQSGPELVKPGASVKISCKASGYSFTDHNMNWVKQSNGKSLEWIGVMNPKFG
TINYNQKFKDKATLTVDQSSSTAYMQLNSLTSEDSAVYYCVRDDYPNYFDYWGQGT
TLTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSR
WQQGNVFSCSVMHEALHNHYTQKSLSLSPGK
M81CH1 light chain:
SEQ ID NO: 355
QIVLTQSPAIMSASPGEKVTISCSASSSVNYMYWYQQKPGSSPKSWIYRTSNLASGVS
ARFSGSGSGTSYSLTISSMEAEDAATYYCQQYHDYPPTFGGGTKLEIKRTVAAPSVFIF
PPSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
P3-C7 CH1 heavy chain:
SEQ ID NO: 356
EVQLQQSGPVLLKPGASVKMSCKASGYTFTDHYIHWVKLSHGKSLEWIGVINPYND
GATYNQKFKGKATLTVDKSSSTAYMELNSLTSEDSAVYYCARPPRAYGNYAGYWGQ
GTTLTVSSASTKGPSVFPLAPSSKSTSGGTAALGCLVKDYFPEPVTVSWNSGALTSGVHTFP
AVLQSSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVDKS
RWQQGNVFSCSVMHEALHNHYTQKSLSLSPGK
P3-C7 CH1 light chain:
SEQ ID NO: 357
DVVMTQTPLSLPVSLGDQASISCRSSQSLVHSNGNTYLHWYLQKPGQSPKLLIYKVS
NRFSGVPDRFIGSGSRTDFTLKISRVEAEDLGVYFCSQSTHVPYTFGGGTRLEIKRTVA
APSVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC

### Example 3: Humanization of Anti-GUCY2C Monoclonal Antibodies

Humanization of the murine antibodies was performed according to the method described in many publications in the art. In brief, based on the typical structures of the VH/VL CDRs of the murine antibodies obtained, a human germline database was searched for homologous sequences of the light chain variable regions (VLs) and the heavy chain variable regions (VHs), the sequences were ranked in a descending manner according to the FR homology, and the germline with the highest FR homology was selected as the template; the CDRs of the murine antibodies were grafted onto a human template, and certain amino acids of the variable regions were mutated; the constant regions of the murine antibodies were replaced with a human constant region to obtain the final humanized antibodies.

### 3-1. Humanization of Antibody M81

For the humanized antibodies of the antibody M81, IGKV3-11*01, IGKV6-21*02 or IGKV1-9*01, and IGKJ4*01 were selected as the light chain framework region templates, and IGHV1-3*01 and IGHJ6*01 were selected as the heavy chain framework region templates. The CDR regions of the murine antibody were grafted onto the selected humanization templates, and certain amino acids in the variable regions were substituted to obtain the light chain variable regions and heavy chain variable regions of the humanized antibody; specific framework replacements and amino acid substitutions are as follows:

TABLE 16
Framework replacements and amino acid substitutions
for humanization of antibody M81

| VL | | VH | |
|---|---|---|---|
| L1 | Graft(IGKV3-11*01) + R45K, | H1 | Graft(IGHV1-3*01) + |
| | L46S, L47W, I58V, F71Y | | Q1E, R38K, R66K, |
| | | | V67A + D96N |
| L2 | Graft(IGKV6-21*02) + K16G, | H2 | Graft(IGHV1-3*01) + |
| | L46S, L47W, K49Y, N76S | | Q1E, R38K, R66K, |
| | | | V67A |
| L3 | Graft(IGKV1-9*01) + V15P, | | |
| | K42S, A43S, L46S, L47W, F71Y | | |
| L4 | Graft(IGKV1-9*01) + V15P, | | |
| | K42S, A43S, L46S, L47W, | | |
| | F71Y + L54D | | |
| L5 | Graft(IGKV1-9*01) + V15P, | | |
| | K42S, A43S, L46S, L47W, | | |
| | F71Y + S56Q | | |
| L6 | Graft(IGKV1-9*01) + V15P, | | |
| | K42S, A43S, L46S, L47W, | | |
| | F71Y + S52Q | | |
| FR4 | IGKJ4*01 | FR4 | IGHJ6*01 |

Note:
the numbering of amino acids is according to the Kabat numbering scheme.

The CDRs of the humanized antibodies of M81 are as follows:

TABLE 17
CDRs of humanized antibodies of M81

| Variable region | CDR | Sequence | SEQ ID NO |
|---|---|---|---|
| h81 H1 | HCDR3 | 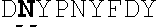 | 27 |
| h81 L4 | LCDR2 | 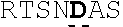 | 28 |
| h81 L5 | LCDR2 | 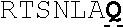 | 29 |
| h81 L6 | LCDR2 | 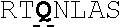 | 30 |

The variable region sequences of the humanized antibodies of M81 are as follows:

>h81 L1:
SEQ ID NO: 31

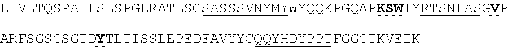

>h81 L2:
SEQ ID NO: 32

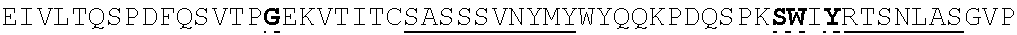

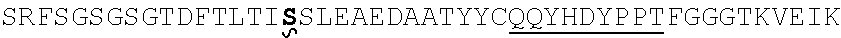

>h81 L3:
SEQ ID NO: 33

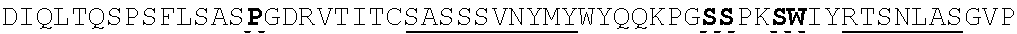

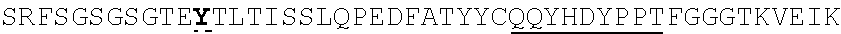

>h81 L4:
SEQ ID NO: 34

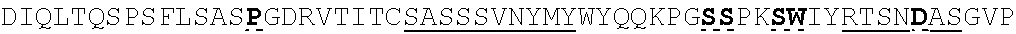

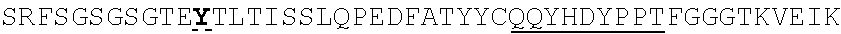

>h81 L5:
SEQ ID NO: 35

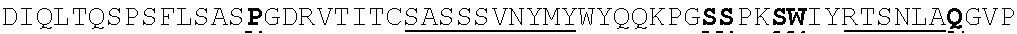

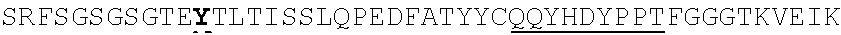

>h81 L6:
SEQ ID NO: 36

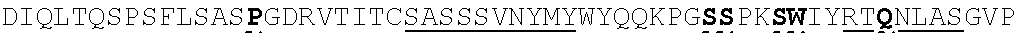

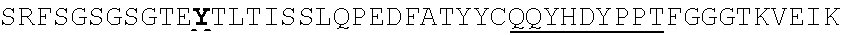

>h81 H1:
SEQ ID NO: 37

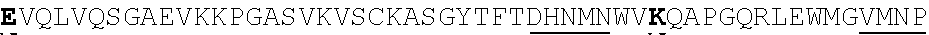

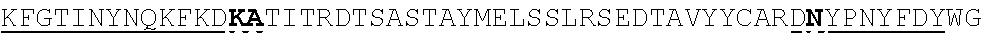

QGTTVTVSS
>h81 H2:
SEQ ID NO: 38

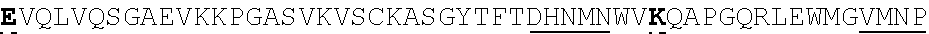

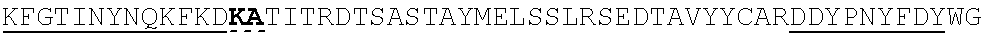

QGTTVTVSS

Note:
the single underlined parts are CDRs, the dotted underlined bold parts are the amino acid substitution sites, and the rest are FRs.

3-2. Humanization of antibody M146

For the humanized antibodies of the antibody M146, IGKV3-11*01 and IGKJ4*01 were selected as the light chain framework region templates, and IGHV1-3*01 and IGHJ6*01 were selected as the heavy chain framework region templates. The CDR regions of the murine antibody were grafted onto the selected humanization templates, and certain amino acids in the variable regions were substituted to obtain the light chain variable regions and heavy chain variable regions of the humanized antibody; specific framework replacements and amino acid substitutions are as follows:

TABLE 18
Framework replacements and amino acid substitutions
for humanization of antibody M146

| VL | | VH | |
|---|---|---|---|
| L1 | Graft(IGKV3-11*01) + Q42S, | H1 | Graft(IGHV1-3*01) + Q1E, |
| | A43S, L46P, L47W, I58V, | | V2F, T28S, I69L, R71V, |
| | F71Y + N53Q | | T73Q, A75S + D96N |
| FR4 | IGKJ4*01 | FR4 | IGHJ6*01 |

Note:
the numbering of amino acids is according to the Kabat numbering scheme.

The CDRs of the humanized antibodies of M146 were as follows:

TABLE 19
CDRs of humanized antibodies of M146

| Variable region | CDR | Sequence | SEQ ID NO |
|---|---|---|---|
| h146 L1 | LCDR2 | 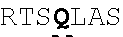 | 39 |
| h146 H1 | HCDR3 | 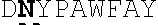 | 40 |

The variable region sequences of the humanized antibodies of M146 are as follows:

>h146 L1:
SEQ ID NO: 41

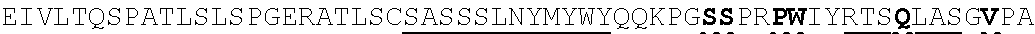

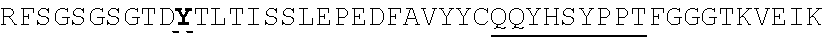

>h146 H1:
SEQ ID NO: 42

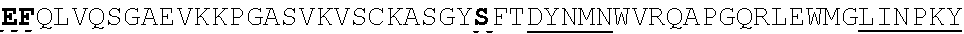

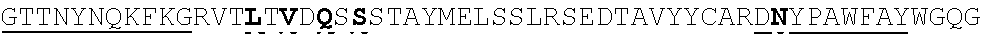

TTVTVSS

Note: the single underlined parts are CDRs, the dotted underlined bold parts are the amino acid substitution sites, and the rest are FRs. 3-3. Humanization of antibody P3-C7

For the humanized antibodies of the antibody P3-C7, IGKV240*01 or IGKV1G39*01 and IGKJ4*01 were selected as the light chain framework region templates, and IGHV1-46*01 and IGHJ6*01 were selected as the heavy chain framework region templates. The CDR regions of the murine antibody were grafted onto the selected humanization templates, and certain amino acids in the variable regions were substituted to obtain the light chain variable regions and heavy chain variable regions of the humanized antibody; specific framework replacements and amino acid substitutions are as follows:

TABLE 20
Framework replacements and amino acid substitutions
for humanization of antibody P3-C7

| VL | | VH | |
|---|---|---|---|
| L1 | Graft(IGKV2-40*01) | H1 | Graft(IGHV1-46*01) + |
| | | | Q1E, R71V, T73K |
| L2 | Graft(IGKV2-40*01) + | H2 | Graft(IGHV1-46*01) + |
| | I2V, G68R | | Q1E, Q39L, Q43K, |
| | | | M69L, R71V, T73K |
| L3 | Graft(IGKV2-40*01) + | | |
| | I2V, G68R + N28Q | | |
| L4 | Graft(IGKV2-40*01) + | | |
| | I2V, G68R + N28S | | |
| L5 | Graft(IGKV2-40*01) + | | |
| | I2V, G68R + N28T | | |
| L6 | Graft(IGKV2-40*01) + | | |
| | I2V, G68R + G29V | | |
| L7 | Graft(IGKV2-40*01) + | | |
| | I2V, G68R + G29Q | | |
| L8 | Graft(IGKV1-39*01) + | | |
| | I2V, Q3V, G68R, | | |
| | T74K + G29Q | | |
| FR4 | IGKJ4*01 | FR4 | IGHJ6*01 |

Note:
the numbering of amino acids is according to the Kabat numbering scheme.

The CDRs of the humanized antibodies of P3-C7 were as follows:

TABLE 21
CDRs of humanized antibodies of P3-C7

| Variable | | | SEQ |
|---|---|---|---|
| region | CDR | Sequence | ID NO |
| hP3-C7 L3 | LCDR1 | 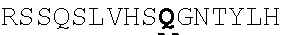 | 43 |
| hP3-C7 L4 | LCDR1 | 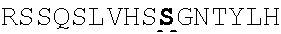 | 44 |
| hP3-C7 L5 | LCDR1 | 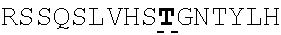 | 45 |
| hP3-C7 L6 | LCDR1 | 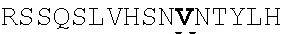 | 46 |
| hP3-C7 L7/ hP3-C7 L8 | LCDR1 | 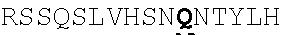 | 47 |

The variable region sequences of the humanized antibodies of P3-C7 are as follows:

>hP3-C7 L1:
SEQ ID NO: 48
DIVMTQTPLSLPVTPGEPASISCRSSQSLVHSNGNTYLHWYLQKPGQSPQLLIYKVSN
RFSGVPDRFSGSGSGTDFTLKISRVEAEDVGVYYCSQSTHVPYTFGGGTKVEIK
>hP3-C7 L2:
SEQ ID NO: 49

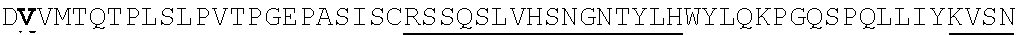

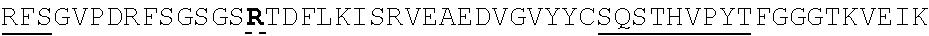

>hP3-C7 L3:
SEQ ID NO: 50

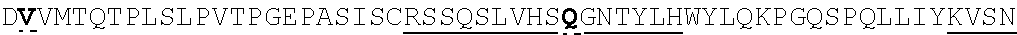

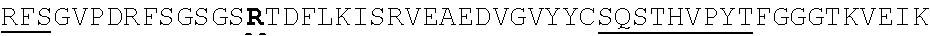

>hP3-C7 L4:
SEQ ID NO: 51

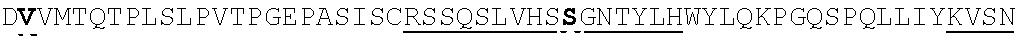

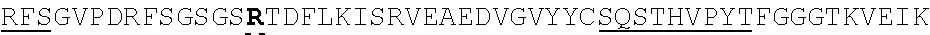

>hP3-C7 L5:
SEQ ID NO: 52

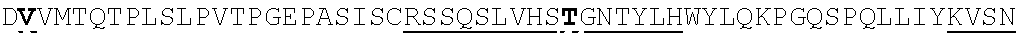

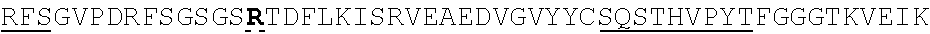

>hP3-C7 L6:
SEQ ID NO: 53

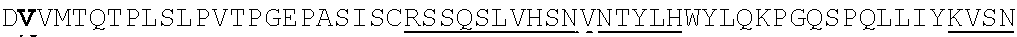

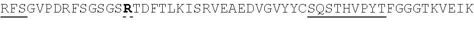

>hP3-C7 L7:
SEQ ID NO: 54

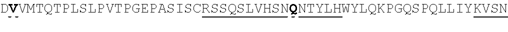

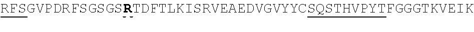

>hP3-C7 L8:
SEQ ID NO: 55

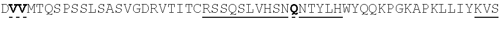

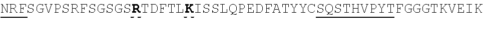

>hP3-C7 H1:
SEQ ID NO: 56

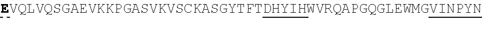

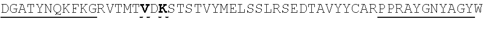

GQGTTVTVSS
>hP3-C7 H2:
SEQ ID NO: 57

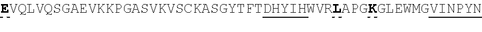

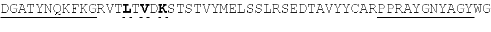

QGTTVTVSS

Note:
the single underlined parts are CDRs, the dotted underlined parts are the amino acid substitution sites, and the rest are FRs.

The heavy chain variable region and the light chain variable region of the anti-GUCY2C humanized antibody were recombined with the heavy chain constant region hIgG1:CH1-Fc and the light chain constant region CL, respectively, to obtain the full-length humanized antibody.

>hIgG1: CH1-Fc:
SEQ ID NO: 58
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSCDKTHTCPPCPAPE
LLGGPSVFLFPPKPKDTLMISRTPEVTCVVVDVSHEDPEVKFNWYVDGVEVHNAKT
KPREEQYNSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPPVLDSDGSF
FLYSKLTVDKSRWQQGNVFSCSVMHEALHNHYTQKSLSLSPGK
>CL:
SEQ ID NO: 59
RTVAAPSVFIFPPSDEQLKSGTASVVCLLNNFYPREAKVQWKVDNALQSGNSQESVT
EQDSKDSTYSLSSTLTLSKADYEKHKVYACEVTHQGLSSPVTKSFNRGEC

Anti-GUCY2C humanized antibodies obtained by the present disclosure were as follows:

TABLE 22-1
Humanized antibodies of the series 81

| Variable region | h81 L1 | h81 L2 | h81 L3 | h81 L4 | h81 L5 | h81 L6 |
|---|---|---|---|---|---|---|
| h81 H1 | 81 L1H1 | 81 L2H1 | 81 L3H1 | 81 L4H1 | 81 L5H1 | 81 L6H1 |
| h81 H2 | 81 L1H2 | 81 L2H2 | 81 L3H2 | 81 L4H2 | 81 L5H2 | 81 L6H2 |

TABLE 22-2
Humanized antibodies of the series 146

| Variable region | h146 L1 |
|---|---|
| h146 H1 | 146 L1H1 |

TABLE 22-3
Humanized antibodies of the series P3-C7

| Variable region | hP3-C7 L1 | hP3-C7 L2 | hP3-C7 L3 | hP3-C7 L4 | hP3-C7 L5 | hP3-C7 L6 | hP3-C7 L7 | hP3-C7 L8 |
|---|---|---|---|---|---|---|---|---|
| hP3-C7 | P3-C7 | P3-C7 | P3-C7 | P3-C7 | P3-C7 | P3-C7 | P3-C7 | P3-C7 |
| H1 | L1H1 | L2H1 | L3H1 | L4H1 | L5H1 | L6H1 | L7H1 | L8H1 |
| hP3-C7 | P3-C7 | P3-C7 | P3-C7 | P3-C7 | P3-C7 | P3-C7 | P3-C7 | P3-C7 |
| H2 | L1H2 | L2H2 | L3H2 | L4H2 | L5H2 | L6H2 | L7H2 | L8H2 |

Illustratively, the full-length sequences of the anti-GUCY2C humanized antibodies are as follows:

81 L3H1 heavy chain:
SEQ ID NO: 60

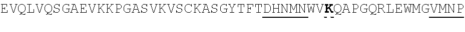

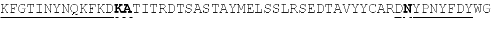

QGTTVTVSSASTKGPSVFPLAPSSKSTSGGTAALGCLVKDYFPEPVTVSWNSGALTSGVHT
FPAVLOSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTKP
REEQYNSTYRVVSVLTVLHODWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVD
KSRWQQGNVFSCSVMHEALHNHYTOKSLSLSPGK
81 L3H1 light chain:
SEQ ID NO: 61

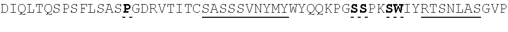

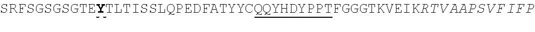

PSDEQLKSGTASVVCLLNNFYPREAKVQWKVDNALQSGNSQESVTEQDSKDSTYSLSSTLT
LSKADYEKHKVYACEVTHQGLSSPVTKSFNRGEC
146 L1H1 heavy chain:
SEQ ID NO: 62

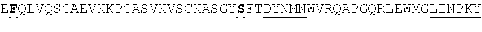

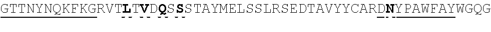

TTVTVSSASTKGPSVFPLAPSSKSTSGGTAALGCLVKDYFPEPVTVSWNSGALTSGVHTFPA
VLQSSGLYSLSSVVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSR
WQQGNVFSCSVMHEALHNHYTQKSLSLSPGK
146 L1H1 light chain:
SEQ ID NO: 63

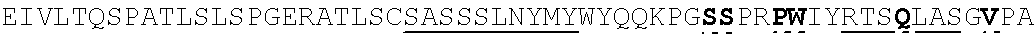

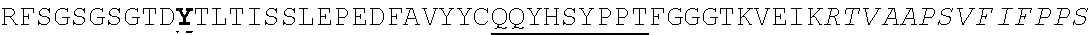

DEQLKSGTASVVCLLNNFYPREAKVQWKVDNALQSGNSQESVTEQDSKDSTYSLSSTLTLS
KADYEKHKVYACEVTHQGLSSPVTKSFNRGEC
P3-C7 L7H1 heavy chain:
SEQ ID NO: 64
EVQLVQSGAEVKKPGASVKVSCKASGYTFTDHYIHWVRQAPGQGLEWMGVINPYN

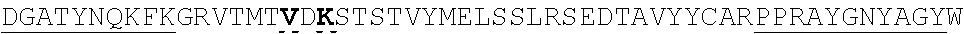

GQGTTVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHTCPPCP
APELLGGPSVFLFPPKPKDTLMISRTPEVTCVVVDVSHEDPEVKFNWYVDGVEVHNAKTK
PREEQYNSTYRVVSVLTVLHODWLNGKEYKCKVSNKALPAPIEKTISKAKGOPREPQVYTL
PPSRDELTKNQVSLTCLVKGFYPSDIAVEWESNGQPENNYKTTPPVLDSDGSFFLYSKLTV
DKSRWQQGNVFSCSVMHEALHNHYTQKSLSLSPGK
P3-C7 L7H1 light chain:
SEQ ID NO: 65

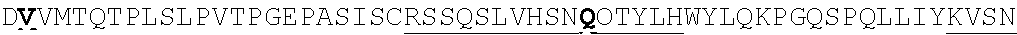

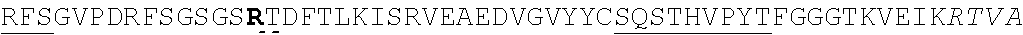

APSVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC
P3-C7 L8H1 heavy chain (same as P3-C7 L7H1 heavy chain, SEQ ID NO: 64)
P3-C7 L8H1 light chain:
SEQ ID NO: 66

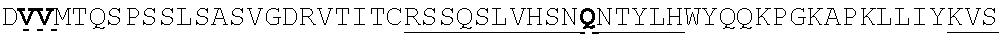

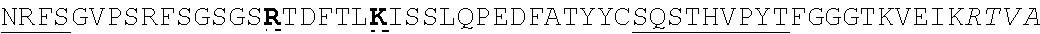

APSVFIFPPSDEQLKSGTASVVCLLNNFYPREAKVQWKVDNALQSGNSQESVTEQDSKDST
YSLSSTLTLSKADYEKHKVYACEVTHQGLSSPVTKSFNRGEC

Note:
the single underlined parts are CDRs, the dotted underlined bold parts are the amino acid substitution sites, and the italicized parts are constant regions.

The control antibodies of the anti-GUCY2C antibodies used in the present disclosure are as follows:
1608 (variable region sequences were constructed with reference to GUCY2c end of GUCY2c-1608 bispecific antibody in patent No. WO2019224716A2, and constant region sequences are set forth in SEQ ID NO: 58 and SEQ ID NO: 59), the full-length sequence of which is as follows:

>1608 heavy chain:
SEQ ID NO: 67
EVQLVESGGGLVQPGGSLRLSCAASGFTFSSYWMHWVRQAPGKGLEWIGEIKPSNEL
TNVHEKFKDRFTISVDKAKNSAYLQMNSLRAEDTAVYYCTRTITTTEGYWFFDVWG
QGTLVTVSSASTKGPSVFPLAPSSKSTSGGTAALGCLVKDYFPEPVTVSWNSGALTSGVHT
FPAVLQSSGLYSLSSVVTVPSSSLGTQTYICNVNHKPSNTKVDKKVEPKSCDKTHTCPPCPA
PELLGGPSVFLFPPKPKDTLMISRTPEVTCVVVDVSHEDPEVKFNWYVDGVEVHNAKTKP
REEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
>1608 light chain:
SEQ ID NO: 68
DIQLTQSPSSLSASVGDRVTITCRASESVDYYGSSLLQWYQQKPGKAPKLLIYAASKL
ASGVPSRFSGSGSGTDFTLTISSLQPEDFATYYCQQTRKAYTFGQGTKLEIKRTVAAPSV
FIFPPSDEQLKSGTASVVCLLNNFYPREAKVQWKVDNALQSGNSQESVTEQDSKDSTYSLS
STLTLSKADYEKHKVYACEVTHQGLSSPVTKSFNRGEC

Antibody 5F9 (variable region sequences were prepared with reference to patent No. WO2014134311A1, and constant region sequences are set forth in SEQ ID NO: 58 and SEQ ID NO: 59), the specific sequences of which are as follows:

>5F9 heavy chain:
SEQ ID NO: 69
QVQLQQWGAGLLKPSETLSLTCAVFGGSFSGYYWSWIRQPPGKGLEWIGEINHRGNT
NDNPSLKSRVTISVDTSKNQFALKLSSVTAADTAVYYCARERGYTYGNFDHWGQGT
LVTVSSASTKGPSVFPLAPSSKSTSGGTAALGCLVKDYFPEPVTVSWNSGALTSGVHTFPAV
LQSSGLYSLSSVVTVPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSR
DELTKNQVSLTCLVKGFYPSDIAVEWESNGQPENNYKTTPPVLDSDGSFFLYSKLTVDKSR
WQQGNVFSCSVMHEALHNHYTQKSLSLSPGK
>5F9 light chain:
EIVMTQSPATLSVSPGERATLSCRASQSVSRNLAWYQQKPGQAPRLLIYGASTRATGIP
ARFSGSGSGTEFTLTIGSLQSEDFAVYYCQQYKTWPRTFGQGTNVEIKRTVAAPSVFIFP
PSDEQLKSGTASVVCLLNNFYPREAKVQWKVDNALQSGNSQESVTEQDSKDSTYSLSSTLT
LSKADYEKHKVYACEVTHQGLSSPVTKSFNRGEC

### Example 4. Preparation of Anti-GUCY2C-CD3 Bispecific Antibodies

The GUCY2C arm of the GUCY2C-CD3 bispecific antibody of the present disclosure is the anti-GUCY2C antibody in the present disclosure, and the CD3 arm may be derived from any suitable antibody. Specifically, the CD3 arm used in the present disclosure is S107E or N103QN106Q.

TABLE 23
CDRs of S107E

| Antibody | Heavy chain | | Light chain | |
|---|---|---|---|---|
| S107E | HCDR1 | KYAMN | LCDR1 | GSSTGAVTSGNYPN |
| | | (SEQ ID NO: 71) | | (SEQ ID NO: 74) |
| | HCDR2 | RIRSK YNNYATYYADSVKD | LCDR2 | GTKFLAP |
| | | (SEQ ID NO: 72) | | (SEQ ID NO: 75) |
| | HCDR3 | HGNFGNEYISYWAY | LCDR3 | VLWYSNRWV |
| | | (SEQ ID NO: 73) | | (SEQ ID NO: 76) |

The variable region sequences of S107E are as follows:

>S107E-VH:
SEQ ID NO: 77
EVQLVESGGGLVQPGGSLKLSCAASGFTFNKYAMNWVRQAPGKGLEWVARIRSKYN
NYATYYADSVKDRFTISRDDSKNTAYLQMNNLKTEDTAVYYCVRHGNFGNEYISYW
AYWGQGTLVTVSS
>S107E-VL:
SEQ ID NO: 78
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVL

TABLE 24
CDRs of N103QN106Q

| Antibody | Heavy chain | | Light chain | |
|---|---|---|---|---|
| N103Q | HCDR1 | KYAMN | LCDR1 | GSSTGAVTSGNYPN |
| N106Q | | (SEQ ID NO: 71) | | (SEQ ID NO: 74) |
| | HCDR2 | RIRSKYNNYATYYADSVKD | LCDR2 | GTKFLAP |
| | | (SEQ ID NO: 72) | | (SEQ ID NO: 75) |
| | HCDR3 | HGQFGQSYISYWAY | LCDR3 | VLWYSNRWV |
| | | (SEQ ID NO: 88) | | (SEQ ID NO: 76) |

The variable region sequences of N103QN106Q are as follows:

>N103QN106Q-VH:
SEQ ID NO: 89
EVQLVESGGGLVQPGGSLKLSCAASGFTFNKYAMNWVRQAPGKGLEWVARIRSKYN
NYATYYADSVKDRFTISRDDSKNTAYLQMNNLKTEDTAVYYCVRHGQFGQSYISYW
AYWGQGTLVTVSS
>N103QN106Q-VL (same as S107E-VL, SEQ ID NO: 78)

The GUCY2C-CD3 bispecific antibody in the present disclosure has the following molecular structures of Format 1 to Format 5:
Format 1 is an asymmetrically structured molecule; the complete molecule comprises two chains, wherein the two chains are different and are specifically as follows:

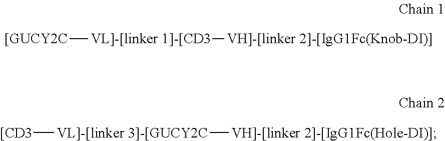

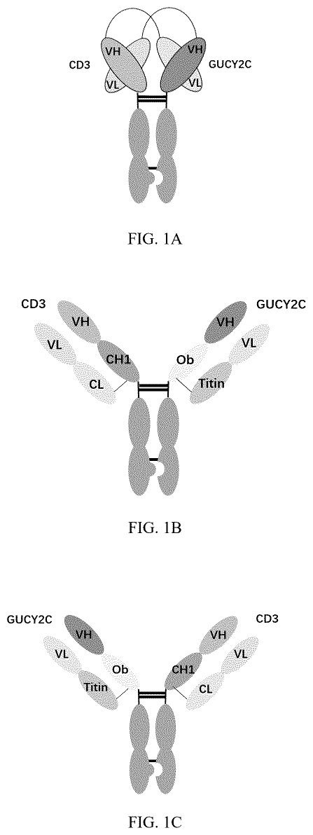

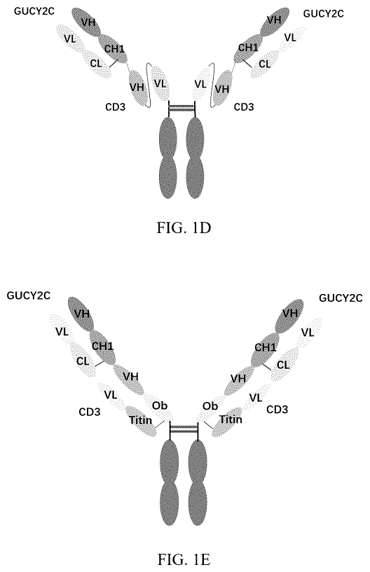

its schematic diagram is shown in FIG. 1A.

The related sequences are as follows:

>IgG1Fc (Knob-DI, L234A/L235A/G237A/Y349C/T366W):
SEQ ID NO: 79

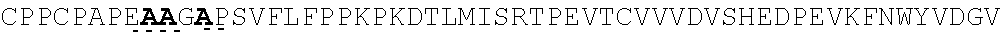

EVHNAKTKPREEQYNSTYRVVSVLTVLHQDWLNGKEYKCKVSNKALPAPIEKTISKA

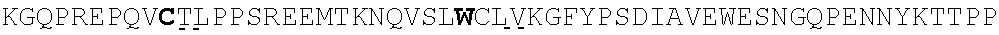

VLDSDGSFFLYSKLTVDKSRWQQGNVFSCSVMHEALHNHYTQKSLSLSPG
>IgG1Fc (Hole-DI, L234A/L235A/G237A/S354C/T366S/L368A/Y407V):
SEQ ID NO: 80

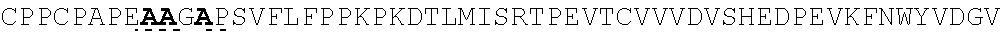

EVHNAKTKPREEQYNSTYRVVSVLTVLHQDWLNGKEYKCKVSNKALPAPIEKTISKA

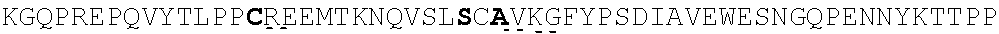

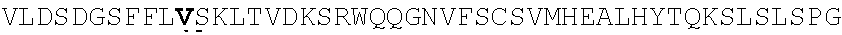

>Linker 1:
SEQ ID NO: 81
GGGSGGGG
>Linker 2:
SEQ ID NO: 82
G
>Linker 3:
SEQ ID NO: 83
GGGGSGGGG

Format 2 is an asymmetrically structured molecule; the complete molecule comprises four chains, wherein the four chains are different and are specifically as follows:

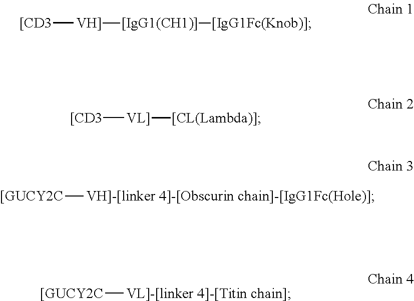

its schematic diagram is shown in FIG. 1B (where Ob stands for Obscurin).

The related sequences are as follows:

>IgG1(CH1):
SEQ ID NO: 84
ASTKGPSVFPLAPSSKSTSGGTAALGCLVKDYFPEPVTVSWNSGALTSGVHTFPAVLQ
SSGLYSLSSVVTVPSSSLGTQTYICNVNHKPSNTKVDKKVEPKSC
>IgG1Fc (Knob, L234A/L235A/S354C/T366W):
SEQ ID NO: 85

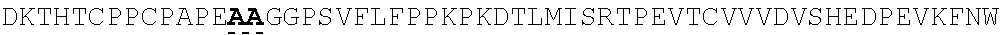

YVDGVEVHNAKTKPREEQYNSTYRVVSVLTVLHQDWLNGKEYKCKVSNKALPAPIE

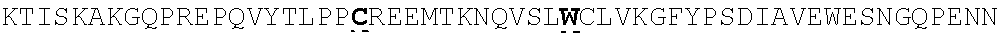

YKTTPPVLDSDGSFFLYSKLTVDKSRWQQGNVFSCSVMHEALHNHYTQKSLSLSPGK
>IgG1Fc (Hole, L234A/L235A/Y349C/T366S/L368A/Y407V):
SEQ ID NO: 86

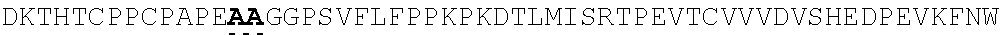

YVDGVEVHNAKTKPREEQYNSTYRVVSVLTVLHQDWLNGKEYKCKVSNKALPAPIE

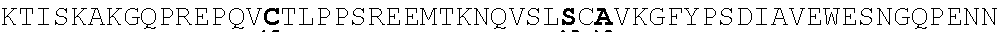

YKTTPPVLDSDGSFFLYSKLTVDKSRWQQGNVFSCSVMHEALHNHYTQKSLSLSPGK
>CL(Lambda):
SEQ ID NO: 87
GQPKANPTVTLFPPSSEELQANKATLVCLISDFYPGAVTVAWKADGSPVKAGVETTKP
SKQSNNKYAASSYLSLTPEQWKSHRSYSCQVTHEGSTVEKTVAPTEC
>Titin(T.16):
SEQ ID NO: 198

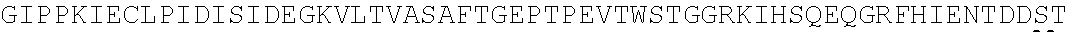

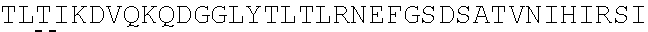

>Obscurin(O.28):
SEQ ID NO: 236

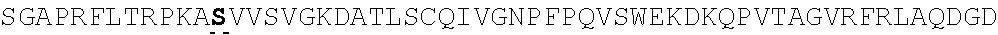

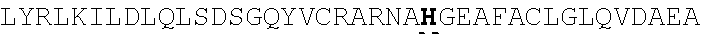

>Linker 4:
SEQ ID NO: 90
GGGGS

Format 3 is an asymmetrically structured molecule; the complete molecule comprises four chains, wherein the four chains are different and are specifically as follows:

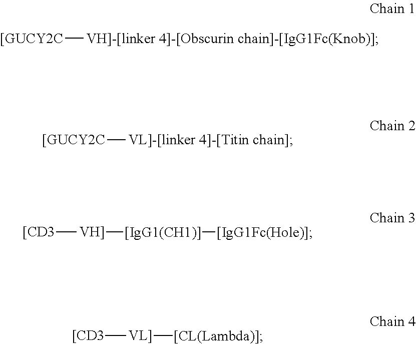

its schematic diagram is shown in FIG. 1C (where Ob stands for Obscurin).

The related sequences are as follows:

>IgG1(CH1)
(SEQ ID NO: 84)
>IgG1Fc(Knob)
(SEQ ID NO: 85)
>IgG1Fc(Hole)
(SEQ ID NO: 86)
>CL(Lambda)
(SEQ ID NO: 87)
>Titin(T.16)
(SEQ ID NO: 198)
>Obscurin(0.28)
(SEQ ID NO: 236)
>Linker 4
(SEQ ID NO: 90)
>chain 3 (Format 3-3, CD3-VH-IgGI(CH1)-IgG1Fc(Hole)):
SEQ ID NO: 93
EVQLVESGGGLVQPGGSLKLSCAASGFTFNKYAMNWVRQAPGKGLEWVARIRSKYN
NYATYYADSVKDRFTISRDDSKNTAYLQMNNLKTEDTAVYYCVRHGNFGNEYISYW
AYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIE
KTISKAKGQPREPQVCTLPPSREEMTKNQVSLSCAVKGFYPSDIAVEWESNGQPENN
YKTTPPVLDSDGSFFLVSKLTVDKSRWQQGNVFSCSVMHEALHNHYTQKSLSLSPGK
>chain 4 (Format 3-4, same as Format 2-2, SEQ ID NO: 92)

Format 4 is a symmetrically structured molecule; the complete molecule comprises four chains, wherein the four chains consist of two identical chains 1 and two identical chains 2 and are specifically as follows:

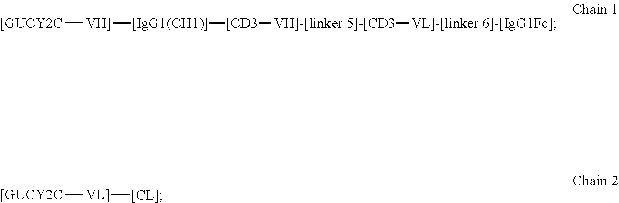

its schematic diagram is shown in FIG. 1D.

The related sequences are as follows:

>IgG1(CH1) (SEQ ID NO: 84)
>CL (SEQ ID NO: 59)
>IgG1Fc (L234A/L235A):
SEQ ID NO: 94

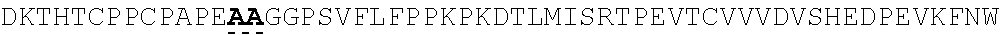

YVDGVEVHNAKTKPREEQYNSTYRVVSVLTVLHQDWLNGKEYKCKVSNKALPAPIE
KTISKAKGQPREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKSLSLSPGK
>Linker 5:
SEQ ID NO: 95
GGGGSGGGGSGGGGS
>Linker 6
SEQ ID NO: 96
GGG

Format 5 is a symmetrically structured molecule; the complete molecule comprises six chains, wherein the six chains consist of two identical chains 1, two identical chains 2, and two identical chains 3, and are specifically as follows:

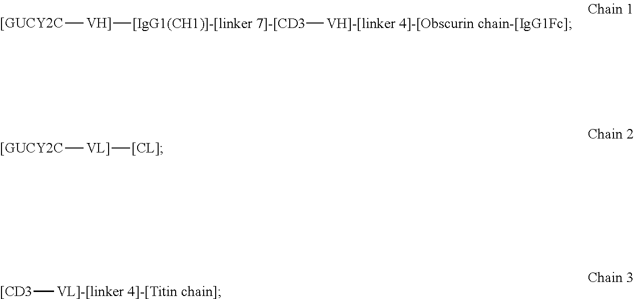

its schematic diagram is shown in FIG. 1E (where Ob stands for Obscurin).

The related sequences are as follows:

>IgG1(CH1)
(SEQ ID NO: 84)
>IgG1Fc
(SEQ ID NO: 86)
>CL
(SEQ ID NO: 59)
>Titin(T.16)
(SEQ ID NO: 198)
>Obscurin(0.28)
(SEQ ID NO: 236)
>Linker 4
(SEQ ID NO: 90)
>Linker 7:
SEQ ID NO: 97
GGGGSGGGGS
>Chain 3 (Format 5-3, CD3-VL-Linker 4-Titin)
SEQ ID NO: 98
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGGGG
SGIPPKIECLPIDISIDEGKVLTVASAFTGEPTPEVTWSTGGRKIHSQEQGRFHIENTDDS
TTLTIKDVQKQDGGLYTLTLRNEFGSDSATVNIHIRSI

Antigen-binding molecules were constructed according to Table 25, and the variable region of S107E or N103QN106Q was used as the CD3 arm of all bispecific antibody molecules. For the numbering of the anti-GUCY2C antibodies, the variable region is shown in the name of the antibody, the number followed indicates the Format used, the note (N103QN106Q) behind the Format indicates that the CD3 arm used by the bispecific antibody 15 N103QN106Q, and the absence of note behind the Format indicates that the CD3 arm used by the bispecific antibody is S107E. For example, P3-C7 L7HT-1 represents that the molecule uses the variable region of antibody P3-C7 L7H1 as the GUCY2C binding domain, Format 1 as the molecular structure, and S1007E as the CD3 arm. This applies similarly to other bispecific antibodies.

TABLE 25
GUCY2C-CD3 bispecific antibodies of the present disclosure

| No. | Chain 1 | Chain 2 | Chain 3 | Chain 4 |
|---|---|---|---|---|
| P3-C7 L7H1-1 | P3-C7 L7H1-1-1 | P3-C7 L7H1-1-2 | | |
| P3-C7 L8H1-1 | P3-C7 L8H1-1-1 | P3-C7 L8H1-1-2 | | |
| P3-C7 L7H1-2 | Format 2-1 | Format 2-2 | P3-C7 L7H1-2-3 | P3-C7 L7H1-2-4 |
| P3-C7 L7H1-3 | P3-C7 L7H1-3-1 | P3-C7 L7H1-3-2 | Format 3-3 | Format 3-4 |
| P3-C7 L7H1-4 | P3-C7 L7H1-4-1 | P3-C7 L7H1-4-2 | | |
| P3-C7 L8H1-4 | P3-C7 L8H1-4-1 | P3-C7 L8H1-4-2 | | |
| P3-C7 L7H1-5 | P3-C7 L7H1-5-1 | P3-C7 L7H1-5-2 | P3-C7 L7H1-5-3 | |
| P3-C7 L8H1-5 | P3-C7 L8H1-5-1 | P3-C7 L8H1-5-2 | P3-C7 L8H1-5-3 | |
| M81-1 | M81-1-1 | M81-1-2 | | |
| M81-2 | Format 2-1 | Format 2-2 | M81-2-3 | M81-2-4 |
| 81 L1H1-2 | Format 2-1 | Format 2-2 | 81 L1H1-2-3 | 81 L1H1-2-4 |
| 81 L2H1-2 | Format 2-1 | Format 2-2 | 81 L2H1-2-3 | 81 L2H1-2-4 |
| 81 L3H1-2 | Format 2-1 | Format 2-2 | 81 L3H1-2-3 | 81 L3H1-2-4 |
| 81 L3H1-2 | Format 2-1 | Format 2-2 | 81 L3H1-2-3 | 81 L3H1-2-4 |
| (N103QN106Q) | (N103QN106Q) | | | |
| 81 L4H1-2 | Format 2-1 | Format 2-2 | 81 L4H1-2-3 | 81 L4H1-2-4 |
| 81 L5H1-2 | Format 2-1 | Format 2-2 | 81 L5H1-2-3 | 81 L5H1-2-4 |
| 81 L6H1-2 | Format 2-1 | Format 2-2 | 81 L6H1-2-3 | 81 L6H1-2-4 |
| 81 L1H1-3 | 81 L1H1-3-1 | 81 L1H1-3-2 | Format 3-3 | Format 3-4 |
| 81 L2H1-3 | 81 L2H1-3-1 | 81 L2H1-3-2 | Format 3-3 | Format 3-4 |
| 81 L3H1-3 | 81 L3H1-3-1 | 81 L3H1-3-2 | Format 3-3 | Format 3-4 |
| 81 L4H1-3 | 81 L4H1-3-1 | 81 L4H1-3-2 | Format 3-3 | Format 3-4 |
| 81 L5H1-3 | 81 L5H1-3-1 | 81 L5H1-3-2 | Format 3-3 | Format 3-4 |
| 81 L6H1-3 | 81 L6H1-3-1 | 81 L6H1-3-2 | Format 3-3 | Format 3-4 |
| M146-1 | M146-1-1 | M146-1-2 | | |
| M146-2 | Format 2-1 | Format 2-2 | M146-2-3 | M146-2-4 |
| 146 L1H1-2 | Format 2-1 | Format 2-2 | 146 L1H1-2-3 | 146 L1H1-2-4 |
| 146 L1H1-3 | 146 L1H1-3-1 | 146 L1H1-3-2 | Format 3-3 | Format 3-4 |

Illustratively, the specific amino acid sequences of bispecific antibodies constructed with Format 1 in the present disclosure are as follows:

1. Full-length sequence of P3-C7 L7H1-1
>Chain 1 (P3-C7 L7H1-1-1):
DVVMTQTPLSLPVTPGEPASISCRSSQSLVHSNQNTYLHWYLQKPGQSPQLLIYKVSN
RFSGVPDRFSGSGSRTDFTLKISRVEAEDVGVYYCSQSTHVPYTFGGGTKVEIKGGGS
GGGGEVOLVESGGGLVQPGGSLKLSCAASGFTFNKYAMNWVRQAPGKGLEWVARI
RSKYNNYATYYADSVKDRFTISRDDSKNTAYLQMNNLKTEDTAVYYCVRHGNFGNE
YISYWAYWGQGTLVTVSSGCPPCPAPEAAGAPSVFLFPPKPKDTLMISRTPEVTCVVV
DVSHEDPEVKFNWYVDGVEVHNAKTKPREEQYNSTYRVVSVLTVLHQDWLNGKEY
KCKVSNKALPAPIEKTISKAKGQPREPQVCTLPPSREEMTKNQVSLWCLVKGFYPSDI
AVEWESNGQPENNYKTTPPVLDSDGSFFLYSKLTVDKSRWQQGNVFSCSVMHEALH
NHYTQKSLSLSPG
SEQ ID NO: 99
>Chain 2 (P3-C7 L7H1-1-2):
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGGGG
SGGGGEVQLVQSGAEVKKPGASVKVSCKASGYTFTDHYIHWVRQAPGQGLEWMG
VINPYNDGATYNQKFKGRVTMTVDKSTSTVYMELSSLRSEDTAVYYCARPPRAYGN
YAGYWGQGTTVTVSSGCPPCPAPEAAGAPSVFLFPPKPKDTLMISRTPEVTCVVVDV
SHEDPEVKFNWYVDGVEVHNAKTKPREEQYNSTYRVVSVLTVLHQDWLNGKEYKC
KVSNKALPAPIEKTISKAKGQPREPQVYTLPPCREEMTKNQVSLSCAVKGFYPSDIAV
EWESNGQPENNYKTTPPVLDSDGSFFLVSKLTVDKSRWQQGNVFSCSVMHEALHNH
YTQKSLSLSPG
SEQ ID NO: 100
2. Full-length sequence of P3-C7 L8H1-1
>Chain 1 (P3-C7 L8H1-1-1):
DVVMTQSPSSLSASVGDRVTITCRSSQSLVHSNQNTYLHWYQQKPGKAPKLLIYKVS
NRFSGVPSRFSGSGSRTDFTLKISSLQPEDFATYYCSQSTHVPYTFGGGTKVEIKGGGS
GGGGEVQLVESGGGLVQPGGSLKLSCAASGFTFNKYAMNWVRQAPGKGLEWVARI
RSKYNNYATYYADSVKDRFTISRDDSKNTAYLQMNNLKTEDTAVYYCVRHGNFGNE
YISYWAYWGQGTLVTVSSGCPPCPAPEAAGAPSVFLFPPKPKDTLMISRTPEVTCVVV
DVSHEDPEVKFNWYVDGVEVHNAKTKPREEQYNSTYRVVSVLTVLHQDWLNGKEY
KCKVSNKALPAPIEKTISKAKGQPREPQVCTLPPSREEMTKNQVSLWCLVKGFYPSDI
AVEWESNGQPENNYKTTPPVLDSDGSFFLYSKLTVDKSRWQQGNVFSCSVMHEALH
NHYTQKSLSLSPG
SEQ ID NO: 101
>Chain 2 (P3-C7 L8H1-1-2, same as P3-C7 L7H1-1-2, SEQ ID NO: 100)
3. Full-length sequence of M81-1
>Chain 1 (M81-1-1):
QIVLTQSPAIMSASPGEKVTISCSASSSVNYMYWYQQKPGSSPKSWIYRTSNLASGVS
ARFSGSGSGTSYSLTISSMEAEDAATYYCQQYHDYPPTFGGGTKLEIKGGGSGGGGE
VQLVESGGGLVQPGGSLKLSCAASGFTFNKYAMNWVRQAPGKGLEWVARIRSKYN
NYATYYADSVKDRFTISRDDSKNTAYLQMNNLKTEDTAVYYCVRHGNFGNEYISYW
AYWGQGTLVTVSSGCPPCPAPEAAGAPSVFLFPPKPKDTLMISRTPEVTCVVVDVSHE
DPEVKFNWYVDGVEVHNAKTKPREEQYNSTYRVVSVLTVLHQDWLNGKEYKCKVS
NKALPAPIEKTISKAKGQPREPQVCTLPPSREEMTKNQVSLWCLVKGFYPSDIAVEWE
SNGQPENNYKTTPPVLDSDGSFFLYSKLTVDKSRWQQGNVFSCSVMHEALHNHYTQ
KSLSLSPG
SEQ ID NO: 102
>Chain 2 (M81-1-2):
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGGGG
SGGGGEFQLQQSGPELVKPGASVKISCKASGYSFTDHNMNWVKQSNGKSLEWIGVM
NPKFGTINYNQKFKDKATLTVDQSSSTAYMOLNSLTSEDSAVYYCVRDDYPNYEDY
WGQGTTLTVSSGCPPCPAPEAAGAPSVFLFPPKPKDTLMISRTPEVTCVVVDVSHEDP
EVKFNWYVDGVEVHNAKTKPREEQYNSTYRVVSVLTVLHQDWLNGKEYKCKVSN
KALPAPIEKTISKAKGQPREPQVYTLPPCREEMTKNQVSLSCAVKGFYPSDIAVEWES
NGQPENNYKTTPPVLDSDGSFFLVSKLTVDKSRWQQGNVFSCSVMHEALHNHYTQK
SLSLSPG
SEQ ID NO: 103
4. Full-Length sequence of M146-1
>Chain 1 (M146-1-1):
QIVLTQSPAIMSASPGEKVTISCSASSSLNYMYWYQQKPGSSPKSWIYRTSNLASGVP
ARFSGSGSGTSYSLTISSMEAEDAATYYCQQYHSYPPTFGAGTKLELKGGGSGGGGE
VQLVESGGGLVQPGGSLKLSCAASGFTFNKYAMNWVRQAPGKGLEWVARIRSKYN
NYATYYADSVKDRFTISRDDSKNTAYLQMNNLKTEDTAVYYCVRHGNFGNEYISYW
AYWGQGTLVTVSSGCPPCPAPEAAGAPSVFLFPPKPKDTLMISRTPEVTCVVVDVSHE
DPEVKFNWYVDGVEVHNAKTKPREEQYNSTYRVVSVLTVLHQDWLNGKEYKCKVS
NKALPAPIEKTISKAKGQPREPQVCTLPPSREEMTKNQVSLWCLVKGFYPSDIAVEWE
SNGQPENNYKTTPPVLDSDGSFFLYSKLTVDKSRWQQGNVFSCSVMHEALHNHYTQ
KSLSLSPG
SEQ ID NO: 104
>Chain 2 (M146-1-2):
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGGGG
SGGGGEFQLQQSGPELVKPGASVKISCKASGYSFTDYNMNWVKQSNGKSLEWIGLIN
PKYGTTNYNQKFKGKATLTVDQSSSTAYMQLNSLTSEDSAVYYCAIDDYPAWFAYW
GQGTLVTVSAGCPPCPAPEAAGAPSVFLFPPKPKDTLMISRTPEVTCVVVDVSHEDPE
VKFNWYVDGVEVHNAKTKPREEQYNSTYRVVSVLTVLHQDWLNGKEYKCKVSNK
ALPAPIEKTISKAKGQPREPQVYTLPPCREEMTKNQVSLSCAVKGFYPSDIAVEWESN
GQPENNYKTTPPVLDSDGSFFLVSKLTVDKSRWQQGNVFSCSVMHEALHNHYTQKS
LSLSPG
SEQ ID NO: 105

Illustratively, the specific amino acid sequences of bispecific antibodies constructed with Format 2 in the present disclosure are as follows:

1. Full-length sequence of P3-C7 L7H1-2
>Chain 1 (Format 2-1):
SEQ ID NO: 91
EVQLVESGGGLVQPGGSLKLSCAASGFTFNKYAMNWVRQAPGKGLEWVARIRSKYN
NYATYYADSVKDRFTISRDDSKNTAYLQMNNLKTEDTAVYYCVRHGNFGNEYISYW
AYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIE
KTISKAKGQPREPQVYTLPPCREEMTKNQVSLWCLVKGFYPSDIAVEWESNGQPENN
YKTTPPVLDSDGSFFLYSKLTVDKSRWQQGNVFSCSVMHEALHNHYTQKSLSLSPGK
>Chain 2 (Format 2-2):
SEQ ID NO: 92
QTVVTQEPSLTVSPGGTVTLTCGSSTGAVTSGNYPNWVQQKPGQAPRGLIGGTKFLA
PGTPARFSGSLLGGKAALTLSGVQPEDEAEYYCVLWYSNRWVFGGGTKLTVLGQPK
ANPTVTLFPPSSEELQANKATLVCLISDFYPGAVTVAWKADGSPVKAGVETTKPSKQS
NNKYAASSYLSLTPEQWKSHRSYSCQVTHEGSTVEKTVAPTEC
>Chain 3 (P3-C7 L7H1-2-3):
SEQ ID NO: 106
EVQLVQSGAEVKKPGASVKVSCKASGYTFTDHYIHWVRQAPGQGLEWMGVINPYN

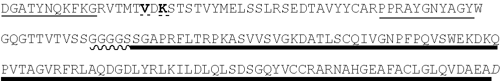

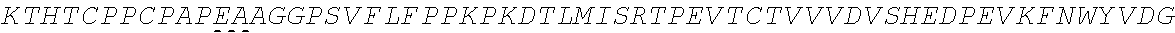

VEVHNAKTKPREEQYNSTYRVVSVLTVLHODWLNGKEYKCKVSNKALPAPIEKTISKAKGO

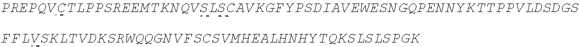

>Chain 4 (P3-C7 L7H1-2-4):
SEQ ID NO: 107

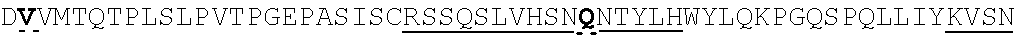

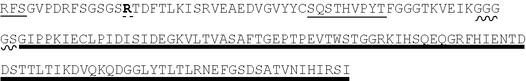

2. Full-length sequence of M81-2
>Chain 1 (Format 2-1, SEQ ID NO: 91)
>Chain 2 (Format 2-2, SEQ ID NO: 92)
>Chain 3 (M81-2-3):
SEQ ID NO: 108
EFQLQQSGPELVKPGASVKISCKASGYSFTDHNMNWVKQSNGKSLEWIGVMNPKFG
TINYNQKFKDKATLTVDQSSSTAYMQLNSLTSEDSAVYYCVRDDYPNYFDYWGQGT

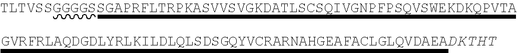

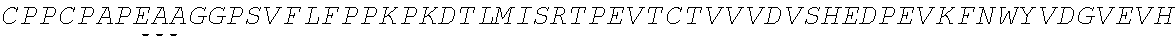

NAKTKPREEQYNSTYRVVSVLTVLHQDWLNGKEYKCKVSNKALPAPIEKTISKAKGOPREP

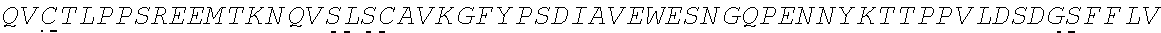

SKLTVDKSRWQQGNVFSCSVMHEALHNHYTQKSLSLSPGK
>Chain 4 (M81-2-4):
SEQ ID NO: 109
QIVLTQSPAIMSASPGEKVTISCSASSSVNYMYWYQQKPGSSPKSWIYRTSNLASGVS

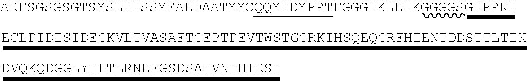

3. Full-length sequence of 81 L1H1-2
>Chain 1 (Format 2-1, SEQ ID NO: 91)
>Chain 2 (Format 2-2, SEQ ID NO: 92)
>Chain 3 (81 L1H1-2-3):
SEQ ID NO: 110

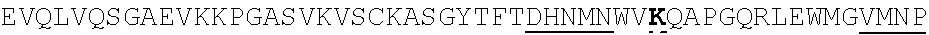

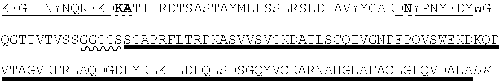

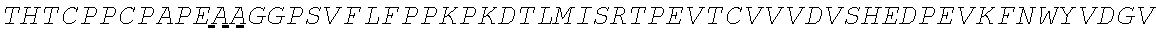

EVHNAKTKPREEQYNSTYRVVSVLTVLHQDWLNGKEYKCKVSNKALPAPIEKTISKAKGOP

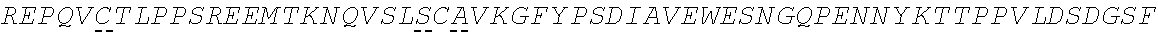

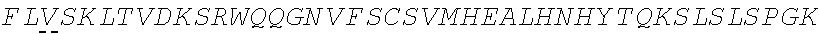

>Chain 4 (81 L1H1-2-4):
SEQ ID NO: 111

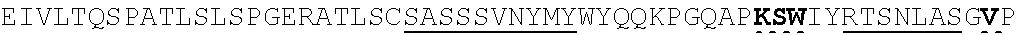

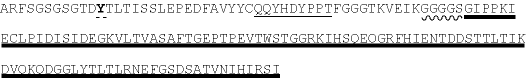

4. Full-length sequence of 81 L2H1-2
>Chain 1 (Format 2-1, SEQ ID NO: 91)
>Chain 2 (Format 2-2, SEQ ID NO: 92)
>Chain 3 (81 L2H1-2-3, same as 81 L1H1-2-3, SEQ ID NO: 110)
>Chain 4 (81 L2H1-2-4):
SEQ ID NO: 112

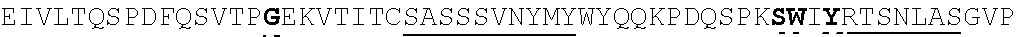

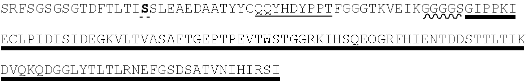

5. Full-length sequence of 81 L3H1-2
>Chain 1 (Format 2-1, SEQ ID NO: 91)
>Chain 2 (Format 2-2, SEQ ID NO: 92)
>Chain 3 (81 L3H1-2-3, same as 81 L1H1-2-3, SEQ ID NO: 110)
>Chain 4 (81 L3H1-2-4):
SEQ ID NO: 113

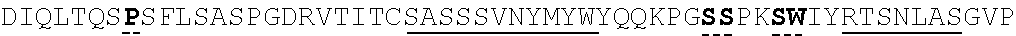

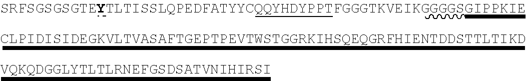

6. Full-length sequence of 81 L3H1-2 (N103QN106Q)
>Chain 1 (Format 2-1 (N103QN106Q))
SEQ ID NO: 242
EVQLVESGGGLVQPGGSLKLSCAASGFTFNKYAMNWVRQAPGKGLEWVARIRSKYN
NYATYYADSVKDRFTISRDDSKNTAYLQMNNLKTEDTAVYYCVRHGQFGQSYISYW
AYWGQGTLVTVSSASTKGPSVFPLAPSSKSTSGGTAALGCLVKDYFPEPVTVSWNSG
ALTSGVHTFPAVLQSSGLYSLSSVVTVPSSSLGTQTYICNVNHKPSNTKVDKKVEPKS
CDKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIE
KTISKAKGQPREPQVYTLPPCREEMTKNQVSLWCLVKGFYPSDIAVEWESNGQPENN
YKTTPPVLDSDGSFFLYSKLTVDKSRWQQGNVFSCSVMHEALHNHYTQKSLSLSPGK
>Chain 2 (Format 2-2, SEQ ID NO: 92)
>Chain 3 (81 L3H1-2-3, same as 81 L1H1-2-3, SEQ ID NO: 110)
>Chain 4 (81 L3H1-2-4, SEQ ID NO: 113):
6. Full-length sequence of 81 L4H1-2
>Chain 1 (Format 2-1, SEQ ID NO: 91)
>Chain 2 (Format 2-2, SEQ ID NO: 92)
>Chain 3 (81 L4H1-2-3, same as 81 L1H1-2-3, SEQ ID NO: 110)
>Chain 4 (81 L4H1-2-4):
SEQ ID NO: 114

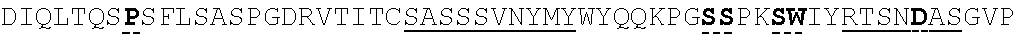

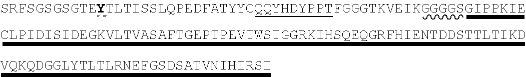

7. Full-length sequence of 81 L5H1-2
>Chain 1 (Format 2-1, SEQ ID NO: 91)
>Chain 2 (Format 2-2, SEQ ID NO: 92)
>Chain 3 (81 L5H1-2-3, same as 81 L1H1-2-3, SEQ ID NO: 110)
>Chain 4 (81 L5H1-2-4):
SEQ ID NO: 115

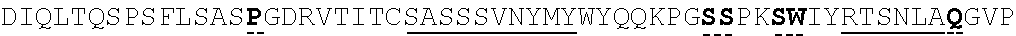

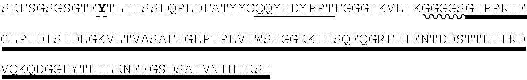

8. Full-length sequence of 81 L6H1-2
>Chain 1 (Format 2-1, SEQ ID NO: 91)
>Chain 2 (Format 2-2, SEQ ID NO: 92)
>Chain 3 (81 L6H1-2-3, same as 81 L1H1-2-3, SEQ ID NO: 110)
>Chain 4 (81 L6H1-2-4)
SEQ ID NO: 116

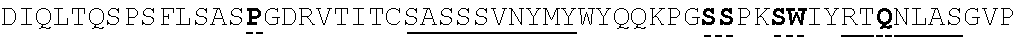

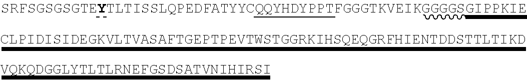

9. Full-Length sequence of M146-2
>Chain 1 (Format 2-1, SEQ ID NO: 91)
>Chain 2 (Format 2-2, SEQ ID NO: 92)
>Chain 3 (M146-2-3):
SEQ ID NO: 117
EFQLQQSGPELVKPGASVKISCKASGYSFTDYNMNWVKQSNGKSLEWIGLINPKYGT
TNYNQKFKGKATLTVDQSSSTAYMQLNSLTSEDSAVYYCAIDDYPAWFAYWGQGTLV
TVSAGGGGSSGAPRFLTRPKASVVSVGKDATLSCQIVGNPFPQVSWEKDKQPVTAGV
RFRLAQDGDLYRLKILDLOLSDSGQYVCRARNAHGEAFACLGLQVDAEADKTHTCP

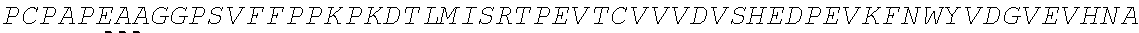

KTKPREEQYNSTYRVVSVLTVLHQDWLNGKEYKCKVSNKALPAPIEKTISKAKGQPREPQV

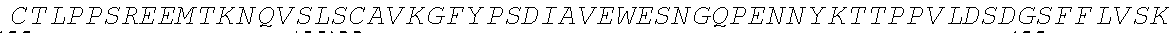

LTVDKSRWQQGNVFSCSVMHEALHNHYTQKSLSLSPGK
>Chain 4 (M146-2-4):
SEQ ID NO: 118
QIVLTQSPAIMSASPGEKVTISCSASSSLNYMYWYQQKPGSSPKSWIYRTSNLASGVP

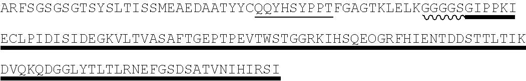

10. Full-length sequence of 146 L1H1-2
>Chain 1 (Format 2-1, SEQ ID NO: 91)
>Chain 2 (Format 2-2, SEQ ID NO: 92)
>Chain 3 (146 L1H1-2-3):
SEQ ID NO: 119

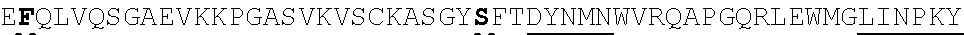

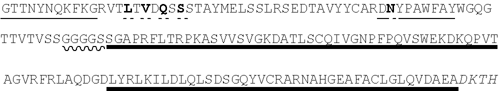

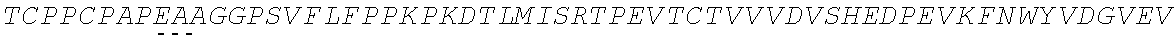

HNAKTKPREEQYNSTYRVVSVLTVLHODWLNGKEYKCKVSNKALPAPIEKTISKAKGOPRE

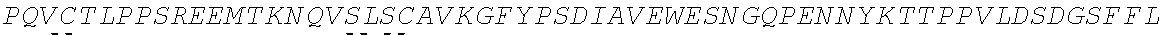

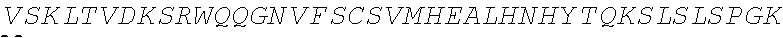

>Chain 4 (146 L1H1-2-4):
SEQ ID NO: 120

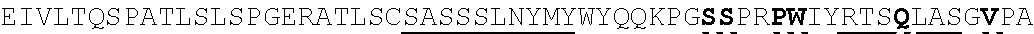

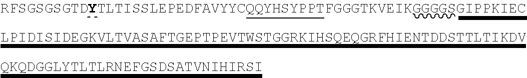

Illustratively, the specific amino acid sequences of bispecific antibodies constructed with Format 3 in the present disclosure are as follows:

1. Full-length sequence of P3-C7 L7H1-3
>Chain 1 (P3-C7 L7H1-3-1):
SEQ ID NO: 121
EVQLVQSGAEVKKPGASVKVSCKASGYTFTDHYIHWVRQAPGQGLEWMGVINPYN
DGATYNQKFKGRVTMTVDKSTSTVYMELSSLRSEDTAVYYCARPPRAYGNYAGYW
GQGTTVTVSSGGGGSSGAPRFLTRPKASVVSVGKDATLSCQIVGNPFPQVSWEKDKQ
PVTAGVRFRLAQDGDLYRLKILDLQLSDSGQYVCRARNAHGEAFACLGLQVDAEAD
KTHTCPPCPAPEAAGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEK
TISKAKGQPREPQVYTLPPCREEMTKNQVSLWCLVKGFYPSDIAVEWESNGQPENNY
KTTPPVLDSDGSFFLYSKLTVDKSRWQQGNVFSCSVMHEALHNHYTQKSLSLSPGK
>Chain 2 (P3-C7 L7H1-3-2, same as P3-C7 L7H1-2-4, SEQ ID NO: 107)
>Chain 3 (Format 3-3, SEQ ID NO: 93)
>Chain 4 (Format 3-4, same as Format 2-2; SEQ ID NO: 92)
2. Full-length sequence of 81 L1H1-3
>Chain 1 (81 L1H1-3-1):
SEQ ID NO: 122

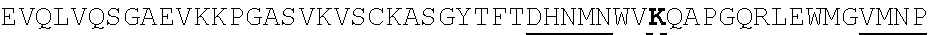

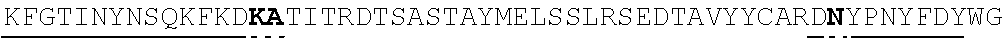

QGTTVTVSSGGGGSSGAPRFLTRPKASVVSVGKDATLSCQIVGNPFPQVSWEKDKQP
VTAGVRFRLAQDGDLYRLKILDLQLSDSGQYVCRARNAHGEAFACLGLQVDAEADK
THTCPPCPAPEAAGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKALPAPIEKT
ISKAKGQPREPQVYTLPPCREEMTKNQVSLWCLVKGFYPSDIAVEWESNGQPENNYK
TTPPVLDSDGSFFLYSKLTVDKSRWQQGNVFSCSVMHEALHNHYTQKSLSLSPGK
>Chain 2 (81 L1H1-3-2, same as 81 L1H1-2-4, SEQ ID NO: 111)
>Chain 3 (Format 3-3, SEQ ID NO: 93)
>Chain 4 (Format 3-4, same as Format 2-2; SEQ ID NO: 92)
3. Full-length sequence of 81 L2H1-3
>Chain 1 (81 L2H1-3-1, same as 81 L1H1-3-1, SEQ ID NO: 122)
>Chain 2 (81 L2H1-3-2, same as 81 L2H1-2-4, SEQ ID NO: 112)
>Chain 3 (Format 3-3, SEQ ID NO: 93)
>Chain 4 (Format 3-4, same as Format 2-2; SEQ ID NO: 92)
4. Full-length sequence of 81 L3H1-3
>Chain 1 (81 L3H1-3-1, same as 81 L1H1-3-1, SEQ ID NO: 122)
>Chain 2 (81 L3H1-3-2, same as 81 L3H1-2-4, SEQ ID NO: 113)
>Chain 3 (Format 3-3, SEQ ID NO: 93)
>Chain 4 (Format 3-4, same as Format 2-2; SEQ ID NO: 92)
5. Full-length sequence of 81 L4H1-3
>Chain 1 (81 L4H1-3-1, same as 81 L1H1-3-1, SEQ ID NO: 122)
>Chain 2 (81 L4H1-3-2, same as 81 L4H1-2-4, SEQ ID NO: 114)
>Chain 3 (Format 3-3, SEQ ID NO: 93)
>Chain 4 (Format 3-4, same as Format 2-2; SEQ ID NO: 92)
6. Full-length sequence of 81 L5H1-3
>Chain 1 (81 L5H1-3-1, same as 81 L1H1-3-1, SEQ ID NO: 122)
>Chain 2 (81 L5H1-3-2, same as 81 L5H1-2-4, SEQ ID NO: 115)
>Chain 3 (Format 3-3, SEQ ID NO: 93)
>Chain 4 (Format 3-4, same as Format 2-2; SEQ ID NO: 92)
7. Full-length sequence of 81 L6H1-3
>Chain 1 (81 L6H1-3-1, same as 81 L1H1-3-1, SEQ ID NO: 122)
>Chain 2 (81 L6H1-3-2, same as 81 L6H1-2-4, SEQ ID NO: 116)
>Chain 3 (Format 3-3, SEQ ID NO: 93)
>Chain 4 (Format 3-4, same as Format 2-2; SEQ ID NO: 92)
8. Full-length sequence of 146 L1H1-3
>Chain 1 (146 L1H1-3-1):
SEQ ID NO: 123

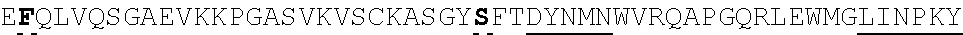

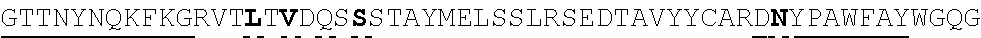

TTVTVSSGGGGSSGAPRFLTRPKASVVSVGKDATLSCQIVGNPFPQVSWEKDKQPVT
AGVRFRLAQDGDLYRLKILDLQLSDSGQYVCRARNAHGEAFACLGLQVDAEADKTH
TCPPCPAPEAAGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISK
AKGQPREPQVYTLPPCREEMTKNQVSLWCLVKGFYPSDIAVEWESNGQPENNYKTTP
PVLDSDGSFFLYSKLTVDKSRWQQGNVFSCSVMHEALHNHYTQKSLSLSPGK
>Chain 2 (146 L1H1-3-2, same as 146 L1H1-2-4, SEQ ID NO: 120)
>Chain 3 (Format 3-3, SEQ ID NO: 93)
>Chain 4 (Format 3-4, same as Format 2-2; SEQ ID NO: 92)

Illustratively, the specific amino acid sequences of bispecific antibodies constructed with Format 4 in the present disclosure are as follows:

1. Full-length sequence of P3-C7 L7H1-4
>Chain 1 (P3-C7 L7H1-4-1):
SEQ ID NO: 124
EVQLVQSGAEVKKPGASVKVSCKASGYTFTDHYIHWVRQAPGQGLEWMGVINPYN
DGATYNQKFKGRVTMTVDKSTSTVYMELSSLRSEDTAVYYCARPPRAYGNYAGYW
GQGTTVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCEVQLVESGGG
LVQPGGSLKLSCAASGFTFNKYAMNWVRQAPGKGLEWVARIRSKYNNYATYYADSV
KDRFTISRDDSKNTAYLQMNNLKTEDTAVYYCVRHGNFGNEYISYWAYWGQGTLVT

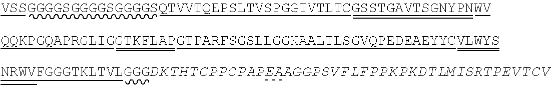

VVDVSHEDPEVKFNWYVDGVEVHNAKTKPREEQYNSTYRVVSVLTVLHQDWLNGKEYKC
KVSNKALPAPIEKTISKAKGQPREPQVYTLPPSREEMTKNQVSLTCLVKGFYPSDIAVEWES
NGQPENNYKTTPPVLDSDGSFFLYSKLTVDKSRWQQGNVFSCSVMHEALHNHYTOKSLSL
SPGK
>Chain 2 (P3-C7 L7H1-4-2, same as P3-C7 L7H1 light chain, SEQ ID NO: 65)
2. Full-length sequence of P3-C7 L8H1-4
>Chain 1 (P3-C7 L8H1-4-1, same as P3-C7 L7H1-4-1, SEQ ID NO: 124)
>Chain 2 (P3-C7 L8H1-4-2, same as P3-C7 L8H1 light chain, SEQ ID NO: 66)

Illustratively, the specific amino acid sequences of bispecific antibodies constructed with Format 5 in the present disclosure are as follows:

1. Full-length sequence of P3-C7 L7H1-5
>Chain 1 (P3-C7 L7H1-5-1):
SEQ ID NO: 125
EVQLVQSGAEVKKPGASVKVSCKASGYTFTDHYIHWVRQAPGQGLEWMGVINPYN
DGATYNQKFKGRVTMTVDKSTSTVYMELSSLRSEDTAVYYCARPPRAYGNYAGYW
GQGTTVTVSSASTKGPSVFPLAPSSKSTSGGTAALGCLVKDYFPEPVTVSWNSGALTSGVH

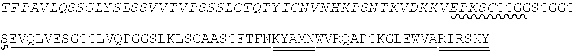

NNYATYYADSVKDRFTISRDDSKNTAYLQMNNLKTEDTAVYYCVRHGNFGNEYISY

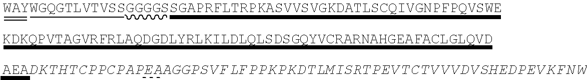

YVDGVEVHNAKTKPREEQYNSTYRVVSVLTVLHQDWLNGKEYKCKVSNKALPAPIEKTISK
AKGQPREPQVYTLPPSREEMTKNQVSLTCLVKGFYPSDIAVEWESNGQPENNYKTTPPVL
DSDGSFFLYSKLTVDKSRWQQGNVFSCSVMHEALHNHYTQKSLSLSPGK
>Chain 2 (P3-C7 L7H1-5-2, same as P3-C7 L7H1 light chain, SEQ ID NO: 65)
>Chain 3 (Format 5-3, SEQ ID NO: 98)
2. Full-length sequence of P3-C7 L8H1-5
>Chain 1 (P3-C7 L8H1-5-1, same as P3-C7 L7H1-5-1, SEQ ID NO: 125)
>Chain 2 (P3-C7 L8H1-5-2, same as P3-C7 L8H1 light chain, SEQ ID NO: 66)
>Chain 3 (Format 5-3, SEQ ID NO: 98)

In addition, the GUCY2C-CD3 bispecific antibody GUCY2c-1608 (named 1608-BsAb in the present disclosure), which is prepared with reference to patent No. WO2019224716A2, is also used in the present disclosure; it has a DIABODY structure, and the complete molecule comprises two chains, which are specifically as follows:

>Chain 1 (GUCY2c-1608-Knob sequence):
SEQ ID NO: 126
DIQLTQSPSSLSASVGDRVTITCRASESVDYYGSSLLQWYQQKPGKAPKLLIYAASKL

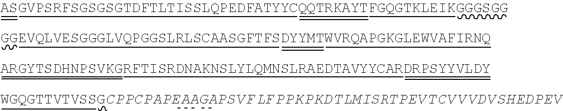

KFNWYVDGVEVHNAKTKPREEQYNSTYRVVSVLTVLHODWLNGKEYKCKVSNKALPAPIE

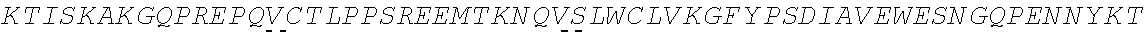

TPPVLDSDGSFFLYSKLTVDKSRWQQGNVFSCSVMHEALHNHYTOKSLSLSPG
>Chain 2 (GUCY2c-1608-Hole sequence):
SEQ ID NO: 127
DIQMTQSPSSLSASVGDRVTITCTSSQSLENVRSQKNYLAWYQQKPGKAPKLLIYWAS

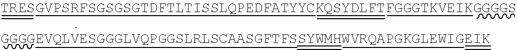

PSNELTNVHEKFKDRFTISVDKAKNSAYLQMNSLRAEDTAVYYCTRTITTTEGYWFF

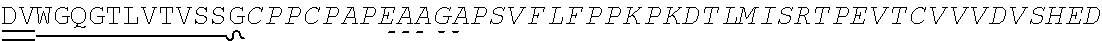

PEVKFNWYVDGVEVHNAKTKPREEQYNSTYRVVSVLTVLHQDWLNGKEYKCKVSNKALP

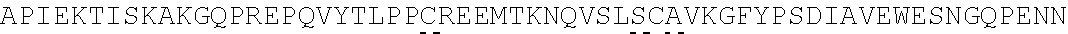

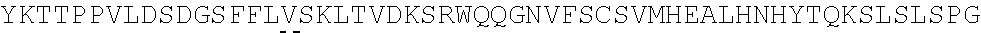

TABLE 26
CDR sequences of 1608-BsAb

| | | |
|---|---|---|
| GUCY2C | HCDR1 | SYWMH (SEQ ID NO: 128) |
| | HCDR2 | EIKPSNELTNVHEKFKD (SEQ ID NO: 129) |
| | HCDR3 | TITTTEGYWFFDV (SEQ ID NO: 130) |
| | LCDR1 | RASESVDYYGSSLLQ (SEQ ID NO: 131) |
| | LCDR2 | AASKLAS (SEQ ID NO: 132) |
| | LCDR3 | QQTRKAYT (SEQ ID NO: 133) |
| CD3 | HCDR1 | DYYMT (SEQ ID NO: 134) |
| | HCDR2 | FIRNQARGYTSDHNPSVKG (SEQ ID NO: 135) |
| | HCDR3 | DRPSYYVLDY (SEQ ID NO: 136) |
| | LCDR1 | TSSQSLFNVRSQKNYLA (SEQ ID NO: 137) |
| | LCDR2 | WASTRES (SEQ ID NO: 138) |
| | LCDR3 | KQSYDLFT (SEQ ID NO: 139) |

In addition, the EGFR-CD28 bispecific antibody 97H2L30T-ZAL, which is prepared with reference to patent No. CN202210371538.8, is also used in the present disclosure, and its sequence is specifically as follows:

TABLE 27
CDRs of EGFR-CD28 bispecific antibody 97H2L3OT-ZAL

| | Heavy chain | | Light chain | |
|---|---|---|---|---|
| EGFR | HCDR1 | TYGMH (SEQ ID NO: 140) | LCDR1 | RASQDISSALV (SEQ ID NO: |
| | | | | 143) |
| | HCDR2 | VIWDDGSYKYYGDSVKG | LCDR2 | DASSLES (SEQ ID NO: 144) |
| | | (SEQ ID NO: 141) | | |
| | HCDR3 | DGITMVRGVMKDYFDY (SEQ ID | LCDR3 | QQFNSYPLT (SEQ ID NO: |
| | | NO: 142) | | 145) |
| CD28 | HCDR1 | SYWIT | LCDR1 | RASQDISNYLN (SEQ ID NO: |
| | | (SEQ ID NO: 146) | | 149) |
| | HCDR2 | DIYPGSGRTNYNEMFKN (SEQ ID | LCDR2 | YTSRLHS (SEQ ID NO: 150) |
| | | NO: 147) | | |
| | HCDR3 | RHYGTGYESFDV | LCDR3 | QQGNTLPWT (SEQ ID NO: |
| | | (SEQ ID NO: 148) | | 151) |

EGFR-VH:
SEQ ID NO: 152
QVQLVESGGGVVQPGRSLRLSCAASGFTFSTYGMHWVRQAPGKGLEWVAVIWDDG
SYKYYGDSVKGRFTISRDNSKNTLYLQMNSLRAEDTAVYYCARDGITMVRGVMKD
YFDYWGQGTLVTVSS
EGFR-VL:
SEQ ID NO: 153
DIQLTQSPSSLSASVGDRVTITCRASQDISSALVWYQQKPGKAPKLLIYDASSLESGVP
SRFSGSESGTDFTLTISSLQPEDFATYYCQQFNSYPLTFGGGTKVEIK
CD28-VH:
SEQ ID NO: 154
EVQLVQSGAEVKKPGASVKVSCKASGYTLTSYWITWVRQAPGQGLEWMGDIYPGS
GRTNYNEMFKNRVTMTVDTSTSTAYMELSSLRSEDTAVYYCARRHYGTGYESFDVW
GQGTTVTVSS
CD28-VL:
SEQ ID NO: 155
DIQMTQSPSSLSASVGDRVTITCRASQDISNYLNWYQQKPDGTIKLLIYYTSRLHSGV
PSRFSGSGSGTDYTFTISSLQPEDIATYYCQQGNTLPWTFGGGTKVEIK
Full-length sequence of EGFR-CD28 bispecific antibody 97H2L3OT-ZAL:
>Chain 1:
SEQ ID NO: 156
EVQLVQSGAEVKKPGASVKVSCKASGYTLTSYWITWVRQAPGOGLEWMGDIYPGS
GRTNYNEMFKNRVTMTVDTSTSTAYMELSSLRSEDTAVYYCARRHYGTGYESFDVW
GQGTTVTVSSGGGGSGIPPKIECLPIDISIDEGKVLTVASAFTGEPTPEVTWSTGGRKIH
SQEQGRFHIENTDDSTTLTIKDVQKQDGGLYTLTLRNEFGSDSATVNIHIRSIDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPCRDELTKNQVSLWCLVKGFYPSDIAVEWESNGQPENNYKTTPP
VLDSDGSFFLYSKLTVDKSRWQQGNVFSCSVMHEALHNHYTQKSLSLSPGK
>Chain 2:
SEQ ID NO: 157
DIQMTQSPSSLSASVGDRVTITCRASQDISNYLNWYQQKPDGTIKLLIYYTSRLHSGV
PSRFSGSGSGTDYTFTISSLQPEDIATYYCQQGNTLPWTFGGGTKVEIKGGGGSSGAP
RFLTRPKASVVSVGKDATLSCQIVGNPFPQVSWEKDKQPVTAGVRFRLAQDGDLYRL
KILDLQLSDSGQYVCRARNAHGEAFACLGLQVDAEA
>Chain 3:
SEQ ID NO: 158
QVQLVESGGGVVQPGRSLRLSCAASGFTFSTYGMHWVRQAPGKGLEWVAVIWDDG
SYKYYGDSVKGRFTISRDNSKNTLYLOMNSLRAEDTAVYYCARDGITMVRGVMKD
YFDYWGQGTLVTVSSASTKGPSVFPLAPSSKSTSGGTAALGCLVKDYFPEPVTVSWN
SGALTSGVHTFPAVLQSSGLYSLSSVVTVPSSSLGTQTYICNVNHKPSNTKVDKKVEP
KSCDKTHTCPPCPAPEAAGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALP
APIEKTISKAKGQPREPQVCTLPPSRDELTKNQVSLSCAVKGFYPSDIAVEWESNGQPE
NNYKTTPPVLDSDGSFFLVSKLTVDKSRWQQGNVFSCSVMHEALHNHYTQKSLSLSP
GK
>Chain 4:
SEQ ID NO: 159
DIQLTQSPSSLSASVGDRVTITCRASQDISSALVWYQQKPGKAPKLLIYDASSLESGVP
SRFSGSESGTDFTLTISSLQPEDFATYYCQQFNSYPLTFGGGTKVEIKRTVAAPSVFIFPP
SDEQLKSGTASVVCLLNNFYPREAKVQWKVDNALQSGNSQESVTEQDSKDSTYSLS
STLTLSKADYEKHKVYACEVTHQGLSSPVTKSFNRGEC

In addition, the EGFR-CD28 bispecific antibodies 129H4L10T-ZAL and 94H6L12-ZALOT, both of which were prepared with reference to patent No. CN202210371538.8, are used in the present disclosure, and their sequences are specifically as follows:

TABLE 28
CDRs of EGFR-CD28 bispecific antibody 129H4L1OT-ZAL

| | Heavy chain | | Light chain | |
|---|---|---|---|---|
| EGFR | HCDR1 | TYGMH (SEQ ID NO: 140) | LCDR1 | RASQDISSALV (SEQ ID NO: |
| | | | | 143) |
| | HCDR2 | VIWDDGSYKYYGDSVKG (SEQ ID | LCDR2 | DASSLES (SEQ ID NO: 144) |
| | | NO: 141) | | |
| | HCDR3 | DGITMVRGVMKDYFDY (SEQ ID | LCDR3 | QQFNSYPLT (SEQ ID NO: |
| | | NO: 142) | | 145) |
| CD28 | HCDR1 | SHWIQ (SEQ ID NO: 160) | LCDR1 | RASQDISNYLN (SEQ ID NO: |
| | | | | 149) |
| | HCDR2 | EIFPGSGSTHYNEKFKG (SEQ ID | LCDR2 | YTSRLHS (SEQ ID NO: 150) |
| | | NO: 161) | | |
| | HCDR3 | LHYGTSYDAMDY (SEQ ID NO: | LCDR3 | QQGYTLPRT (SEQ ID NO: |
| | | 162) | | 163) |

EGFR-VH (same as EGFR-VH of 129H4L1OT-ZAL, SEQ ID NO: 152)
EGFR-VL (same as EGFR-VL of 129H4L1OT-ZAL, SEQ ID NO: 153)
CD28-VH:
SEQ ID NO: 164
EVQLVQSGAEVKKPGASVKVSCKAPGYTFTSHWIQWVRQAPGQGLEWMGEIFPGSG
STHYNEKFKGRVTMTVDTSTSTAYMELSSLRSEDTAVYYCARLHYGTSYDAMDYWG
QGTTVTVSS
CD28-VL:
SEQ ID NO: 165
DIQMTQSPSSLSASVGDRVTITCRASQDISNYLNWYQQKPGKAVKLLIYYTSRLHSGV
PSRFSGSGSGTDFTFTISSLQPEDIATYYCQQGYTLPRTFGGGTKVEIK
Full-length sequence of EGFR-CD28 bispecific antibody 129H4L1OT-ZAL:
>Chain 1 (SEQ ID NO: 166):
EVQLVQSGAEVKKPGASVKVSCKAPGYTFTSHWIQWVRQAPGQGLEWMGEIFPGSG
STHYNEKFKGRVTMTVDTSTSTAYMELSSLRSEDTAVYYCARLHYGTSYDAMDYWG
QGTTVTVSSGGGGSGIPPKIECLPIDISIDEGKVLTVASAFTGEPTPEVTWSTGGRKIHS
QEQGRFHIENTDDSTTLTIKDVQKQDGGLYTLTLRNEFGSDSATVNIHIRSIDKTHTCP
PCPAPEAAGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAK
GQPREPQVYTLPPCRDELTKNQVSLWCLVKGFYPSDIAVEWESNGQPENNYKTTPPV
LDSDGSFFLYSKLTVDKSRWQQGNVFSCSVMHEALHNHYTQKSLSLSPGK
>Chain 2 (SEQ ID NO: 167):
DIQMTQSPSSLSASVGDRVTITCRASQDISNYLNWYQQKPGKAVKLLIYYTSRLHSGV
PSRFSGSGSGTDFTFTISSLQPEDIATYYCQQGYTLPRTFGGGTKVEIKGGGGSSGAPR
FLTRPKASVVSVGKDATLSCQIVGNPFPQVSWEKDKQPVTAGVRFRLAQDGDLYRLK
ILDLQLSDSGQYVCRARNAHGEAFACLGLQVDAEA
>Chain 3 (same as chain 3 of 97H2L3OT-ZAL, SEQ ID NO: 158)
>Chain 4 (same as chain 4 of 97H2L3OT-ZAL, SEQ ID NO: 159)

TABLE 29
CDRs of EGFR-CD28 bispecific antibody 94H6L12-ZALOT

| | Heavy chain | | Light chain | |
|---|---|---|---|---|
| EGFR | HCDR1 | TYGMH (SEQ ID NO: 140) | LCDR1 | RASQDISSALV (SEQ ID NO: |
| | | | | 143) |
| | HCDR2 | VIWDDGSYKYYGDSVKG (SEQ ID | LCDR2 | DASSLES (SEQ ID NO: 144) |
| | | NO: 141) | | |
| | HCDR3 | DGITMVRGVMKDYFDY (SEQ ID | LCDR3 | QQFNSYPLT (SEQ ID NO: |
| | | NO: 142) | | 145) |
| CD28 | HCDR1 | GYYMN (SEQ ID NO: 168) | LCDR1 | LASQTIGTWLA (SEQ ID NO: |
| | | | | 171) |
| | HCDR2 | EINPSTGDTTYNQKFKA | LCDR2 | AATTGAD (SEQ ID NO: 172) |
| | | (SEQ ID NO: 169) | | |
| | HCDR3 | PYYDLTPFLY (SEQ ID NO: | LCDR3 | QQLYSTPYT (SEQ ID NO: |
| | | 170) | | 173) |

EGFR-VH (same as EGFR-VH of 129H4L1OT-ZAL, SEQ ID NO: 152)
EGFR-VL (same as EGFR-VL of 129H4L1OT-ZAL, SEQ ID NO: 153)
CD28-VH:
SEQ ID NO: 174
EVQLVQSGAEVKKPGASVKVSCKASGYSFTGYYMNWVRQAPGQRLEWMGEINPST
GDTTYNQKFKAKVTLTVDKSASTAYMELSSLRSEDTAVYYCASPYYDLTPFLYWGQG
TLVTVSS
CD28-VL:
SEQ ID NO: 175
DIQMTQSPSSVSASVGDRVTITCLASQTIGTWLAWYQQKPGKSPKLLIYAATTGADGV
PSRFSGSGSGTKFTLTISSLQPEDFATYYCQQLYSTPYTFGGGTKVEIK
Full-length sequence of EGFR-CD28 bispecific antibody 94H6L12-ZALOT:
>Chain 1:
SEQ ID NO: 176
EVQLVQSGAEVKKPGASVKVSCKASGYSFTGYYMNWVRQAPGQRLEWMGEINPST
GDTTYNQKFKAKVTLTVDKSASTAYMELSSLRSEDTAVYYCASPYYDLTPFLYWGQG
TLVTVSSASTKGPSVFPLAPSSKSTSGGTAALGCLVKDYFPEPVTVSWNSGALTSGVH
TFPAVLQSSGLYSLSSVVTVPSSSLGTQTYICNVNHKPSNTKVDKKVEPKSCDKTHTC
PPCPAPEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKA
KGQPREPQVYTLPPCRDELTKNQVSLWCLVKGFYPSDIAVEWESNGQPENNYKTTPP
VLDSDGSFFLYSKLTVDKSRWQQGNVFSCSVMHEALHNHYTQKSLSLSPGK
>Chain 2:
SEQ ID NO: 177
DIQMTQSPSSVSASVGDRVTITCLASQTIGTWLAWYQQKPGKSPKLLIYAATTGADGV
PSRFSGSGSGTKFTLTISSLQPEDFATYYCQQLYSTPYTFGGGTKVEIKRTVAAPSVFIFP
PSDEQLKSGTASVVCLLNNFYPREAKVQWKVDNALQSGNSQESVTEQDSKDSTYSL
SSTLTLSKADYEKHKVYACEVTHQGLSSPVTKSFNRGEC
>Chain 3:
SEQ ID NO: 178
QVQLVESGGGVVQPGRSLRLSCAASGFTFSTYGMHWVRQAPGKGLEWVAVIWDDG
SYKYYGDSVKGRFTISRDNSKNTLYLOMNSLRAEDTAVYYCARDGITMVRGVMKD
YFDYWGQGTLVTVSSGGGGSSGAPRFLTRPKASVVSVGKDATLSCQIVGNPFPQVSW
EKDKQPVTAGVRFRLAQDGDLYRLKILDLQLSDSGQYVCRARNAHGEAFACLGLQV
DAEADKTHTCPPCPAPEAAGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALP
APIEKTISKAKGQPREPQVCTLPPSRDELTKNQVSLSCAVKGFYPSDIAVEWESNGQPE
NNYKTTPPVLDSDGSFFLVSKLTVDKSRWQQGNVFSCSVMHEALHNHYTQKSLSLSP
GK
>Chain 4:
SEQ ID NO: 179
DIQLTQSPSSLSASVGDRVTITCRASQDISSALVWYQQKPGKAPKLLIYDASSLESGVP
SRFSGSESGTDFTLTISSLQPEDFATYYCQQFNSYPLTFGGGTKVEIKGGGGSGIPPKIE
CLPIDISIDEGKVLTVASAFTGEPTPEVTWSTGGRKIHSQEQGRFHIENTDDSTTLTIKD
VQKQDGGLYTLTLRNEFGSDSATVNIHIRSI
>IgG1Fc-Knob (L234A/L235A/S354C/T366W)
SEQ ID NO: 248
DKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIE
KTISKAKGQPREPQVYTLPPCRDELTKNQVSLWCLVKGFYPSDIAVEWESNGQPENN
YKTTPPVLDSDGSFFLYSKLTVDKSRWQQGNVFSCSVMHEALHNHYTQKSLSLSPGK
>IgG1Fc-Hole (L234A/L235A/Y349C/T366S/L368A/Y407V)
SEQ ID NO: 249
DKTHTCPPCPAPEAAGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIE
KTISKAKGQPREPQVCTLPPSRDELTKNQVSLSCAVKGFYPSDIAVEWESNGQPENNY
KTTPPVLDSDGSFFLVSKLTVDKSRWQQGNVFSCSVMHEALHNHYTQKSLSLSPGK
>Linker 8
(SEQ ID NO: 90)

### TEST EXAMPLES

### Test Example 1. Affinity of GUCY2C Monoclonal Antibodies of the Present Disclosure for Antigen

To test the affinity of the GUCY2C monoclonal antibodies of the present disclosure for GUCY2C, in this test example, the instruments Biacore 8K and Biacore T200 were used to detect the ability of the antibodies to bind to hGUCY2C-ECD-His (SEQ ID NO: 3). The specific method is as follows: The antibody was affinity-captured using a Protein A biosensor chip conjugated with an anti-human antibody, then a solution containing GUCY2C Protein flowed on the surface of the chip, and reaction signals were detected in real time with an instrument to obtain association and dissociation curves. After dissociation was completed for each cycle, the biosensor chip was washed with 10 mM glycine-hydrochloric acid (pH 1.5) for regeneration. Data fitting was performed using a 1:1 model. The affinity data for the binding of GUCY2C monoclonal antibodies of the present disclosure to the corresponding antigen are shown in the tables below.

TABLE 30-1
Binding of monoclonal antibodies to human GUCY2C

| Antibody | ka (1/Ms) | kd (1/s) | KD (nM) |
|---|---|---|---|
| 81 L3H1 | 1.81E+05 | 5.19E−05 | 0.29 |
| 146 L1H1 | 1.27E+05 | 1.17E−04 | 0.92 |

TABLE 30-2
Binding of monoclonal antibodies to human GUCY2C

| Antibody | ka (1/Ms) | kd (1/s) | KD (nM) |
|---|---|---|---|
| 81 L2H2 | 2.74E+05 | 1.27E−04 | 0.46 |
| 81 L3H2 | 2.51E+05 | 1.03E−04 | 0.41 |
| 81 L4H2 | 2.12E+05 | 2.07E−04 | 0.98 |
| 81 L6H2 | 2.41E+05 | 1.14E−04 | 0.47 |
| M81 CHI | 2.16E+05 | 5.90E−05 | 0.27 |
| M146 CHI | 2.03E+05 | 9.22E−05 | 0.46 |

TABLE 30-3
Binding of monoclonal antibodies to human GUCY2C

| Antibody | ka (1/Ms) | kd (1/s) | KD (nM) |
|---|---|---|---|
| P3-C7 CHI | 2.26E+04 | 1.49E−04 | 6.61 |
| P3-C7 L1H1 | 2.37E+04 | 1.56E−04 | 6.55 |
| P3-C7 L2H1 | 3.10E+04 | 1.66E−04 | 5.35 |
| P3-C7 L3H1 | 2.84E+04 | 1.35E−03 | 47.50 |
| P3-C7 L4H1 | 2.85E+04 | 2.09E−03 | 73.40 |
| P3-C7 L5H1 | 2.69E+04 | 7.19E−04 | 26.70 |
| P3-C7 L7H1 | 2.39E+04 | 1.86E−04 | 7.77 |

TABLE 30-4
Binding of monoclonal antibodies to human GUCY2C

| Antibody | ka (1/Ms) | kd (1/s) | KD (nM) |
|---|---|---|---|
| M42 CHI | 3.73E+04 | 3.35E−04 | 8.99 |
| M61 CHI | 5.08E+04 | 2.28E−04 | 4.49 |
| M64 CHI | 1.71E+04 | 3.48E−04 | 20.40 |
| M93 CHI | 2.12E+04 | 3.60E−04 | 17.00 |

TABLE 30-5
Binding of monoclonal antibodies to human GUCY2C

| Antibody | ka (1/Ms) | kd (1/s) | KD (nM) |
|---|---|---|---|
| P1-D2 CHI | 1.95E+05 | 1.80E−04 | 0.92 |
| P1-D3 CHI | 4.60E+04 | 2.33E−04 | 5.06 |
| P1-C6 CHI | 7.07E+04 | 1.81E−04 | 2.57 |
| P1-D9 CHI | 2.03E+04 | 3.99E−04 | 19.60 |
| P1-F10 CHI | 3.23E+04 | 2.79E−04 | 8.64 |
| P2-C8 CHI | 3.49E+04 | 1.78E−04 | 5.12 |
| P2-B9 CHI | 5.81E+04 | 1.75E−04 | 3.02 |
| P3-D2 CHI | 3.86E+04 | 1.73E−04 | 4.50 |
| P3-B4 CHI | 2.58E+04 | 2.14E−04 | 8.28 |
| P3-E4 CHI | 1.77E+04 | 2.28E−04 | 12.90 |
| P3-F6 CHI | 6.61E+03 | 2.45E−04 | 37.00 |
| P3-D9 CHI | 1.90E+04 | 5.04E−04 | 26.50 |

The results show that the GUCY2C monoclonal antibodies of the present disclosure exhibit good binding affinity for human GUCY2C.

### Test Example 2. Binding Activity of Anti-GUCY2C Monoclonal Antibodies for Cell Surface Antigen

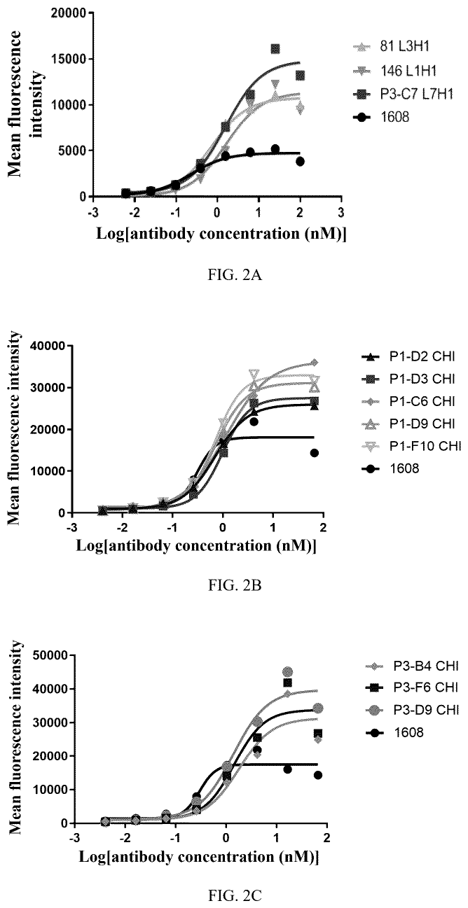

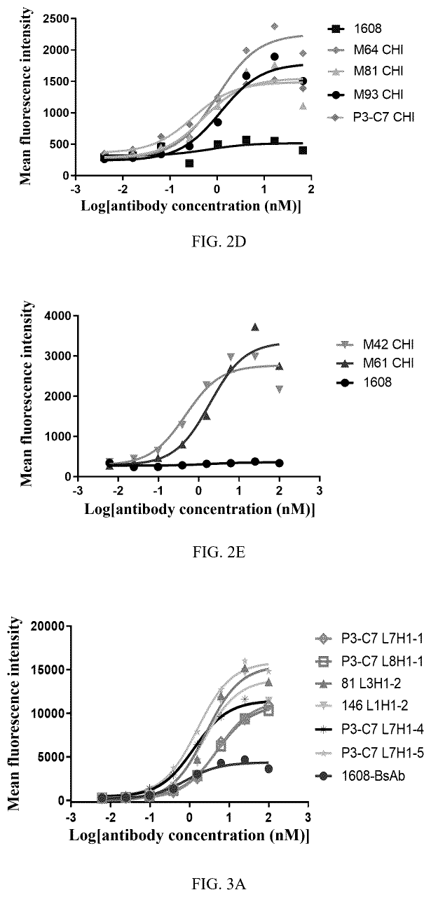

In this test example, the binding activity of GUCY2C monoclonal antibodies for human GUCY2C on the surface of stably transfected cell strain HCT116/hGUCY2C and intestinal cancer cells HT-55 and T84 that overexpress human GUCY2C was detected by flow cytometry. The specific procedures are as follows:
1E5 cells resuspended in 1% BSA were added to the reaction wells, and the supernatant was removed after centrifugation. Diluted antibody of the corresponding concentration was added, and the mixture was incubated at room temperature for 30 min and then washed once with 1% BSA. Then, PE-labeled F(ab′)2-goat anti-human IgG Fc secondary antibody (Cat. #H10104, Invitrogen) was added, and the mixture was resuspended and incubated at room temperature for 30 min in the dark. After the plate was washed twice with 1% BSA, fluorescence signals were read using a flow cytometer. Exemplary FACS results are shown in FIGS. 2A-2E.

The result shows that the GUCY2C monoclonal antibodies of the present disclosure exhibit good specific binding for GUCY2C on the cell surface, and the binding activity is better than that of 1608.

### Test Example 3. Non-Specific Binding of GUCY2C Humanized Antibody of the Present Disclosure

In this test example, the non-specific binding of the GUCY2C humanized antibody for non-target cells (e.g., T cells) is detected. The specific procedures are as follows:

Experiment 1: The flow cytometry was used; 1E5 Jurkat cells (ATCC #TIB-152) resuspended in 1% BSA were added to each well, and the supernatant was removed after centrifugation. Diluted antibody of the corresponding concentration was added, and the mixture was incubated at room temperature for 30 min and then washed once with 1% BSA. Then, PE-labeled F(ab′)2-goat anti-human IgG Fc secondary antibody (Cat. #H10104, Invitrogen) was added, and the mixture was resuspended and incubated at room temperature for 30 min in the dark. After the plate was washed twice with 1% BSA, fluorescence signals were read using a flow cytometer. The relative mean fluorescence intensity value was obtained by subtracting the background fluorescence intensity of the cells without incubated antibody from the mean fluorescence intensity of antibody binding. The results are shown in Table 31 below.

TABLE 31
Binding of GUCY2C humanized monoclonal antibodies to Jurkat

Relative mean fluorescence intensity value

| Antibody | 100 nM | 25 nM |
|---|---|---|
| P3-C7 L7H1 | 27 | No binding |
| 1608 | 1714 | 970 |
| 1608-BsAb | 1352 | 613 |

The results show that the GUCY2C humanized monoclonal antibody of the present disclosure does not non-specifically bind to Jurkat cells that do not express GUCY2C; 1608 significantly binds to Jurkat cells.

Experiment 2: The flow cytometry was used to detect whether the GUCY2C humanized monoclonal antibody of the present disclosure non-specifically bound to human PBMCs and CD3+ T cells in PBMCs; 3E5 human peripheral blood cells resuspended 1% BSA were added to each well, and the supernatant was removed after centrifugation. Diluted antibody of the corresponding concentration was added, and the mixture was incubated at room temperature for 30 min and then washed once with 1% BSA. Then, a secondary antibody working solution of PE-Cy7 anti-human CD3 (BD #557851) and APC anti-human IgG Fc (BioLegend #409306) was added, and the mixture was resuspended and incubated at room temperature for 30 min in the dark. After the plate was washed twice with 1% BSA, fluorescence signals were read using a flow cytometer. The relative mean fluorescence intensity value was obtained by subtracting the background fluorescence intensity of the cells without incubated antibody from the mean fluorescence intensity of antibody binding. The results are shown in Table 32 below.

TABLE 32
Binding of GUCY2C humanized monoclonal
antibodies to human PBMC

| | Relative mean fluorescence intensity value | |
|---|---|---|
| Antibody | PBMC | CD3⁺ T cells |

| | | | | |
|---|---|---|---|---|
| P3-C7 L7H1 | 1 | No binding | 3 | No binding |
| 1608 | 560 | 541 | 503 | 499 |

The results show that the GUCY2C humanized monoclonal antibody of the present disclosure does not non-specifically bind to PBMCs and T cells that do not express GUCY2C, and the control antibody 1608 does significantly non-specifically bind to PBMCs and T cells. The target specificity of the GUCY2C humanized monoclonal antibody is better than that of 1608.

### Test Example 4. Epitope Analysis of GUCY2C Monoclonal Antibodies of the Present Disclosure

To test the epitopes involved in the binding of the GUCY2C monoclonal antibodies of the present disclosure to an antigen, the ELISA assay was used to detect differences in the antibody epitopes to which the antibodies bound. The specific procedures are as follows:
The plate was coated with the monoclonal antibody tested (100 ng) and incubated overnight at 4° C. After plate washing, the antibody was blocked with 1% BSA. Then, 50 ng of hGUCY2C ECD-His antigen was added, and the mixture was incubated at 37° C. for 1 h. The plate was washed, and then 100 ng of biotinylated antibody was added. The resulting mixture was incubated at 37° C. for 1 h, and finally the signals were detected by using HRP-streptavidin. Relative competition coefficient=(signal value of antibody self-competition/signal value of other antibody competition)×100. Two antibodies with identical epitopes have a competition coefficient of 100 in competitive binding, and the larger the difference between the binding epitopes, the smaller the competition coefficient between antibodies. Specific results are shown in Table 33 below:

TABLE 33
Competitive binding of GUCY2C monoclonal antibodies to antigen

| | Competitive binding | | | |
|---|---|---|---|---|
| | 1608- | P3-C7 L7H1- | 81 L3H1- | 146 L1H1- |
| | biotinylated | biotinylated | biotinylated | biotinylated |
| | Coating | | | |
| | 100 ng | 100 ng | 100 ng | 100 ng |
| 1608 | 100 | 3 | 6 | 12 |
| P3-C7 L7H1 | 10 | 100 | 6 | 13 |
| 81 L3H1 | 11 | 3 | 100 | 81 |
| 146 L1H1 | 12 | 4 | 99 | 100 |

The results show that the antigen epitopes bound by the GUCY2C monoclonal antibodies of the present disclosure are significantly different from that bound by 1608, and thus the GUCY2C monoclonal antibodies can not compete with 1608 for binding to the GUCY2C antigen.

### Test Example 5. Protein Binding Activity of GUCY2C-CD3 Bispecific Antibodies for Antigen

In this test example, the binding activity of the GUCY2C-CD3 bispecific antibodies for human GUCY2C was detected by ELISA. The specific procedures are as follows:

The plate was coated by adding 100 ng of streptavidin (ACRO #STN-N5116) to the reaction wells and incubated overnight at 4° C. After the blocking with 1% BSA (PBS), 100 ng of hGUCY2C ECD-His was added, and the mixture was incubated at 37° C. for 1 h. After plate washing, the gradiently diluted antibody was added, and the mixture was incubated at 37° C. for 1 h. Then, Peroxidase AffiniPure goat anti-human IgG (H+L) (Cat. #109-035-003, Jackson Immuno Research) was added, and the resulting mixture was incubated at 37° C. for 1 h. After plate washing, a substrate solution was added, and a stop solution was then added after sufficient color development to stop the reaction. The signal values at 450 nM were read using a microplate reader. The binding activity of the antibodies is shown in Table 34 below.

TABLE 34
Binding activity of GUCY2C-CD3 bispecific antibodies
for human GUCY2C protein (absorbance values)

| Antibody | Top | EC₅₀ (nM) |
|---|---|---|
| P3-C7 L7H1-1 | 2.402 | 0.37 |
| 81 L3H1-2 | 2.334 | 0.12 |
| 146 L1H1-2 | 2.353 | 0.06 |
| 1608-BsAb | 2.153 | 1.23 |

The results show that the GUCY2C-CD3 bispecific antibodies of the present disclosure exhibit good binding activity for human GUCY2C protein.

### Test Example 6. Binding Activity of GUCY2C-CD3 Bispecific Antibodies for Cell Surface Antigen

In this test example, the binding activity of GUCY2C-CD3 bispecific antibodies for the stably transfected cell strain HCT116/hGUCY2C and the intestinal cancer cells HT55 (Nanjing Cobioer, #CBP60012) overexpressing human GUCY2C and the Jurkat cells (ATCC #TIB-152) expressing CD3 was detected by flow cytometry. The expression levels of GUCY2C in HCT116/hGUCY2C cells and intestinal cancer cells HT55 were high expression (+++) and moderate expression (++), respectively. The specific procedures are as follows:

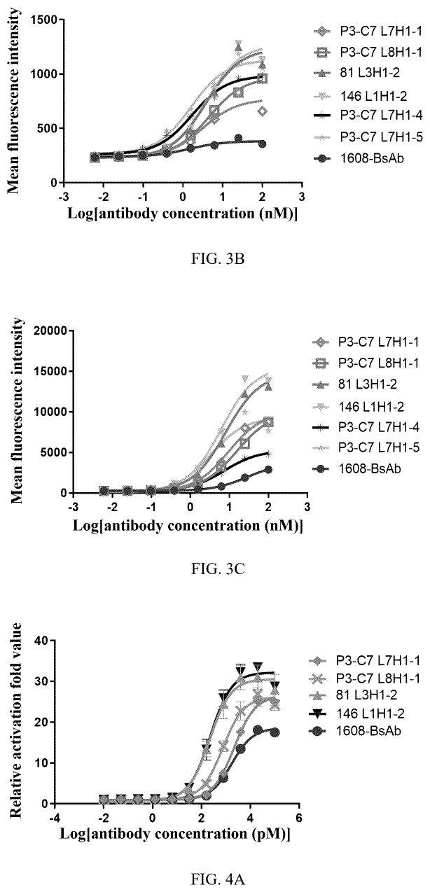

Experiment 1: 1E5 cells resuspended in 1% BSA were added to the reaction wells, and the supernatant was removed after centrifugation. Diluted antibody of the corresponding concentration was added, and the mixture was incubated at room temperature for 30 min and then washed twice with 1% BSA. Then, 1:400 diluted goat anti-human IgG(γ) R-PE conjugate F(ab′) secondary antibody (life technologies #H10104) was added, and the resulting mixture was resuspended and incubated at room temperature for 30 min in the dark. After the plate was washed twice with 10% BSA, fluorescence signals were read using a flow cytometer. Exemplary FACS results are shown in FIGS. 3A-3B.

The results show that the GUCY2C-CD3 bispecific antibodies of the present disclosure exhibit good specific binding activity for all the cells expressing GUCY2C at different levels, and the binding activity is better than that of 1608-BsAb.

Experiment 2: the same method as in experiment 1 was used. The binding activity of the antibodies for Jurkat cells is shown in FIG. 3C.

The results show that the GUCY2C-CD3 bispecific antibodies of the present disclosure exhibit good specific binding activity for Jurkat cells expressing CD3, and the binding activity is better than that of 1608-BsAb.

### Test Example 7. Activation of T Cells by GUCY2C-CD3 Bispecific Antibodies

To test the activation of T cells by the GUCY2C-CD3 bispecific antibodies of the present disclosure under the combined action of target cells HCT116/GUCY2C and LS1034 cells, Jurkat-Lucia™ NFAT (InvivoGen, jktl-nfat) was used for cell detection in this test example. The method is as follows:

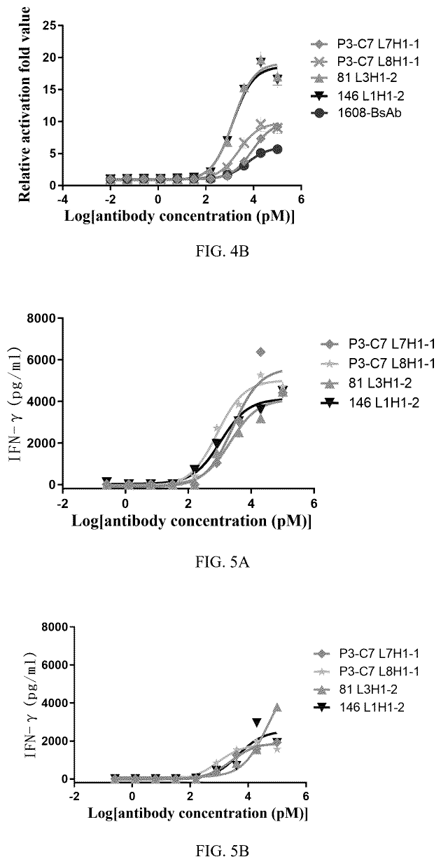

Jurkat Lucia™ NFAT cells and target cells were subcultured in complete medium and corresponding media, respectively. Jurkat-NFAT lum cells were collected by centrifugation, resuspended in the 1640+10% FBS medium and counted, and the number of cells was adjusted to 1E6 cells/mL; the target cells HCT116/GUCY2C and LS1034 were resuspended and counted, and then adjusted to 2E5 cells/mL and 4E5 cells/mL, respectively. The Jurkat Lucia™ NFAT and target cells described above were mixed in equal volume, and the mixture was added at 100 μL/well; the E:T ratios of the HCT116/GUCY2C and LS1034 cells were ensured to be 5:1 and 2.5:1, respectively. The antibody was diluted with 1640+10% FBS medium, and the diluted antibody was added at 20 L/well. The mixture was left to stand at 37° C. for 5 h. The QUANTI-Luc™ Gold reagent was dissolved in 25 mL of ddH₂O as required by the instructions. After 50 μL of QUANTI-Luc™ Gold was added, the resulting mixture was incubated at room temperature for 5 min. The luminescence was detected by Vendor, and the activation of Jurkat Lucia™ NFAT cells by antibodies at different concentrations was calculated. A graph was fitted by using the relative fold value obtained by dividing the Lumin value after antibody action by the background Lumin value. The relative fold values and the EC₅₀ results are shown in FIGS. 4A-4B.

The results show that the GUCY2C-CD3 bispecific antibodies of the present disclosure exhibit good activation effect on T cells under the combined action with target cells, and the activation effect is better than that of 1608-BsAb.

### Test Example 8. Cytotoxic Activity of GUCY2C-CD3 Bispecific Antibodies of the Present Disclosure

In this test example, the killing activity of the GUCY2C-CD3 bispecific antibodies of the present disclosure as T cell engager molecules for tumor cells was investigated. The specific procedures are as follows:

The target-specific cytotoxic activity of the bispecific antibodies of the present disclosure was detected by using stably transfected LucG cell strains constructed with the intestinal cancer cell strains LS1034, HT-55, and LS174T (ATCC #CL-188) expressing GUCY2C as high-expression, moderate-expression, and low-expression target cells, respectively. Cryopreserved PBMCs (purchased from Milestone) were thawed, resuspended in 1640+10% FBS complete medium, and then incubated in a T75 culture flask for 4 h (density: 2E6 cells/mL). The PBMCs were collected, centrifuged, resuspended in 1640+10% FBS, and counted, and the number of cells was adjusted to 1.5E6 cells/mL. Target cells were collected, centrifuged at 1000 rpm for 3 min, resuspended, and counted, and the cell concentration was adjusted to 1.5E5 cells/mL. The PBMC suspension and the target cell suspension described above were mixed in equal volume, and the mixture was added at 100 L/well; the E:T ratio was ensured to be 10:1. A PBMC ONLY group was set up separately; the PBMC suspension described above and the 1640+10% FBS complete medium were mixed in equal volume. The antibody was 5-fold diluted in 1640+10% FBS medium from an initial concentration of 600 nM (6× final concentration) to obtain 9 concentration points, and the diluted antibody was added at 20 μL/well. The treated cells were incubated in an incubator at 37° C. and 5% CO₂ for 48 h before the luminescence detection. Dose-response graphs were plotted based on the log concentrations and signal values of the antibody using Graphpad Prism 8.0 software and fitted to obtain the maximum inhibition (Imax) and IC₅₀ of the antibody-mediated killing. The maximum inhibition of the antibody was calculated by setting the inhibition to 0% for the wells containing only the target cells and PBMC but no antibody. The results are shown in Tables 35-1, 35-2, and 35-3 below.

TABLE 35-1
Cytotoxic activity of GUCY2C-CD3 bispecific
antibodies for target cells

| | LS1034(+++) | | HT-55(++) | | LS174T(+) | |
|---|---|---|---|---|---|---|
| | Imax | IC₅₀ | Imax | IC₅₀ | Imax | IC₅₀ |
| Antibody | (%) | (nM) | (%) | (nM) | (%) | (nM) |
| P3-C7 L7H1-1 | 87 | 0.75 | 39 | 0.85 | 65 | 0.75 |
| P3-C7 L8H1-1 | 86 | 0.17 | 41 | 0.60 | 62 | 0.33 |
| 81 L3H1-2 | 92 | 0.31 | 47 | 0.39 | 67 | 0.58 |
| 146 L1H1-2 | 87 | 0.17 | 46 | 0.56 | 69 | 0.49 |

Note:
+ represents the expression level of GUCY2C, where the more the +, the higher the expression level of GUCY2C.

TABLE 35-2
Cytotoxic activity of GUCY2C-CD3 bispecific
antibodies for target cells

LS174T

| Antibody | Max (%) | IC50 (nM) |
|---|---|---|
| P3-C7 L7H1-3 | 92 | 0.49 |

TABLE 35-3
Cytotoxic activity of GUCY2C-CD3 bispecific
antibodies for target cells

LS174T

| Antibody | Max (%) | IC₅₀ (nM) |
|---|---|---|
| 81 L3H1-2 (N103QN106Q) | 82 | 0.16 |

The results show that the GUCY2C-CD3 bispecific antibodies of the present disclosure exhibit good cytotoxic activity for target cells with different expression levels.

### Test Example 9. Effect of GUCY2C-CD3 Bispecific Antibodies of the Present Disclosure on Release of Cytokines

In this test example, the changes in the secretion of cytokines IFN-γ and IL-6 in PBMCs under the combined action of the GUCY2C-CD3 bispecific antibody and target cells (LS1034 cells and LS174T cells in Test Example 8) were detected.

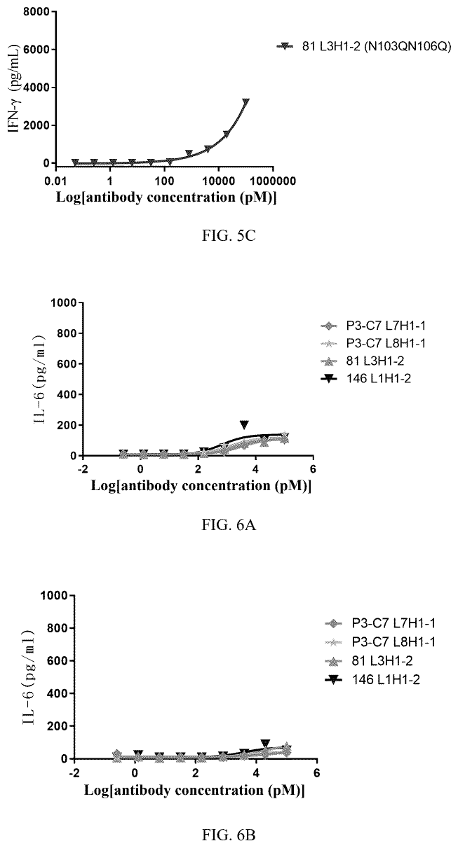

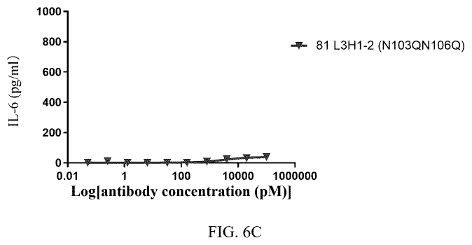

The supernatant collected in the cell killing experiment (Test Example 8) and cryopreserved in a freezer at −20° C. was thawed at room temperature and shaken for mixing well, and the sample was 20-fold diluted with the 1640+4% FBS medium for later use. Powders of IL-6 and IFN-γ standards were dissolved in sterile water and the standards were diluted to the desired concentration using the 1640+4% FBS medium. The kits for the cytokines to be detected, including a human IL6 ELISA kit (NeoBioscience, EHC007.96) and a human IFN-γ kit (CISBIO, 62HIFNGPEG), were taken out and equilibrated to normal temperature, and the detection was carried out according to the operations described in the instructions. The results are shown in FIGS. 5A-5C and FIGS. 6A-6C.

The results show that the GUCY2C-CD3 bispecific antibodies of the present disclosure are able to efficiently induce the release of IFN-γ associated with cytotoxic activity, and the release level of the cytokine IL-6 associated with inflammation is relatively low.

### Biological Evaluation of In Vivo Activity

### Test Example 10. Efficacy of GUCY2C-CD3 Bispecific Antibodies of the Present Disclosure in LS1034 Subcutaneous Xenograft Tumor Model

In the present disclosure, the in vivo efficacy of the GUCY2C-CD3 bispecific antibodies was evaluated using a human colon cancer cell LS1034 mouse subcutaneous xenograft tumor model.

Female NOG mice were purchased from Beijing Vital River (Shanghai branch). The mice were 6-8 weeks old when purchased. The human PBMCs used in this test example were purchased from Milestone.

LS1034 cells were subcutaneously inoculated into the right flank of the NOG mice at 1×10⁶ cells/mouse/200 μL (containing 50% matrigel), and human PBMCs in good state after thawing were injected into the abdominal cavity of the NOG mice at 5×10⁶ cells/mouse/100 μL the next day after inoculation. When the tumor volume of the tumor-bearing mice reached about 150 mm³, the mice with overlarge volume and too small body weight were excluded, and the remaining mice were randomly divided into groups of 8. The day of grouping was defined as the day 0 of the experiment. From day 0, equimolar amounts of antibodies were each administered intraperitoneally twice weekly for a total of two to three weeks. The tumor volume and body weight of animals were monitored twice a week, and data were recorded. The tumor-bearing animals were euthanized as the experimental endpoint when the tumor volume of the experimental animals exceeded 1000 mm³ or when most tumors showed rupture or when the animals showed 20% of body weight loss.

The tumor volume (V) was calculated as follows: V=1/2×a×b², where a and b represent length and width, respectively.

Relative tumor proliferation rate T/C (%)=(T−T₀)/(C−C₀)×100%, where T and C are the tumor volume at the end of the experiment in the treatment group and control group, respectively; T₀ and C₀ are the tumor volume at the beginning of the experiment.

Tumor growth inhibition rate TGI (%)=1−T/C (%).

TABLE 36-1
Anti-tumor activity of GUCY2C-CD3 bispecific antibodies

| | Day 0 | Day 21 | |
|---|---|---|---|
| | Mean ± SEM | Mean ± SEM | TGI |
| Group | (mm³) | (mm³) | (%) |
| Vehicle control | 140.5 ± 9.7 | 1327.6 ± 103.5 | — |
| P3-C7 L7H1-1 | 140.5 ± 11.8 | 123.2 ± 15.7*** | >100% |
| (0.3 mpk) | | | |
| P3-C7 L7H1-1 | 138.3 ± 10.8 | 363.5 ± 52*** | 81.03% |
| (0.06 mpk) | | | |
| P3-C7 L7H1-4 | 138.4 ± 11.7 | 160.1 ± 25.5*** | 98.17% |
| (0.12 mpk) | | | |
| P3-C7 L7H1-5 | 139.1 ± 9.8 | 321.6 ± 157.3*** | 84.63% |
| (0.12 mpk) | | | |

Note:
*p < 0.05,
**p < 0.01,
***p < 0.001 vs vehicle control, T-test.

TABLE 36-2
Anti-tumor activity of GUCY2C-CD3 bispecific antibodies

| | Day 0 | Day 21 | |
|---|---|---|---|
| | Mean ± SEM | Mean ± SEM | TGI |
| Group | (mm³) | (mm³) | (%) |
| Vehicle control | 159 ± 15.3 | 921.7 ± 80.4 | — |
| P3-C7 L8H1-1 | 158.7 ± 15.9 | 77.4 ± 15.9*** | >100% |
| (0.2 mpk) | | | |
| P3-C7 L8H1-1 | 158.4 ± 15.0 | 422.4 ± 96.3** | 65.39% |
| (0.05 mpk) | | | |
| 81 L3H1-2 | 158.4 ± 13.7 | 94.5 ± 8.26*** | >100% |
| (0.3 mpk) | | | |
| 81 L3H1-2 | 160.8 ± 15.2 | 135.2 ± 10.4*** | >100% |
| (0.075 mpk) | | | |

Note:
*p < 0.05,
**p < 0.01,
***p < 0.001 vs vehicle control, T-test.

The results show that the GUCY2C-CD3 bispecific antibodies of the present disclosure exhibit significant tumor inhibition effect on this model.

### Test Example 11. Efficacy of Bispecific Antibodies of the Present Disclosure in LS174T Subcutaneous Xenograft Tumor Model

In the present disclosure, the in vivo efficacy of the GUCY2C-CD3 bispecific antibodies was evaluated using a human colon cancer cell LS174T mouse subcutaneous xenograft tumor model.

Female NOG mice were purchased from Beijing Vital River (Shanghai branch). The mice were 6-8 weeks old when purchased. The human PBMCs used in this test example were purchased from Milestone.

A mixture of LS174T cells (1×10⁶ cells/mouse/100 μL) and thawed PBMCs after cryopreservation (E:T=1:1) was inoculated subcutaneously into the right flank of NOG mice. When the tumor volume of tumor-bearing mice reached about 150 mm³, mice with too large or too small tumor volume and with too small body weight were excluded, and the remaining mice were randomly divided into groups of 8. The day of grouping was defined as day 0 of the experiment, and each antibody was intraperitoneally injected on days 0, 3, 7, and 10 for a total of 4 times. The tumor volume and body weight of animals were monitored twice a week, and data were recorded. The tumor-bearing animals were euthanized as the experimental endpoint when the tumor volume of the experimental animals exceeded 1000 mm³ or when most tumors showed rupture or when the animals showed 20% of body weight loss.

TABLE 37-1
Anti-tumor activity of GUCY2C-CD3 bispecific antibodies

| | Day 0 | Day 14 |
|---|---|---|
| | Mean ± SEM | Mean ± SEM |
| Group | (mm³) | (mm³) |
| Vehicle control | 193.4 ± 21.9 | — |
| P3-C7 L7H1-1 (0.5 mpk) | 193.7 ± 20.9 | 481.5 ± 76.5 |
| 1608-BsAb (0.5 mpk) | 194.4 ± 23.5 | 809.9 ± 227 |

Note:
*p < 0.05,
**p < 0.01,
***p < 0.001 vs vehicle control, T-test.

TABLE 37-2
Anti-tumor activity of GUCY2C-CD3 bispecific antibodies

| | Day 0 | Day13 |
|---|---|---|
| | Mean ± SEM | Mean ± SEM |
| Group | (mm³) | (mm³) |
| Vehicle control | 181.0 ± 13.8 | 1506.3 ± 237.3 |
| 81 L3H1-2 (0.75 mpk) | 180.2 ± 15.7 | 377.2 ± 70.5*** |
| 81 L3H1-2 (0.375 mpk) | 181.1 ± 14.7 | 346.2 ± 63.8*** |
| 81 L3H1-2 (0.188 mpk) | 181.1 ± 14.9 | 415.1 ± 92.5*** |

Note:
*p < 0.05,
**p < 0.01,
***p < 0.001 vs vehicle control, T-test.

The results show that the GUCY2C-CD3 bispecific antibodies of the present disclosure exhibit significant tumor inhibition effect on this model. During the experiment, the tumor weight was basically consistent with the trend of tumor volume.

### Test Example 12. Anti-Tumor Activity of Combined Use of GUCY2C-CD3 Bispecific Antibody and EGFR-CD28 Bispecific Antibody

In the present disclosure, the anti-tumor activity of the combined used of the GUCY2C-CD3 bispecific antibody and the EGFR-CD28 bispecific antibody (97H2L30T-ZAL, prepared with reference to patent No. CN202210371538.8) was studied using the LS174T subcutaneous tumor model.

Female NOG mice were purchased from Beijing Vital River (Shanghai branch). The mice were 6-8 weeks old when purchased. The human PBMCs used in this test example were purchased from Milestone.

A mixture of LS174T cells (1×10⁶ cells/mouse/100 μL) and thawed PBMCs after cryopreservation (E:T=1:1) was inoculated subcutaneously into the right flank of NOG mice. When the tumor volume of tumor-bearing mice reached about 150 mm³, mice with too large or too small tumor volume and with too small body weight were excluded, and the remaining mice were randomly divided into groups of 8. The day of grouping was defined as day 0 of the experiment, and each antibody was intraperitoneally injected on days 0, 3, 7, and 11 for a total of 4 times. For the combined use group, the drugs were administered simultaneously after being formulated separately. The tumor volume and body weight of animals were monitored twice a week, and data were recorded. The tumor-bearing animals were euthanized as the experimental endpoint when the tumor volume of the experimental animals exceeded 1000 mm³ or when most tumors showed rupture or when the animals showed 20% of body weight loss. The results are shown in Table 38 below.

TABLE 38
Anti-tumor activity of combined use of GUCY2C-CD3 bispecific
antibody and EGFR-CD28 bispecific antibody

| | Day 0 | Day13 | |
|---|---|---|---|
| | Mean ± SEM | Mean ± SEM | TGI |
| Group | (mm³) | (mm³) | (%) |
| Vehicle control | 181.0 ± 13.8 | 1506.3 ± 237.3 | — |
| P3-C7 L7H1-1 (0.25 mpk) | 181.4 ± 15.7 | 520.5 ± 91.3** | 74.41% |
| 81 L3H1-2 (0.188 mpk) | 181.1 ± 14.9 | 415.1 ± 92.5*** | 82.34% |
| 97H2L3OT-ZAL (2.5 mpk) | 180.8 ± 13.7 | 742.3 ± 104.9* | 57.63% |
| P3-C7 L7H1-1 (0.25 mpk) + | 180.5 ± 12.3 | 241.3 ± 95.2*** | 95.41% |
| 97H2L3OT-ZAL (2.5 mpk) | | | |
| 81 L3H1-2 (0.188 mpk) + | 181.0 ± 12.2 | 195.5 ± 34.4*** | 98.91% |
| 97H2L3OT-ZAL (2.5 mpk) | | | |

Note:
*p < 0.05,
**p < 0.01,
***p < 0.001 vs vehicle control, T-test.

The results show that the combined use of the GUCY2C-CD3 bispecific antibody and the EGFR-CD28 bispecific antibody of the present disclosure can effectively improve the respective drug effect of the two antibodies.

Although the foregoing invention has been described in detail by way of drawings and examples for purposes of clarity of understanding, the description and examples should not be construed as limiting the scope of the present disclosure. The disclosures of all the patents and scientific literature cited herein are clearly incorporated by reference in their entireties.

1. An antigen-binding molecule that specifically binds to GUCY2C and CD3, comprising at least one antigen-binding moiety that specifically binds to GUCY2C and at least one antigen-binding moiety that specifically binds to CD3, wherein the antigen-binding moiety that specifically binds to GUCY2C comprises a heavy chain variable region GUCY2C-VH and a light chain variable region GUCY2C-VL, and the antigen-binding moiety that specifically binds to CD3 comprises a heavy chain variable region CD3-VH and a light chain variable region CD3-VL;
wherein:
i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 47, 22, 43, 44, 45, or 46, a GUCY2C-LCDR2 comprising the amino acid sequence of SEQ ID NO: 23, and a GUCY2C-LCDR3 comprising the amino acid sequence of SEQ ID NO: 24; or
ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27 or 6; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, 28, 29, or 30, and a GUCY2C-LCDR3 comprising the amino acid sequence of SEQ ID NO: 9; or
iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40 or 12; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39 or 8, and a GUCY2C-LCDR3 comprising the amino acid sequence of SEQ ID NO: 14; or
iv-1) the GUCY2C-VH comprises: a GUCY2C-HCDR1 comprising the amino acid sequence of SEQ ID NO: 250, a GUCY2C-HCDR2 comprising the amino acid sequence of SEQ ID NO: 251, and a GUCY2C-HCDR3 comprising the amino acid sequence of SEQ ID NO: 252; and the GUCY2C-VL comprises: a GUCY2C-LCDR1 comprising the amino acid sequence of SEQ ID NO: 253, a GUCY2C-LCDR2 comprising the amino acid sequence of SEQ ID NO: 254, and a GUCY2C-LCDR3 comprising the amino acid sequence of SEQ ID NO: 255; or
iv-2) the GUCY2C-VH comprises: a GUCY2C-HCDR1 comprising the amino acid sequence of SEQ ID NO: 256, a GUCY2C-HCDR2 comprising the amino acid sequence of SEQ ID NO: 257, and a GUCY2C-HCDR3 comprising the amino acid sequence of SEQ ID NO: 258; and the GUCY2C-VL comprises: a GUCY2C-LCDR1 comprising the amino acid sequence of SEQ ID NO: 259, a GUCY2C-LCDR2 comprising the amino acid sequence of SEQ ID NO: 8, and a GUCY2C-LCDR3 comprising the amino acid sequence of SEQ ID NO: 260; or
iv-3) the GUCY2C-VH comprises: a GUCY2C-HCDR1 comprising the amino acid sequence of SEQ ID NO: 261, a GUCY2C-HCDR2 comprising the amino acid sequence of SEQ ID NO: 262, and a GUCY2C-HCDR3 comprising the amino acid sequence of SEQ ID NO: 263; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 265, and a GUCY2C-LCDR3 comprising the amino acid sequence of SEQ ID NO: 266; or
iv-4) the GUCY2C-VH comprises: a GUCY2C-HCDR1 comprising the amino acid sequence of SEQ ID NO: 267, a GUCY2C-HCDR2 comprising the amino acid sequence of SEQ ID NO: 268, and a GUCY2C-HCDR3 comprising the amino acid sequence of SEQ ID NO: 269; and the GUCY2C-VL comprises: a GUCY2C-LCDR1 comprising the amino acid sequence of SEQ ID NO: 270, a GUCY2C-LCDR2 comprising the amino acid sequence of SEQ ID NO: 271, and a GUCY2C-LCDR3 comprising the amino acid sequence of SEQ ID NO: 272; or
iv-5) the GUCY2C-VH comprises: a GUCY2C-HCDR1 comprising the amino acid sequence of SEQ ID NO: 281, a GUCY2C-HCDR2 comprising the amino acid sequence of SEQ ID NO: 282, and a GUCY2C-HCDR3 comprising the amino acid sequence of SEQ ID NO: 283; and the GUCY2C-VL comprises: a GUCY2C-LCDR1 comprising the amino acid sequence of SEQ ID NO: 284, a GUCY2C-LCDR2 comprising the amino acid sequence of SEQ ID NO: 8, and a GUCY2C-LCDR3 comprising the amino acid sequence of SEQ ID NO: 285; or
iv-6) the GUCY2C-VH comprises: a GUCY2C-HCDR1 comprising the amino acid sequence of SEQ ID NO: 286, a GUCY2C-HCDR2 comprising the amino acid sequence of SEQ ID NO: 287, and a GUCY2C-HCDR3 comprising the amino acid sequence of SEQ ID NO: 288; and the GUCY2C-VL comprises: a GUCY2C-LCDR1 comprising the amino acid sequence of SEQ ID NO: 289, a GUCY2C-LCDR2 comprising the amino acid sequence of SEQ ID NO: 265, and a GUCY2C-LCDR3 comprising the amino acid sequence of SEQ ID NO: 290; or
iv-7) the GUCY2C-VH comprises: a GUCY2C-HCDR1 comprising the amino acid sequence of SEQ ID NO: 291, a GUCY2C-HCDR2 comprising the amino acid sequence of SEQ ID NO: 292, and a GUCY2C-HCDR3 comprising the amino acid sequence of SEQ ID NO: 293; and the GUCY2C-VL comprises: a GUCY2C-LCDR1 comprising the amino acid sequence of SEQ ID NO: 294, a GUCY2C-LCDR2 comprising the amino acid sequence of SEQ ID NO: 295, and a GUCY2C-LCDR3 comprising the amino acid sequence of SEQ ID NO: 296; or
iv-8) the GUCY2C-VH comprises: a GUCY2C-HCDR1 comprising the amino acid sequence of SEQ ID NO: 261, a GUCY2C-HCDR2 comprising the amino acid sequence of SEQ ID NO: 297, and a GUCY2C-HCDR3 comprising the amino acid sequence of SEQ ID NO: 298; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 299, and a GUCY2C-LCDR3 comprising the amino acid sequence of SEQ ID NO: 300; or
iv-9) the GUCY2C-VH comprises: a GUCY2C-HCDR1 comprising the amino acid sequence of SEQ ID NO: 261, a GUCY2C-HCDR2 comprising the amino acid sequence of SEQ ID NO: 301, and a GUCY2C-HCDR3 comprising the amino acid sequence of SEQ ID NO: 302; and the GUCY2C-VL comprises: a GUCY2C-LCDR1 comprising the amino acid sequence of SEQ ID NO: 303, a GUCY2C-LCDR2 comprising the amino acid sequence of SEQ ID NO: 299, and a GUCY2C-LCDR3 comprising the amino acid sequence of SEQ ID NO: 304; or
iv-10) the GUCY2C-VH comprises: a GUCY2C-HCDR1 comprising the amino acid sequence of SEQ ID NO: 305, a GUCY2C-HCDR2 comprising the amino acid sequence of SEQ ID NO: 306, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 24; or
iv-11) the GUCY2C-VH comprises: a GUCY2C-HCDR1 comprising the amino acid sequence of SEQ ID NO: 128, a GUCY2C-HCDR2 comprising the amino acid sequence of SEQ ID NO: 307, and a GUCY2C-HCDR3 comprising the amino acid sequence of SEQ ID NO: 308; and the GUCY2C-VL comprises: a GUCY2C-LCDR1 comprising the amino acid sequence of SEQ ID NO: 309, a GUCY2C-LCDR2 comprising the amino acid sequence of SEQ ID NO: 310, and a GUCY2C-LCDR3 comprising the amino acid sequence of SEQ ID NO: 311; or
iv-12) the GUCY2C-VH comprises: a GUCY2C-HCDR1 comprising the amino acid sequence of SEQ ID NO: 312, a GUCY2C-HCDR2 comprising the amino acid sequence of SEQ ID NO: 313, and a GUCY2C-HCDR3 comprising the amino acid sequence of SEQ ID NO: 314; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 315; or
iv-13) the GUCY2C-VH comprises: a GUCY2C-HCDR1 comprising the amino acid sequence of SEQ ID NO: 286, a GUCY2C-HCDR2 comprising the amino acid sequence of SEQ ID NO: 287, and a GUCY2C-HCDR3 comprising the amino acid sequence of SEQ ID NO: 316; and the GUCY2C-VL comprises: a GUCY2C-LCDR1 comprising the amino acid sequence of SEQ ID NO: 317, a GUCY2C-LCDR2 comprising the amino acid sequence of SEQ ID NO: 265, and a GUCY2C-LCDR3 comprising the amino acid sequence of SEQ ID NO: 300; or
iv-14) the GUCY2C-VH comprises: a GUCY2C-HCDR1 comprising the amino acid sequence of SEQ ID NO: 318, a GUCY2C-HCDR2 comprising the amino acid sequence of SEQ ID NO: 319, and a GUCY2C-HCDR3 comprising the amino acid sequence of SEQ ID NO: 320; and the GUCY2C-VL comprises: a GUCY2C-LCDR1 comprising the amino acid sequence of SEQ ID NO: 321, a GUCY2C-LCDR2 comprising the amino acid sequence of SEQ ID NO: 322, and a GUCY2C-LCDR3 comprising the amino acid sequence of SEQ ID NO: 323; or
iv-15) the GUCY2C-VH comprises: a GUCY2C-HCDR1 comprising the amino acid sequence of SEQ ID NO: 324, a GUCY2C-HCDR2 comprising the amino acid sequence of SEQ ID NO: 325, and a GUCY2C-HCDR3 comprising the amino acid sequence of SEQ ID NO: 326; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 327; or
iv-16) the GUCY2C-VH comprises: a GUCY2C-HCDR1 comprising the amino acid sequence of SEQ ID NO: 261, a GUCY2C-HCDR2 comprising the amino acid sequence of SEQ ID NO: 328, and a GUCY2C-HCDR3 comprising the amino acid sequence of SEQ ID NO: 302; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 329, and a GUCY2C-LCDR3 comprising the amino acid sequence of SEQ ID NO: 300;
preferably,
i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 47, a GUCY2C-LCDR2 comprising the amino acid sequence of SEQ ID NO: 23, and a GUCY2C-LCDR3 comprising the amino acid sequence of SEQ ID NO: 24; or
ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, and a GUCY2C-LCDR3 comprising the amino acid sequence of SEQ ID NO: 9; or
iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39, and a GUCY2C-LCDR3 comprising the amino acid sequence of SEQ ID NO: 14.

2. The antigen-binding molecule according to claim 1, wherein:
the CD3-VH comprises: a CD3-HCDR1 comprising the amino acid sequence of SEQ ID NO: 71, a CD3-HCDR2 comprising the amino acid sequence of SEQ ID NO: 72, and a CD3-HCDR3 comprising the amino acid sequence of SEQ ID NO: 73 or 88; and the CD3-VL comprises: a CD3-LCDR1 comprising the amino acid sequence of SEQ ID NO: 74, a CD3-LCDR2 comprising the amino acid sequence of SEQ ID NO: 75, and a CD3-LCDR3 comprising the amino acid sequence of SEQ ID NO: 76;
preferably, the CD3-VH comprises: a CD3-HCDR1 comprising the amino acid sequence of SEQ ID NO: 71, a CD3-HCDR2 comprising the amino acid sequence of SEQ ID NO: 72, and a CD3-HCDR3 comprising the amino acid sequence of SEQ ID NO: 73; and the CD3-VL comprises: a CD3-LCDR1 comprising the amino acid sequence of SEQ ID NO: 74, a CD3-LCDR2 comprising the amino acid sequence of SEQ ID NO: 75, and a CD3-LCDR3 comprising the amino acid sequence of SEQ ID NO: 76.

3. The antigen-binding molecule according to claim 1-Gr-2, wherein:
i) the GUCY2C-VH comprises the amino acid sequence of SEQ ID NO: 56, 25, or 57, and the GUCY2C-VL comprises the amino acid sequence of SEQ ID NO: 54, 55, 26, 48, 49, 50, 51, 52, or 53; or
ii) the GUCY2C-VH comprises the amino acid sequence of SEQ ID NO: 37, 15, or 38, and the GUCY2C-VL comprises the amino acid sequence of SEQ ID NO: 33, 16, 31, 32, 34, 35, or 36; or
iii) the GUCY2C-VH comprises the amino acid sequence of SEQ ID NO: 42 or 17, and the GUCY2C-VL comprises the amino acid sequence of SEQ ID NO: 41 or 18;
preferably,
i) the GUCY2C-VH comprises the amino acid sequence of SEQ ID NO: 56, and the GUCY2C-VL comprises the amino acid sequence of SEQ ID NO: 54, 55, 48, 49, 50, 51, or 52, or
the GUCY2C-VH comprises the amino acid sequence of SEQ ID NO: 25, and the GUCY2C-VL comprises the amino acid sequence of SEQ ID NO: 26, or
ii) the GUCY2C-VH comprises the amino acid sequence of SEQ ID NO: 37, and the GUCY2C-VL comprises the amino acid sequence of SEQ ID NO: 33, or
the GUCY2C-VH comprises the amino acid sequence of SEQ ID NO: 38, and the GUCY2C-VL comprises the amino acid sequence of SEQ ID NO: 33, 32, 34, or 36, or
the GUCY2C-VH comprises the amino acid sequence of SEQ ID NO: 15, and the GUCY2C-VL comprises the amino acid sequence of SEQ ID NO: 16, or
iii) the GUCY2C-VH comprises the amino acid sequence of SEQ ID NO: 42, and the GUCY2C-VL comprises the amino acid sequence of SEQ ID NO: 41, or
the GUCY2C-VH comprises the amino acid sequence of SEQ ID NO: 17, and the GUCY2C-VL comprises the amino acid sequence of SEQ ID NO: 18, or
more preferably:
i) the GUCY2C-VH comprises the amino acid sequence of SEQ ID NO: 56, and the GUCY2C-VL comprises the amino acid sequence of SEQ ID NO: 54, or
the GUCY2C-VH comprises the amino acid sequence of SEQ ID NO: 56, and the GUCY2C-VL comprises the amino acid sequence of SEQ ID NO: 55; or
ii) the GUCY2C-VH comprises the amino acid sequence of SEQ ID NO: 37, and the GUCY2C-VL comprises the amino acid sequence of SEQ ID NO: 33; or
iii) the GUCY2C-VH comprises the amino acid sequence of SEQ ID NO: 42, and the GUCY2C-VL comprises the amino acid sequence of SEQ ID NO: 41.

4. The antigen-binding molecule according to claim 1, wherein:
the CD3-VH comprises the amino acid sequence of SEQ ID NO: 77 or 89, and the CD3-VL comprises the amino acid sequence of SEQ ID NO: 78:
preferably,
the CD3-VH comprises the amino acid sequence of SEQ ID NO: 77, and the CD3-VL comprises the amino acid sequence of SEQ ID NO: 78.

5. The antigen-binding molecule according to claim 1, wherein the antigen-binding moiety that specifically binds to GUCY2C or the antigen-binding moiety that specifically binds to CD3 comprises a Titin chain and an Obscurin chain capable of forming a dimer;
preferably,
the Titin chain comprises an amino acid sequence selected from the group consisting of SEQ ID NO: 182 to SEQ ID NO: 200, and the Obscurin chain comprises an amino acid sequence selected from the group consisting of SEQ ID NO: 201 to SEQ ID NO: 241;
more preferably,
the Titin chain comprises the amino acid sequence of SEQ ID NO: 198, and the Obscurin chain comprises the amino acid sequence of SEQ ID NO: 236.

6. The antigen-binding molecule according to claim 1, comprising an Fc region, wherein the Fc region is preferably an IgG Fc region, and the Fc region is further preferably an IgG1 Fc region;
more preferably, the Fc region comprises one or more amino acid substitutions capable of reducing the binding of the Fc region to an Fcγ receptor;
most preferably, the Fc region is a human IgG1 Fc region, and the amino acids at positions 234 and 235 are A, as numbered according to the EU index,
and/or wherein the Fc region comprises a first subunit Fc1 and a second subunit Fc2 capable of associating with each other, and the Fc1 and the Fc2 each independently have one or more amino acid substitutions that reduce homodimerization of the Fc region;
preferably, the Fc1 has a knob structure according to the knob-into-hole technique, and the Fc2 has a hole structure according to the knob-into-hole technique;
more preferably, for the Fc1, the amino acid at position 366 is W, and for the Fc2, the amino acid at position 366 is S, the amino acid at position 368 is A, and the amino acid at position 407 is V, as numbered according to the EU index;
most preferably, the Fc1 comprises the amino acid sequence of SEQ ID NO: 79 or 85; the Fc2 comprises the amino acid sequence of SEQ ID NO: 80 or 86.

7. (canceled)

8. The antigen-binding molecule according to claim 6, comprising one antigen-binding moiety that specifically binds to GUCY2C and one antigen-binding moiety that specifically binds to CD3, the antigen-binding moieties that specifically bind to GUCY2C and CD3 being both scFvs, wherein
preferably,
the antigen-binding molecule comprises one first chain having a structure represented by formula (a) and one second chain having a structure represented by formula (b): 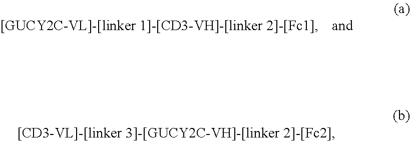
wherein the linker 1, the linker 2, and the linker 3 are identical or different peptide linkers; or the linker 1, the linker 2, and the linker 3 are absent;
the structures represented by formulas (a) and (b) are arranged from N-terminus to C-terminus;
more preferably,
i) the GUCY2C-VH comprises the amino acid sequence of SEQ ID NO: 56, and the GUCY2C-VL comprises the amino acid sequence of SEQ ID NO: 54; and the CD3-VH comprises the amino acid sequence of SEQ ID NO: 77, and the CD3-VL comprises the amino acid sequence of SEQ ID NO: 78; or
the GUCY2C-VH comprises the amino acid sequence of SEQ ID NO: 56, and the GUCY2C-VL comprises the amino acid sequence of SEQ ID NO: 55; and the CD3-VH comprises the amino acid sequence of SEQ ID NO: 77, and the CD3-VL comprises the amino acid sequence of SEQ ID NO: 78; or
ii) the GUCY2C-VH comprises the amino acid sequence of SEQ ID NO: 15, and the GUCY2C-VL comprises the amino acid sequence of SEQ ID NO: 16; and the CD3-VH comprises the amino acid sequence of SEQ ID NO: 77, and the CD3-VL comprises the amino acid sequence of SEQ ID NO: 78; or
iii) the GUCY2C-VH comprises the amino acid sequence of SEQ ID NO: 17, and the GUCY2C-VL comprises the amino acid sequence of SEQ ID NO: 18; and the CD3-VH comprises the amino acid sequence of SEQ ID NO: 77, and the CD3-VL comprises the amino acid sequence of SEQ ID NO: 78;
most preferably,
i) the antigen-binding molecule comprises:
one first chain comprising the amino acid sequence of SEQ ID NO: 99 and one second chain comprising the amino acid sequence of SEQ ID NO: 100; or
one first chain comprising the amino acid sequence of SEQ ID NO: 101 and one second chain comprising the amino acid sequence of SEQ ID NO: 100; or
ii) the antigen-binding molecule comprises:
one first chain comprising the amino acid sequence of SEQ ID NO: 102 and one second chain comprising the amino acid sequence of SEQ ID NO: 103; or
iii) the antigen-binding molecule comprises:
one first chain comprising the amino acid sequence of SEQ ID NO: 104 and one second chain comprising the amino acid sequence of SEQ ID NO: 105.

9. The antigen-binding molecule according to claim 6, comprising one antigen-binding moiety that specifically binds to CD3 and one antigen-binding moiety that specifically binds to GUCY2C, the antigen-binding moiety that specifically binds to CD3 being a Fab, and the antigen-binding moiety that specifically binds to GUCY2C being a replaced Fab comprising a Titin chain and an Obscurin chain capable of forming a dimer, wherein preferably,
the antigen-binding molecule comprises one first chain having a structure represented by formula (c), one second chain having a structure represented by formula (d), one third chain having a structure represented by formula (e), and one fourth chain having a structure represented by formula (f): 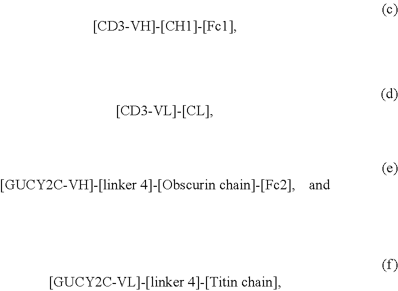
wherein the linker 4 is a peptide linker; or the linker 4 is absent;
the structures represented by formulas (c), (d), (e), and (f) are arranged from N-terminus to C-terminus;
more preferably,
i) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 33; or
the CD3-VH comprises the amino acid sequence of SEQ ID NO: 89, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 33; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 15, and the GUCY2C-VL comprises the amino acid sequence of SEQ ID NO: 16; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 31; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 32; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 34; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 35; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 36; or
ii) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 54; or
iii) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 42, and the GUCY2C-VL comprises the amino acid sequence of SEQ ID NO: 41; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 17, and the GUCY2C-VL comprises the amino acid sequence of SEQ ID NO: 18;
most preferably,
the antigen-binding molecule comprises:
i) one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 113; or
one first chain comprising the amino acid sequence of SEQ ID NO: 242, one second chain comprising the amino acid sequence of SEQ ID NO: 92, one third chain comprising the amino acid sequence of SEQ ID NO: 110, and one fourth chain comprising the amino acid sequence of SEQ ID NO: 113; or
one first chain comprising the amino acid sequence of SEQ ID NO: 91, one second chain comprising the amino acid sequence of SEQ ID NO: 92, one third chain comprising the amino acid sequence of SEQ ID NO: 108, and one fourth chain comprising the amino acid sequence of SEQ ID NO: 109; or
one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 111; or
one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 112; or
one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 114; or
one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 115; or
one first chain comprising the amino acid sequence of SEQ ID NO: 91, one second chain comprising the amino acid sequence of SEQ ID NO: 92, one third chain comprising the amino acid sequence of SEQ ID NO: 110, and one fourth chain comprising the amino acid sequence of SEQ ID NO: 116; or
ii) one first chain comprising the amino acid sequence of SEQ ID NO: 91, one second chain comprising the amino acid sequence of SEQ ID NO: 92, one third chain comprising the amino acid sequence of SEQ ID NO: 106, and one fourth chain comprising the amino acid sequence of SEQ ID NO: 107; or
iii) one first chain comprising the amino acid sequence of SEQ ID NO: 91, one second chain comprising the amino acid sequence of SEQ ID NO: 92, one third chain comprising the amino acid sequence of SEQ ID NO: 119, and one fourth chain comprising the amino acid sequence of SEQ ID NO: 120; or
one first chain comprising the amino acid sequence of SEQ ID NO: 91, one second chain comprising the amino acid sequence of SEQ ID NO: 92, one third chain comprising the amino acid sequence of SEQ ID NO: 117, and one fourth chain comprising the amino acid sequence of SEQ ID NO: 118.

10. The antigen-binding molecule according to claim 6, comprising one antigen-binding moiety that specifically binds to GUCY2C and one antigen-binding moiety that specifically binds to CD3, the antigen-binding moiety that specifically binds to GUCY2C being a replaced Fab comprising a Titin chain and an Obscurin chain capable of forming a dimer, and the antigen-binding moiety that specifically binds to CD3 being a Fab, wherein preferably, the antigen-binding molecule comprises one first chain having a structure represented by formula (g), one second chain having a structure represented by formula (f), one third chain having a structure represented by formula (h), and one fourth chain having a structure represented by formula (d): 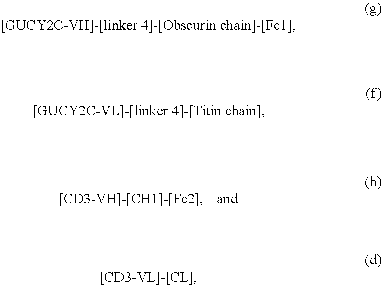
wherein the linker 4 is a peptide linker; or the linker 4 is absent;
the structures represented by formulas (g), (f), (h), and (d) are arranged from N-terminus to C-terminus;
more preferably,
i) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 54; or
ii) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 31; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 32; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 33; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 34; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 35; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 37, and the GUCY2C-VL comprises the amino acid sequence of SEQ ID NO: 36; or
iii) the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 42, and the GUCY2C-VL comprises the amino acid sequence of SEQ ID NO: 41;
most preferably,
the antigen-binding molecule comprises:
i) one first chain comprising the amino acid sequence of SEQ ID NO: 121, one second chain comprising the amino acid sequence of SEQ ID NO: 107, one third chain comprising the amino acid sequence of SEQ ID NO: 93, and one fourth chain comprising the amino acid sequence of SEQ ID NO: 92; or
ii) one first chain comprising the amino acid sequence of SEQ ID NO: 122, one second chain comprising the amino acid sequence of SEQ ID NO: 111, one third chain comprising the amino acid sequence of SEQ ID NO: 93, and one fourth chain comprising the amino acid sequence of SEQ ID NO: 92; or
one first chain comprising the amino acid sequence of SEQ ID NO: 122, one second chain comprising the amino acid sequence of SEQ ID NO: 112, one third chain comprising the amino acid sequence of SEQ ID NO: 93, and one fourth chain comprising the amino acid sequence of SEQ ID NO: 92; or
one first chain comprising the amino acid sequence of SEQ ID NO: 122, one second chain comprising the amino acid sequence of SEQ ID NO: 113, one third chain comprising the amino acid sequence of SEQ ID NO: 93, and one fourth chain comprising the amino acid sequence of SEQ ID NO: 92; or
one first chain comprising the amino acid sequence of SEQ ID NO: 122, one second chain comprising the amino acid sequence of SEQ ID NO: 114, one third chain comprising the amino acid sequence of SEQ ID NO: 93, and one fourth chain comprising the amino acid sequence of SEQ ID NO: 92; or
one first chain comprising the amino acid sequence of SEQ ID NO: 122, one second chain comprising the amino acid sequence of SEQ ID NO: 115, one third chain comprising the amino acid sequence of SEQ ID NO: 93, and one fourth chain comprising the amino acid sequence of SEQ ID NO: 92; or
one first chain comprising the amino acid sequence of SEQ ID NO: 122, one second chain comprising the amino acid sequence of SEQ ID NO: 116, one third chain comprising the amino acid sequence of SEQ ID NO: 93, and one fourth chain comprising the amino acid sequence of SEQ ID NO: 92; or
iii) one first chain comprising the amino acid sequence of SEQ ID NO: 123, one second chain comprising the amino acid sequence of SEQ ID NO: 120, one third chain comprising the amino acid sequence of SEQ ID NO: 93, and one fourth chain comprising the amino acid sequence of SEQ ID NO: 92.

11. The antigen-binding molecule according to claim 6, comprising two antigen-binding moieties that specifically bind to GUCY2C and two antigen-binding moieties that specifically bind to CD3, the antigen-binding moieties that specifically bind to GUCY2C and CD3 being all scFvs, wherein
preferably,
the antigen-binding molecule comprises two first chains having a structure represented by formula (i) and two second chains having a structure represented by formula (j): 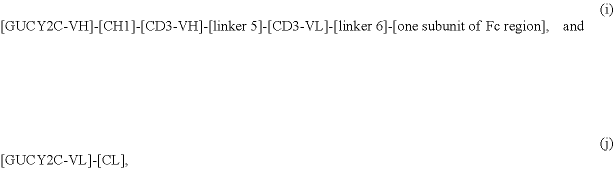
wherein the linker 5 and the linker 6 are peptide linkers; or the linker 5 and the linker 6 are absent;
the structures represented by formulas (i) and (j) are arranged from N-terminus to C-terminus;
more preferably,
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 54; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 55;
most preferably,
the antigen-binding molecule comprises:
two first chains comprising the amino acid sequence of SEQ ID NO: 124 and two second chains comprising the amino acid sequence of SEQ ID NO: 65; or
two first chains comprising the amino acid sequence of SEQ ID NO: 124 and two second chains comprising the amino acid sequence of SEQ ID NO: 66.

12. The antigen-binding molecule according to claim 6, comprising two antigen-binding moieties that specifically bind to GUCY2C and two antigen-binding moieties that specifically bind to CD3, the antigen-binding moieties that specifically bind to GUCY2C being Fabs, and the antigen-binding moieties that specifically bind to CD3 being replaced Fabs comprising a Titin chain and an Obscurin chain capable of forming a dimer; wherein
preferably,
the antigen-binding molecule comprises two first chains having a structure represented by formula (k), two second chains having a structure represented by formula (j), and two third chains having a structure represented by formula (I): 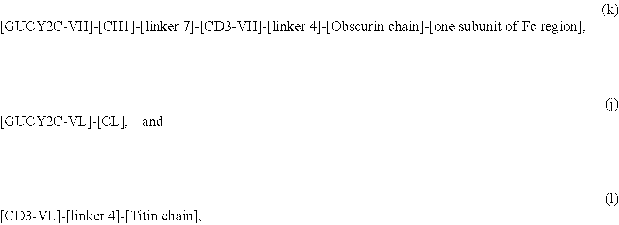
wherein the linker 4 and the linker 7 are peptide linkers; or the linker 4 and the linker 7 are absent;
the structures represented by formulas (k), (j), and (l) are arranged from N-terminus to C-terminus;
more preferably,
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 54; or
the CD3-VH comprises the amino acid sequence of SEQ ID NO: 77, and the CD3-VL comprises the amino acid sequence of SEQ ID NO: 78; and the GUCY2C-VH comprises the amino acid sequence of SEQ ID NO: 56, and the GUCY2C-VL comprises the amino acid sequence of SEQ ID NO: 55;
most preferably,
the antigen-binding molecule comprises:
two first chains comprising the amino acid sequence of SEQ ID NO: 125, two second chains comprising the amino acid sequence of SEQ ID NO: 65, and two third chains comprising the amino acid sequence of SEQ ID NO: 98; or
two first chains comprising the amino acid sequence of SEQ ID NO: 125, two second chains comprising the amino acid sequence of SEQ ID NO: 66, and two third chains comprising the amino acid sequence of SEQ ID NO: 98.

13. An anti-GUCY2C antibody capable of specifically binding to GUCY2C, comprising a heavy chain variable region GUCY2C-VH and a light chain variable region GUCY2C-VL, wherein:
(i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 47, 22, 43, 44, 45, or 46, a GUCY2C-LCDR2 comprising the amino acid sequence of SEQ ID NO: 23, and a GUCY2C-LCDR3 comprising the amino acid sequence of SEQ ID NO: 24; or
(ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27 or 6; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, 28, 29, or 30, and a GUCY2C-LCDR3 comprising the amino acid sequence of SEQ ID NO: 9; or
iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40 or 12; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39 or 8, and a GUCY2C-LCDR3 comprising the amino acid sequence of SEQ ID NO: 14; or
iv-1) the GUCY2C-VH comprises: a GUCY2C-HCDR1 comprising the amino acid sequence of SEQ ID NO: 250, a GUCY2C-HCDR2 comprising the amino acid sequence of SEQ ID NO: 251, and a GUCY2C-HCDR3 comprising the amino acid sequence of SEQ ID NO: 252; and the GUCY2C-VL comprises: a GUCY2C-LCDR1 comprising the amino acid sequence of SEQ ID NO: 253, a GUCY2C-LCDR2 comprising the amino acid sequence of SEQ ID NO: 254, and a GUCY2C-LCDR3 comprising the amino acid sequence of SEQ ID NO: 255; or
iv-2) the GUCY2C-VH comprises: a GUCY2C-HCDR1 comprising the amino acid sequence of SEQ ID NO: 256, a GUCY2C-HCDR2 comprising the amino acid sequence of SEQ ID NO: 257, and a GUCY2C-HCDR3 comprising the amino acid sequence of SEQ ID NO: 258; and the GUCY2C-VL comprises: a GUCY2C-LCDR1 comprising the amino acid sequence of SEQ ID NO: 259, a GUCY2C-LCDR2 comprising the amino acid sequence of SEQ ID NO: 8, and a GUCY2C-LCDR3 comprising the amino acid sequence of SEQ ID NO: 260; or
iv-3) the GUCY2C-VH comprises: a GUCY2C-HCDR1 comprising the amino acid sequence of SEQ ID NO: 261, a GUCY2C-HCDR2 comprising the amino acid sequence of SEQ ID NO: 262, and a GUCY2C-HCDR3 comprising the amino acid sequence of SEQ ID NO: 263; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 265, and a GUCY2C-LCDR3 comprising the amino acid sequence of SEQ ID NO: 266; or
iv-4) the GUCY2C-VH comprises: a GUCY2C-HCDR1 comprising the amino acid sequence of SEQ ID NO: 267, a GUCY2C-HCDR2 comprising the amino acid sequence of SEQ ID NO: 268, and a GUCY2C-HCDR3 comprising the amino acid sequence of SEQ ID NO: 269; and the GUCY2C-VL comprises: a GUCY2C-LCDR1 comprising the amino acid sequence of SEQ ID NO: 270, a GUCY2C-LCDR2 comprising the amino acid sequence of SEQ ID NO: 271, and a GUCY2C-LCDR3 comprising the amino acid sequence of SEQ ID NO: 272; or
iv-5) the GUCY2C-VH comprises: a GUCY2C-HCDR1 comprising the amino acid sequence of SEQ ID NO: 281, a GUCY2C-HCDR2 comprising the amino acid sequence of SEQ ID NO: 282, and a GUCY2C-HCDR3 comprising the amino acid sequence of SEQ ID NO: 283; and the GUCY2C-VL comprises: a GUCY2C-LCDR1 comprising the amino acid sequence of SEQ ID NO: 284, a GUCY2C-LCDR2 comprising the amino acid sequence of SEQ ID NO: 8, and a GUCY2C-LCDR3 comprising the amino acid sequence of SEQ ID NO: 285; or
iv-6) the GUCY2C-VH comprises: a GUCY2C-HCDR1 comprising the amino acid sequence of SEQ ID NO: 286, a GUCY2C-HCDR2 comprising the amino acid sequence of SEQ ID NO: 287, and a GUCY2C-HCDR3 comprising the amino acid sequence of SEQ ID NO: 288; and the GUCY2C-VL comprises: a GUCY2C-LCDR1 comprising the amino acid sequence of SEQ ID NO: 289, a GUCY2C-LCDR2 comprising the amino acid sequence of SEQ ID NO: 265, and a GUCY2C-LCDR3 comprising the amino acid sequence of SEQ ID NO: 290; or
iv-7) the GUCY2C-VH comprises: a GUCY2C-HCDR1 comprising the amino acid sequence of SEQ ID NO: 291, a GUCY2C-HCDR2 comprising the amino acid sequence of SEQ ID NO: 292, and a GUCY2C-HCDR3 comprising the amino acid sequence of SEQ ID NO: 293; and the GUCY2C-VL comprises: a GUCY2C-LCDR1 comprising the amino acid sequence of SEQ ID NO: 294, a GUCY2C-LCDR2 comprising the amino acid sequence of SEQ ID NO: 295, and a GUCY2C-LCDR3 comprising the amino acid sequence of SEQ ID NO: 296; or
iv-8) the GUCY2C-VH comprises: a GUCY2C-HCDR1 comprising the amino acid sequence of SEQ ID NO: 261, a GUCY2C-HCDR2 comprising the amino acid sequence of SEQ ID NO: 297, and a GUCY2C-HCDR3 comprising the amino acid sequence of SEQ ID NO: 298; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 299, and a GUCY2C-LCDR3 comprising the amino acid sequence of SEQ ID NO: 300; or
iv-9) the GUCY2C-VH comprises: a GUCY2C-HCDR1 comprising the amino acid sequence of SEQ ID NO: 261, a GUCY2C-HCDR2 comprising the amino acid sequence of SEQ ID NO: 301, and a GUCY2C-HCDR3 comprising the amino acid sequence of SEQ ID NO: 302; and the GUCY2C-VL comprises: a GUCY2C-LCDR1 comprising the amino acid sequence of SEQ ID NO: 303, a GUCY2C-LCDR2 comprising the amino acid sequence of SEQ ID NO: 299, and a GUCY2C-LCDR3 comprising the amino acid sequence of SEQ ID NO: 304; or
iv-10) the GUCY2C-VH comprises: a GUCY2C-HCDR1 comprising the amino acid sequence of SEQ ID NO: 305, a GUCY2C-HCDR2 comprising the amino acid sequence of SEQ ID NO: 306, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 24; or
iv-11) the GUCY2C-VH comprises: a GUCY2C-HCDR1 comprising the amino acid sequence of SEQ ID NO: 128, a GUCY2C-HCDR2 comprising the amino acid sequence of SEQ ID NO: 307, and a GUCY2C-HCDR3 comprising the amino acid sequence of SEQ ID NO: 308; and the GUCY2C-VL comprises: a GUCY2C-LCDR1 comprising the amino acid sequence of SEQ ID NO: 309, a GUCY2C-LCDR2 comprising the amino acid sequence of SEQ ID NO: 310, and a GUCY2C-LCDR3 comprising the amino acid sequence of SEQ ID NO: 311; or
iv-12) the GUCY2C-VH comprises: a GUCY2C-HCDR1 comprising the amino acid sequence of SEQ ID NO: 312, a GUCY2C-HCDR2 comprising the amino acid sequence of SEQ ID NO: 313, and a GUCY2C-HCDR3 comprising the amino acid sequence of SEQ ID NO: 314; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 315; or
iv-13) the GUCY2C-VH comprises: a GUCY2C-HCDR1 comprising the amino acid sequence of SEQ ID NO: 286, a GUCY2C-HCDR2 comprising the amino acid sequence of SEQ ID NO: 287, and a GUCY2C-HCDR3 comprising the amino acid sequence of SEQ ID NO: 316; and the GUCY2C-VL comprises: a GUCY2C-LCDR1 comprising the amino acid sequence of SEQ ID NO: 317, a GUCY2C-LCDR2 comprising the amino acid sequence of SEQ ID NO: 265, and a GUCY2C-LCDR3 comprising the amino acid sequence of SEQ ID NO: 300; or
iv-14) the GUCY2C-VH comprises: a GUCY2C-HCDR1 comprising the amino acid sequence of SEQ ID NO: 318, a GUCY2C-HCDR2 comprising the amino acid sequence of SEQ ID NO: 319, and a GUCY2C-HCDR3 comprising the amino acid sequence of SEQ ID NO: 320; and the GUCY2C-VL comprises: a GUCY2C-LCDR1 comprising the amino acid sequence of SEQ ID NO: 321, a GUCY2C-LCDR2 comprising the amino acid sequence of SEQ ID NO: 322, and a GUCY2C-LCDR3 comprising the amino acid sequence of SEQ ID NO: 323; or
iv-15) the GUCY2C-VH comprises: a GUCY2C-HCDR1 comprising the amino acid sequence of SEQ ID NO: 324, a GUCY2C-HCDR2 comprising the amino acid sequence of SEQ ID NO: 325, and a GUCY2C-HCDR3 comprising the amino acid sequence of SEQ ID NO: 326; and the GUCY2C-VL comprises: a GUCY2C-LCDR1 comprising the amino acid sequence of SEQ ID NO: 22, a GUCY2C-LCDR2 comprising the amino acid sequence of SEQ ID NO: 23, and a GUCY2C-LCDR3 comprising the amino acid sequence of SEQ ID NO: 327; or
iv-16) the GUCY2C-VH comprises: a GUCY2C-HCDR1 comprising the amino acid sequence of SEQ ID NO: 261, a GUCY2C-HCDR2 comprising the amino acid sequence of SEQ ID NO: 328, and a GUCY2C-HCDR3 comprising the amino acid sequence of SEQ ID NO: 302; and the GUCY2C-VL comprises: a GUCY2C-LCDR1 comprising the amino acid sequence of SEQ ID NO: 264, a GUCY2C-LCDR2 comprising the amino acid sequence of SEQ ID NO: 329, and a GUCY2C-LCDR3 comprising the amino acid sequence of SEQ ID NO: 300;
preferably,
i) the GUCY2C-VH comprises: a GUCY2C-HCDR1 comprising the amino acid sequence of SEQ ID NO: 19, a GUCY2C-HCDR2 comprising the amino acid sequence of SEQ ID NO: 20, and a GUCY2C-HCDR3 comprising the amino acid sequence of SEQ ID NO: 21; and the GUCY2C-VL comprises: a GUCY2C-LCDR1 comprising the amino acid sequence of SEQ ID NO: 47, a GUCY2C-LCDR2 comprising the amino acid sequence of SEQ ID NO: 23, and a GUCY2C-LCDR3 comprising the amino acid sequence of SEQ ID NO: 24; or
ii) the GUCY2C-VH comprises: a GUCY2C-HCDR1 comprising the amino acid sequence of SEQ ID NO: 4, a GUCY2C-HCDR2 comprising the amino acid sequence of SEQ ID NO: 5, and a GUCY2C-HCDR3 comprising the amino acid sequence of SEQ ID NO: 27; and the GUCY2C-VL comprises: a GUCY2C-LCDR1 comprising the amino acid sequence of SEQ ID NO: 7, a GUCY2C-LCDR2 comprising the amino acid sequence of SEQ ID NO: 8, and a GUCY2C-LCDR3 comprising the amino acid sequence of SEQ ID NO: 9; or
iii) the GUCY2C-VH comprises: a GUCY2C-HCDR1 comprising the amino acid sequence of SEQ ID NO: 10, a GUCY2C-HCDR2 comprising the amino acid sequence of SEQ ID NO: 11, and a GUCY2C-HCDR3 comprising the amino acid sequence of SEQ ID NO: 40; and the GUCY2C-VL comprises: a GUCY2C-LCDR1 comprising the amino acid sequence of SEQ ID NO: 13, a GUCY2C-LCDR2 comprising the amino acid sequence of SEQ ID NO: 39, and a GUCY2C-LCDR3 comprising the amino acid sequence of SEQ ID NO: 14.

14. The anti-GUCY2C antibody according to claim 13, wherein:
i) the GUCY2C-VH comprises the amino acid sequence of SEQ ID NO: 56, 25, or 57, and the GUCY2C-VL comprises the amino acid sequence of SEQ ID NO: 54, 55, 26, 48, 49, 50, 51, 52, or 53; or
ii) the GUCY2C-VH comprises the amino acid sequence of SEQ ID NO: 37, 15, or 38, and the GUCY2C-VL comprises the amino acid sequence of SEQ ID NO: 33, 16, 31, 32, 34, 35, or 36; or
iii) the GUCY2C-VH comprises the amino acid sequence of SEQ ID NO: 42 or 17, and the GUCY2C-VL comprises the amino acid sequence of SEQ ID NO: 41 or 18; or
iv-1) the GUCY2C-VH comprises the amino acid sequence of SEQ ID NO: 273, and the GUCY2C-VL comprises the amino acid sequence of SEQ ID NO: 274; or
iv-2) the GUCY2C-VH comprises the amino acid sequence of SEQ ID NO: 275, and the GUCY2C-VL comprises the amino acid sequence of SEQ ID NO: 276; or
iv-3) the GUCY2C-VH comprises the amino acid sequence of SEQ ID NO: 277, and the GUCY2C-VL comprises the amino acid sequence of SEQ ID NO: 278; or
iv-4) the GUCY2C-VH comprises the amino acid sequence of SEQ ID NO: 279, and the GUCY2C-VL comprises the amino acid sequence of SEQ ID NO: 280; or
iv-5) the GUCY2C-VH comprises the amino acid sequence of SEQ ID NO: 330, and the GUCY2C-VL comprises the amino acid sequence of SEQ ID NO: 331; or
iv-6) the GUCY2C-VH comprises the amino acid sequence of SEQ ID NO: 332, and the GUCY2C-VL comprises the amino acid sequence of SEQ ID NO: 333; or
iv-7) the GUCY2C-VH comprises the amino acid sequence of SEQ ID NO: 334, and the GUCY2C-VL comprises the amino acid sequence of SEQ ID NO: 335; or
iv-8) the GUCY2C-VH comprises the amino acid sequence of SEQ ID NO: 336, and the GUCY2C-VL comprises the amino acid sequence of SEQ ID NO: 337; or
iv-9) the GUCY2C-VH comprises the amino acid sequence of SEQ ID NO: 338, and the GUCY2C-VL comprises the amino acid sequence of SEQ ID NO: 339; or
iv-10) the GUCY2C-VH comprises the amino acid sequence of SEQ ID NO: 340, and the GUCY2C-VL comprises the amino acid sequence of SEQ ID NO: 341; or
iv-11) the GUCY2C-VH comprises the amino acid sequence of SEQ ID NO: 342, and the GUCY2C-VL comprises the amino acid sequence of SEQ ID NO: 343; or
iv-12) the GUCY2C-VH comprises the amino acid sequence of SEQ ID NO: 344, and the GUCY2C-VL comprises the amino acid sequence of SEQ ID NO: 345; or
iv-13) the GUCY2C-VH comprises the amino acid sequence of SEQ ID NO: 346, and the GUCY2C-VL comprises the amino acid sequence of SEQ ID NO: 347; or
iv-14) the GUCY2C-VH comprises the amino acid sequence of SEQ ID NO: 348, and the GUCY2C-VL comprises the amino acid sequence of SEQ ID NO: 349; or
iv-15) the GUCY2C-VH comprises the amino acid sequence of SEQ ID NO: 350, and the GUCY2C-VL comprises the amino acid sequence of SEQ ID NO: 351; or
iv-16) the GUCY2C-VH comprises the amino acid sequence of SEQ ID NO: 352, and the GUCY2C-VL comprises the amino acid sequence of SEQ ID NO: 353;
preferably,
i) the GUCY2C-VH comprises the amino acid sequence of SEQ ID NO: 56, and the GUCY2C-VL comprises the amino acid sequence of SEQ ID NO: 54, 55, 48, 49, 50, 51, or 52, or
the GUCY2C-VH comprises the amino acid sequence of SEQ ID NO: 25, and the GUCY2C-VL comprises the amino acid sequence of SEQ ID NO: 26, or
ii) the GUCY2C-VH comprises the amino acid sequence of SEQ ID NO: 37, and the GUCY2C-VL comprises the amino acid sequence of SEQ ID NO: 33, or
the GUCY2C-VH comprises the amino acid sequence of SEQ ID NO: 38, and the GUCY2C-VL comprises the amino acid sequence of SEQ ID NO: 33, 32, 34, or 36, or
the GUCY2C-VH comprises the amino acid sequence of SEQ ID NO: 15, and the GUCY2C-VL comprises the amino acid sequence of SEQ ID NO: 16, or
iii) the GUCY2C-VH comprises the amino acid sequence of SEQ ID NO: 42, and the GUCY2C-VL comprises the amino acid sequence of SEQ ID NO: 41, or
the GUCY2C-VH comprises the amino acid sequence of SEQ ID NO: 17, and the GUCY2C-VL comprises the amino acid sequence of SEQ ID NO: 18;
more preferably:
i) the GUCY2C-VH comprises the amino acid sequence of SEQ ID NO: 56, and the GUCY2C-VL comprises the amino acid sequence of SEQ ID NO: 54, or
the GUCY2C-VH comprises the amino acid sequence of SEQ ID NO: 56, and the GUCY2C-VL comprises the amino acid sequence of SEQ ID NO: 55; or
ii) the GUCY2C-VH comprises the amino acid sequence of SEQ ID NO: 37, and the GUCY2C-VL comprises the amino acid sequence of SEQ ID NO: 33; or
iii) the GUCY2C-VH comprises the amino acid sequence of SEQ ID NO: 42, and the GUCY2C-VL comprises the amino acid sequence of SEQ ID NO: 41.

15. The anti-GUCY2C antibody according to claim 13, wherein the anti-GUCY2C antibody is a murine antibody, a chimeric antibody, or a humanized antibody; or wherein the anti-GUCY2C antibody is an antibody fragment; preferably, the antibody fragment is Fab, Fab′, F(ab′)2, Fd, Fv, scFv, dsFv, or dAb.

16. (canceled)

17. The anti-GUCY2C antibody according to claim 13, comprising a heavy chain and a light chain, wherein:
the anti-GUCY2C antibody comprising a heavy chain constant region and a light chain constant region, wherein preferably, the heavy chain constant region is a human IgG1 constant region, and the light chain constant region is selected from the group consisting of constant regions of human antibody κ and λ chains: more preferably, the heavy chain constant region comprises the amino acid sequence of SEQ ID NO: 58, and the light chain constant region comprises the amino acid sequence of SEQ ID NO: 59;
most preferably,
the heavy chain comprises an amino acid sequence having at least 85% sequence identity to SEQ ID NO: 64, and the light chain comprises an amino acid sequence having at least 85% sequence identity to SEQ ID NO: 65 or 66; or
the heavy chain comprises an amino acid sequence having at least 85% sequence identity to SEQ ID NO: 60, and the light chain comprises an amino acid sequence having at least 85% sequence identity to SEQ ID NO: 61; or
the heavy chain comprises an amino acid sequence having at least 85% sequence identity to SEQ ID NO: 62, and the light chain comprises an amino acid sequence having at least 85% sequence identity to SEQ ID NO: 63;
preferably, the heavy chain comprises the amino acid sequence of SEQ ID NO: 64, and the light chain comprises the amino acid sequence of SEQ ID NO: 65 or 66; or
the heavy chain comprises the amino acid sequence of SEQ ID NO: 60, and the light chain comprises the amino acid sequence of SEQ ID NO: 61; or
the heavy chain comprises the amino acid sequence of SEQ ID NO: 62, and the light chain comprises the amino acid sequence of SEQ ID NO: 63.

18. (canceled)

19. A pharmaceutical composition, comprising:
a therapeutically effective amount of the antigen-binding molecule according to claim 1 and one or more pharmaceutically acceptable carriers, diluents, buffers, or excipients.

20. An isolated nucleic acid encoding the antigen-binding molecule according to claim 1.

21. (canceled)

22. A method for treating a disease, comprising administering to a subject a therapeutically effective amount of the antigen-binding molecule according to claim 1; wherein preferably, the disease is a tumor; more preferably, the tumor is gastric cancer, gastrointestinal cancer, intestinal cancer, gastric adenocarcinoma, small intestine cancer, colorectal cancer, esophageal cancer, anal cancer, liver cancer, gallbladder cancer, bile duct cancer, pancreatic cancer, prostate cancer, renal cancer, urothelial cancer, breast cancer, bladder cancer, thymus cancer, melanoma, glioma, sarcoma, osteosarcoma, ovarian cancer, thyroid cancer, lung squamous cell cancer, lung adenocarcinoma, cervical squamous cell cancer, endometrial cancer, lung cancer, skin cancer, head and neck cancer, brain cancer, glioblastoma multiforme, gastroesophageal cancer, gastroesophageal adenocarcinoma, adenocarcinomas metastatic to the liver, multiple myeloma, lymphoma, leukemia, or B-cell lymphoma; most preferably, cells of the tumor or vascular endothelial cells close to the tumor express GUCY2C.

23. The method for treating a disease according to claim 22, further comprising using a second therapeutic agent, wherein the second therapeutic agent comprises an anti-tumor agent, radiation therapy, an antibody-drug conjugate, a bispecific antibody, a bispecific antibody conjugated to an anti-tumor agent, an immune checkpoint inhibitor, or a combination thereof; wherein, preferably,
the second therapeutic agent is a bispecific antibody that specifically binds to CD28 and EGFR, and the bispecific antibody comprises at least one antigen-binding moiety that specifically binds to CD28 and at least one antigen-binding moiety that specifically binds to EGFR, wherein the antigen-binding moiety that specifically binds to CD28 comprises a heavy chain variable region CD28-VH and a light chain variable region CD28-VL, and the antigen-binding moiety that specifically binds to EGFR comprises a heavy chain variable region EGFR-VH and a light chain variable region EGFR-VL:
more preferably,
a-i) the CD28-VH comprises: a CD28-HCDR1 comprising the amino acid sequence of SEQ ID NO: 146, a CD28-HCDR2 comprising the amino acid sequence of SEQ ID NO: 147, and a CD28-HCDR3 comprising the amino acid sequence of SEQ ID NO: 148; and the CD28-VL comprises:
a CD28-LCDR1 comprising the amino acid sequence of SEQ ID NO: 149, a CD28-LCDR2 comprising the amino acid sequence of SEQ ID NO: 150, and a CD28-LCDR3 comprising the amino acid sequence of SEQ ID NO: 151; or
a-ii) the CD28-VH comprises: a CD28-HCDR1 comprising the amino acid sequence of SEQ ID NO: 160, a CD28-HCDR2 comprising the amino acid sequence of SEQ ID NO: 161, and a CD28-HCDR3 comprising the amino acid sequence of SEQ ID NO: 162; and the CD28-VL comprises:
a CD28-LCDR1 comprising the amino acid sequence of SEQ ID NO: 149, a CD28-LCDR2 comprising the amino acid sequence of SEQ ID NO: 150, and a CD28-LCDR3 comprising the amino acid sequence of SEQ ID NO: 163; or
a-iii) the CD28-VH comprises: a CD28-HCDR1 comprising the amino acid sequence of SEQ ID NO: 168, a CD28-HCDR2 comprising the amino acid sequence of SEQ ID NO: 169, and a CD28-HCDR3 comprising the amino acid sequence of SEQ ID NO: 170; and the CD28-VL comprises:
a CD28-LCDR1 comprising the amino acid sequence of SEQ ID NO: 171, a CD28-LCDR2 comprising the amino acid sequence of SEQ ID NO: 172, and a CD28-LCDR3 comprising the amino acid sequence of SEQ ID NO: 173;
and
b) the EGFR-VH comprises: an EGFR-HCDR1 comprising the amino acid sequence of SEQ ID NO: 140, an EGFR-HCDR2 comprising the amino acid sequence of SEQ ID NO: 141, and an EGFR-HCDR3 comprising the amino acid sequence of SEQ ID NO: 142; and the EGFR-VL comprises: an EGFR-LCDR1 comprising the amino acid sequence of SEQ ID NO: 143, an EGFR-LCDR2 comprising the amino acid sequence of SEQ ID NO: 144, and an EGFR-LCDR3 comprising the amino acid sequence of SEQ ID NO: 145;
most preferably,
a-i) the CD28-VH comprises the amino acid sequence of SEQ ID NO: 154, and the CD28-VL comprises the amino acid sequence of SEQ ID NO: 155; or
a-ii) the CD28-VH comprises the amino acid sequence of SEQ ID NO: 164, and the CD28-VL comprises the amino acid sequence of SEQ ID NO: 165; or
a-iii) the CD28-VH comprises the amino acid sequence of SEQ ID NO: 174, and the CD28-VL comprises the amino acid sequence of SEQ ID NO: 175;
and
b) the EGFR-VH comprises the amino acid sequence of SEQ ID NO: 152, and the EGFR-VL comprises the amino acid sequence of SEQ ID NO: 153.

24.-26. (canceled)

27. The method for treating a disease according to claim 23, wherein the bispecific antibody that specifically binds to CD28 and EGFR comprises one antigen-binding moiety that specifically binds to CD28 and one antigen-binding moiety that specifically binds to EGFR, the antigen-binding moiety that specifically binds to CD28 being a replaced Fab comprising a Titin chain and an Obscurin chain capable of forming a dimer, and the antigen-binding moiety that specifically binds to EGFR being a Fab, wherein
preferably,
the bispecific antibody that specifically binds to CD28 and EGFR comprises one first chain having a structure represented by formula (m), a second chain having a structure represented by formula (n), a third chain having a structure represented by formula (o), and a fourth chain having a structure represented by formula (p): 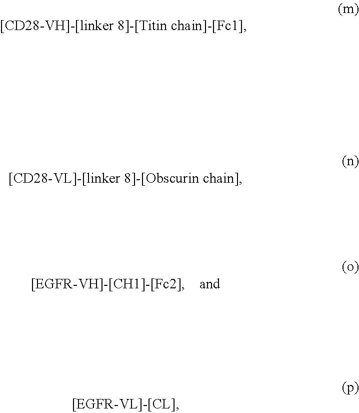
wherein the linker 8 is a peptide linker; or the linker 8 is absent;
the structures represented by formulas (m), (n), (o), and (p) are arranged from N-terminus to C-terminus;
most preferably,
the bispecific antibody that specifically binds to CD28 and EGFR comprises: one first chain comprising the amino acid sequence of SEQ ID NO: 156, one second chain comprising the amino acid sequence of SEQ ID NO: 157, one third chain comprising the amino acid sequence of SEQ ID NO: 158, and one fourth chain comprising the amino acid sequence of SEQ ID NO: 159; or
the bispecific antibody that specifically binds to CD28 and EGFR comprises: one first chain comprising the amino acid sequence of SEQ ID NO: 166, one second chain comprising the amino acid sequence of SEQ ID NO: 167, one third chain comprising the amino acid sequence of SEQ ID NO: 158, and one fourth chain comprising the amino acid sequence of SEQ ID NO: 159;
or
wherein the bispecific antibody that specifically binds to CD28 and EGFR comprises one antigen-binding moiety that specifically binds to CD28 and one antigen-binding moiety that specifically binds to EGFR, the antigen-binding moiety that specifically binds to CD28 being a Fab, and the antigen-binding moiety that specifically binds to EGFR being a replaced Fab comprising a Titin chain and an Obscurin chain capable of forming a dimer, wherein
preferably,
the bispecific antibody that specifically binds to CD28 and EGFR comprises one first chain having a structure represented by formula (q), a second chain having a structure represented by formula (r), a third chain having a structure represented by formula (s), and a fourth chain having a structure represented by formula (t): 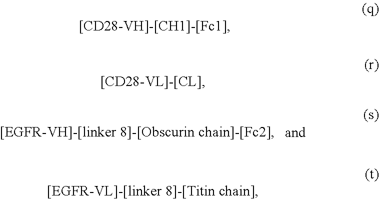
wherein the linker 8 is a peptide linker; or the linker 8 is absent:
the structures represented by formulas (q), (r), (s), and (t) are arranged from N-terminus to C-terminus;
more preferably, the bispecific antibody that specifically binds to CD28 and EGFR comprises: one first chain comprising the amino acid sequence of SEQ ID NO: 176, one second chain comprising the amino acid sequence of SEQ ID NO: 177, one third chain comprising the amino acid sequence of SEQ ID NO: 178, and one fourth chain comprising the amino acid sequence of SEQ ID NO: 179.

28. (canceled)
